SEQUENCE LISTING (4 sequences)

SEQ ID NO: 1 - protein, 188 aa, Artificial
  Synthetic Protein
MASSHHHHHHSSENLYFQGMTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 2 - protein, 188 aa, Artificial
  Synthetic Protein
MASSHHHHHHSSENLYFQGMTEYKLVVVGADGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 3 - protein, 210 aa, Artificial
  Synthetic Protein
MGSSHHHHHHSSGLVPRGSHMASMTGGQQMGRGSENLYFQGMTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 4 - protein, 487 aa, Artificial
  Synthetic Protein
GEEQMRLPSADVYRFAEPDSEENIIFEENMQPKAGIPIIKAGTVIKLIERLTYHMYADPNFVRTFLTTYRSFCKPQELLSLIIERFEIPEPEPTEADRIAIENGDQPLSAELKRFRKEYIQPVQLRVLNVCRHWVEHHFYDFERDAYLLQRMEEFIGTVRGKAMKKWVESITKIIQRKKIARDNGPGHNITFQSSPPTVEWHISRPGHIETFDLLTLHPIEIARQLTLLESDLYRAVQPSELVGSVWTKEDKEINSPNLLKMIRHTTNLTLWFEKCIVETENLEERVAVVSRIIEILQVFQELNNFNGVLEVVSAMNSSPVYRLDHTFEQIPSRQKKILEEAHELSEDHYKKYLAKLRSINPPCVPFFGIYLTNILKTEEGNPEVLKRHGKELINFSKRRKVAEITGEIQQYQNQPYCLRVESDIKRFFENLNPMGNSMEKEFTDYLFNKSLEIEPRNPKPLPRFPKKYSYPLKSPGVRPSNPRPGT

(19) United States
(12) Patent Application Publication
(10) Pub. No.: US 2024/0317759 A1
(43) Pub. Date: Sep. 26, 2024

(54) SMALL MOLECULE INHIBITORS OF KRAS MUTATED PROTEINS

(71) Applicants: TAIHO PHARMACEUTICAL CO., LTD., Tokyo (JP); ASTEX THERAPEUTICS LIMITED, Milton Road (GB)

(72) Inventors: YU KOBAYAKAWA, Ibaraki (JP); TSUYOSHI OSHIMA, Ibaraki (JP); SATORU ITO, Ibaraki (JP); PATRICK SCHÖPF, London (GB)

(21) Appl. No.: 18/564,866

(22) PCT Filed: May 27, 2022
(86) PCT No.: PCT/JP2022/023210

§ 371 Date: Nov. 28, 2023

Related U.S. Application Data
(60) Provisional application No. 63/194,852, filed on May 28, 2021.

(51) Int. Cl. C07D 487/04; A61K 31/519; A61K 31/541; A61K 31/551; A61K 31/553; A61K 31/554; A61P 35/00; C07D 519/00

(52) U.S. Cl. CPC C07D 487/04; A61K 31/519; A61K 31/541; A61K 31/551; A61K 31/553; A61K 31/554; A61P 35/00; C07D 519/00

(57) ABSTRACT

Compounds of Formula (I) or their pharmaceutically acceptable salts can inhibit the G12C, G12D and/or G12V mutants of Kirsten rat sarcoma (KRAS) protein and are expected to have utility as therapeutic agents, for example, for treating cancer. The disclosure also provides pharmaceutical compositions which comprise compounds of Formula (I) or pharmaceutically acceptable salts thereof. The disclosure also relates to methods for use of the compounds or their pharmaceutically acceptable salts in the therapy and prophylaxis of cancer and for preparing pharmaceuticals for this purpose.

### CROSS REFERENCE TO RELATED APPLICATION

The present application claims the benefit of U.S. Provisional Application No. 63/194,852, filed May 28, 2021, the entirety of which is incorporated by reference herein.

### FIELD OF THE INVENTION

The present invention relates to small molecule inhibitors of KRAS that inhibit, for example, the G12C mutant, G12D mutant and G12V mutant of Kirsten rat sarcoma (KRAS) protein and relates to a pharmaceutical composition comprising a compound of Formula (I) as well as methods of using such a compound for treatment of diseases, including cancers.

### BACKGROUND OF THE INVENTION

RAS, which is a small monomeric GTP-binding protein having a molecular weight of about 21 kDa, acts as a molecular on/off switch. RAS can bind to GTP by binding to proteins of a guanine nucleotide exchange factor (GEF) (e.g., SOS1), which forces the release of a bound nucleotide, and releases GDP. When RAS binds to GTP, it becomes activated (turned on) and recruits and activates proteins necessary for the propagation of other receptors' signals, such as c-Raf and PI 3-kinase. RAS also possesses enzymatic activity with which it cleaves the terminal phosphate of the GTP nucleotide and converts the nucleotide into GDP. The rate of conversion is usually slow, but can be dramatically sped up by a protein of the GTPase-activating protein (GAP) class, such as RasGAP. When GTP is converted into GDP, RAS is deactivated (turned off).

The commonly known members of the RAS subfamily include HRAS, KRAS, and NRAS. Of these, mutations of KRAS are observed in many malignant tumors: in 86% of pancreatic ductal adenocarcinoma (PDAC), in 41% of colorectal cancers (CRC), and in 32% of lung adenocarcinoma (LUAD; a subtype of non-small-cell lung cancer (NSCLC)). The mutations often occur in the glycine residue at position 12 of KRAS (“G12”); the mutation at G12 dominates 91% (PDAC), 68% (CRC) and 85% (LUAD) of the total KRAS mutations, respectively. The distributions of amino acid substitutions at G12 vary among each tissue type. The most prevalent mutation in LUAD is the mutation into cysteine (“G12C”) (46%), while the predominant mutation in PDAC (45%) and CRC (45%) is the mutation into aspartic acid (“G12D”). The mutation at G12 into valine (“G12V”) is observed in a significant portion of G12 mutations in all of PDAC (35%), CRC (30%) and LUAD (23%). (Nature Reviews Drug Discovery, 19, 533-552, 2020).

Intense efforts in developing KRAS-G12C inhibitors are underway. Several covalent inhibitors which focus on the cysteine residue have been reported, and some of them have been subjected to clinical studies, such as AMG510 (NCT03600883), MRTX849 (NCT03785249) and JNJ-74699157 (NCT04006301). However, the KRAS-G12C mutation only accounts for a fraction of all KRAS mutations and is primarily found in LUAD. To effectively inhibit the other commonly-occurring KRAS mutated proteins, such as KRAS-G12D and KRAS-G12V, different approaches are needed as these mutants lack reactive cysteines in the active site (Nature Reviews Drug Discovery, 19, 533-552, 2020).

### SUMMARY OF THE DISCLOSURE

The present disclosure provides small molecule inhibitors which modulate mutant KRAS, HRAS, and/or NRAS proteins and may be valuable pharmaceutically active compounds for the treatment of cancer. In some embodiments the disclosed compounds selectively inhibit the KRAS-G12C, KRAS-G12D and/or KRAS-G12V proteins. The compounds of Formula (I):

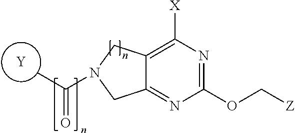

and their pharmaceutically acceptable salts, can modulate the activity of KRAS, HRAS and/or NRAS activity and thereby affect the signaling pathway which regulates cell growth, differentiation, and proliferation associated with oncological disorders. In certain embodiments, the compounds of Formula (I) can inhibit the KRAS-G12C, KRAS-G12D and/or KRAS-G12V proteins. The disclosure furthermore provides processes for preparing compounds of Formula (I), methods for using such compounds to treat oncological disorders, and pharmaceutical compositions which comprise compounds of Formula (I).

### DETAILED DESCRIPTION OF THE INVENTION

### Compounds of the Disclosure

In one embodiment, the present disclosure provides a compound having structural Formula (I), or a pharmaceutically acceptable salt thereof, as shown above, wherein:
X is selected from the group consisting of
(i) a 6- to 9-membered monocyclic- or fused bicyclic- or bridged bicyclic-heterocycloalkyl, wherein said heterocycloalkyl is saturated and contains 1 to 2 heteroatoms selected from the group consisting of N, S, and O;
(ii) an 8- to 10-membered spiroheterocycloalkyl, wherein said spiroheterocycloalkyl is saturated and contains 1 to 2 heteroatoms selected from the group consisting of N and O;

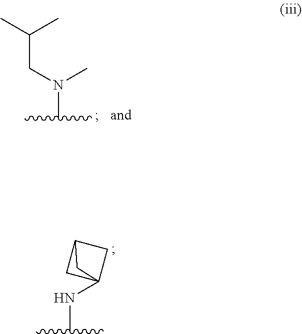

wherein, when X is (i) or (ii), X is unsubstituted or independently substituted by 1 to 4 R^{X} substituents selected from the group consisting of halo, hydroxy, C₁-C₆ alkyl, C₁-C₃ hydroxyalkyl, C₁-C₆ fluoroalkyl, carboxy, carbamoyl, C₁-C₃ carboxyalkyl, oxo, cyano, cyanomethyl, amino, pyrazolyl, oxadiazolonyl, —NHC(O)C₁-C₃alkoxyC₁-C₃alkyl, —NHC(O) C₁-C₃alkoxyC₆-C₁₀aryl, C₁-C₃alkoxy, methoxy(C₁-C₃)alkyl, amino(C₁-C₃)alkyl, C₁-C₃ alkylamino(C₁-C₃)alkyl, C₁-C₃ dialkylamino, C₁-C₃ dialkylamino(C₁-C₃)alkyl, and NHC(O)C₅-C₁₀heteroaryl, where heteroaryl may be substituted by C₁-C₃alkyl;
Ring Y is a 9- to 10-membered bicyclic ring system, wherein the ring system is partially unsaturated or aromatic, and wherein Ring Y contains 0 to 2 nitrogen heteroatoms;
wherein Ring Y is unsubstituted or independently substituted by 1 to 4 R^{y} substituents selected from the group consisting of halo, hydroxy, amino, C₁-C₃ alkyl, C₂-C₃ alkynyl, and C₁-C₃ fluoroalkyl;
Z is selected from the group consisting of
(i) a 5- to 8-membered monocyclic- or bicyclic-heterocycloalkyl, wherein said heterocycloalkyl is saturated and contains 1 nitrogen heteroatom and wherein said heterocycloalkyl is unsubstituted or substituted with 1 substituent R^{ZHC} selected from the group consisting of halo, C₁-C₃ alkyl, and methylene(C₁-C₃ alkyl)(C₁-C₃ alkyl)carbamate;

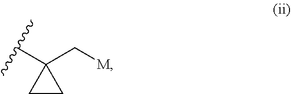

wherein M is selected from the group consisting of hydroxy, C₁-C₃ dialkylamino, and C₁-C₄ alkylamino, and wherein the cyclopropyl group is unsubstituted or substituted with up to 2 halo groups;

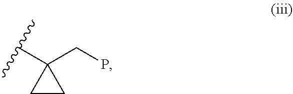

wherein P is a 5- to 8-membered monocyclic- or fused bicyclic- or bridged bicyclic-heterocycloalkyl, wherein said heterocycloalkyl is saturated and contains 1 to 2 heteroatoms selected from the group consisting of N and O, wherein said heterocycloalkyl is unsubstituted or substituted with 1 R^{P} substituent selected from the group consisting of halo, hydroxy, C₁-C₃ hydroxyalkyl, C₁-C₃ cyanoalkyl, carbamoyl, C₁-C₃ alkoxy, cyano, —NHC(O) C₁-C₃alkyl, and oxadiazolonyl, and wherein the cyclopropyl group is unsubstituted or substituted with up to 2 halo groups;
subscript m is 0 or 1; and
subscript n is 1 or 2.

In another embodiment, the present disclosure provides a compound of Formula (I), wherein Ring Y is

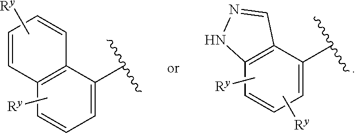

In another embodiment, the present disclosure provides a compound of Formula (I), wherein Ring Y is

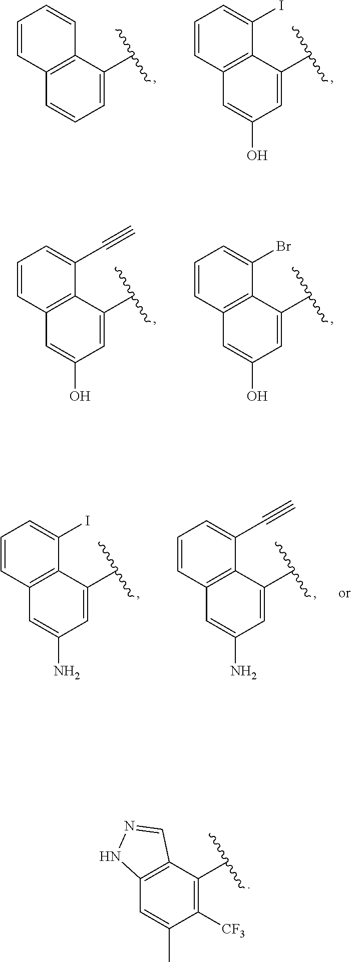

In another embodiment, the present disclosure provides a compound of Formula (I), wherein X is

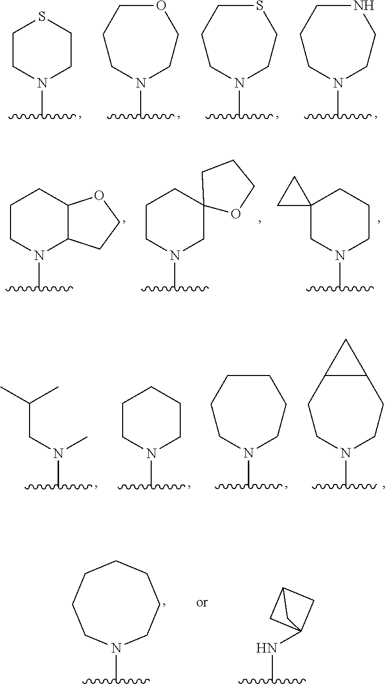

and wherein X is unsubstituted or independently substituted by 1 to 4 R^{X} substituents.

In another embodiment, the present disclosure provides a compound of Formula (I), wherein X is substituted by 1 to 4 R^{X} substituents selected from the group consisting of halo, hydroxy, C₁-C₆ alkyl, C₁-C₃ hydroxyalkyl, C₁-C₆ fluoroalkyl, carboxy, carbamoyl, C₁-C₃ carboxyalkyl, oxo, cyano, cyanomethyl, amino, pyrazolyl, oxadiazolonyl, —NHC(O)C₁-C₃alkoxyC₁-C₃alkyl, —NHC(O)C₁-C₃alkoxyC₆-C₁₀aryl, and NHC(O)C₅-C₁₀heteroaryl, where heteroaryl may be substituted by C₁-C₃alkyl.

In another embodiment, the present disclosure provides a compound of Formula (I), wherein X is

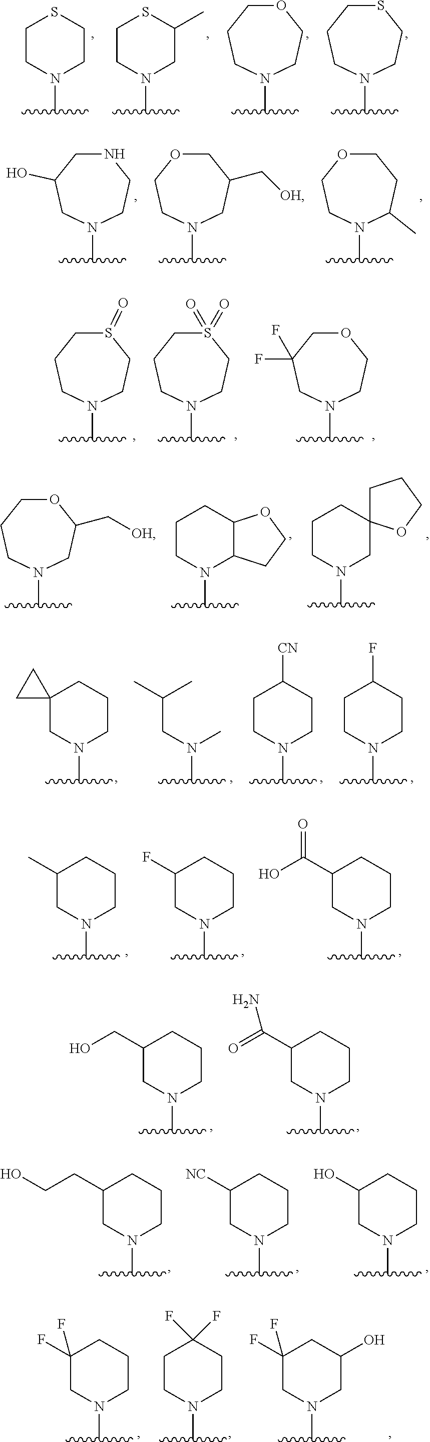

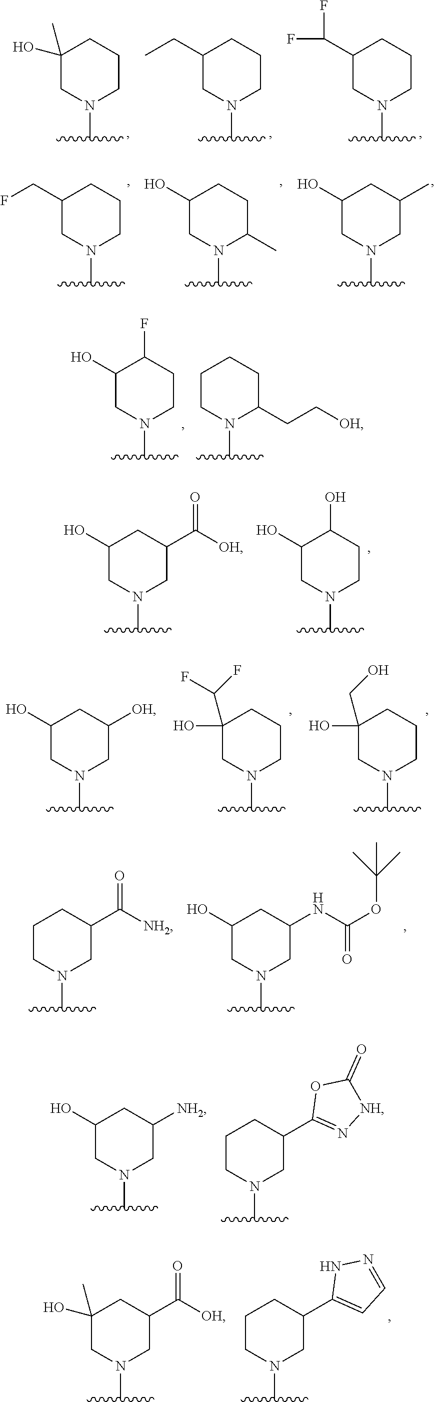

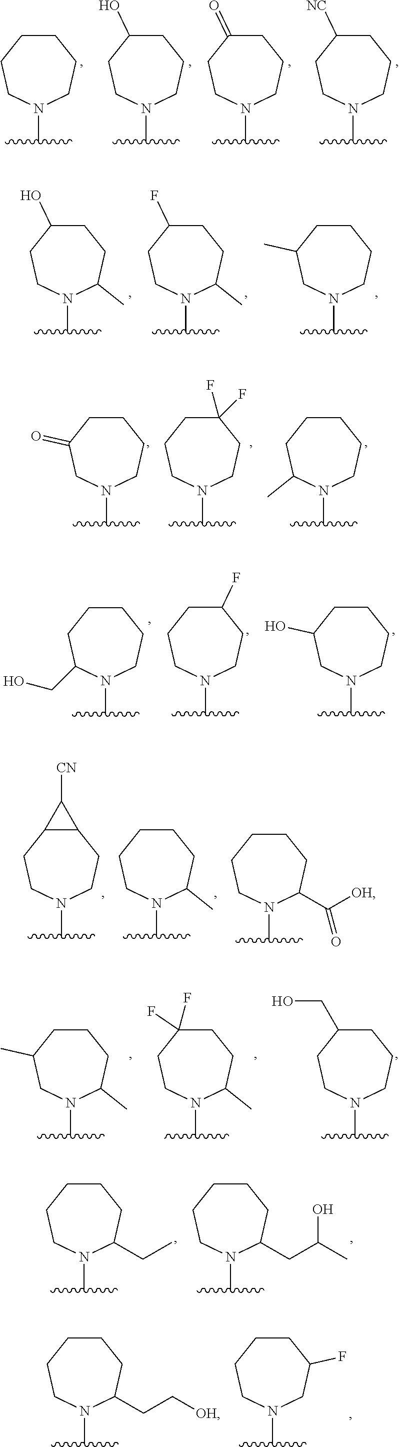

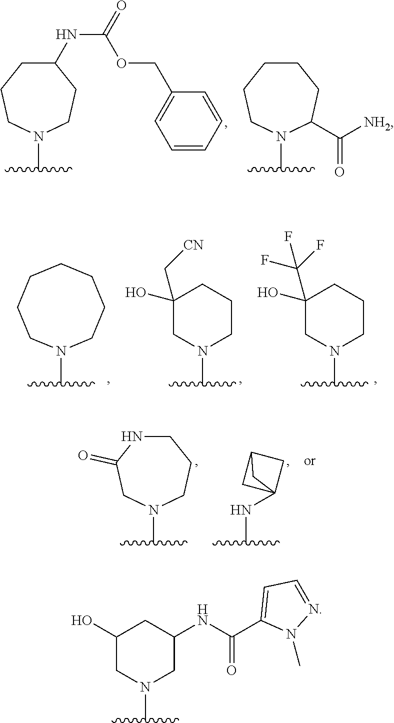

In another embodiment, the present disclosure provides a compound of Formula (I), wherein X is

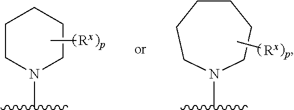

wherein subscript p is 0, 1, or 2.

In another embodiment, the present disclosure provides a compound of Formula (I), wherein X is

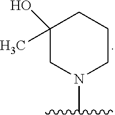 [SMILES: *N1CCCC(C)(O)C1]

In another embodiment, the present disclosure provides a compound of Formula (I), wherein Z is

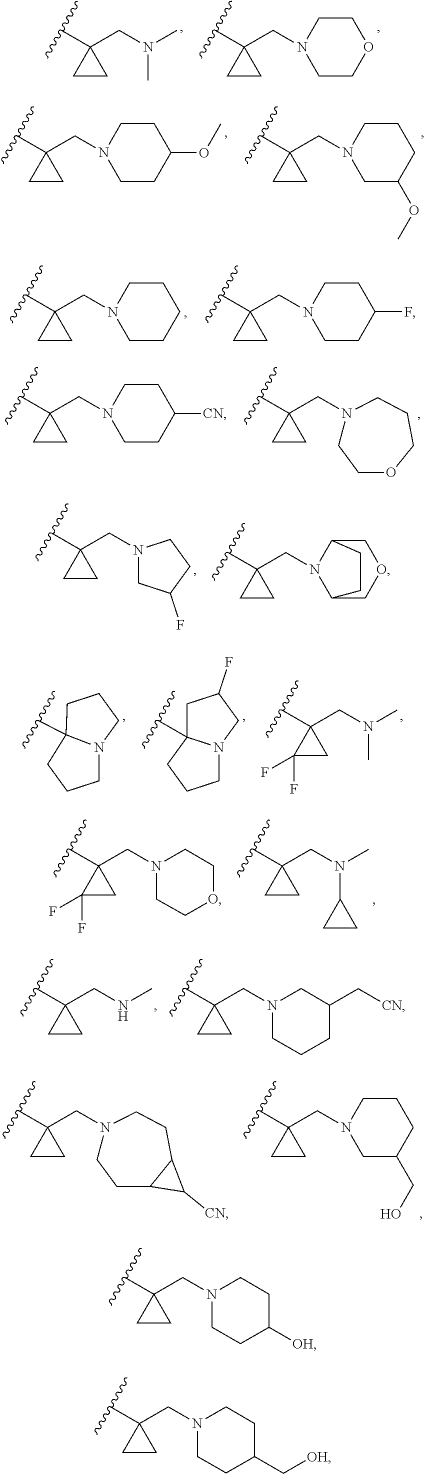

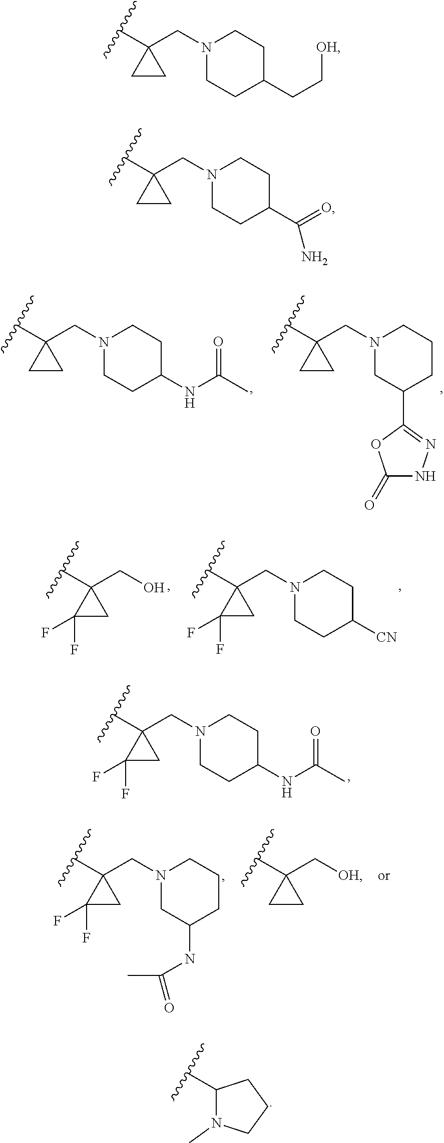

In another embodiment, the present disclosure provides a compound of Formula (I), wherein subscript m is 1.

In another embodiment, the present disclosure provides a compound of Formula (I), wherein subscript n is 1.

In another embodiment, the present disclosure provides a compound of Formula (I), wherein subscript n is 2.

In specific embodiments, the present disclosure provides a compound as described in any one of Examples 1-151 as set forth below, or a pharmaceutically acceptable salt thereof.

The present disclosure includes the pharmaceutically acceptable salts of the compounds defined herein, including the pharmaceutically acceptable salts of all structural formulas, embodiments and classes defined herein.

### Definitions

Unless defined otherwise, all technical and scientific terms used herein have the same meaning as is commonly understood by one of skill in the art to which this invention belongs.

As used throughout this disclosure, “a compound of Formula (I)” is to be understood to include “a compound of Formula (I) or a pharmaceutically acceptable salt thereof”. Likewise “a compound of Formula (I)”, “compound(s) disclosed herein”, “compound(s) described herein”, “compound(s) of the disclosure”, etc., are used interchangeably and include both the compound, as well as a pharmaceutically acceptable salt thereof.

“Alkyl”, as well as other groups having the prefix “alk”, such as alkoxy, and the like, means carbon chains which may be linear or branched, or combinations thereof, containing the indicated number of carbon atoms. For instance, a C₁-C₆ alkyl means an alkyl group having one (i.e., methyl) up to 6 carbon atoms (i.e., hexyl). In particular embodiments, linear alkyl groups have 1-6 carbon atoms and branched alkyl groups have 3-7 carbon atoms. Examples of alkyl groups include methyl, ethyl, propyl, isopropyl, butyl, sec- and tert-butyl, pentyl, hexyl, heptyl, octyl, nonyl and the like.

“Alkoxy” and “alkyl-O—” are used interchangeably and refer to an alkyl group linked to oxygen.

“Alkoxyalkyl” means an alkoxy-alkyl group in which the alkoxy and alkyl groups are as previously defined. The bond to the parent moiety is through a carbon atom of the alkyl component. Non-limiting examples of suitable alkyoxyalkyl groups include methoxyalkyl groups such as methoxymethyl and methoxyethyl.

“Alkynyl” means an aliphatic hydrocarbon group containing at least one carbon-carbon triple bond and which may be straight or branched. Non-limiting examples include ethynyl, propynyl, and butynyl.

“Aryl” means a monocyclic, bicyclic or tricyclic carbocyclic aromatic ring or ring system containing 5-14 carbon atoms, wherein at least one of the rings is aromatic. Non-limiting examples include phenyl and naphthyl.

“Alkoxyaryl” means an alkoxy-aryl group in which the alkoxy and aryl groups are as previously defined. The bond to the parent moiety is through a carbon atom of the aryl component. Non-limiting examples of suitable alkyoxyaryl groups include methoxyphenyl.

“Aminoalkyl” means-alkyl-NH₂ group in which the alkyl is as previously defined. The bond to the parent moiety is through a carbon atom of the alkyl component. Non-limiting examples of suitable aminoalkyl groups include aminomethyl and aminoethyl. “Alkylamino” means-NH-alkyl group in which the alkyl is as previously defined. The bond to the parent moiety is through the nitrogen of the amino component.

“Bicyclic ring system” refers to two joined rings. The rings may be fused, i.e., share two adjacent atoms, or “spirocyclic”, i.e., share only a single atom, or bridged”, i.e., share three or more atoms with two bridgehead atoms being connected by a bridge containing at least one atom. Likewise the bicyclic rings may be aryl rings, heterocyclic rings, cycloalkyl rings, etc.

“Carbamoyl” means a H₂N—C(O)— group, which is the univalent group formed by loss of —OH group of carbamic acid. The bond to the parent group is through the carbon atom of the carbonyl component.

“Carboxyalkyl” means carboxy(COOH)-alkyl group in which the alkyl group is previously defined. The bond to the parent group is through the carbon atom of the alkyl component.

“Cyanoalkyl” means an -alkyl-CN group in which the alkyl is as previously defined. The bond to the parent moiety is through a carbon atom of the alkyl component. Non-limiting examples of suitable cyanoalkyl groups include cyanomethyl and 3-cyanopropyl.

“Cycloalkyl” means a saturated cyclic hydrocarbon radical. In particular embodiments, the cycloalkyl group has 3-12 carbon atoms, forming 1-3 carbocyclic rings that are fused. Examples of cycloalkyl include cyclopropyl, cyclobutyl, cyclopentyl, cyclohexyl, cycloheptyl, adamantyl, and the like.

“Dialkylamino” means an alkylamino as previously defined, wherein the amino atom is substituted by two alkyl substituents, which substitutions can be the same or different, e.g., —N(CH₃)₂ or —N(CH₃)(CH₂CH₃).

“Dialkylaminoalkyl” means an aminoalkyl as previously defined, wherein the amino atom is also substituted by two alkyl substituents. The alkyl groups substituted on the amino atom can be the same or different. Non-limiting examples of suitable dialkylaminoalkyl groups include dimethyl aminomethyl [(CH₃)₂ NCH₂-] and N-ethyl-N-methylaminoethyl [(CH₃CH₂)(CH₃)N—CH₂CH₂—].

“Fluoroalkyl” includes mono-substituted as well as multiple fluoro-substituted alkyl groups, up to perfluoro substituted alkyl. For example, fluoromethyl, 1,1-difluoroethyl, trifluoromethyl or 1,1,1,2,2-pentafluorobutyl are included.

“Halogen” or “halo”, unless otherwise indicated, includes fluorine (fluoro), chlorine (chloro), bromine (bromo) and iodine (iodo). In one embodiment, halo is fluoro (—F) or chloro (—Cl).

“Heteroaryl” refers to aromatic monocyclic, bicyclic and tricyclic ring structures in which one or more atoms in the ring, the heteroatom(s), is an element other than carbon. Heteroatoms are typically O, S, or N atoms. Examples of heteroaryl groups include pyrazolyl, oxadiazolonyl, pyridinyl, pyrimidinyl, pyrrolyl, pyridazinyl, isoxazolyl, thiazolyl, oxazolyl, indolyl, benzoxazolyl, benzothiazolyl, and imidazolyl.

“Heterocycloalkyl” or “heterocyclic ring” or “heterocycle” means a non-aromatic monocyclic, bicyclic, tricyclic or bridged ring system comprising about 3 to about 10 ring atoms, preferably about 5 to about 10 ring atoms, in which one or more of the atoms in the ring system is an element other than carbon, for example, nitrogen, oxygen, phosphorus or sulfur, alone or in combination. There are no adjacent oxygen and/or sulfur atoms present in the ring system. In some embodiments, heterocycloalkyls contain about 5 to about 6 ring atoms. The prefix aza, oxa, phospha or thia before the heterocyclyl root name means that at least a nitrogen, oxygen, phosphorus or sulfur atom respectively is present as a ring atom. In some embodiments, the nitrogen or sulfur atom of the heterocycloalkyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, phosphorinane, phosphinane, 1-oxophosphinan-1-ium and the like. “Spiroheterocycloalkyl” refers to a fused ring system in which the rings share only a single atom and at least one of the rings is a heterocycloalkyl.

“Hydroxyalkyl” means a HO-alkyl-group in which alkyl is as previously defined. The bond to the parent moiety is through a carbon atom of the alkyl group. Preferred hydroxyalkyls contain lower alkyl. Non-limiting examples of suitable hydroxyalkyl groups include hydroxymethyl and 2-hydroxyethyl.

“Methylene(C₁-C₃ alkyl)(C₁-C₃ alkyl)carbamate” means having the structure of

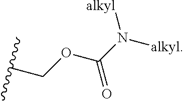

In other words, the carbamate group has alkyl groups, as previously defined, attached to the nitrogen atom.

When any variable (e.g., R^{y}) occurs more than one time in any constituent or in Formula (I) or other generic formulas herein, its definition on each occurrence is independent of its definition at every other occurrence. Combinations of substituents and/or variables are permissible only if such combinations result in stable compounds. In choosing compounds of the present disclosure, one of ordinary skill in the art will recognize that the various substituents, e.g., R^{y}, are to be chosen in conformity with well-known principles of chemical structure connectivity and stability. Unless expressly stated to the contrary, substitution by a named substituent is permitted on any atom in a ring (e.g., aryl, a heteroaryl ring, or a saturated heteroaryl ring) provided such ring substitution is chemically allowed and results in a stable compound. A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

The term “substituted” shall be deemed to include multiple degrees of substitution by a named substituent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

Unless expressly depicted or described otherwise, variables depicted in a structural formula with a “floating” bond, such as R^{X}, are permitted on any available carbon atom in the ring to which the variable is attached. When a moiety is noted as being “optionally substituted” in Formula (I) or any embodiment thereof, it means that Formula (I) or the embodiment thereof encompasses compounds that contain the noted substituent (or substituents) on the moiety and also compounds that do not contain the noted substituent (or substituents) on the moiety.

The wavy line [symbol] , as used herein, indicates a point of attachment to the rest of the compound.

The compounds of Formula (I) may contain one or more asymmetric centers and can thus occur as racemates and racemic mixtures, single enantiomers, diastereoisomeric mixtures and individual diastereoisomers. Centers of asymmetry that are present in the compounds of Formula (I) can all independently of one another have S configuration or R configuration. The compounds of Formula (I) include all possible enantiomers and diastereomers and mixtures of two or more stereoisomers, for example, mixtures of enantiomers and/or diastereomers, in all ratios. Thus, enantiomers are a subject of the disclosure in enantiomerically pure form, both as levorotatory and as dextrorotatory antipodes, in the form of racemates and in the form of mixtures of the two enantiomers in all ratios. In the case of a cis/trans isomerism, the disclosure includes both the cis form and the trans form as well as mixtures of these forms in all ratios. The present disclosure is meant to comprehend all such stereoisomeric forms of the compounds of Formula (I). Where a structural formula or chemical name specifies a particular configuration at a stereocenter, the enantiomer or stereoisomer of the compound resulting from that specified stereocenter is intended. Where a structural formula of the compounds of Formula (I) indicates a straight line at a chiral center, the structural formula includes both the S and R stereoisomers associated with the chiral center and mixtures thereof.

The compounds of Formula (I) may be separated into their individual diastereoisomers by, for example, fractional crystallization from a suitable solvent, for example, methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration. Vibrational circular dichroism (VCD) may also be used to determine the absolute stereochemistry. Alternatively, any stereoisomer or isomers of the compounds of Formula (I) may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereoisomeric mixture, followed by separation of the individual diastereoisomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

The compounds of Formula (I) which contain olefinic double bonds, unless specified otherwise, they are meant to include both E and Z geometric isomers.

Some of the compounds described herein may exist as tautomers which have different points of attachment of hydrogen accompanied by one or more double bond shifts. For example, a ketone and its enol form are keto-enol tautomers. The individual tautomers as well as mixtures thereof are encompassed by the compounds of Formula (I).

Some of the compounds of Formula (I) described herein may exist as atropisomers when the rotational energy barrier around a single bond is sufficiently high to prevent free rotation at a given temperature, thus allowing isolation of individual conformers with distinct properties. The individual atropisomers as well as mixtures thereof are encompassed with compounds of Formula (I) of the present disclosure. When resolved, individual atropisomers can be designated by established conventions such as those specified by the International Union of Pure Applied Chemistry (IUPAC) 2013 Recommendations.

In the compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure as described and claimed herein is meant to include all suitable isotopic variations of the compounds of Formula (I) and embodiments thereof. For example, different isotopic forms of hydrogen (H) include protium (¹H) and deuterium (²H, also denoted herein as D). Protium is the predominant hydrogen isotope found in nature. Enriching for deuterium may afford certain therapeutic advantages, such as increasing in vivo half-life or reducing dosage requirements or may provide a compound useful as a standard for characterization of biological samples. Isotopically-enriched compounds can be prepared without undue experimentation by conventional techniques well known to those skilled in the art or by processes analogous to those described in the Schemes and Examples herein using appropriate isotopically-enriched reagents and/or intermediates.

The term “pharmaceutically acceptable salts” refers to salts prepared from pharmaceutically acceptable non-toxic bases or acids. When a compound of Formula (I) is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Preferred are the ammonium, calcium, magnesium, potassium and sodium salts. Salts prepared from pharmaceutically acceptable organic non-toxic bases include salts of primary, secondary, and tertiary amines derived from both naturally occurring and synthetic sources. Pharmaceutically acceptable organic non-toxic bases from which salts can be formed include, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, dicyclohexylamine, lysine, methylglucamine, morpholine, piperazine, piperidine, polyamine resins, procaine, purines, theobromine, triethylamine, trimethylamine, tripropylamine, tromethamine and the like.

When a compound of Formula (I) is basic, its corresponding salt can be conveniently prepared from pharmaceutically acceptable non-toxic inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids. If a compound of Formula (I) simultaneously contain acidic and basic groups in the molecule, the disclosure also includes, in addition to the salt forms mentioned, inner salts or betaines (zwitterions). Salts can be obtained from the compounds of Formula (I) by customary methods which are known to the person skilled in the art, for example, by combination with an organic or inorganic acid or base in a solvent or dispersant, or by anion exchange or cation exchange from other salts. The present disclosure also includes all salts of the compounds of Formula (I) which, owing to low physiological compatibility, are not directly suitable for use in pharmaceuticals but which can be used, for example, as intermediates for chemical reactions or for the preparation of pharmaceutically acceptable salts.

Furthermore, the compounds of Formula (I) may exist in amorphous form and/or one or more crystalline forms, and as such all amorphous and crystalline forms and mixtures thereof of the compounds of Formula (I), including the Examples, are intended to be included within the scope of the present disclosure. In addition, some of the compounds of Formula (I) may form solvates with water (i.e., a hydrate) or common organic solvents such as but not limited to ethyl acetate. Such solvates and hydrates, particularly the pharmaceutically acceptable solvates and hydrates, of the instant compounds are likewise encompassed within the scope of this disclosure, along with un-solvated and anhydrous forms.

Any pharmaceutically acceptable pro-drug modification of a compound of Formula (I) which results in conversion in vivo to a compound within the scope of this disclosure is also within the scope of this disclosure.

The terms “therapeutically effective (or efficacious) amount” and similar descriptions such as “an amount efficacious for treatment” or “an effective dose” are intended to mean that amount of a compound of Formula (I) that will elicit the biological or medical response of a tissue, a system, animal or human that is being sought by a researcher, veterinarian, medical doctor or other clinician. In a preferred embodiment, the term “therapeutically effective amount” means an amount of a compound of Formula (I) that alleviates at least one clinical symptom in a human patient. The terms “prophylactically effective (or efficacious) amount” and similar descriptions such as “an amount efficacious for prevention” are intended to mean that amount of a compound of Formula (I) that will prevent or reduce the risk of occurrence of the biological or medical event that is sought to be prevented in a tissue, a system, animal or human by a researcher, veterinarian, medical doctor or other clinician.

Dosages of the compounds of Formula (I)

The dosage regimen utilizing a compound of Formula (I) is selected in accordance with a variety of factors including type, species, age, weight, sex and medical condition of the patient: the severity of the condition to be treated: the potency of the compound chosen to be administered: the route of administration; and the renal and hepatic function of the patient. A consideration of these factors is well within the purview of the ordinarily skilled clinician for the purpose of determining the therapeutically effective or prophylactically effective dosage amount needed to prevent, counter, or arrest the progress of the condition. It is understood that a specific daily dosage amount can simultaneously be both a therapeutically effective amount, e.g., for treatment of an oncological condition, and a prophylactically effective amount, e.g., for prevention of an oncological condition.

While individual needs vary, determination of optimal ranges of effective amounts of the compounds of Formula (I) is within the skill of the art. For administration to a human in, for example, the curative or prophylactic treatment of the conditions and disorders identified herein, the typical dosages of the compounds of Formula (I) can be about 0.05 mg/kg/day to about 50 mg/kg/day, or at least 0.05 mg/kg, or at least 0.08 mg/kg, or at least 0.1 mg/kg, or at least 0.2 mg/kg, or at least 0.3 mg/kg, or at least 0.4 mg/kg, or at least 0.5 mg/kg, and any amount therebetween, to about 50 mg/kg or less, or about 40 mg/kg or less, or about 30 mg/kg or less, or about 20 mg/kg or less, or about 10 mg/kg or less and any amount therebetween, which can be, for example, about 2.5 mg/day (0.5 mg/kg x 5 kg) to about 5000 mg/day (50 mg/kg x 100 kg). For example, dosages of the compounds can be about 0.1 mg/kg/day to about 50 mg/kg/day, or about 0.05 mg/kg/day to about 10 mg/kg/day, or about 0.05 mg/kg/day to about 5 mg/kg/day, or about 0.05 mg/kg/day to about 3 mg/kg/day, or about 0.07 mg/kg/day to about 3 mg/kg/day, or about 0.09 mg/kg/day to about 3 mg/kg/day, or about 0.05 mg/kg/day to about 0.1 mg/kg/day, or about 0.1 mg/kg/day to about 1 mg/kg/day, or about 1 mg/kg/day to about 10 mg/kg/day, or about 1 mg/kg/day to about 5 mg/kg/day, or about 1 mg/kg/day to about 3 mg/kg/day, or about 3 mg/day to about 500 mg/day, or about 5 mg/day to about 250 mg/day, or about 10 mg/day to about 100 mg/day, or about 3 mg/day to about 10 mg/day, or about 100 mg/day to about 250 mg/day. Such doses may be administered in a single dose or may be divided into multiple doses.

### Pharmaceutical Compositions

The compounds of Formula (I) and their pharmaceutically acceptable salts can be administered to animals, preferably to mammals, and in particular to humans, as pharmaceuticals by themselves, in mixtures with one another or in the form of pharmaceutical compositions. The term “subject” or “patient” includes animals, preferably mammals and especially humans, who use the instant active agents for the prevention or treatment of a medical condition. Administering of the drug to the subject includes both self-administration and administration to the patient by another person. The subject may be in need of, or desire, treatment for an existing disease or medical condition, or may be in need of or desire prophylactic treatment to prevent or reduce the risk of occurrence of said disease or medical condition. As used herein, a subject “in need” of treatment of an existing condition or of prophylactic treatment encompasses both a determination of need by a medical professional as well as the desire of a patient for such treatment.

The present disclosure therefore also provides the compounds of Formula (I) and their pharmaceutically acceptable salts for use as pharmaceuticals, their use for modulating the activity of mutant KRAS, HRAS and/or NRAS proteins and in particular their use in the therapy and prophylaxis of the below-mentioned diseases or disorders as well as their use for preparing medicaments for these purposes. In certain embodiments, the compounds of Formula (I) and their pharmaceutically acceptable salts inhibit the KRAS-G12C, KRAS-G12D and/or KRAS-G12V proteins.

Furthermore, the present disclosure provides pharmaceutical compositions which comprise as active component an effective dose of at least one compound of Formula (I) and/or a pharmaceutically acceptable salt thereof and a customary pharmaceutically acceptable carrier, i.e., one or more pharmaceutically acceptable carrier substances and/or additives.

Thus, the present disclosure provides, for example, said compound and its pharmaceutically acceptable salts for use as pharmaceutical compositions which comprise as active component an effective dose of at least one compound of Formula (I) and/or a pharmaceutically acceptable salt thereof and a customary pharmaceutically acceptable carrier, and the uses of said compound and/or a pharmaceutically acceptable salt thereof in the therapy or prophylaxis of the below-mentioned diseases or disorders, e.g., cancer, as well as their use for preparing medicaments for these purposes.

The pharmaceutical compositions according to the disclosure can be administered orally, for example, in the form of pills, tablets, lacquered tablets, sugar-coated tablets, granules, hard and soft gelatin capsules, aqueous, alcoholic or oily solutions, syrups, emulsions or suspensions, or rectally, for example, in the form of suppositories. Administration can also be carried out parenterally, for example subcutaneously, intramuscularly or intravenously in the form of solutions for injection or infusion.

Other suitable administration forms are, for example, percutaneous or topical administration, for example, in the form of ointments, tinctures, sprays or transdermal therapeutic systems, or, for example, microcapsules, implants or rods. The preferred administration form depends, for example, on the disease to be treated and on its severity.

The amount of active compound of a compound described herein and/or its pharmaceutically acceptable salts in the pharmaceutical composition normally is from 0.01 to 200 mg, or from 0.1 to 200 mg, or from 1 to 200 mg, per dose, but depending on the type of the pharmaceutical composition, it can also be higher. In some embodiments, the amount of active compound of a compound of Formula (I) and/or its pharmaceutically acceptable salts in the pharmaceutical composition is from 0.01 to 10 mg per dose. The pharmaceutical compositions usually comprise 0.5 to 90 percent by weight of at least one compound of Formula (I) and/or its pharmaceutically acceptable salts. The preparation of the pharmaceutical compositions can be carried out in a manner known per se. For this purpose, one or more compounds of Formula (I) and/or their pharmaceutically acceptable salts, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage form which can then be used as a pharmaceutical in human or veterinary medicine.

For the production of pills, tablets, sugar-coated tablets and hard gelatin capsules, it is possible to use, for example, lactose, starch, for example, maize starch, or starch derivatives, talc, stearic acid or its salts, etc. Carriers for soft gelatin capsules and suppositories are, for example, fats, waxes, semisolid and liquid polyols, natural or hardened oils, etc. Suitable carriers for the preparation of solutions, for example, of solutions for injection, or of emulsions or syrups are, for example, water, physiologically acceptable sodium chloride solution, alcohols such as ethanol, glycerol, polyols, sucrose, invert sugar, glucose, mannitol, vegetable oils, etc. It is also possible to lyophilize the compounds of Formula (I) and their pharmaceutically acceptable salts and to use the resulting lyophilisates, for example, for preparing preparations for injection or infusion. Suitable carriers for microcapsules, implants or rods are, for example, copolymers of glycolic acid and lactic acid.

Besides the active compounds and carriers, the pharmaceutical compositions can also contain customary additives, for example, fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents and/or antioxidants.

### Methods of Using the Compounds of Formula (I)

The present application provides a method of inhibiting RAS-mediated cell signaling comprising contacting a cell with a compound of Formula (I) or a pharmaceutically acceptable salt thereof. Inhibition of RAS-mediated signal transduction can be assessed and demonstrated by a wide variety of ways known in the art. Non-limiting examples include (a) a decrease in GTPase activity of RAS; (b) a decrease in GTP binding affinity or an increase in GDP binding affinity: (c) an increase in Koff of GTP or a decrease in Koff of GDP: (d) a decrease in the levels of signaling transduction molecules downstream in the RAS pathway, such as a decrease in pMEK, pERK, or pAKT levels; and/or (e) a decrease in binding of RAS complex to downstream signaling molecules including but not limited to Raf. Kits and commercially available assays can be utilized for determining one or more of the above.

The present application also provides methods of using the compounds of Formula (I) (or their pharmaceutically acceptable salts) or pharmaceutical compositions containing such compounds to treat disease conditions, including but not limited to, conditions implicated by mutant KRAS, HRAS and/or NRAS proteins (e.g., cancer), and in some embodiments the KRAS-G12C, KRAS-G12D and/or KRAS-G12V mutants.

In some embodiments, a method for treatment of cancer is provided, the method comprising administering a therapeutically effective amount a compound of Formula (I) (or a pharmaceutically acceptable salt thereof) or any of the foregoing pharmaceutical compositions comprising such a compound to a subject in need of such treatment. In some embodiments, the cancer is mediated by a KRAS, HRAS or NRAS mutation, e.g., the KRAS-G12C, KRAS-G12D and/or KRAS-G12V mutations. In various embodiments, the cancer is pancreatic cancer, colorectal cancer or lung cancer. In some embodiments, the cancer is gall bladder cancer, thyroid cancer, or bile duct cancer.

In some embodiments the present disclosure provides a method of treating a disorder in a subject in need thereof, wherein said method comprises determining if the subject has a KRAS, HRAS or NRAS mutation (e.g., KRAS-G12C, KRAS-G12D and/or KRAS-G12V mutations) and if the subject is determined to have the KRAS, HRAS or NRAS mutation, then administering to the subject a therapeutically effective amount of a compound of Formula (I) or a pharmaceutically acceptable salt thereof.

The disclosed compounds inhibit anchorage-independent cell growth and therefore have the potential to inhibit tumor metastasis. Accordingly, another embodiment of the present disclosure provides a method for inhibiting tumor metastasis, the method comprising administering an effective amount a compound of Formula (I).

KRAS, HRAS or NRAS mutations have also been identified in hematological malignancies (e.g., cancers that affect blood, bone marrow and/or lymph nodes). Accordingly, certain embodiments are directed to administration of the compounds of Formula (I) (e.g., in the form of a pharmaceutical composition) to a subject in need of treatment of a hematological malignancy. Such malignancies include, but are not limited to leukemias and lymphomas. For example, the presently disclosed compounds can be used for treatment of diseases such as acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMOL) and/or other leukemias. In other embodiments, the compounds are useful for treatment of lymphomas such as Hodgkin's lymphoma or non-Hodgkin's lymphoma. In various embodiments, the compounds are useful for treatment of plasma cell malignancies such as multiple myeloma, mantle cell lymphoma, and Waldenstrom's macroglubunemia.

Determining whether a tumor or cancer comprises a KRAS, HRAS or NRAS mutation (e.g., the KRAS-G12C, KRAS-G12D and/or KRAS-G12V mutations) can be undertaken by assessing the nucleotide sequence encoding the KRAS, HRAS or NRAS protein, by assessing the amino acid sequence of the KRAS, HRAS or NRAS protein, or by assessing the characteristics of a putative KRAS, HRAS or NRAS mutant protein. The sequences of wild-type human KRAS, HRAS or NRAS are known in the art.

Methods for detecting a mutation in a KRAS, HRAS or NRAS nucleotide sequence are also known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples are evaluated for KRAS, HRAS or NRAS mutations (e.g., the KRAS-G12C, KRAS-G12D and/or KRAS-G12V mutations) by real-time PCR. In real-time PCR, fluorescent probes specific for the KRAS, HRAS or NRAS mutation are used. When a mutation is present, the probe binds and fluorescence is detected. In some embodiments, the KRAS, HRAS or NRAS mutation is identified using a direct sequencing method of specific regions (e.g., exon 2 and/or exon 3) in the KRAS, HRAS or NRAS gene.

Methods for detecting a mutation in a KRAS, HRAS or NRAS protein (e.g., the KRAS-G12C, KRAS-G12D and/or KRAS-G12V mutations) are known by those of skill in the art. These methods include, but are not limited to, detection of a KRAS, HRAS or NRAS mutant using a binding agent (e.g., an antibody) specific for the mutant protein, protein electrophoresis and Western blotting, and direct peptide sequencing.

A number of tissue samples can be assessed for determining whether a tumor or cancer comprises a KRAS, HRAS or NRAS mutation (e.g., the KRAS-G12C, KRAS-G12D and/or KRAS-G12V mutations). In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is a circulating tumor cell (CTC) sample. In some embodiments, the sample is processed to a cell lysate. In some embodiments, the sample is processed to DNA or RNA.

The present application also provides a method of treating a hyperproliferative disorder comprising administering a therapeutically effective amount of a compound of Formula (I), or a pharmaceutically acceptable salt thereof to a subject in need thereof. In some embodiments, said method relates to the treatment of a subject who suffers from a cancer such as acute myeloid leukemia, cancer in adolescents, adrenocortical carcinoma childhood, AIDS-related cancers (e.g., lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, Burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, Ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin's lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer: multiple endocrine neoplasia syndromes, multiple myeloma/plasma cell neoplasm, mycosis fungoides, myelodysplasia syndromes, myelodysplastic/myeloproliferative neoplasms, multiple myeloma, Merkel cell carcinoma, malignant mesothelioma, malignant fibrous histiocytoma of bone and osteosarcoma, nasal cavity and paranasal sinus cancer, nasopharyngeal cancer, neuroblastoma, non-Hodgkin's lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer: small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or viral-induced cancer. In some embodiments, said method relates to the treatment of a non-cancerous hyperproliferative disorder such as benign hyperplasia of the skin (e.g., psoriasis), restenosis, or prostate (e.g., benign prostatic hypertrophy (BPH)).

In some embodiments, the methods for treatment are directed to treating lung cancers, and the methods comprise administering a therapeutically effective amount of the compounds of Formula (I) (or pharmaceutical composition comprising such compounds) to a subject in need thereof. In certain embodiments, the lung cancer is a non-small cell lung carcinoma (NSCLC), for example, adenocarcinoma, squamous-cell lung carcinoma or large-cell lung carcinoma. In some embodiments, the lung cancer is a small cell lung carcinoma. Other lung cancers which the compounds of Formula (I) may provide therapeutic benefit for include, but are not limited to, glandular tumors, carcinoid tumors and undifferentiated carcinomas.

The present disclosure also provides methods of modulating a mutant KRAS, HRAS or NRAS protein activity (e.g., activity resulting from the KRAS-G12C, KRAS-G12D and/or KRAS-G12V mutations) by contacting the protein with an effective amount of a compound of Formula (I). Modulation can be inhibiting or activating protein activity. In some embodiments, the present disclosure provides methods of inhibiting protein activity by contacting the mutant KRAS, HRAS or NRAS protein (e.g., KRAS-G12C, KRAS-G12D and/or KRAS-G12V mutants) with an effective amount of a compound of Formula (I) in solution. In some embodiments, the present disclosure provides methods of inhibiting the mutant KRAS, HRAS or NRAS protein activity by contacting a cell, tissue, or organ that expresses the protein of interest. In some embodiments, the disclosure provides methods of inhibiting protein activity in subjects including, but not limited to, rodents and mammals (e.g., humans) by administering into the subjects an effective amount of a compound of Formula (I).

### Combination Therapies

One or more additional pharmacologically active agents may be administered in combination with a compound of Formula (I) (or a pharmaceutically acceptable salt thereof). An additional active agent (or agents) is intended to mean a pharmaceutically active agent (or agents) that is active in the body, including pro-drugs that convert to pharmaceutically active form after administration, which are different from the compound of Formula (I). The additional active agents also include free-acid, free-base and pharmaceutically acceptable salts of said additional active agents. Generally, any suitable additional active agent or agents, including chemotherapeutic agents or therapeutic antibodies, may be used in any combination with the compound of Formula (I) in a single dosage formulation (e.g., a fixed dose drug combination), or in one or more separate dosage formulations which allows for concurrent or sequential administration of the active agents (co-administration of the separate active agents) to subjects. In addition, the compounds of Formula (I) (or pharmaceutically acceptable salts thereof) can be administered in combination with radiation therapy, hormone therapy, surgery or immunotherapy.

The present application also provides methods for combination therapies in which the additional active agent is known to modulate other pathways, or other components of the same pathway, or even overlapping sets of target enzymes which are used in combination with a compound of Formula (I), or a pharmaceutically acceptable salt thereof. In one embodiment, such therapy includes, but is not limited to, the combination of one or more compounds of Formula (I) with chemotherapeutic agents, immunotherapeutic agents, hormonal and anti-hormonal agents, targeted therapy agents, and anti-angiogenesis agents, to provide a synergistic or additive therapeutic effect. In another embodiment, such therapy includes radiation treatment to provide a synergistic or additive therapeutic effect.

Examples of additional active agents (i.e., additional anti-cancer agents) include chemotherapeutic agents (e.g., cytotoxic agents), immunotherapeutic agents, hormonal and anti-hormonal agents, targeted therapy agents, and anti-angiogenesis agents. Many anti-cancer agents can be classified within one or more of these groups. While certain anti-cancer agents have been categorized within a specific group(s) or subgroup(s) herein, many of these agents can also be listed within one or more other group(s) or subgroup(s), as would be presently understood in the art. It is to be understood that the classification herein of a particular agent into a particular group is not intended to be limiting. Many anti-cancer agents are presently known in the art and can be used in combination with the compounds of the present disclosure.

Further, an agent can be an agonist, antagonist, allosteric modulator, toxin or, more generally, may act to inhibit or stimulate its target (e.g., receptor or enzyme activation or inhibition). For example, suitable for use are one or more agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor “c-met”.

In an embodiment, the additional anti-cancer agent is a chemotherapeutic agent, an immunotherapeutic agent, a hormonal agent, an anti-hormonal agent, a targeted therapy agent, or an anti-angiogenesis agent (or angiogenesis inhibitor). In an embodiment, the additional anti-cancer agent is selected from the group consisting of a chemotherapeutic agent, a mitotic inhibitor, a plant alkaloid, an alkylating agent, an anti-metabolite, a platinum analog, an enzyme, a topoisomerase inhibitor, a retinoid, an aziridine, an antibiotic, a hormonal agent, an anti-hormonal agent, an anti-estrogen, an anti-androgen, an anti-adrenal, an androgen, a targeted therapy agent, an immunotherapeutic agent, a biological response modifier, a cytokine inhibitor, a tumor vaccine, a monoclonal antibody, an immune checkpoint inhibitor, an anti-PD-1 agent, an anti-PD-L1 agent, a colony-stimulating factor, an immunomodulator, an immunomodulatory imide (IMiD), an anti-CTLA4 agent, an anti-LAGI agent, an anti-OX40 agent, a GITR agonist, a CAR-T cell, a BiTE, a signal transduction inhibitor, a growth factor inhibitor, a tyrosine kinase inhibitor, an EGFR inhibitor, a histone deacetylase (HDAC) inhibitor, a proteasome inhibitor, a cell-cycle inhibitor, an anti-angiogenesis agent, a matrix-metalloproteinase (MMP) inhibitor, a hepatocyte growth factor inhibitor, a TOR inhibitor, a KDR inhibitor, a VEGF inhibitor, a HIF-1α inhibitor, a HIF-2α inhibitor, a fibroblast growth factor (FGF) inhibitor, a RAF inhibitor, a MEK inhibitor, an ERK inhibitor, a PI3K inhibitor, an AKT inhibitor, an MCL-1 inhibitor, a BCL-2 inhibitor, an SHP2 inhibitor, a HER-2 inhibitor, a BRAF-inhibitor, a gene expression modulator, an autophagy inhibitor, an apoptosis inducer, an antiproliferative agent, and a glycolysis inhibitor.

In one embodiment, the additional anti-cancer agent(s) is a chemotherapeutic agent. Non-limiting examples of chemotherapeutic agents include mitotic inhibitors and plant alkaloids, alkylating agents, anti-metabolites, platinum analogs, enzymes, topoisomerase inhibitors, retinoids, aziridines, and antibiotics.

Non-limiting examples of mitotic inhibitors and plant alkaloids include taxanes such as cabazitaxel, docetaxel, larotaxel, ortataxel, paclitaxel, and tesetaxel; demecolcine; epothilone; eribulin; etoposide (VP-16); etoposide phosphate; navelbine; noscapine; teniposide; thaliblastine; vinblastine; vincristine; vindesine; vinflunine; and vinorelbine.

Non-limiting examples of alkylating agents include nitrogen mustards such as chlorambucil, chlornaphazine, cholophosphamide, cytophosphane, estramustine, ifosfamide, mannomustine, mechlorethamine, mechlorethamine oxide hydrochloride, melphalan, novembichin, phenesterine, prednimustine, tris(2-chloroethyl)amine, trofosfamide, and uracil mustard; alkyl sulfonates such as busulfan, improsulfan, and piposulfan; nitrosoureas such as carmustine, chlorozotocin, fotemustine, lomustine, nimustine, ranimustine, streptozotocin, and TA-07; ethylenimines and methylamelamines such as altretamine, thiotepa, triethylenemelamine, triethylenethiophosphaoramide, trietylenephosphoramide, and trimethylolomelamine; ambamustine; bendamustine; dacarbazine; etoglucid; irofulven; mafosfamide; mitobronitol; mitolactol; pipobroman; procarbazine; temozolomide; treosulfan; and triaziquone.

Non-limiting examples of anti-metabolites include folic acid analogues such as aminopterin, denopterin, edatrexate, methotrexate, pteropterin, raltitrexed, and trimetrexate; purine analogs such as 6-mercaptopurine, 6-thioguanine, fludarabine, forodesine, thiamiprine, and thioguanine; pyrimidine analogs such as 5-fluorouracil (5-FU), 6-azauridine, ancitabine, azacytidine, capecitabine, carmofur, cytarabine, decitabine, dideoxyuridine, doxifiuridine, doxifluridine, enocitabine, floxuridine, galocitabine, gemcitabine, and sapacitabine; 3-aminopyridine-2-carboxaldehyde thiosemicarbazone; broxuridine; cladribine; cyclophosphamide; cytarabine; emitefur; hydroxyurea; mercaptopurine; nelarabine; pemetrexed; pentostatin; tegafur; and troxacitabine.

Non-limiting examples of platinum analogs include carboplatin, cisplatin, dicycloplatin, heptaplatin, lobaplatin, nedaplatin, oxaliplatin, satraplatin, and triplatin tetranitrate.

Non-limiting examples of enzymes include asparaginase and pegaspargase.

Non-limiting examples of topoisomerase inhibitors include acridine carboxamide, amonafide, amsacrine, belotecan, elliptinium acetate, exatecan, indolocarbazole, irinotecan, lurtotecan, mitoxantrone, razoxane, rubitecan, SN-38, sobuzoxane, and topotecan.

Non-limiting examples of retinoids include alitretinoin, bexarotene, fenretinide, isotretinoin, liarozole, RII retinamide, and tretinoin.

Non-limiting examples of aziridines include benzodopa, carboquone, meturedopa, and uredopa.

Non-limiting examples of antibiotics include intercalating antibiotics; anthracenediones; anthracycline antibiotics such as aclarubicin, amrubicin, daunomycin, daunorubicin, doxorubicin, epirubicin, idarubicin, menogaril, nogalamycin, pirarubicin, and valrubicin; 6-diazo-5-oxo-L-norleucine; aclacinomysins; actinomycin; authramycin; azaserine; bleomycins; cactinomycin; calicheamicin; carabicin; carminomycin; carzinophilin; chromomycins; dactinomycin; detorubicin; esorubicin; esperamicins; geldanamycin; marcellomycin; mitomycins; mitomycin C; mycophenolic acid; olivomycins; novantrone; peplomycin; porfiromycin; potfiromycin; puromycin; quelamycin; rebeccamycin; rodorubicin; streptonigrin; streptozocin; tanespimycin; tubercidin; ubenimex; zinostatin; zinostatin stimalamer; and zorubicin.

In one embodiment, the additional anti-cancer agent(s) is a hormonal and/or anti-hormonal agent (i.e., hormone therapy). Non-limiting examples of hormonal and anti-hormonal agents include anti-androgens such as abiraterone, apalutamide, bicalutamide, darolutamide, enzalutamide, flutamide, goserelin, leuprolide, and nilutamide; anti-estrogens such as 4-hydroxy tamoxifen, aromatase inhibiting 4(5)-imidazoles, EM-800, fosfestrol, fulvestrant, keoxifene, LY 117018, onapristone, raloxifene, tamoxifen, toremifene, and trioxifene; anti-adrenals such as aminoglutethimide, dexaminoglutethimide, mitotane, and trilostane; androgens such as calusterone, dromostanolone propionate, epitiostanol, mepitiostane, and testolactone; abarelix; anastrozole; cetrorelix; deslorelin; exemestane; fadrozole; finasteride; formestane; histrelin (RL 0903); human chorionic gonadotropin; lanreotide; LDI 200 (Milkhaus); letrozole; leuprorelin; mifepristone; nafarelin; nafoxidine; osaterone; prednisone; thyrotropin alfa; and triptorelin.

In one embodiment, the additional anti-cancer agent(s) is an immunotherapeutic agent (i.e., immunotherapy). Non-limiting examples of immunotherapeutic agents include biological response modifiers, cytokine inhibitors, tumor vaccines, monoclonal antibodies, immune checkpoint inhibitors, colony-stimulating factors, and immunomodulators.

Non-limiting examples of biological response modifiers, including cytokine inhibitors (cytokines) such as interferons and interleukins, include interferon alfa/interferon alpha such as interferon alfa-2, interferon alfa-2a, interferon alfa-2b, interferon alfa-n1, interferon alfa-n3, interferon alfacon-1, peginterferon alfa-2a, peginterferon alfa-2b, and leukocyte alpha interferon; interferon beta such as interferon beta-1a, and interferon beta-1b; interferon gamma such as natural interferon gamma-1a, and interferon gamma-1b; aldesleukin; interleukin-1 beta; interleukin-2; oprelvekin; sonermin; tasonermin; and virulizin.

Non-limiting examples of tumor vaccines include APC8015, AVICINE, bladder cancer vaccine, cancer vaccine (Biomira), gastrin 17 immunogen, Maruyama vaccine, melanoma lysate vaccine, melanoma oncolysate vaccine (New York Medical College), melanoma vaccine (New York University), melanoma vaccine (Sloan Kettering Institute), TICE® BCG (Bacillus Calmette-Guerin), and viral melanoma cell lysates vaccine (Royal Newcastle Hospital).

Non-limiting examples of monoclonal antibodies include abagovomab, adecatumumab, aflibercept, alemtuzumab, blinatumomab, brentuximab vedotin, CA 125 MAb (Biomira), cancer MAb (Japan Pharmaceutical Development), daclizumab, daratumumab, denosumab, edrecolomab, gemtuzumab zogamicin, HER-2 and Fc MAb (Medarex), ibritumomab tiuxetan, idiotypic 105AD7 MAb (CRC Technology), idiotypic CEA MAb (Trilex), ipilimumab, lintuzumab, LYM-1-iodine 131 MAb (Techni clone), mitumomab, moxetumomab, ofatumumab, polymorphic epithelial mucin-yttrium 90 MAb (Antisoma), ranibizumab, rituximab, and trastuzumab.

Non-limiting examples of immune checkpoint inhibitors include anti-PD-1 agents or antibodies such as cemiplimab, nivolumab, and pembrolizumab; anti-PD-L1 agents or antibodies such as atezolizumab, avelumab, and durvalumab; anti-CTLA-4 agents or antibodies such as ipilumumab; anti-LAGI agents; and anti-OX40 agents.

Non-limiting examples of colony-stimulating factors include darbepoetin alfa, epoetin alfa, epoetin beta, filgrastim, granulocyte macrophage colony stimulating factor, lenograstim, leridistim, mirimostim, molgramostim, nartograstim, pegfilgrastim, and sargramostim.

Non-limiting examples of additional immunotherapeutic agents include BiTEs, CAR-T cells, GITR agonists, imiquimod, immunomodulatory imides (IMiDs), mismatched double stranded RNA (Ampligen), resiquimod, SRL 172, and thymalfasin.

In one embodiment, the additional anti-cancer agent(s) is a targeted therapy agent (i.e., targeted therapy). Targeted therapy agents include, for example, monoclonal antibodies and small molecule drugs. Non-limiting examples of targeted therapy agents include signal transduction inhibitors, growth factor inhibitors, tyrosine kinase inhibitors, EGFR inhibitors, histone deacetylase (HDAC) inhibitors, proteasome inhibitors, cell-cycle inhibitors, angiogenesis inhibitors, matrix-metalloproteinase (MMP) inhibitors, hepatocyte growth factor inhibitors, TOR inhibitors, KDR inhibitors, VEGF inhibitors, fibroblast growth factors (FGF) inhibitors, MEK inhibitors, ERK inhibitors, PI3K inhibitors, AKT inhibitors, MCL-1 inhibitors, BCL-2 inhibitors, SHP2 inhibitors, HER-2 inhibitors, BRAF-inhibitors, gene expression modulators, autophagy inhibitors, apoptosis inducers, antiproliferative agents, and glycolysis inhibitors.

Non-limiting examples of signal transduction inhibitors include tyrosine kinase inhibitors, multiple-kinase inhibitors, anlotinib, avapritinib, axitinib, dasatinib, dovitinib, imatinib, lenvatinib, lonidamine, nilotinib, nintedanib, pazopanib, pegvisomant, ponatinib, vandetanib, and EGFR inhibitory agents.

Non-limiting examples of EGFR inhibitory agents include small molecule antagonists of EGFR such as afatinib, brigatinib, erlotinib, gefitinib, lapatinib, and osimertinib; and antibody-based EGFR inhibitors, including any anti-EGFR antibody or antibody fragment that can partially or completely block EGFR activation by its natural ligand. Antibody-based EGFR inhibitory agents may include, for example, those described in Modjtahedi, H., et al., 1993, Br. J. Cancer 67:247-253; Teramoto, T., et al., 1996, Cancer 77:639-645; Goldstein et al, 1995, Clin. Cancer Res. 1: 1311-1318; Huang, S. M., et al., 1999, Cancer Res. 15:59(8): 1935-40; and Yang, X., et al., 1999, Cancer Res. 59: 1236-1243; monoclonal antibody Mab E7.6.3 (Yang, 1999 supra); Mab C225 (ATCC Accession No. HB-8508), or an antibody or antibody fragment having the binding specificity thereof; specific antisense nucleotide or siRNA; afatinib, cetuximab; matuzumab; necitumumab; nimotuzumab; panitumumab; and zalutumumab.

Non-limiting examples of histone deacetylase (HDAC) inhibitors include belinostat, panobinostat, romidepsin, and vorinostat.

Non-limiting examples of proteasome inhibitors include bortezomib, carfilzomib, ixazomib, marizomib (salinosporamide a), and oprozomib.

Non-limiting examples of cell-cycle inhibitors, including CDK inhibitors, include abemaciclib, alvocidib, palbociclib, and ribociclib.

In one embodiment, the additional anti-cancer agent(s) is an anti-angiogenic agent (or angiogenesis inhibitor) including, but not limited to, matrix-metalloproteinase (MMP) inhibitors; VEGF inhibitors; EGFR inhibitors; TOR inhibitors such as everolimus and temsirolimus; PDGFR kinase inhibitory agents such as crenolanib; HIF-1α inhibitors such as PX 478; HIF-2α inhibitors such as belzutifan and the HIF-2α inhibitors described in WO 2015/035223; fibroblast growth factor (FGF) or FGFR inhibitory agents such as B-FGF and RG 13577; hepatocyte growth factor inhibitors; KDR inhibitors; anti-Ang1 and anti-Ang2 agents; anti-Tie2 kinase inhibitory agents; Tek antagonists (US 2003/0162712; U.S. Pat. No. 6,413,932); anti-TWEAK agents (U.S. Pat. No. 6,727,225); ADAM distintegrin domain to antagonize the binding of integrin to its ligands (US 2002/0042368); anti-eph receptor and/or anti-ephrin antibodies or antigen binding regions (U.S. Pat. Nos. 5,981,245; 5,728,813; 5,969,110; 6,596,852; 6,232,447; and 6,057,124); and anti-PDGF-BB antagonists as well as antibodies or antigen binding regions specifically binding to PDGF-BB ligands.

Non-limiting examples of matrix-metalloproteinase (MMP) inhibitors include MMP-2 (matrix-metalloproteinase 2) inhibitors, MMP-9 (matrix-metalloproteinase 9) inhibitors, prinomastat, RO 32-3555, and RS 13-0830. Examples of useful matrix metalloproteinase inhibitors are described, for example, in WO 96/33172, WO 96/27583, EP 1004578, WO 98/07697, WO 98/03516, WO 98/34918, WO 98/34915, WO 98/33768, WO 98/30566, EP 0606046, EP 0931788, WO 90/05719, WO 99/52910, WO 99/52889, WO 99/29667, WO 1999/007675, EP 1786785, EP 1181017, US 2009/0012085, U.S. Pat. Nos. 5,863,949, 5,861,510, and EP 0780386. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13).

Non-limiting examples of VEGF and VEGFR inhibitory agents include bevacizumab, cediranib, CEP 7055, CP 547632, KRN 633, orantinib, pazopanib, pegaptanib, pegaptanib octasodium, semaxanib, sorafenib, sunitinib, VEGF antagonist (Borean, Denmark), and VEGF-TRAP™.

The additional anti-cancer agent(s) may also be another anti-angiogenic agent including, but not limited to, 2-methoxyestradiol, AE 941, alemtuzumab, alpha-D148 Mab (Amgen, US), alphastatin, anecortave acetate, angiocidin, angiogenesis inhibitors, (SUGEN, US), angiostatin, anti-Vn Mab (Crucell, Netherlands), atiprimod, axitinib, AZD 9935, BAY RES 2690 (Bayer, Germany, BC1 (Genoa Institute of Cancer Research, Italy), beloranib, benefin (Lane Labs, US), cabozantinib, CDP 791 (Celltech Group, UK), chondroitinase AC, cilengitide, combretastatin A4 prodrug, CP 564959 (OSI, US), CV247, CYC381 (Harvard University, US), E 7820, EHT 0101, endostatin, enzastaurin hydrochloride, ER-68203-00 (IVAX, US), fibrinogen-E fragment, Flk-1 (ImClone Systems, US), forms of FLT 1 (VEGFR 1), FR-111142, GCS-100, GW 2286 (GlaxoSmithKline, UK), IL-8, ilomastat, IM-862, irsogladine, KM-2550 (Kyowa Hakko, Japan), lenalidomide, lenvatinib, MAb alpha5beta3 integrin, second generation (Applied Molecular Evolution, USA and MedImmune, US), MAb VEGF (Xenova, UK), marimastat, maspin (Sosei, Japan), metastatin, motuporamine C, M-PGA, ombrabulin, OXI4503, PI 88, platelet factor 4, PPI 2458, ramucirumab, rBPI 21 and BPI-derived antiangiogenic (XOMA, US), regorafenib, SC-236, SD-7784 (Pfizer, US), SDX 103 (University of California at San Diego, US), SG 292 (Telios, US), SU-0879 (Pfizer, US), TAN-1120, TBC-1635, tesevatinib, tetrathiomolybdate, thalidomide, thrombospondin 1 inhibitor, Tie-2 ligands (Regeneron, US), tissue factor pathway inhibitors (EntreMed, US), tumor necrosis factor-alpha inhibitors, tumstatin, TZ 93, urokinase plasminogen activator inhibitors, vadimezan, vandetanib, vasostatin, vatalanib, VE-cadherin-2 antagonists, xanthorrhizol, XL 784 (Exelixis, US), ziv-aflibercept, and ZD 6126.

In embodiments, the additional anti-cancer agent(s) is an additional active agent that disrupts or inhibits RAS-RAF-ERK or PI3K-AKT-TOR signaling pathways or is a PD-1 and/or PD-L1 antagonist. In embodiments, the additional anti-cancer agent(s) is a RAF inhibitor, EGFR inhibitor, MEK inhibitor, ERK inhibitor, PI3K inhibitor, AKT inhibitor, TOR inhibitor, MCL-1 inhibitor, BCL-2 inhibitor, SHP2 inhibitor, proteasome inhibitor, or immune therapy, including monoclonal antibodies, immunomodulatory imides (IMiDs), anti-PD-1, anti-PDL-1, anti-CTLA4, anti-LAGI, and anti-OX40 agents, GITR agonists, CAR-T cells, and BiTEs.

Non-limiting examples of RAF inhibitors include dabrafenib, encorafenib, regorafenib, sorafenib, and vemurafenib.

Non-limiting examples of MEK inhibitors include binimetinib, CI-1040, cobimetinib, PD318088, PD325901, PD334581, PD98059, refametinib, selumetinib, and trametinib.

Non-limiting examples of ERK inhibitors include LY3214996, LTT462, MK-8353, SCH772984, ravoxertinib, ulixertinib, and an ERKi as described in WO 2017/068412.

Non-limiting examples of PI3K inhibitors include 17-hydroxywortmannin analogs (e.g., WO 06/044453); AEZS-136; alpelisib; AS-252424; buparlisib; CAL263; copanlisib; CUDC-907; dactolisib (WO 06/122806); demethoxyviridin; duvelisib; GNE-477; GSK1059615; IC87114; idelalisib; INK1117; LY294002; Palomid 529; paxalisib; perifosine; PI-103; PI-103 hydrochloride; pictilisib (e.g., WO 09/036,082; WO 09/055,730); PIK 90; PWT33597; SF1126; sonolisib; TGI 00-115; TGX-221; XL147; XL-765; wortmannin; and ZSTK474.

Non-limiting examples of AKT inhibitors include Akt-1-1 (inhibits Aktl) (Barnett et al. (2005) Biochem. J., 385 (Pt. 2), 399-408); Akt-1-1,2 (Barnett et al. (2005) Biochem. J. 385 (Pt. 2), 399-408); API-59CJ-Ome (e.g., Jin et al. (2004) Br. J. Cancer 91, 1808-12); 1-H-imidazo[4,5-c]pyridinyl compounds (e.g., WO05011700); indole-3-carbinol and derivatives thereof (e.g., U.S. Pat. No. 6,656,963; Sarkar and Li (2004) J Nutr. 134(12 Suppl), 3493S-3498S); perifosine, Dasmahapatra et al. (2004) Clin. Cancer Res. 10(15), 5242-52, 2004); phosphatidylinositol ether lipid analogues (e.g., Gills and Dennis (2004) Expert. Opin. Investig. Drugs 13, 787-97); triciribine (Yang et al. (2004) Cancer Res. 64, 4394-9); imidazooxazone compounds including trans-3-amino-1-methyl-3-[4-(3-phenyl-5H-imidazo[1,2-c]pyrido[3,4-e][1,3]oxazin-2-yl)phenyl]-cyclobutanol hydrochloride (WO 2012/137870); afuresertib; capivasertib; MK2206; patasertib, and those disclosed in WO 2011/082270 and WO 2012/177844.

Non-limiting examples of TOR inhibitors include deforolimus; ATP-competitive TORC1/TORC2 inhibitors, including PI-103, PP242, PP30, and Torin 1; TOR inhibitors in FKBP12 enhancer, rapamycins and derivatives thereof, including temsirolimus, everolimus, WO 9409010; rapalogs, e.g. as disclosed in WO 98/02441 and WO 01/14387, e.g. AP23573, AP23464, or AP23841; 40-(2-hydroxyethyl)rapamycin, 40-[3-hydroxy (hydroxymethyl)methylpropanoate]-rapamycin; 40-epi-(tetrazolyl)-rapamycin (also called ABT578); 32-deoxorapamycin; 16-pentynyloxy-32(S)-dihydrorapanycin, and other derivatives disclosed in WO 05/005434; derivatives disclosed in U.S. Pat. No. 5,258,389, WO 94/090101, WO 92/05179, U.S. Pat. Nos. 5,118,677, 5,118,678, 5,100,883, 5,151,413, 5,120,842, WO 93/111130, WO 94/02136, WO 94/02485, WO 95/14023, WO 94/02136, WO 95/16691, WO 96/41807, WO 96/41807 and U.S. Pat. No. 5,256,790; and phosphorus-containing rapamycin derivatives (e.g., WO 05/016252).

Non-limiting examples of MCL-1 inhibitors include AMG-176, MIK665, and S63845.

Non-limiting examples of SHP2 inhibitors include SHP2 inhibitors described in WO 2019/167000 and WO 2020/022323.

Additional non-limiting examples of anti-cancer agents that are suitable for use include 2-ethylhydrazide, 2,2′,2″-trichlorotriethylamine, ABVD, aceglatone, acemannan, aldophosphamide glycoside, alpharadin, amifostine, aminolevulinic acid, anagrelide, ANCER, ancestim, anti-CD22 immunotoxins, antitumorigenic herbs, apaziquone, arglabin, arsenic trioxide, azathioprine, BAM 002 (Novelos), bcl-2 (Genta), bestrabucil, biricodar, bisantrene, bromocriptine, brostallicin, bryostatin, buthionine sulfoximine, calyculin, cell-cycle nonspecific antineoplastic agents, celmoleukin, clodronate, clotrimazole, cytarabine ocfosfate, DA 3030 (Dong-A), defofamine, denileukin diftitox, dexrazoxane, diaziquone, dichloroacetic acid, dilazep, discodermolide, docosanol, doxercalciferol, edelfosine, eflornithine, EL532 (Elan), elfomithine, elsamitrucin, eniluracil, etanidazole, exisulind, ferruginol, folic acid replenisher such as frolinic acid, gacytosine, gallium nitrate, gimeracil/oteracil/tegafur combination (S-1), glycopine, histamine dihydrochloride, HIT diclofenac, HLA-B7 gene therapy (Vical), human fetal alpha fetoprotein, ibandronate, ibandronic acid, ICE chemotherapy regimen, imexon, iobenguane, IT-101 (CRLX101), laniquidar, LC 9018 (Yakult), leflunomide, lentinan, levamisole+fluorouracil, lovastatin, lucanthone, masoprocol, melarsoprol, metoclopramide, miltefosine, miproxifene, mitoguazone, mitozolomide, mopidamol, motexafin gadolinium, MX6 (Galderma), naloxone+pentazocine, nitracrine, nolatrexed, NSC631570 octreotide (Ukrain), olaparib, P-30 protein, PAC-1, palifermin, pamidronate, pamidronic acid, pentosan polysulfate sodium, phenamet, picibanil, pixantrone, platinum, podophyllinic acid, porfimer sodium, PSK (Polysaccharide-K), rabbit antithymocyte polyclonal antibody, rasburiembodiment, retinoic acid, rhenium Re 186 etidronate, romurtide, samarium (153 Sm) lexidronam, sizofiran, sodium phenylacetate, sparfosic acid, spirogermanium, strontium-89 chloride, suramin, swainsonine, talaporfin, tariquidar, tazarotene, tegafur-uracil, temoporfin, tenuazonic acid, tetrachlorodecaoxide, thrombopoietin, tin ethyl etiopurpurin, tirapazamine, TLC ELL-12, tositumomab-iodine 131, trifluridine and tipiracil combination, troponin I (Harvard University, US), urethan, valspodar, verteporfin, zoledronic acid, and zosuquidar.

The present disclosure further provides a method for using the compounds of Formula (I) or pharmaceutical compositions provided herein, in combination with radiation therapy to treat cancer. Techniques for administering radiation therapy are known in the art, and these techniques can be used in the combination therapy described herein. The administration of the compound of Formula (I) in this combination therapy can be determined as described herein.

Radiation therapy can be administered through one of several methods, or a combination of methods, including, without limitation, external-beam therapy, internal radiation therapy, implant radiation, stereotactic radiosurgery, systemic radiation therapy, radiotherapy and permanent or temporary interstitial brachy therapy. The term “brachytherapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive microspheres.

The present disclosure also provides methods for combination therapies in which the additional active agent is known to modulate other pathways, or other components of the same pathway, or even overlapping sets of target enzymes which are used in combination with a compound of Formula (I), or a pharmaceutically acceptable salt thereof. In one embodiment, such therapy includes, but is not limited to, the combination of one or more compounds of Formula (I) with chemotherapeutic agents, immunotherapeutic agents, hormonal therapy agents, therapeutic antibodies, targeted therapy agents, and radiation treatment, to provide a synergistic or additive therapeutic effect.

The compounds of the disclosure can be used in combination with the agents disclosed herein or other suitable agents, depending on the condition being treated. Hence, in some embodiments the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound of Formula (I) and any of the agents described above can be formulated together in the same dosage form and administered simultaneously. Alternatively, a compound of Formula (I) and any of the agents described above can be simultaneously administered, wherein both the agents are present in separate formulations. In another alternative, a compound of Formula (I) can be administered just followed by and any of the agents described above, or vice versa. In some embodiments of the separate administration protocol, a compound of Formula (I) and any of the agents described above are administered a few minutes apart, or a few hours apart, or a few days apart.

As one aspect of the present disclosure contemplates the treatment of the disease/conditions with a combination of pharmaceutically active compounds that may be administered separately, the disclosure further relates to combining separate pharmaceutical compositions in kit form. The kit comprises two separate pharmaceutical compositions: a compound of Formula (I), and a second pharmaceutical compound. The kit comprises a container for containing the separate compositions such as a divided bottle or a divided foil packet. Additional examples of containers include syringes, boxes, and bags. In some embodiments, the kit comprises directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

The present disclosure also provides for the compound of Formula (I), or the pharmaceutically acceptable salt thereof, for use in therapy, or use of the compound of Formula (I), or the pharmaceutically acceptable salt thereof, in therapy. The present disclosure also provides for the compound of Formula (I), or the pharmaceutically acceptable salt thereof, for use in treating cancer, or use of a compound of Formula (I), or the pharmaceutically acceptable salt thereof, for treating cancer. The present disclosure also provides for the compound of Formula (I), or the pharmaceutically acceptable salt thereof, for the preparation of a medicament for the treatment of cancer, or use of the compound of Formula (I), or the pharmaceutically acceptable salt thereof, for the preparation of a medicament for the treatment of cancer. The present disclosure also provides for the compound of Formula (I), or the pharmaceutically acceptable salt thereof, and an additional anti-cancer agent, for use in the treatment of cancer, or use of the compound of Formula (I), or the pharmaceutically acceptable salt thereof, and the additional anti-cancer agent for treating cancer. The disclosure also provides the compound of Formula (I), or the pharmaceutically acceptable salt thereof, and an additional anti-cancer agent, for the preparation of a medicament for the treatment of cancer, or use of the compound of Formula (I), or the pharmaceutically acceptable salt thereof, and the additional anti-cancer agent, for the preparation of a medicament for the treatment of cancer. The present disclosure also provides for a pharmaceutical composition comprising the compound of Formula (I), or the pharmaceutically acceptable salt thereof, for use in the treatment of cancer, or use of the pharmaceutical composition comprising the compound of Formula (I), or the pharmaceutically acceptable salt thereof, for treating cancer. The present disclosure also provides for a pharmaceutical composition comprising the compound of Formula (I), or the pharmaceutically acceptable salt thereof, and an additional anti-cancer agent, for use in the treatment of cancer, or use of the pharmaceutical composition comprising the compound of Formula (I), or the pharmaceutically acceptable salt thereof, and the additional anti-cancer agent, for treating cancer.

### Methods of Preparing the Compounds of the Disclosure

The compounds described herein can be prepared according to the procedures of the following schemes and examples, using appropriate materials and are further exemplified by the following specific examples. The examples further illustrate details for the preparation of the compounds of the present disclosure. Those skilled in the art will readily understand that known variations of the conditions and processes of the following preparative procedures can be used to prepare these compounds. For instance, in some cases, the order of carrying out the steps of reaction schemes may be varied to facilitate the reaction or to avoid unwanted reaction products. These examples are provided for the purpose of further illustration only and are not intended to be limitations on the disclosure.

Throughout the synthetic schemes and examples, abbreviations and acronyms may be used with the following meanings unless otherwise indicated: s: singlet, d: doublet, t: triplet, q: quartet, sep: septet, dd: double doublet, dt: double triplet, td: triple doublet, tt: triple triplet, ddd: double double doublet, ddt: double double triplet, dtd: double triple doublet, tdd: triple double doublet, m: multiplet, br: broad, brs: broad singlet, tert: tertiary, DMSO-d₆: deuterated dimethyl sulfoxide, CDCl₃: deuterated chloroform, CD₃OD: deuterated methanol, THF: tetrahydrofuran, DMF: N,N-dimethylformamide, DMSO: dimethyl sulfoxide, DCM: dichloromethane, IPE: diisopropyl ether, MTBE: methyl tert-butyl ether, EtOAc: ethyl acetate, AcOH: acetic acid, TFA: trifluoroacetic acid, MeOH: methanol, EtOH: ethanol, DIAD: diisopropyl azodicarboxylate, TMAD: N,N,N′,N′-tetramethylazodicarboxamide, Et₃N: triethylamine, DIEA: N,N-diisopropylethylamine, RT: room temperature, NIS: N-iodosuccinimide, Pd(dppf)Cl₂: [1,1′-Bis(diphenylphosphino)ferrocene]dichloropalladium(II), and HATU: 1-[Bis(dimethylamino)methylene]-1H-1,2,3-triazolo[4,5-b]pyridinium 3-Oxide Hexafluorophosphate, KOAc: potassium acetate.

The reagents used in the Examples are commercially available products unless indicated otherwise. Prepacked columns manufactured by Shoko Scientific Co., Ltd., or Biotage were used in silica gel column chromatography and basic silica gel column chromatography. AVANCE NEO 400 spectrometer (400 MHz; BRUKER) and AVANCE III HD 500 spectrometer (500 MHz; BRUKER) were used for NMR spectra. For a deuterated solvent containing tetramethylsilane, tetramethylsilane was used as the internal reference. For other cases, measurement was performed using an NMR solvent as the internal reference. All δ values are indicated in ppm. Microwave reaction was performed using an Initiator (trademark) manufactured by Biotage. XSelect CSH C18 OBD Prep Columns manufactured by Waters were used for preparative reversed-phase HPLC.

### Benzyl 2-methyl-5-oxoazepane-1-carboxylate (Int-J003-P1)

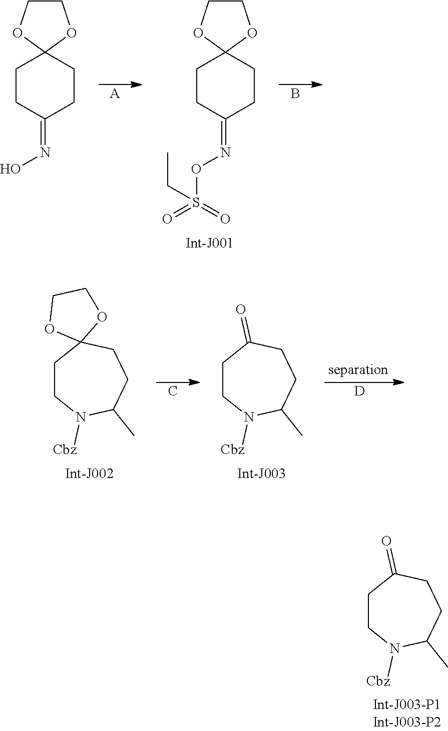

### Step A: 1,4-dioxaspiro[4.5]decan-8-one O-ethylsulfonyl oxime (Int-J001)

To a solution of 1,4-dioxaspiro[4.5]decan-8-one oxime (3.00 g, 17.5 mmol) and trimethylamine (2.96 mL, 21.0 mmol) in dichloromethane (30 mL) was added ethanesulfonyl chloride (1.84 mL, 19.3 mmol) at −15° C., the mixture was stirred at same temperature for 0.5 h. Water (15 mL) was added to the reaction mixture, and the organic layer was separated. The organic layer was washed with hydrochloric acid (1M, 15 mL), saturated sodium bicarbonate solution (15 mL) and brine (15 mL), and the organic layer was dried with sodium sulfate. The product solution was filtered, and the filtrate used for next step without any purification. ESI-MS m/z [M+H]⁺ 264.

### Step B: benzyl 9-methyl-1,4-dioxa-8-azaspiro[4.6]undecane-8-carboxylate (Int-J002)

To a stirred solution of trimethylaluminum (1.40 M in hexane, 25.0 mL, 35.0 mmol) was added the above 1,4-dioxaspiro[4.5]decan-8-one O-ethylsulfonyl oxime (Int-J001) solution in dichloromethane at −78° C. dropwise. The reaction mixture was warmed to 0° C. and stirred for 1 h. To the mixture was added diisobutylaluminum hydride (1.00 M in hexane, 26.3 mL, 26.3 mmol) dropwise, the mixture was stirred at 0° C. for 1 h. The reaction mixture was diluted with dichloromethane (15 mL), and sodium fluoride (10.5 g, 0.25 mol) and water (4.4 mL) were added to the reaction mixture. The mixture was stirred vigorously at room temperature for 1 h, and filtered. The filtrate was concentrated under reduce pressure, and ethyl acetate (50 mL) and water (50 mL) were added to the residue. To the mixture were added sodium bicarbonate (30 g, 0.36 mol) and benzyl chloroformate (11.9 g, 70.0 mmol) at room temperature, and the reaction mixture stirred for 1 h. The organic layer was separated and washed with water and concentrated under reduced pressure. The residue was purified by flash silica gel chromatography (0-40%, ethyl acetate gradient in hexane) to give benzyl 9-methyl-1,4-dioxa-8-azaspiro[4.6]undecane-8-carboxylate (Int-J002) (3.60 g). ESI-MS m/z [M+H]⁺ 306.

### Step C: benzyl 2-methyl-5-oxoazepane-1-carboxylate (Int-J003)

To a solution of benzyl 9-methyl-1,4-dioxa-8-azaspiro[4.6]undecane-8-carboxylate (Int-J002) (21.0 g, 68.8 mmol) in acetone (300 mL) and water (100 mL) was added toluene-4-sulfonic acid hydrate (19.6 g, 103 mmol). After stirring the mixture at 40° C. for 7 h, the reaction mixture was extracted with EtOAc, washed with brine, dried over Na₂SO₄, filtered and concentrated in vacuo. The residue was purified by flash silica gel chromatography (0-50%, EtOAc gradient in hexane) to afford benzyl 2-methyl-5-oxoazepane-1-carboxylate (Int-J003) (14.7 g). ESI-MS m/z [M+H]+ 262.

### Step D: benzyl 2-methyl-5-oxoazepane-1-carboxylate (Int-J003-P1 and Int-J003-P2))

Racemic benzyl 2-methyl-5-oxoazepane-1-carboxylate (Int-J003) was separated and the chiral benzyl 2-methyl-5-oxoazepane-1-carboxylate (Int-J003-P1, under the analytical condition below) is used for the synthesis.

Analytical condition: column (CHIRALCEL OD-H, 4.6 mmID×250 mmL, 5 μL), elutant (hexane/2-propanol, 700/300(v/v)), flow rate (1.0 mL/min), temperature (30° C.), concentration (0.5 mg/mL), injection volume (10 μL)

Retention time of benzyl 2-methyl-5-oxoazepane-1-carboxylate (Int-J003-P1): 6.053 min

Retention time of benzyl 2-methyl-5-oxoazepane-1-carboxylate (Int-J003-P2): 7.945 min

### 5,5-difluoro-2-methylazepane (Int-K002)

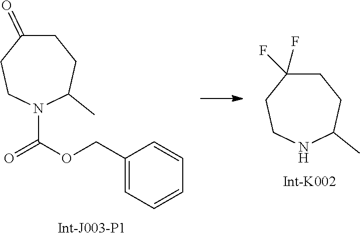

To a solution of benzyl 2-methyl-5-oxoazepane-1-carboxylate (Int-J003-P1) (200 mg, 0.765 mmol) in dichloromethane (1.5 mL) was added dimethylaminosulfur trifluoride (0.700 mL, 6.41 mmol) at 0° C. After stirring the mixture at room temperature for 2 h, the reaction was quenched by the addition of saturated aqueous Na₂CO₃. The reaction mixture was extracted with CHCl₃, washed with brine, dried over Na₂SO₄, filtered and concentrated in vacuo. The residue was purified by flash silica gel chromatography (0-50%, EtOAc gradient in hexane) to afford difluorinated compound.

To a solution of difluorinated compound in ethanol (10 mL) was added Pd(OH)₂/C (269 mg, 0.383 mmol). The reaction mixture was purged with H₂ gas and vacuum three times and stirred at room temperature for 1 h. The reaction mixture was filtered and concentrated in vacuo to afford 5,5-difluoro-2-methylazepane (Int-K002) (20.0 mg). ESI-MS m/z [M+H]⁺ 150.

### 7-methylazepan-4-ol hydrochloride (Int-K003-A)

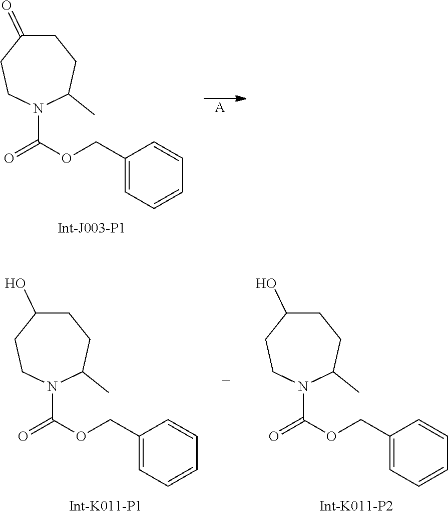

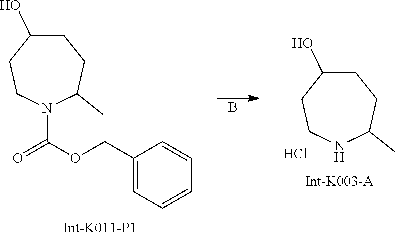

### Step A: benzyl 5-hydroxy-2-methylazepane-1-carboxylate (Int-K011-P1 and P2)

To a solution of benzyl 2-methyl-5-oxoazepane-1-carboxylate (Int-J003P1) (400 mg, 1.53 mmol) in MeOH (16 mL) was added 1.0 M THF solution of Lithium tri-sec-butylborohydride (1.53 mL, 1.53 mmol) at 0° C. After stirring the mixture at room temperature for 1 h, the reaction was quenched by the addition of H₂O. The reaction mixture was extracted with EtOAc, washed with brine, dried over Na₂SO₄, filtered and concentrated in vacuo. The residue was purified by flash silica gel chromatography (0-50%, EtOAc gradient in hexane) to afford benzyl 5-hydroxy-2-methylazepane-1-carboxylate (Int-K011-P1, first elution). ESI-MS m/z [M+H]⁺ 264. And benzyl 5-hydroxy-2-methylazepane-1-carboxylate (Int-K011-P2, second elution). ESI-MS m/z [M+H]⁺ 264.

### Step B: 7-methylazepan-4-ol hydrochloride (Int-K003-A)

To a solution of benzyl 5-hydroxy-2-methylazepane-1-carboxylate (Int-K011-P1, first elution) (79.0 mg, 0.300 mmol) in ethanol (3.0 mL) was added Pd(OH)₂/C (105 mg, 0.150 mmol). The reaction mixture was purged with H₂ gas and vacuum three times and stirred at room temperature for 30 min. The reaction mixture was filtered, 4.0 M dioxane solution of HCl (1.0 mL) was added to the filtrate. The filtrate was concentrated in vacuo to afford 7-methylazepan-4-ol hydrochloride (Int-K003-A). ESI-MS m/z [M+H]⁺ 130.

### 7-methylazepan-4-ol hydrochloride (Int-K003-B)

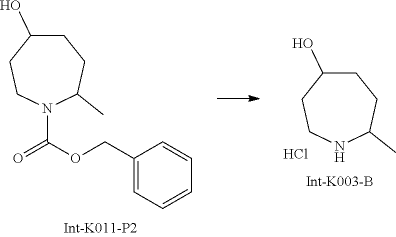

7-methylazepan-4-ol hydrochloride (Int-K003-B) was synthesized via a similar route as 7-methylazepan-4-ol hydrochloride (Int-K003-A) using benzyl 5-hydroxy-2-methylazepane-1-carboxylate (Int-K011-P2, second elution) instead of benzyl 5-hydroxy-2-methylazepane-1-carboxylate (Int-K011-P1, first elution). ESI-MS m/z [M+H]⁺ 130.

### 5-fluoro-2-methylazepane hydrochloride (Int-K004-A)

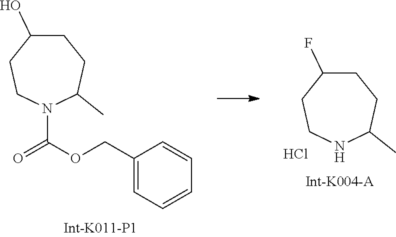

To a solution of benzyl 5-hydroxy-2-methylazepane-1-carboxylate (Int-K011-P1, first elution) (175 mg, 0.665 mmol) in dichloromethane (1.3 mL) was added dimethylaminosulfur trifluoride (0.143 mL, 1.31 mmol) at 0° C. After stirring the mixture at room temperature for 1 h, the reaction was quenched by the addition of saturated aqueous Na₂CO₃. The reaction mixture was extracted with CHCl₃, washed with brine, dried over Na₂SO₄, filtered and concentrated in vacuo. The residue was purified by flash silica gel chromatography (0-50%, EtOAc gradient in hexane) to afford fluorinated compound.

To a solution of fluorinated compound in ethanol (5.0 mL) was added Pd(OH)₂/C (69.3 mg, 0.0987 mmol). The reaction mixture was purged with H₂ gas and vacuum three times and stirred at room temperature for 1 h. The reaction mixture was filtered, 4.0 M dioxane solution of HCl (1.0 mL) was added to the filtrate. The filtrate was concentrated in vacuo to afford 5-fluoro-2-methylazepane hydrochloride (Int-K004-A). ESI-MS m/z [M+H]⁺ 132.

### 5-fluoro-2-methylazepane hydrochloride (Int-K004-B)

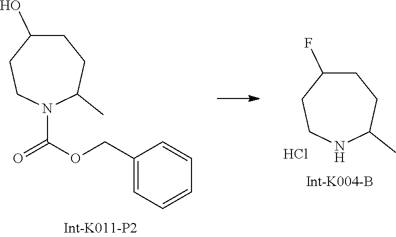

5-fluoro-2-methylazepane hydrochloride (Int-K004-B) was synthesized via a similar route as 5-fluoro-2-methylazepane hydrochloride (Int-K004-A) using benzyl 5-hydroxy-2-methylazepane-1-carboxylate (Int-K011-P2, second elution) instead of benzyl 5-hydroxy-2-methylazepane-1-carboxylate (Int-K011-P1, first elution). ESI-MS m/z [M+H]⁺ 132.

### 2-(3-hydroxypiperidin-3-yl)acetonitrile hydrochloride (Int-XX023)

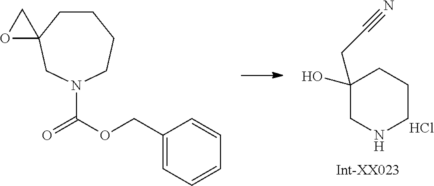 [SMILES: O=C(OCc1ccccc1)N1CCCCC2(CO2)C1 ; N#CCC1(O)CCCNC1 ; Cl]

To a solution of benzyl 1-oxa-5-azaspiro[2.5]octane-5-carboxylate (950 mg, 3.84 mmol) in EtOH (20 mL) and water (20 mL) was added sodium cyanide (282 mg, 5.76 mmol). After stirring the mixture at room temperature for 4 h, the reaction was extracted with EtAOc, washed with brine, dried over Na₂SO₄, filtered and concentrated in vacuo. The residue was purified by flash silica gel chromatography (0-50%, EtOAc gradient in hexane) to cyanated compound.

To a solution of cyanated compound (110 mg, 0.401 mmol) in ethanol (2.0 mL) was added Pd(OH)₂/C (141 mg, 0.200 mmol). The reaction mixture was purged with H₂ gas and vacuum three times and stirred at room temperature for 1 h. The reaction mixture was filtered, 4.0 M dioxane solution of HCl (1.0 mL) was added to the filtrate. The filtrate was concentrated in vacuo to afford 2-(3-hydroxypiperidin-3-yl)acetonitrile hydrochloride (Int-XX023). ESI-MS m/z [M+H]⁺ 141.

### 3-hydroxy-8-iodo-1-naphthoic acid (Int-T002)

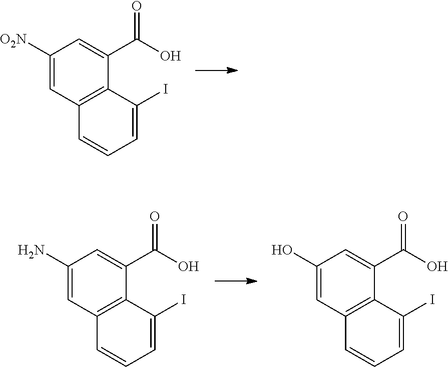 [SMILES: O=C(O)c1cc([N+](=O)[O-])cc2cccc(I)c12 ; Nc1cc(C(=O)O)c2c(I)cccc2c1 ; O=C(O)c1cc(O)cc2cccc(I)c12]

### Step A: 3-amino-8-iodo-1-naphthoic acid (Int-T001)

To a mixture of 8-iodo-3-nitro-1-naphthoic acid (1 g, 2.9 mmol) in ethyl acetate (40 mL) ethanol (15 mL) was added 5% Rh/C (0.5 g) and the flask charged with H₂. The mixture was stirred at room temperature. After 24 h, the reaction mixture was filtered through a pad of celite and the filtrate concentrated to dryness to afford crude 3-amino-8-iodo-1-naphthoic acid (Int-T001) (0.94 g) as a brown solid which was used in the next step without purification. ¹H NMR (400 MHZ, DMSO-d₆) δ=5.71 (brs, 2H), 6.87 (d, J=2.4 Hz, 1H), 7.00 (dd, J=8.1, 7.3 Hz, 1H), 7.12 (d, J=2.4 Hz, 1H), 7.60 (d, J=8.2 Hz, 1H), 7.8 (d, J=7.3 Hz, 1H), 13.14 (brs, 1H). LCMS (ESI): m/z [M+H]⁺ 314.

### Step B: 3-hydroxy-8-iodo-1-naphthoic acid (Int-T002)

To an ice cold mixture of crude 3-amino-8-iodo-1-naphthoic acid (Int-T001) (0.94 g) in 1M aqueous sulfuric acid (38 mL) was slowly added dropwise with a solution of sodium nitrite (0.228 g, 3.3 mmol) in water (1 mL). The mixture was stirred for 1 hour and warmed to room temperature. The reaction mixture was added dropwise to a refluxing solution of 40% aqueous sulfuric acid (108 mL). The reaction mixture was heated under reflux for 1 hour and then quickly hot-filtered through a plug of glass wool to remove insoluble, charred material. The filtrate was cooled to room temperature, and a precipitate was formed. The precipitate was collected by filtration and washed with water to give 3-hydroxy-8-iodo-1-naphthoic acid (Int-T002) (0.54 g). ¹H NMR (400 MHZ, DMSO-d₆) δ=7.13 (dd, J=8.1, 7.3 Hz, 1H), 7.22 (d, J=2.8 Hz, 1H), 7.24 (d, J=2.8 Hz, 1H), 7.82 (dd, J=8.3, 0.8 Hz, 1H), 8.01 (dd, J=7.3, 1.2 Hz, 1H), 10.22 (brs, 1H). LCMS (ESI): m/z [M+H]⁺ 315.

### (2-((1-((dimethylamino)methyl)cyclopropyl)methoxy)-4-hydroxy-5,7-dihydro-6H-pyrrolo[3,4-d]pyrimidin-6-yl)(3-hydroxy-8-iodonaphthalen-1-yl)methanone dihydrochloride (Int-L004)

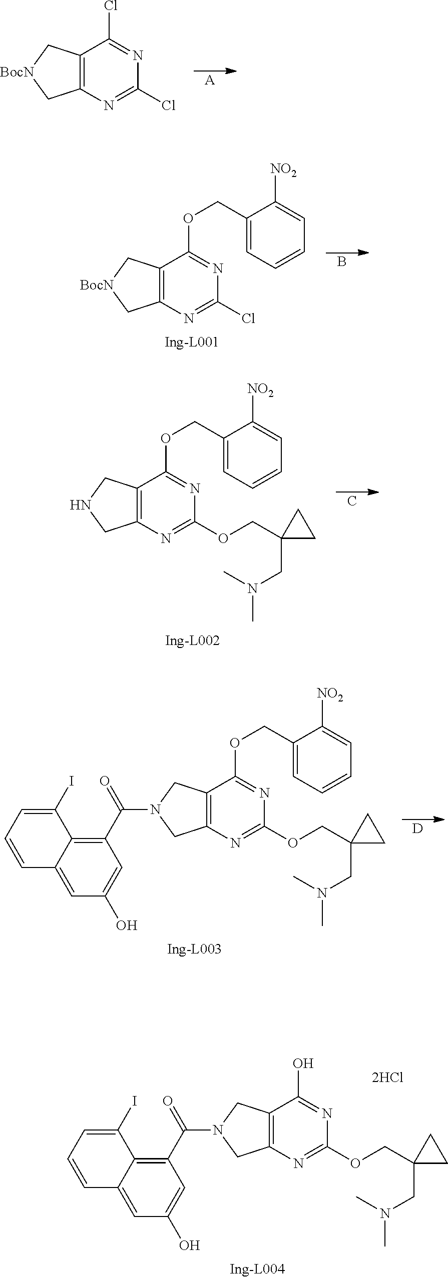

### Step A: tert-butyl 2-chloro-4-((2-nitrobenzyl)oxy)-5,7-dihydro-6H-pyrrolo[3,4-d]pyrimidine-6-carboxylate (Int-L001)

To a stirred solution of tert-butyl 2,4-dichloro-5,7-dihydro-6H-pyrrolo[3,4-d]pyrimidine-6-carboxylate (55.0 g, 0.190 mol) and (2-nitrophenyl)methanol (29.0 g, 0.190 mol) in toluene (550 mL) was added cesium carbonate (92.6 g, 0.284 mol), and the mixture was warmed to 85° C. and stirred for 14 h. The mixture was cooled to room temperature and filtered, and the filtrate was concentrated under reduced pressure. To the residue were added acetonitrile (400 mL) and water (200 mL) and the mixture was suspended for 2 days. The solid was filtered, and the solid was suspended in ethyl acetate (400 mL) at 85° C. for 1 h, then cooled to room temperature. The solid was filtered and dried under reduced pressure to give tert-butyl 2-chloro-4-((2-nitrobenzyl)oxy)-5,7-dihydro-6H-pyrrolo[3,4-d]pyrimidine-6-carboxylate (Int-L001) (21 g). ESI-MS m/z [M+H]⁺ 407, 409.

### Step B: N,N-dimethyl-1-(1-(((4-((2-nitrobenzyl)oxy)-6,7-dihydro-5H-pyrrolo[3,4-d]pyrimidin-2-yl)oxy)methyl)cyclopropyl)methanamine (Int-L002)

To a mixture of tert-butyl 2-chloro-4-((2-nitrobenzyl)oxy)-5,7-dihydro-6H-pyrrolo[3,4-d]pyrimidine-6-carboxylate (Int-L001) (10.0 g, 24.6 mmol), (1-((dimethylamino)methyl)cyclopropyl)methanol (4.13 g, 32.0 mmol), rac-BINAP (2.30 g, 3.69 mmol) and palladium acetate(II) (414 mg, 1.84 mmol) in toluene (100 mL) was added cesium carbonate (20.0 g, 61.5 mmol), and the mixture was warmed to 130° C. and stirred for 3 h. The mixture was cooled to room temperature and filtered, and the filtrate was concentrated under reduced pressure. The residue was purified by flash NH-silica gel chromatography (0-100%, ethyl acetate gradient in hexane) to give the coupling product. To a solution of the coupling product in dichloromethane (50 mL) was added trifluoroacetic acid (30 mL) and the mixture was stirred for 1 h. Trifluoroacetic acid was removed under reduced pressure and the residue was purified by flash NH-silica gel chromatography (0-50%, methanol gradient in ethyl acetate) to give N,N-dimethyl-1-(1-(((4-((2-nitrobenzyl)oxy)-6,7-dihydro-5H-pyrrolo[3,4-d]pyrimidin-2-yl)oxy)methyl)cyclopropyl)methanamine (Int-L002) (7.94 g). ESI-MS m/z [M+H]⁺ 400.

### Step C: (2-((1-((dimethylamino)methyl)cyclopropyl)methoxy)-4-((2-nitrobenzyl)oxy)-5,7-dihydro-6H-pyrrolo[3,4-d]pyrimidin-6-yl)(3-hydroxy-8-iodonaphthalen-1-yl)methanone (Int-L003)

To a mixture of 3-hydroxy-8-iodo-1-naphthoic acid (Int-T002) (5.19 g, 16.5 mmol), 1-hydroxybenzotriazole monohydrate (2.53 g, 16.5 mmol), 1-(3-dimethylaminopropyl)-3-ethylcarbodiimide hydrochloride (3.26 g, 16.5 mmol) and N,N-diisopropylethylamine (7.02 mL, 41.3 mmol) in N,N-dimethylformamide (55 mL) was added N,N-dimethyl-1-(1-(((4-((2-nitrobenzyl)oxy)-6,7-dihydro-5H-pyrrolo[3,4-d]pyrimidin-2-yl)oxy)methyl)cyclopropyl)methanamine (Int-L002) (5.50 g, 13.8 mmol), and the mixture was stirred at room temperature for 7 h. The mixture was diluted with ethyl acetate and the diluted mixture was washed with water and the mixture was concentrated under reduced pressure. The residue was purified by flash NH-silica gel chromatography (0-60%, methanol gradient in ethyl acetate) to give (2-((1-((dimethylamino)methyl)cyclopropyl)methoxy)-4-((2-nitrobenzyl)oxy)-5, 7-dihydro-6H-pyrrolo[3,4-d]pyrimidin-6-yl)(3-hydroxy-8-iodonaphthalen-1-yl)methanone (Int-L003) (8.77 g). ESI-MS m/z [M+H]⁺ 696.

### Step D: (2-((1-((dimethylamino)methyl)cyclopropyl)methoxy)-4-hydroxy-5,7-dihydro-6H-pyrrolo[3,4-d]pyrimidin-6-yl)(3-hydroxy-8-iodonaphthalen-1-yl)methanone dihydrochloride (Int-L004)

To a stirred suspension of (2-((1-((dimethylamino)methyl)cyclopropyl)methoxy)-4-((2-nitrobenzyl)oxy)-5,7-dihydro-6H-pyrrolo[3,4-d]pyrimidin-6-yl)(3-hydroxy-8-iodonaphthalen-1-yl)methanone (Int-L003) (7.00 g, 10.1 mmol) and iron (powder, 2.81 g, 50.3 mmol) in tetrahydrofuran (56 mL) was added hydrochloric acid (1M, 56.0 mL, 5.56 mmol), and the mixture was stirred at room temperature for 2 h. The mixture was diluted with methanol (100 mL) and ethyl acetate (100 mL), and the mixture was filtered and concentrated to dryness. To the residue were added ethyl acetate (30 mL) and methanol (3 mL) and the mixture was suspended for 1 h. The precipitate was filtered and dried under reduced pressure to give (2-((1-((dimethylamino)methyl)cyclopropyl)methoxy)-4-hydroxy-5,7-dihydro-6H-pyrrolo[3,4-d]pyrimidin-6-yl)(3-hydroxy-8-iodonaphthalen-1-yl)methanone dihydrochloride (Int-L004) (6.80 g). ESI-MS m/z [M+H]⁺ 561.

### (4-hydroxy-2-((1-(morpholinomethyl)cyclopropyl)methoxy)-5,7-dihydro-6H-pyrrolo[3,4-d]pyrimidin-6-yl)(3-hydroxy-8-iodonaphthalen-1-yl)methanone dihydrochloride (Int-L005)

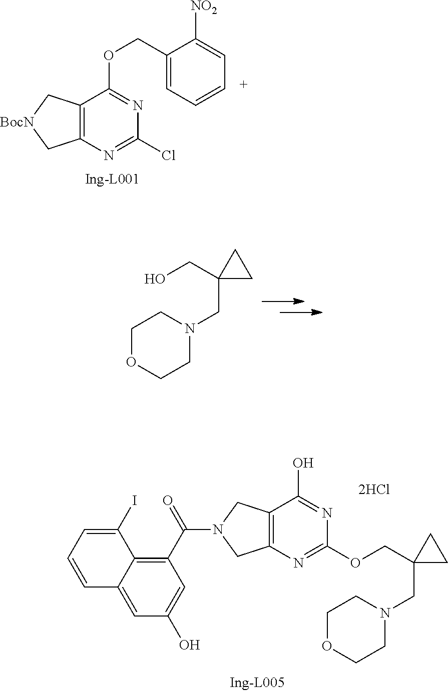 [SMILES: CC(C)(C)OC(=O)N1Cc2nc(Cl)nc(OCc3ccccc3[N+](=O)[O-])c2C1 ; OCC1(CN2CCOCC2)CC1 ; O=C(c1cc(O)cc2cccc(I)c12)N1Cc2nc(OCC3(CN4CCOCC4)CC3)nc(O)c2C1]

(4-hydroxy-2-((1-(morpholinomethyl)cyclopropyl)methoxy)-5,7-dihydro-6H-pyrrolo[3,4-d]pyrimidin-6-yl)(3-hydroxy-8-iodonaphthalen-1-yl)methanone dihydrochloride (Int-L005) was synthesized via a similar route as (2-((1-((dimethylamino)methyl)cyclopropyl)methoxy)-4-hydroxy-5,7-dihydro-6H-pyrrolo[3,4-d]pyrimidin-6-yl)(3-hydroxy-8-iodonaphthalen-1-yl)methanone dihydrochloride (Int-L004) using (1-(morpholinomethyl)cyclopropyl)methanol instead of (1-((dimethylamino)methyl)cyclopropyl)methanol. ESI-MS m/z [M+H]⁺ 603.

### (R)-(1-((dimethylamino)methyl)-2,2-difluorocyclopropyl)methanol (Int-UU8-1)

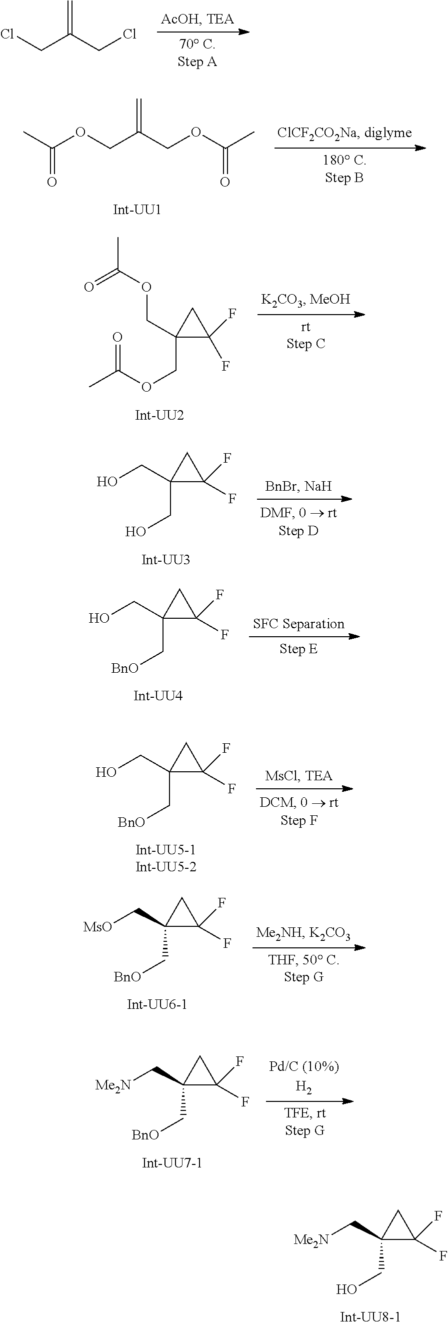

### Step A: 2-[(acetyloxy)methyl]prop-2-en-1-yl acetate (Int-UU1)

A 5 L 4-necked round-bottom flask was charged with 3-chloro-2-(chloromethyl)prop-1-ene (600 g, 4.80 mol), triethylamine (1.46 kg, 14.40 mol), and acetic acid (721 g, 12.0 mol). The resulting solution was stirred overnight at 70° C. The reaction mixture was cooled to room temperature and quenched by the addition of 3 L of water. The resulting solution was extracted with ethyl acetate (3×1 L) and the combined organic layers were washed with brine solution (2×1 L). The organic layers were dried over anhydrous sodium sulfate. The dried solution was filtered and the filtrate was concentrated. The residue was purified by silica gel column with ethyl acetate/petroleum ether (1:6) to provide 2-[(acetyloxy)methyl]prop-2-en-1-yl acetate (Int-UU1).

### Step B: [1-[(acetyloxy)methyl]-2,2-difluorocyclopropyl]methyl acetate (Int-UU2)

Into a 20-L 4-necked round-bottom flask and maintained with an inert atmosphere of nitrogen was placed a solution of 2-[(acetyloxy)methyl]prop-2-en-1-yl acetate (Int-UU1) (600 g, 3.48 mol) in diglyme (5 L). This was followed by the addition of a solution of ClCF₂CO₂Na (2.65 kg, 17.4 mol) in diglyme (5 L) dropwise with stirring at 180° C. over 5 h. The resulting solution was stirred for 1 h at 180° C. The reaction mixture was cooled to room temperature and quenched by the addition of H₂O (5 L). The resulting solution was extracted with petroleum ether (4×2 L) and the organic layers were combined. The combined organic layers were washed with water (3×2 L) and dried over anhydrous sodium sulfate. The dried solution was filtered and the filtrate was concentrated to dryness to afford [1-[(acetyloxy)methyl]-2,2-difluorocyclopropyl]methyl acetate (Int-UU2), which was used directly into the next step without purification.

### Step C: [2,2-difluoro-1-(hydroxymethyl)cyclopropyl]methanol (Int-UU3)

Into a 20-L 4-necked round-bottomed flask were placed [1-[(acetyloxy)methyl]-2,2-difluorocyclopropyl]methyl acetate (Int-UU2) (800 g, 3.60 mol), MeOH (10 L) and K₂CO₃ (995 g, 7.20 mol). The resulting solution was stirred overnight at room temperature. The solids were filtered out. The filtrate was concentrated. The resulting mixture was then diluted by the addition of water (2 L). The resulting solution was extracted with ethyl acetate (5×1 L). The organic layers were combined and dried over anhydrous sodium sulfate. The dried solution was filtered and the filtrate was concentrated under vacuum. The residue was purified by silica gel column chromatography with ethyl acetate/petroleum ether (1:1) to afford [2,2-difluoro-1-(hydroxymethyl)cyclopropyl]methanol (Int-UU3). ¹H NMR (300 MHz, DMSO-d₆) δ 1.30 (t, J=8.8 Hz, 2H), 3.52 (m, 4H), 4.79 (t, J=5.6 Hz, 2H).

### Step D: (1-((benzyloxy)methyl)-2,2-difluorocyclopropyl)methanol (Int-UU4)

A 500 mL single neck round bottom flask fitted with a pour-through nitrogen adapter was purged with nitrogen and then charged with sodium hydride (4.52 g, 113 mmol) and N,N-dimethylformamide (100 mL). The suspension was cooled to 0° C. Solid [2,2-difluoro-1-(hydroxymethyl)cyclopropyl]methanol (Int-UU3) (12.0 g, 87 mmol) was added portionwise. The mixture was stirred while warming to rt for 1 h. The resultant reaction mixture was cooled to 0° C. and treated with a solution of benzyl bromide (10.3 mL, 87 mmol) in N,N-dimethylformamide (10 mL). The mixture was stirred at rt for 1 h and then treated with saturated aqueous ammonium chloride (10 mL) and water (10 mL). The mixture was partitioned between ethyl acetate (75 mL) and water (75 mL). The organic layer was washed with 1 wt % aqueous LiCl (30 mL×3), dried with anhydrous sodium sulfate, filtered and the filtrate was concentrated. Purification by column chromatography on silica gel (220 g, 0 to 40% EtOAc/hexanes) afforded (1-((benzyloxy)methyl)-2,2-difluorocyclopropyl)methanol (Int-UU4). 1H NMR (499 MHz, Methanol-d4) δ 7.39-7.32 (m, 4H), 7.32-7.26 (m, 1H), 4.62-4.49 (m, 2H), 3.79-3.64 (m, 3H), 3.60 (dd, J=10.4, 2.1 Hz, 1H), 1.35 (dddd, J=29.1, 12.5, 8.0, 4.5 Hz, 2H).

### Step E: (1-((benzyloxy)methyl)-2,2-difluorocyclopropyl)methanol (Int-UU5-1)

Racemic (1-((benzyloxy)methyl)-2,2-difluorocyclopropyl)methanol (Int-UU4) was resolved using SFC chiral chromatography (AD-H (21 mm×250 mm, 5 um; conditions: 5% MeOH w/0.1% NH₄OH and 5% H₂O) to yield (1-((benzyloxy)methyl)-2,2-difluorocyclopropyl)methanol (Int-UU5-1, Peak 1). 1H NMR (499 MHz, Methanol-d4) δ 7.39-7.26 (m, 5H), 4.58-4.51 (m, 2H), 3.78-3.67 (m, 3H), 3.60 (dd, J=10.4, 2.0 Hz, 1H), 1.35 (dddd, J=28.4, 12.5, 8.0, 4.5 Hz, 2H). (1-((benzyloxy)methyl)-2,2-difluorocyclopropyl)methanol (Int-UU5-2, Peak 2) was also isolated.

### Step F: (S)-(1-((benzyloxy)methyl)-2,2-difluorocyclopropyl)methyl methanesulfonate (Int-UU6-1)

(1-((benzyloxy)methyl)-2,2-difluorocyclopropyl)methanol (Int-UU5-1) (3.25 g, 14.24 mmol) in DCM (30 mL) was cooled to 0° C. and treated with triethylamine (7.94 mL, 57.0 mmol) and then a solution of methanesulfonyl chloride (2.22 mL, 28.5 mmol) in DCM (2.2 mL). The reaction mixture was stirred while warming to rt over 3 h. The reaction mixture was purified by column chromatography on silica gel (0 to 100% EtOAc/hexanes) to afford (S)-(1-((benzyloxy)methyl)-2,2-difluorocyclopropyl)methyl methanesulfonate (Int-UU6-1). MS (ESI): m/z [M+Na]⁺329.

### Step G: (R)-1-(1-((benzyloxy)methyl)-2,2-difluorocyclopropyl)-N,N-dimethylmethanamine (Int-UU7-1)

(S)-(1-((benzyloxy)methyl)-2,2-difluorocyclopropyl)methyl methanesulfonate (Int-UU6-1) (4.10 g, 13.4 mmol) and dimethylamine (2 M in THF) (33.5 mL, 66.9 mmol) were treated with potassium carbonate (3.70 g, 26.8 mmol). The flask was capped and heated at 50° C. for 24 h. The reaction mixture was cooled to rt, diluted with water (20 mL), and extracted with ethyl acetate. The combined organic layers were dried with anhydrous sodium sulfate, filtered and the filtrate was concentrated. The crude residue was purified by column chromatography on silica gel (120 g, 0 to 100% [1:3 EtOH/EtOAc]/hexanes) to afford (R)-1-(1-((benzyloxy)methyl)-2,2-difluorocyclopropyl)-N,N-dimethylmethanamine (Int-UU7-1). MS (ESI): m/z [M+H]⁺ 256.

### Step H: (R)-(1-((dimethylamino)methyl)-2,2-difluorocyclopropyl)methanol (Int-UU8-1)

(R)-1-(1-((benzyloxy)methyl)-2,2-difluorocyclopropyl)-N,N-dimethylmethanamine (Int-UU7-1) (2.96 g, 11.6 mmol) and Pd/C (10 wt %, wet support) (0.618 g, 0.580 mmol) in 2,2,2-trifluoroethanol (20.0 mL) were charged in a 100 mL recovery flask and stirred at rt under hydrogen gas (1 atm, balloon) for 20 h. The mixture was filtered through a pad of CELITE and the pad was rinsed with methanol (3×10 mL). The combined filtrate and washings were treated with 3M HCl in methanol (12 mL, 36.0 mmol) and then concentrated in vacuo to afford a clear, colorless viscous syrup. Diethyl ether (10 mL) was added and the mixture agitated to initiate precipitation. The mixture was concentrated in vacuo and then treated with diethyl ether (10 mL) and sonicated for 1 minute. The diethyl ether was decanted. The solid was dried under vacuum to afford (R)-(1-((dimethylamino)methyl)-2,2-difluorocyclopropyl)methanol (Int-UU8-1). MS (ESI): m/z [M+H]⁺ 166. (S)-(1-((dimethylamino)methyl)-2,2-difluorocyclopropyl)methanol (Int-UU8-2) was synthesized via a similar reaction sequence as above using Int-UU5-2 as an intermediate. MS (ESI): m/z [M+H]⁺ 166.

### (2R,7aS)-2-fluorotetrahydro-1H-pyrrolizin-7a(5H)-yl)methanol (Int-VV6)

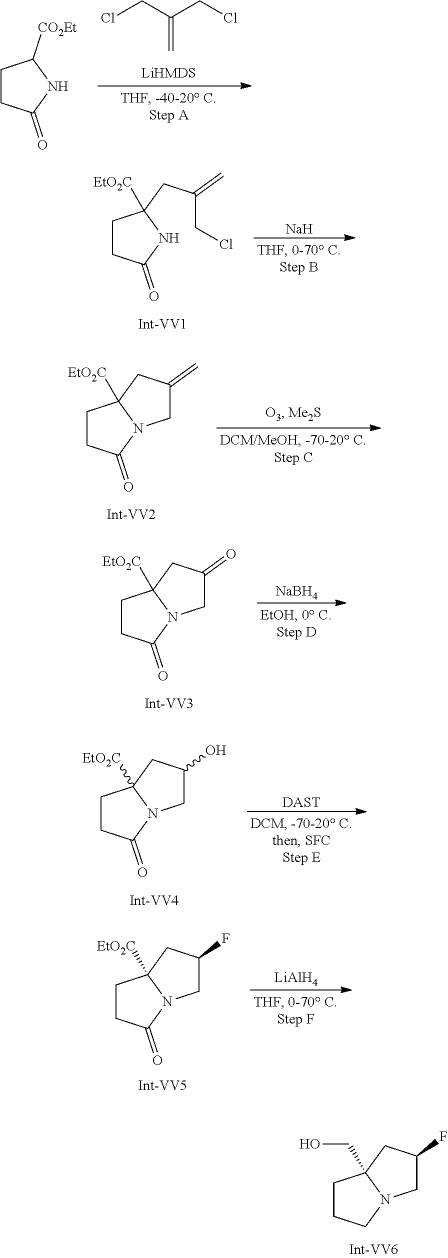

### Step A: ethyl 2-(2-(chloromethyl)allyl)-5-oxopyrrolidine-2-carboxylate (Int-VV1)

LiHMDS (1.00 M, 2.55 L) was added dropwise to a solution of ethyl 5-oxopyrrolidine-2-carboxylate (200, g, 1.27 mol) and 3-chloro-2-(chloromethyl)prop-1-ene (255 g, 2.04 mol, 236 mL) in THF (2.00 L) at −40° C. under N₂. The mixture was stirred at 20° C. for 20 h.

The reaction mixture was poured into sat. NH₄Cl solution (1.00 L) and the pH of the mixture was adjusted to 6˜7 with 1 N HCl. The biphasic solution was extracted with EtOAc (500 mL×3). The organic layers were combined, washed with brine (600 mL), and concentrated under reduced pressure to give a crude residue. The crude material was purified by silica gel column chromatography (Eluent: petroleum ether:ethyl acetate=50:1 to 1:1 gradient) to yield ethyl 2-(2-(chloromethyl)allyl)-5-oxopyrrolidine-2-carboxylate (Int-VV1). ¹H NMR (400 MHz, CDCl₃) δ 5.06 (br d, J=17 Hz, 2H), 4.14-4.38 (m, 3H), 3.73 (br d, J=16 Hz, 1H), 3.06 (br d, J=16 Hz, 1H), 2.70-2.85 (m, 1H), 2.53-2.66 (m, 1H), 2.36-2.50 (m, 2H), 2.09-2.21 (m, 1H), 1.23-1.31 (m, 3H).

### Step B: ethyl 2-methylene-5-oxotetrahydro-1H-pyrrolizine-7a(5H)-carboxylate (Int-VV2)

A solution of ethyl 2-(2-(chloromethyl)allyl)-5-oxopyrrolidine-2-carboxylate (Int-VV1) (500, g, 2.03 mol) in THF (500 mL) was added dropwise to a mixture of sodium hydride (97.7 g, 2.44 mol, 60.0% purity) in THF (3.00 L) at 0° C. under nitrogen. The reaction mixture was stirred at 70° C. for 12 h under nitrogen. The reaction mixture was cooled and poured into sat. ammonium chloride solution (2.00 L) and stirred at 5° C. for 1 h. The biphasic mixture was extracted with EtOAc (600 mL×3). The combined organic layers were washed with brine (500 mL×2), dried over anhydrous Na₂SO₄, filtered, and concentrated under reduced pressure. The crude residue was purified by silica gel column chromatography (Eluent: Petroleum ether:Ethyl acetate=50:1 to 1:1) to yield ethyl 2-methylene-5-oxotetrahydro-1H-pyrrolizine-7a(5H)-carboxylate (Int-VV2). ¹H NMR (400 MHZ, CDCl₃) δ 4.96-5.13 (m, 2H), 4.27 (br d, J=16 Hz, 1H), 4.19 (q, J=7 Hz, 2H), 3.71 (br d, J=16 Hz, 1H), 3.04 (d, J=16 Hz, 1H), 2.69-2.83 (m, 1H), 2.59 (ddd, J=2, 9, 13 Hz, 1H), 2.40-2.52 (m, 2H), 1.96-2.22 (m, 1H), 1.26 (t, J=7 Hz, 3H).

### Step C: ethyl 2,5-dioxotetrahydro-1H-pyrrolizine-7a(5H)-carboxylate (Int-VV3)

Ozone (239 mmol) (0.5˜1 m³/h) was bubbled into a solution of ethyl 2-methylene-5-oxotetrahydro-1H-pyrrolizine-7a(5H)-carboxylate (Int-VV2) (160, g, 765 mmol in DCM (1.60 L) and MeOH (160 mL) at −70° C. for 9 h. Nitrogen was bubbled through the reaction mixture to purge excess ozone. Then, dimethyl sulfide (76.0 g, 1.22 mol) was added to the mixture at −70° C. The reaction mixture was stirred at 20° C. for 14 h. The reaction mixture was concentrated under reduced pressure to give a residue. The crude residue was purified by silica gel column chromatography (Eluent: Petroleum ether:Ethyl acetate=50:1 to 1:1) to yield ethyl 2,5-dioxotetrahydro-1H-pyrrolizine-7a(5H)-carboxylate (Int-VV3). ¹H NMR (400 MHZ, CDCl₃) δ 4.22 (q, J=7 Hz, 2H), 4.07-4.12 (m, 1H), 3.54 (dd, J=1, 18 Hz, 1H), 2.92-3.03 (m, 2H), 2.74-2.88 (m, 1H), 2.42-2.51 (m, 2H), 2.12-2.23 (m, 1H), 1.27 (t, J=7 Hz, 3H)

### Step D: ethyl 2-hydroxy-5-oxotetrahydro-1H-pyrrolizine-7a(5H)-carboxylate (Int-VV4)

To a solution of ethyl 2,5-dioxotetrahydro-1H-pyrrolizine-7a(5H)-carboxylate (Int-VV3) (200 g, 947 mmol) in EtOH (2.00 L) at 0° C. under N₂ was added NaBH₄(10.8 g, 284 mmol). The reaction mixture was stirred at 0° C. for 10 min. The reaction mixture was quenched by addition of sat. NH₄Cl (50.0 mL) at 5° C. and the mixture was stirred at 5° C. for 0.5 h. The reaction mixture was concentrated under reduced pressure. The crude product from four identical reactions was then combined. The crude residue was purified by silica gel column chromatography (Eluent: Petroleum ether:Ethyl acetate=50:1 to 1:1) to yield ethyl 2-hydroxy-5-oxotetrahydro-1H-pyrrolizine-7a(5H)-carboxylate (Int-VV4). ¹H NMR (400 MHZ, CDCl₃) δ 4.54-4.70 (m, 1H), 4.16-4.31 (m, 2H), 3.93 (dd, J=6.0, 13 Hz, 1H), 3.09 (d, J=13 Hz, 1H), 2.75-2.90 (m, 1H), 2.39-2.63 (m, 4H), 2.01-2.13 (m, 1H), 1.83 (dd, J=6, 14 Hz, 1H), 1.29 (t, J=7 Hz, 3H).

### Step E: ethyl (2R,7aS)-2-fluoro-5-oxotetrahydro-1H-pyrrolizine-7a(5H)-carboxylate (Int-VV5)

To a solution of ethyl 2-hydroxy-5-oxotetrahydro-1H-pyrrolizine-7a(5H)-carboxylate (Int-VV4) in DCM (600 mL) was added DAST (90.7 g, 563 mmol, 74.4 mL) dropwise at −70° C. under N₂. The reaction mixture was warmed to 20° C. and stirred for 16 h. The reaction was quenched by the addition of EtOH (50.0 mL) at 10° C., and then diluted with water (300 mL) and extracted with DCM (200 mL×2). The combined organic layers were washed with brine (200 mL), dried over anhydrous Na₂SO₄, filtered, and concentrated under reduced pressure. The crude product was combined from six identical reactions and purified by silica gel column chromatography (Petroleum ether:Ethyl Acetate=50:1 to 1:1). This material was further purifed by prep-HPLC with HCl modifier (MeCN/water with 0.05% HCl modifier). The racemic mixture was resolved using chiral SFC (Daicel Chiralpak AS (50 mm×250 mm, 10 um; conditions: 0.1% NH₄OH in EtOH) to yield ethyl (2R,7aS)-2-fluoro-5-oxotetrahydro-1H-pyrrolizine-7a(5H)-carboxylate (Int-VV5, Peak 2). ¹H NMR (400 MHZ, CDCl₃): δ 5.16-5.43 (m, 1H), 4.14-4.27 (m, 3H), 3.06-3.26 (m, 1H), 2.57-2.85 (m, 3H), 2.38-2.50 (m, 1H), 2.07-2.30 (m, 2H), 1.28 (t, J=7 Hz, 3H).

### Step F: ((2R,7aS)-2-fluorotetrahydro-1H-pyrrolizin-7a(5H)-yl)methanol (Int-VV6)

A solution of ethyl (2R,7aS)-2-fluoro-5-oxotetrahydro-1H-pyrrolizine-7a(5H)-carboxylate (Int-VV5) (82.0 g, 381 mmol) in THF (300 mL) was added to the mixture of LAH (21.7 g, 571 mmol) in THF (520 mL) at 0° C. under nitrogen. The reaction mixture was warmed to 70° C. and stirred for 3 h. The reaction mixture was cooled to 0° C. and quenched by the addition of Na₂SO₄. 10 H₂O at 0° C. under nitrogen. The reaction mixture was stirred at 20° C. for 0.5 h and then filtered. The filter cake was washed with EtOAc (600 mL×5) and the filtrate was dried over anhydrous Mg₂SO₄. The mixture was filtered and the filtrate concentrated under reduced pressure to give a residue. The crude residue was purified by silica gel column chromatography (SiO₂, DCM:Methanol=100:1 to 10:1) to yield ((2R,7aS)-2-fluorotetrahydro-1H-pyrrolizin-7a(5H)-yl)methanol (Int-VV6). ¹H NMR (400 MHZ, CDCl₃): δ 5.06-5.34 (m, 1H), 3.25 (s, 2H), 3.08-3.23 (m, 3H), 2.85-3.08 (m, 2H), 2.00-2.12 (m, 2H), 1.74-1.93 (m, 4H).

### 4-bromo-6-methyl-1-(tetrahydro-2H-pyran-2-yl)-5-(trifluoromethyl)-1H-indazole (Int-HHH5)

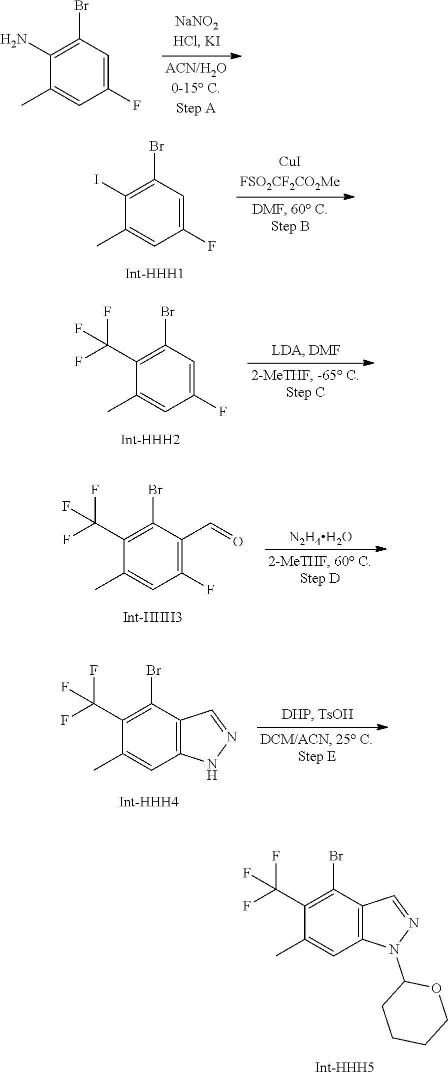

### Step A: 1-bromo-5-fluoro-2-iodo-3-methylbenzene (Int-HHH1)

2-Bromo-4-fluoro-6-methylaniline (200 g, 0.983 mol) was dissolved in MeCN (800 mL). The resulting mixture was cooled down to 0° C. Concentrated HCl (12 M, 245 mL) was added into the reaction mixture while maintaining the reaction temperature at 0° C. A solution of NaNO₂ (81.1 g, 1.18 mol eq) in water (400 mL) was added dropwise into the reaction mixture maintaining the reaction temperature at 0° C. The resulting mixture was stirred for 0.5 h at 0° C. Then a solution of KI (195 g, 1.18 mol) in water (400 mL) was added dropwise into the reaction mixture at 0° C. The resulting mixture was warmed up to room temperature and stirred for 12 h at 20° C. This reaction was repeated in one additional batch using the above conditions. The two batches of reactions were combined. The product mixture was adjusted to pH 8-9 by aq. NaOH and the aqueous phase was extracted with EtOAc (2.00 L×2). The organic phase was dried over Na₂SO₄, filtered, and concentrated. The residue obtained was purified by column chromatography (SiO₂, Petroleum ether:Ethyl acetate=1:0 to 0:1) to afford 1-bromo-5-fluoro-2-iodo-3-methylbenzene (Int-HHH1). ¹H NMR (400 MHz, CDCl₃) δ 7.27-7.22 (m, 1H), 6.95 (dd, J=2.4, 8.8 Hz, 1H), 2.56 (s, 3H).

### Step B: 1-bromo-5-fluoro-3-methyl-2-(trifluoromethyl)benzene (Int-HHH2)

1-bromo-5-fluoro-2-iodo-3-methylbenzene (Int-HHH1) (100 g, 0.317 mol) was dissolved in DMF (1.50 L). To this mixture were added CuI (514 g, 2.70 mol) and methyl 2,2-difluoro-2-(fluorosulfonyl)acetate (518 g, 2.70 mol) at 25° C. The reaction mixture was heated and stirred for 12 h at 60° C. This reaction was repeated in 3 additional batches using the above conditions. The four batches of reactions were combined and quenched with water (24 L). The mixture was extracted with petroleum ether (8.00 L×2). The combined organic layers were washed with brine (4 L×2) and dried over Na₂SO₄. The dried solution was filtered and the filtrate was concentrated in vacuo to afford the crude material containing 1-bromo-5-fluoro-3-methyl-2-(trifluoromethyl)benzene (Int-HHH2), which was used directly into the next step without purification.

### Step C: 2-bromo-6-fluoro-4-methyl-3-(trifluoromethyl)benzaldehyde (Int-HHH3)

1-bromo-5-fluoro-3-methyl-2-(trifluoromethyl)benzene (Int-HHH2) (100 g, 0.382 mol) was dissolved in 2-MeTHF (500 mL). The reaction mixture was cooled down to −65° C. A 2 M solution of LDA (213 mL, 426 mmol) was added into the mixture at −65° C. The reaction mixture was stirred for 0.5 h at −65° C. To this mixture was added dropwise DMF (31.2 g, 0.420 mol) at −65° C. The reaction mixture was stirred for 2 h at −65° C. This reaction was repeated in 2 additional batches using the above conditions. The three batches of reactions were combined. The reaction mixture pH was adjusted to 3-4 by using 1 M HCl and the aqueous phase was extracted with 2-MeTHF (500 mL×2). The organic phase was dried over Na₂SO₄, filtered, and concentrated to obtain 2-bromo-6-fluoro-4-methyl-3-(trifluoromethyl)benzaldehyde (Int-HHH3), which was used in the next step without further purification.

### Step D: 4-bromo-6-methyl-5-(trifluoromethyl)-1H-indazole (Int-HHH4)

2-bromo-6-fluoro-4-methyl-3-(trifluoromethyl)benzaldehyde (Int-HHH3) (100 g, 0.351 mol) was dissolved in THF (800 mL). To this mixture was added N₂H₄·H₂O (53.7 g, 1.05 mol) at 25° C. The mixture was heated and stirred for 2 h at 60° C. The product mixture was quenched with water (400 mL) and extracted with EtOAc (200 mL×2). The combined organic layers were washed with brine (200 mL) and dried over Na₂SO₄. The dried solution was filtered and the filtrate was concentrated in vacuo to give the residue. This reaction was repeated in 2 additional batches using the above conditions. The three batches of reactions were combined. The residue obtained was triturated with DCM (100 mL) at 15° C. for 2 h. The solid was collected by filtration to afford 4-bromo-6-methyl-5-(trifluoromethyl)-1H-indazole (Int-HHH4). ¹H NMR (400 MHZ, CDCl₃) δ 10.61-10.20 (m, 1H), 8.20 (d, J=0.8 Hz, 1H), 7.34 (d, J=0.6 Hz, 1H), 2.67-2.63 (m, 3H).

### Step E: 4-bromo-6-methyl-1-(tetrahydro-2H-pyran-2-yl)-5-(trifluoromethyl)-1H-indazole (Int-HHH5)

4-bromo-6-methyl-5-(trifluoromethyl)-1H-indazole (Int-HHH4) (60.0 g, 0.215 mol) was dissolved in DCM (240 mL) and MeCN (240 mL). DHP (21.7 g, 0.258 mol) and TsOH·H₂O (8.18 g, 0.043 mol) were added to the mixture at 20° C. The reaction mixture was stirred for 12 h at 20° C. Water (200 mL) was added to the product mixture. The resulting mixture was extracted with DCM (200 mL×2). The combined organic layers were washed with brine (200 mL) and dried over Na₂SO₄. The dried solution was filtered and the filtrate was concentrated under reduced pressure. The residue obtained was purified by column chromatography (SiO₂, Petroleum ether:Ethyl acetate=1:0 to 0:1) to afford 4-bromo-6-methyl-1-(tetrahydro-2H-pyran-2-yl)-5-(trifluoromethyl)-1H-indazole (Int-HHH5). 1H NMR (400 MHZ, CDCl₃-d) δ 8.12 (s, 1H), 7.44 (s, 1H), 5.69 (dd, J=3, 9 Hz, 1H), 4.09-3.94 (m, 1H), 3.81-3.69 (m, 1H), 2.69-2.63 (m, 3H), 2.56-2.43 (m, 1H), 2.19-2.14 (m, 1H), 2.12-2.04 (m, 1H), 1.87-1.73 (m, 2H), 1.71-1.63 (m, 1H).

### 1,8-dibromo-3-(methoxymethoxy)naphthalene (Int-W002)

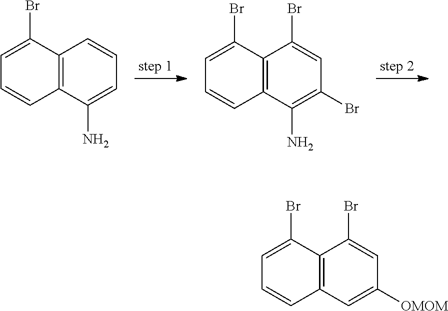

### Step A: 2,4,5-tribromonaphthalen-1-amine (Int-W001)

To a solution of 5-bromonaphthalen-1-amine (63 g, 280 mmol) in DMA (1260 mL) was added NBS (106 g) at 0° C. The mixture was allowed to warm to room temperature and stirred for 3 h. The reaction mixture was diluted with Na₂SO₃ (75 g) in H₂O (380 mL) and NaHCO₃(24 g) in H₂O (1100 mL) and stirred for 1 h. The precipitate was collected by filtration and washed with water to give 2,4,5-tribromonaphthalen-1-amine (Int-W001) (97 g) as a purple solid. ¹H NMR (400 MHZ, CDCl₃) δ 7.99 (s, 1H), 7.97-7.95 (1H, m), 7.86-7.84 (1H, m), 7.31-7.29 (1H, m), 4.65 (2H, brs). LCMS (ESI): m/z [M+H]⁺ 379.9.

### Step B: 1,8-dibromo-3-(methoxymethoxy)naphthalene (Int-W002)

To a suspension of 2,4,5-tribromonaphthalen-1-amine (Int-W001) (60 g, 160 mmol) in AcOH (780 mL) and propionic acid (300 mL) was added NaNO₂ (11 g) portionwise at 0° C. and the reaction mixture was stirred for 20 min. The reaction mixture was diluted with H₂O (1800 mL) at 0° C. and stirred for 1 h. The slurry was filtrated and the solid was washed with H₂O to give 4,5-dibromo-2-hydroxynaphthalene-1-diazonium, which was used without further purification.

To a suspension of 4,5-dibromo-2-hydroxynaphthalene-1-diazonium in EtOH (1600 mL) was added NaBH₄ (15 g) portionwise at 0° C. and the reaction mixture was stirred for 30 min. The mixture was allowed to warm to room temperature and stirred overnight. The reaction mixture was cooled to 0 C and diluted with water (1500 mL) and 5M HCl aq. (79 mL). The mixture was evaporated to remove EtOH and extracted with CHCl₃. The combined organic layer was washed with brine and dried over Na₂SO₄ and evaporated in vacuo to give 4,5-dibromonaphthalen-2-ol, which was used without further purification.

To a solution of 4,5-dibromonaphthalen-2-ol in CH₂Cl₂ (900 mL) was added i-Pr₂NEt (170 mL) and MOMCl (36 m) at 0 C. After stirring at room temperature for 1 h, the reaction mixture was diluted with EtOAc and sat. NaHCO₃. The organic layer was separated, and the aqueous layer was extracted with EtOAc. The combined organic layer was washed with brine, dried over Na₂SO₄, and evaporated. The resulting residue was purified by silica gel column chromatography to give 1,8-dibromo-3-(methoxymethoxy)naphthalene (Int-W002) (17.4 g). ¹H NMR (400 MHZ, CHLOROFORM-d) δ=7.82 (dd, J=1.3, 7.4 Hz, 1H), 7.75-7.71 (m, 2H), 7.41 (d, J=2.6 Hz, 1H), 7.26-7.20 (m, J=8.0, 8.0 Hz, 1H), 5.29 (s, 2H), 3.53 (s, 3H).

### Example 1: (4-(azepan-1-yl)-2-((1-((dimethylamino)methyl)cyclopropyl)methoxy)-5,7-dihydro-6H-pyrrolo[3,4-d]pyrimidin-6-yl)(3-hydroxy-8-iodonaphthalen-1-yl)methanone (Ex. 1)

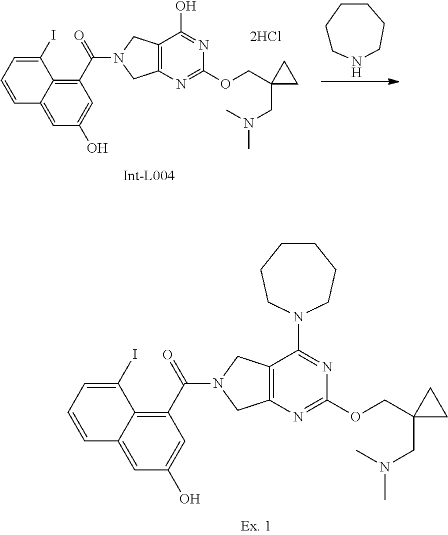 [SMILES: CN(C)CC1(COc2nc(O)c3c(n2)CN(C(=O)c2cc(O)cc4cccc(I)c24)C3)CC1 ; C1CCCNCC1 ; CN(C)CC1(COc2nc3c(c(N4CCCCCC4)n2)CN(C(=O)c2cc(O)cc4cccc(I)c24)C3)CC1]

To a mixture of (2-((1-((dimethylamino)methyl)cyclopropyl)methoxy)-4-hydroxy-5,7-dihydro-6H-pyrrolo[3,4-d]pyrimidin-6-yl)(3-hydroxy-8-iodonaphthalen-1-yl)methanone dihydrochloride (Int-L004) (15.0 mg, 0.0237 mmol) and p-toluenesulfonyl chloride (9.9 mg, 0.0521 mmol) in DMF (0.3 mL) was added N,N-diisopropylethylamine (0.032 mL, 0.189 mmol) and the mixture was stirred at room temperature for 0.5 h. To the mixture was added hexamethyleneimine (0.0107 mL, 0.0947 mmol) and the mixture was stirred at 70° C. for 1 h. Then aqueous NaOH (2M, 0.4 mL) was added to the mixture and the mixture was stirred at 70° C. for another 1 h. The mixture was cooled to room temperature and diluted with DMSO (0.5 mL), and the product mixture was purified by reverse phase HPLC (MeCN/water with 0.1% formic acid) to give (4-(azepan-1-yl)-2-((1-((dimethylamino)methyl)cyclopropyl)methoxy)-5,7-dihydro-6H-pyrrolo[3,4-d]pyrimidin-6-yl)(3-hydroxy-8-iodonaphthalen-1-yl)methanone (Ex. 1). ESI-MS m/z [M+H]⁺ 642. ¹H NMR (400 MHZ, DMSO-d₆) δ 10.2 (s, 1H), 8.03-7.98 (m, 1H), 7.87-7.82 (m, 1H), 7.29-7.26 (m, 1H), 7.16-7.10 (m, 2H), 4.98 (br s, 1H), 4.70-4.45 (m, 2H), 4.19-4.00 (m, 4H), 3.70-3.62 (m, 1H), 2.35-2.18 (m, 2H), 2.16 (s, 3H), 2.12 (s, 3H), 1.83-1.70 (m, 2H), 1.60-1.34 (m, 8H), 0.59-0.56 (m, 2H), 0.54-0.51 (m, 2H).

Compounds in the table below were synthesized via a similar route as Ex. 1 using (2-((1-((dimethylamino)methyl)cyclopropyl)methoxy)-4-hydroxy-5,7-dihydro-6H-pyrrolo[3,4-d]pyrimidin-6-yl)(3-hydroxy-8-iodonaphthalen-1-yl)methanone dihydrochloride (Int-L004) or (1-((dimethylamino)methyl)cyclopropyl)methanol and the corresponding amines.

TABLE 1

| | | | | ESI-MS |
|---|---|---|---|---|
| Example | Structure | Name | NMR | [M + H]⁺ |
| 2 | 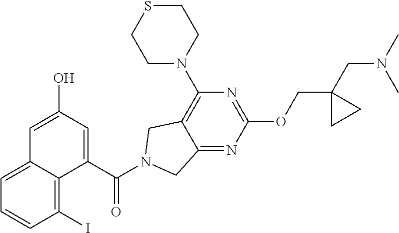 [SMILES: CN(C)CC1(COc2nc3c(c(N4CCSCC4)n2)CN(C(=O)c2cc(O)cc4cccc(I)c24)C3)CC1] | (2-((1- ((dimethylamino)methyl)cyclopropyl) methoxy)-4-thiomorpholino-5,7- dihydro-6H-pyrrolo[3,4-d]pyrimidin- 6-yl)(3-hydroxy-8-iodonaphthalen-1- yl)methanone | | 646 |
| 3 | 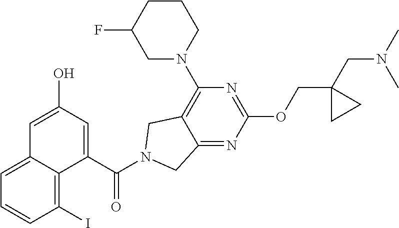 [SMILES: CN(C)CC1(COc2nc3c(c(N4CCCC(F)C4)n2)CN(C(=O)c2cc(O)cc4cccc(I)c24)C3)CC1] | (2-((1- ((dimethylamino)methyl)cyclopropyl) methoxy)-4-(3-fluoropiperidin-1- yl)-5,7-dihydro-6H-pyrrolo[3,4- d]pyrimidin-6-yl)(3-hydroxy-8- iodonaphthalen-1-yl)methanone | | 646 |
| 4 | 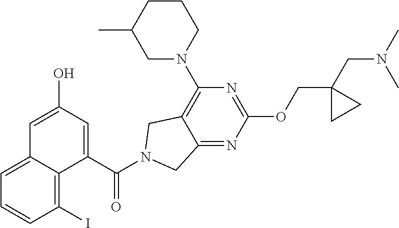 [SMILES: CC1CCCN(c2nc(OCC3(CN(C)C)CC3)nc3c2CN(C(=O)c2cc(O)cc4cccc(I)c24)C3)C1] | (2-((1- ((dimethylamino)methyl)cyclopropyl) methoxy)-4-(3-methylpiperidin-1- yl)-5,7-dihydro-6H-pyrrolo[3,4-d] pyrimidin-6-yl)(3-hydroxy-8- iodonaphthalen-1-yl)methanone | | 642 |
| 5 | 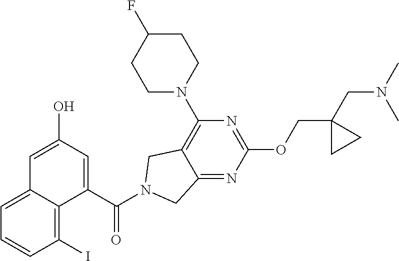 [SMILES: CN(C)CC1(COc2nc3c(c(N4CCC(F)CC4)n2)CN(C(=O)c2cc(O)cc4cccc(I)c24)C3)CC1] | (2-((1- ((dimethylamino)methyl)cyclopropyl) methoxy)-4-(4-fluoropiperidin-1-yl)- 5,7-dihydro-6H-pyrrolo[3,4-d] pyrimidin-6-yl)(3-hydroxy-8- iodonaphthalen-1-yl)methanone | | 646 |
| 6 | 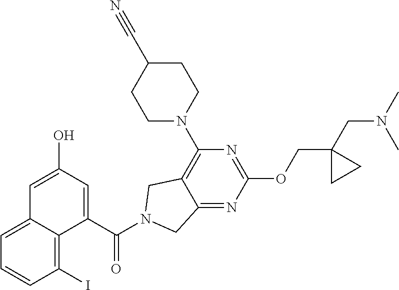 [SMILES: CN(C)CC1(COc2nc3c(c(N4CCC(C#N)CC4)n2)CN(C(=O)c2cc(O)cc4cccc(I)c24)C3)CC1] | 1-(2-((1- ((dimethylamino)methyl)cyclopropyl) methoxy)-6-(3-hydroxy-8-iodo-1- naphthoyl)-6,7-dihydro-5H- pyrrolo[3,4-d]pyrimidin-4-yl) piperidine-4-carbonitrile | | 653 |
| 7 | 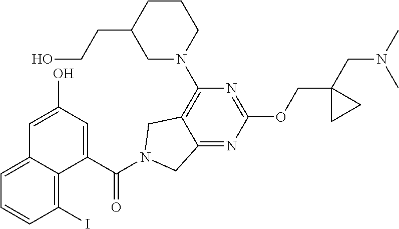 [SMILES: CN(C)CC1(COc2nc3c(c(N4CCCC(CCO)C4)n2)CN(C(=O)c2cc(O)cc4cccc(I)c24)C3)CC1] | (2-((1- ((dimethylamino)methyl)cyclopropyl) methoxy)-4-(3-(2-hydroxyethyl) piperidin-1-yl)-5,7-dihydro-6H- pyrrolo[3,4-d]pyrimidin-6-yl)(3- hydroxy-8-iodonaphthalen-1- yl)methanone | | 672 |
| 8 | 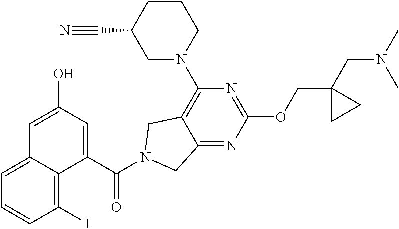 [SMILES: CN(C)CC1(COc2nc3c(c(N4CCC[C@@H](C#N)C4)n2)CN(C(=O)c2cc(O)cc4cccc(I)c24)C3)CC1] | (R)-1-(2-((1- ((dimethylamino)methyl)cyclopropyl) methoxy)-6-(3-hydroxy-8-iodo-1- naphthoyl)-6,7-dihydro-5H-pyrrolo [3,4-d]pyrimidin-4-yl)piperidine-3- carbonitrile | | 653 |
| 9 | 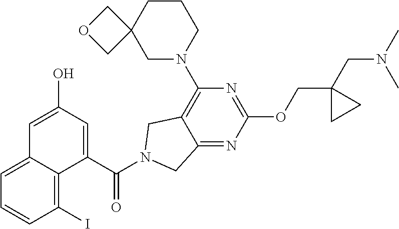 [SMILES: CN(C)CC1(COc2nc3c(c(N4CCCC5(COC5)C4)n2)CN(C(=O)c2cc(O)cc4cccc(I)c24)C3)CC1] | (2-((1- ((dimethylamino)methyl)cyclopropyl) methoxy)-4-(2-oxa-6-azaspiro[3.5] nonan-6-yl)-5,7-dihydro-6H-pyrrolo [3,4-d]pyrimidin-6-yl)(3-hydroxy-8- iodonaphthalen-1-yl)methanone | | 670 |
| 10 | 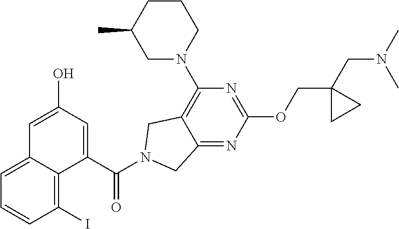 [SMILES: C[C@H]1CCCN(c2nc(OCC3(CN(C)C)CC3)nc3c2CN(C(=O)c2cc(O)cc4cccc(I)c24)C3)C1] | (S)-(2-((1- ((dimethylamino)methyl)cyclopropyl) methoxy)-4-(3-methylpiperidin-1-yl)- 5,7-dihydro-6H-pyrrolo[3,4-d] pyrimidin-6-yl)(3-hydroxy-8- iodonaphthalen-1-yl)methanone | | 642 |
| 11 | 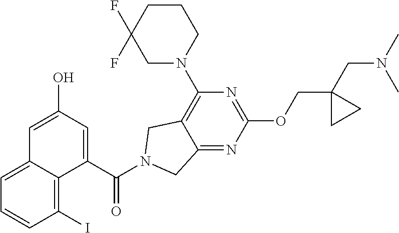 [SMILES: CN(C)CC1(COc2nc3c(c(N4CCCC(F)(F)C4)n2)CN(C(=O)c2cc(O)cc4cccc(I)c24)C3)CC1] | (4-(3,3-difluoropiperidin-1-yl)-2-((1- ((dimethylamino)methyl)cyclopropyl) methoxy)-5,7-dihydro-6H-pyrrolo [3,4-d]pyrimidin-6-yl)(3-hydroxy-8- iodonaphthalen-1-yl)methanone | | 664 |
| 12 | 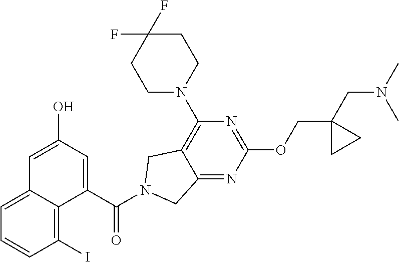 [SMILES: CN(C)CC1(COc2nc3c(c(N4CCC(F)(F)CC4)n2)CN(C(=O)c2cc(O)cc4cccc(I)c24)C3)CC1] | (4-(4,4-difluoropiperidin-1-yl)-2-((1- ((dimethylamino)methyl)cyclopropyl) methoxy)-5,7-dihydro-6H-pyrrolo [3,4-d]pyrimidin-6-yl)(3-hydroxy-8- iodonaphthalen-1-yl)methanone | | 664 |
| 13 | 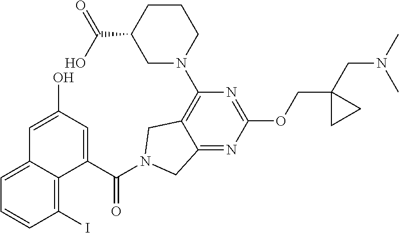 [SMILES: CN(C)CC1(COc2nc3c(c(N4CCC[C@@H](C(=O)O)C4)n2)CN(C(=O)c2cc(O)cc4cccc(I)c24)C3)CC1] | (R)-1-(2-((1- ((dimethylamino)methyl)cyclopropyl) methoxy)-6-(3-hydroxy-8-iodo-1- naphthoyl)-6,7-dihydro-5H-pyrrolo [3,4-d]pyrimidin-4-yl)piperidine-3- carboxylicacid | | 672 |
| 14 | 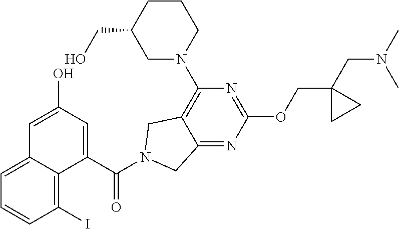 [SMILES: CN(C)CC1(COc2nc3c(c(N4CCC[C@@H](CO)C4)n2)CN(C(=O)c2cc(O)cc4cccc(I)c24)C3)CC1] | (R)-(2-((1- ((dimethylamino)methyl)cyclopropyl) methoxy)-4-(3-(hydroxymethyl) piperidin-1-yl)-5,7-dihydro-6H- pyrrolo[3,4-d]pyrimidin-6-yl)(3- hydroxy-8-iodonaphthalen-1- yl)methanone | | 658 |
| 15 | 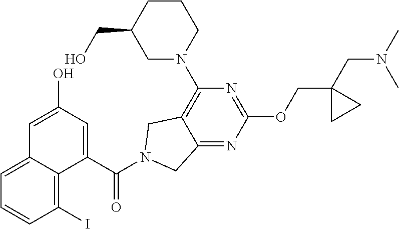 [SMILES: CN(C)CC1(COc2nc3c(c(N4CCC[C@H](CO)C4)n2)CN(C(=O)c2cc(O)cc4cccc(I)c24)C3)CC1] | (S)-(2-((1- ((dimethylamino)methyl)cyclopropyl) methoxy)-4-(3-(hydroxymethyl) piperidin-1-yl)-5,7-dihydro-6H- pyrrolo[3,4-d]pyrimidin-6-yl)(3- hydroxy-8-iodonaphthalen-1- yl)methanone | | 658 |
| 16 | 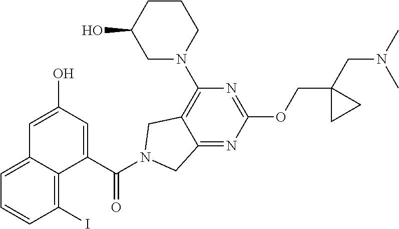 [SMILES: CN(C)CC1(COc2nc3c(c(N4CCC[C@H](O)C4)n2)CN(C(=O)c2cc(O)cc4cccc(I)c24)C3)CC1] | (S)-(2-((1- ((dimethylamino)methyl)cyclopropyl) methoxy)-4-(3-hydroxypiperidin-1-yl)- 5,7-dihydro-6H-pyrrolo[3,4-d] pyrimidin-6-yl)(3-hydroxy-8- iodonaphthalen-1-yl)methanone | | 644 |
| 17 | 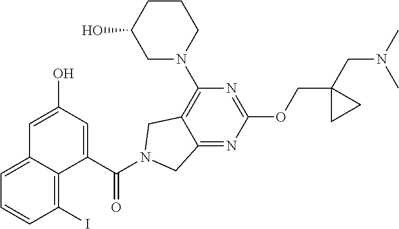 [SMILES: CN(C)CC1(COc2nc3c(c(N4CCC[C@@H](O)C4)n2)CN(C(=O)c2cc(O)cc4cccc(I)c24)C3)CC1] | (R)-(2-((1- ((dimethylamino)methyl)cyclopropyl) methoxy)-4-(3-hydroxypiperidin-1-yl)- 5,7-dihydro-6H-pyrrolo[3,4-d] pyrimidin-6-yl)(3-hydroxy-8- iodonaphthalen-1-yl)methanone | | 644 |
| 18 | 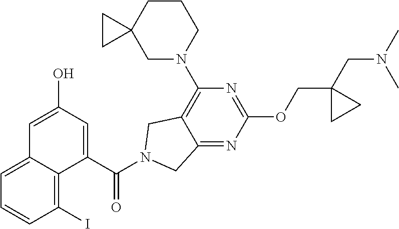 [SMILES: CN(C)CC1(COc2nc3c(c(N4CCCC5(CC5)C4)n2)CN(C(=O)c2cc(O)cc4cccc(I)c24)C3)CC1] | (2-((1- ((dimethylamino)methyl)cyclopropyl) methoxy)-4-(5-azaspiro[2.5]octan-5- yl)-5,7-dihydro-6H-pyrrolo[3,4-d] pyrimidin-6-yl)(3-hydroxy-8- iodonaphthalen-1-yl)methanone | | 654 |
| 19 | 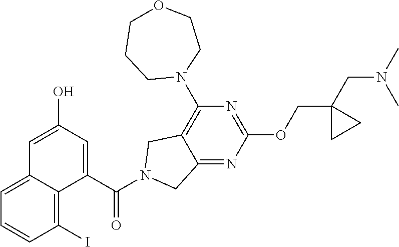 [SMILES: CN(C)CC1(COc2nc3c(c(N4CCCOCC4)n2)CN(C(=O)c2cc(O)cc4cccc(I)c24)C3)CC1] | (2-((1- ((dimethylamino)methyl)cyclopropyl) methoxy)-4-(1,4-oxazepan-4-yl)-5,7- dihydro-6H-pyrrolo[3,4-d]pyrimidin- 6-yl)(3-hydroxy-8-iodonaphthalen-1- yl)methanone | | 644 |
| 20 | 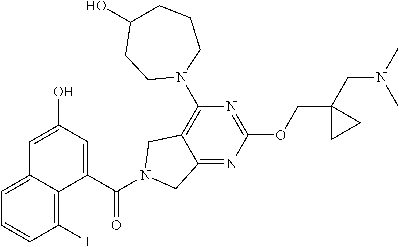 [SMILES: CN(C)CC1(COc2nc3c(c(N4CCCC(O)CC4)n2)CN(C(=O)c2cc(O)cc4cccc(I)c24)C3)CC1] | (2-((1- ((dimethylamino)methyl)cyclopropyl) methoxy)-4-(4-hydroxyazepan-1-yl)- 5,7-dihydro-6H-pyrrolo[3,4-d] pyrimidin-6-yl)(3-hydroxy-8- iodonaphthalen-1-yl)methanone | | 658 |
| 21 | 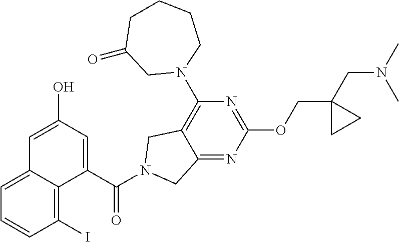 [SMILES: CN(C)CC1(COc2nc3c(c(N4CCCCC(=O)C4)n2)CN(C(=O)c2cc(O)cc4cccc(I)c24)C3)CC1] | 1-(2-((1- ((dimethylamino)methyl)cyclopropyl) methoxy)-6-(3-hydroxy-8-iodo-1- naphthoyl)-6, 7-dihydro-5H-pyrrolo [3,4-d]pyrimidin-4-yl)azepan-3-one | | 656 |
| 22 | 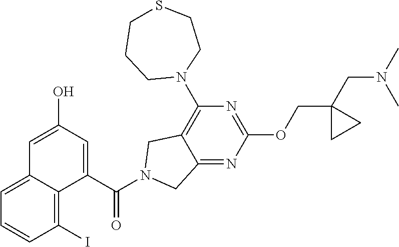 [SMILES: CN(C)CC1(COc2nc3c(c(N4CCCSCC4)n2)CN(C(=O)c2cc(O)cc4cccc(I)c24)C3)CC1] | (2-((1- ((dimethylamino)methyl)cyclopropyl) methoxy)-4-(1,4-thiazepan-4-yl)-5,7- dihydro-6H-pyrrolo[3,4-d]pyrimidin- 6-yl)(3-hydroxy-8-iodonaphthalen-1- yl)methanone | | 660 |
| 23 | 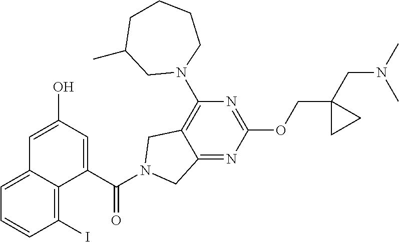 [SMILES: CC1CCCCN(c2nc(OCC3(CN(C)C)CC3)nc3c2CN(C(=O)c2cc(O)cc4cccc(I)c24)C3)C1] | (2-((1- ((dimethylamino)methyl)cyclopropyl) methoxy)-4-(3-methylazepan-1-yl)- 5,7-dihydro-6H-pyrrolo[3,4-d] pyrimidin-6-yl)(3-hydroxy-8- iodonaphthalen-1-yl)methanone | | 656 |
| 24 | 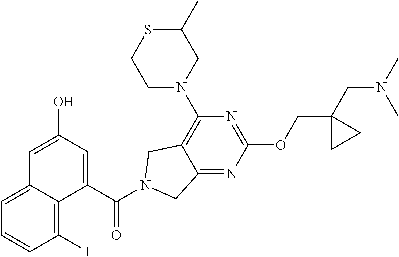 [SMILES: CC1CN(c2nc(OCC3(CN(C)C)CC3)nc3c2CN(C(=O)c2cc(O)cc4cccc(I)c24)C3)CCS1] | (2-((1- ((dimethylamino)methyl)cyclopropyl) methoxy)-4-(2-methylthiomorpholino)- 5,7-dihydro-6H-pyrrolo[3,4-d] pyrimidin-6-yl)(3-hydroxy-8- iodonaphthalen-1-yl)methanone | | 660 |
| 25 | 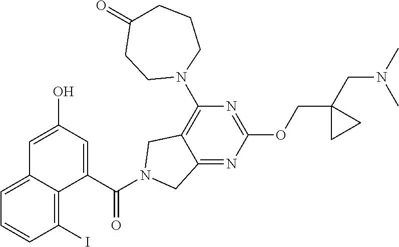 [SMILES: CN(C)CC1(COc2nc3c(c(N4CCCC(=O)CC4)n2)CN(C(=O)c2cc(O)cc4cccc(I)c24)C3)CC1] | 1-(2-((1- ((dimethylamino)methyl)cyclopropyl) methoxy)-6-(3-hydroxy-8-iodo-1- naphthoyl)-6,7-dihydro-5H-pyrrolo [3,4-d]pyrimidin-4-yl)azepan-4-one | | 656 |
| 26 | 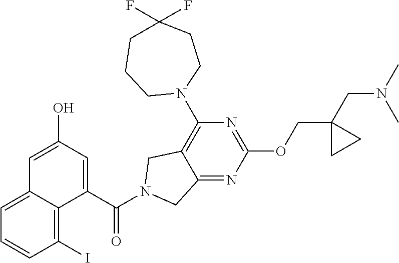 [SMILES: CN(C)CC1(COc2nc3c(c(N4CCCC(F)(F)CC4)n2)CN(C(=O)c2cc(O)cc4cccc(I)c24)C3)CC1] | (4-(4,4-difluoroazepan-1-yl)-2-((1- ((dimethylamino)methyl)cyclopropyl) methoxy)-5,7-dihydro-6H-pyrrolo [3,4-d]pyrimidin-6-yl)(3-hydroxy-8- iodonaphthalen-1-yl)methanone | | 678 |
| 27 | 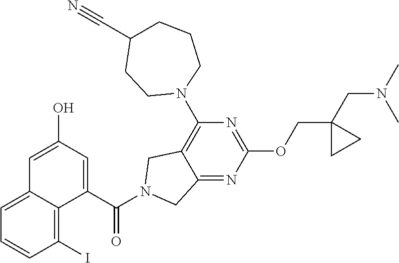 [SMILES: CN(C)CC1(COc2nc3c(c(N4CCCC(C#N)CC4)n2)CN(C(=O)c2cc(O)cc4cccc(I)c24)C3)CC1] | 1-(2-((1- ((dimethylamino)methyl)cyclopropyl) methoxy)-6-(3-hydroxy-8-iodo-1- naphthoyl)-6,7-dihydro-5H-pyrrolo [3,4-d]pyrimidin-4-yl)azepane- 4-carbonitrile | | 667 |
| 28 | 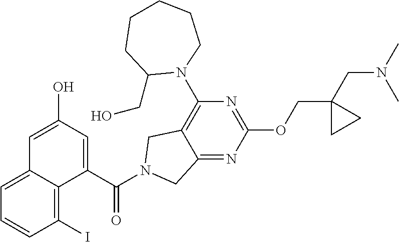 [SMILES: CN(C)CC1(COc2nc3c(c(N4CCCCCC4CO)n2)CN(C(=O)c2cc(O)cc4cccc(I)c24)C3)CC1] | (2-((1- ((dimethylamino)methyl)cyclopropyl) methoxy)-4-(2-(hydroxymethyl) azepan-1-yl)-5,7-dihydro-6H-pyrrolo [3,4-d]pyrimidin-6-yl)(3-hydroxy-8- iodonaphthalen-1-yl)methanone | | 672 |
| 29 | 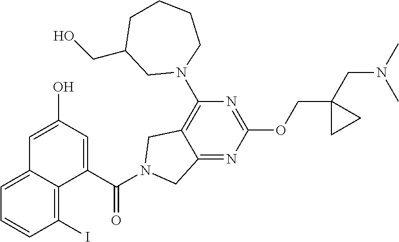 [SMILES: CN(C)CC1(COc2nc3c(c(N4CCCCC(CO)C4)n2)CN(C(=O)c2cc(O)cc4cccc(I)c24)C3)CC1] | (2-((1- ((dimethylamino)methyl)cyclopropyl) methoxy)-4-(3-(hydroxymethyl) azepan-1-yl)-5,7-dihydro-6H-pyrrolo [3,4-d]pyrimidin-6-yl)(3-hydroxy-8- iodonaphthalen-1-yl)methanone | | 672 |
| 30 | 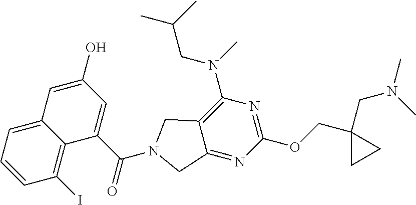 [SMILES: CC(C)CN(C)c1nc(OCC2(CN(C)C)CC2)nc2c1CN(C(=O)c1cc(O)cc3cccc(I)c13)C2] | (2-((1- ((dimethylamino)methyl)cyclopropyl) methoxy)-4-(isobutyl(methyl)amino)- 5,7-dihydro-6H-pyrrolo[3,4-d] pyrimidin-6-yl)(3-hydroxy-8- iodonaphthalen-1-yl)methanone | | 630 |
| 31 | 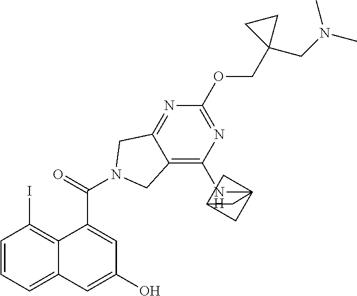 [SMILES: CN(C)CC1(COc2nc3c(c(NC45CC(C4)C5)n2)CN(C(=O)c2cc(O)cc4cccc(I)c24)C3)CC1] | (4-((bicyclo[1.1.1]pentan-1- ylmethyl)amino)-2-((1- ((dimethylamino)methyl) cyclopropyl)methoxy)-5,7- dihydro-6H-pyrrolo[3,4- d]pyrimidin-6-yl)(3-hydroxy-8- iodonaphthalen-1-yl)methanone | | 640 |
| 32 | 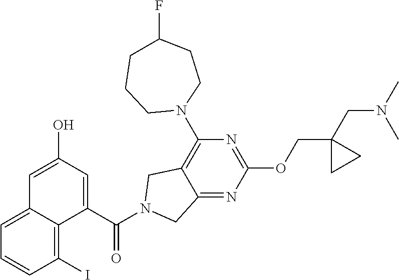 [SMILES: CN(C)CC1(COc2nc3c(c(N4CCCC(F)CC4)n2)CN(C(=O)c2cc(O)cc4cccc(I)c24)C3)CC1] | (2-((1- ((dimethylamino)methyl)cyclopropyl) methoxy)-4-(4-fluoroazepan-1-yl)-5,7- dihydro-6H-pyrrolo[3,4-d]pyrimidin-6- yl)(3-hydroxy-8-iodonaphthalen-1- yl)methanone | | 660 |
| 33 | 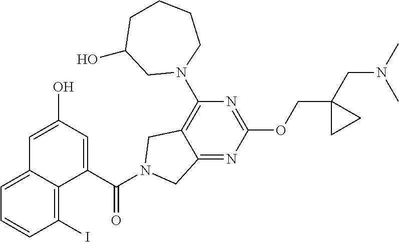 [SMILES: CN(C)CC1(COc2nc3c(c(N4CCCCC(O)C4)n2)CN(C(=O)c2cc(O)cc4cccc(I)c24)C3)CC1] | (2-((1- ((dimethylamino)methyl)cyclopropyl) methoxy)-4-(3-hydroxyazepan-1-yl)- 5,7-dihydro-6H-pyrrolo[3,4-d] pyrimidin-6-yl)(3-hydroxy-8- iodonaphthalen-1-yl)methanone | | 658 |
| 34 | 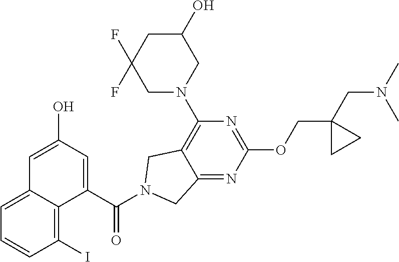 [SMILES: CN(C)CC1(COc2nc3c(c(N4CC(O)CC(F)(F)C4)n2)CN(C(=O)c2cc(O)cc4cccc(I)c24)C3)CC1] | (4-(3,3-difluoro-5-hydroxypiperidin-1- yl)-2-((1- ((dimethylamino)methyl)cyclopropyl) methoxy)-5,7-dihydro-6H-pyrrolo[3,4- d]pyrimidin-6-yl)(3-hydroxy-8- iodonaphthalen-1-yl)methanone | | 680 |
| 35 | 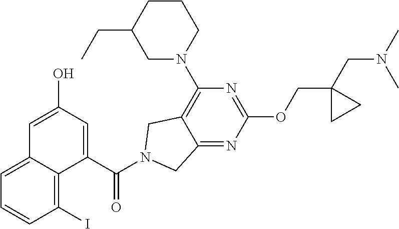 [SMILES: CCC1CCCN(c2nc(OCC3(CN(C)C)CC3)nc3c2CN(C(=O)c2cc(O)cc4cccc(I)c24)C3)C1] | (2-((1- ((dimethylamino)methyl)cyclopropyl) methoxy)-4-(3-ethylpiperidin-1-yl)-5,7- dihydro-6H-pyrrolo[3,4-d]pyrimidin-6- yl)(3-hydroxy-8-iodonaphthalen-1- yl)methanone | | 656 |
| 36 | 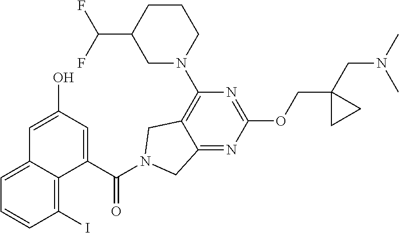 [SMILES: CN(C)CC1(COc2nc3c(c(N4CCCC(C(F)F)C4)n2)CN(C(=O)c2cc(O)cc4cccc(I)c24)C3)CC1] | (4-(3-(difluoromethyl)piperidin-1- yl)-2-((1-((dimethylamino)methyl) cyclopropyl)methoxy)-5,7-dihydro- 6H-pyrrolo[3,4- d]pyrimidin-6-yl)(3-hydroxy-8- iodonaphthalen-1-yl)methanone | | 678 |
| 37 | 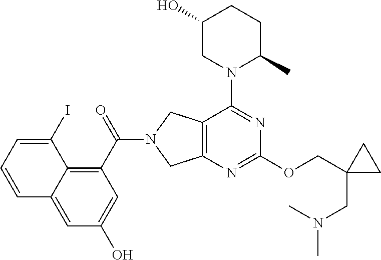 [SMILES: C[C@@H]1CC[C@@H](O)CN1c1nc(OCC2(CN(C)C)CC2)nc2c1CN(C(=O)c1cc(O)cc3cccc(I)c13)C2] | (2-((1- ((dimethylamino)methyl)cyclopropyl) methoxy)-4-((2R,5R)-5-hydroxy-2- methylpiperidin-1-yl)-5,7-dihydro-6H- pyrrolo[3,4-d]pyrimidin-6-yl)(3- hydroxy-8-iodonaphthalen-1- yl)methanone | | 658 |
| 38 | 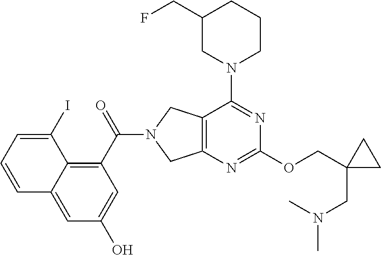 [SMILES: CN(C)CC1(COc2nc3c(c(N4CCCC(CF)C4)n2)CN(C(=O)c2cc(O)cc4cccc(I)c24)C3)CC1] | (2-((1- ((dimethylamino)methyl)cyclopropyl) methoxy)-4-(3-(fluoromethyl) piperidin-1-yl)-5,7-dihydro- 6H-pyrrolo[3,4- d]pyrimidin-6-yl)(3-hydroxy-8- iodonaphthalen-1-yl)methanone | | 660 |
| 39 | 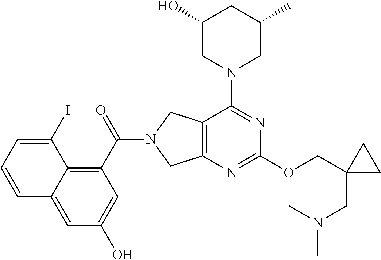 [SMILES: C[C@H]1C[C@@H](O)CN(c2nc(OCC3(CN(C)C)CC3)nc3c2CN(C(=O)c2cc(O)cc4cccc(I)c24)C3)C1] | (2-((1- ((dimethylamino)methyl)cyclopropyl) methoxy)-4-((3R,5S)-3-hydroxy-5- methylpiperidin-1-yl)-5,7-dihydro-6H- pyrrolo[3,4-d]pyrimidin-6-yl)(3- hydroxy-8-iodonaphthalen-1- yl)methanone | | 658 |
| 40 | 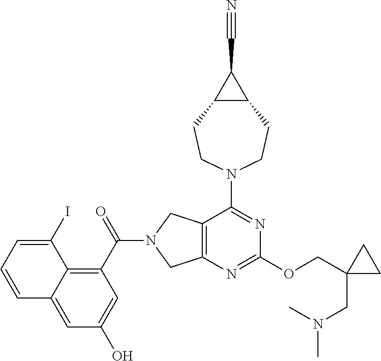 [SMILES: CN(C)CC1(COc2nc3c(c(N4CC[C@@H]5[C@@H](C#N)[C@@H]5CC4)n2)CN(C(=O)c2cc(O)cc4cccc(I)c24)C3)CC1] | (1R,7S,8r)-4-(2-((1- ((dimethylamino)methyl)cyclopropyl) methoxy)-6-(3-hydroxy-8-iodo-1- naphthoyl)-6,7-dihydro-5H-pyrrolo [3,4-d]pyrimidin-4-yl)-4- azabicyclo[5.1.0]octane-8-carbonitrile | | 679 |
| 41 | 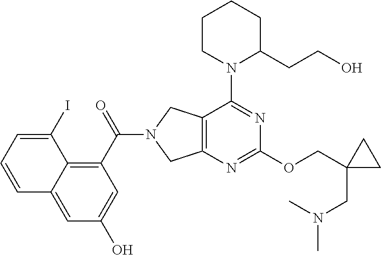 [SMILES: CN(C)CC1(COc2nc3c(c(N4CCCCC4CCO)n2)CN(C(=O)c2cc(O)cc4cccc(I)c24)C3)CC1] | (2-((1- ((dimethylamino)methyl)cyclopropyl) methoxy)-4-(2-(2-hydroxyethyl) piperidin-1- yl)-5,7-dihydro-6H-pyrrolo[3,4- d]pyrimidin-6-yl)(3-hydroxy-8- iodonaphthalen-1-yl)methanone | | 672 |
| 42 | 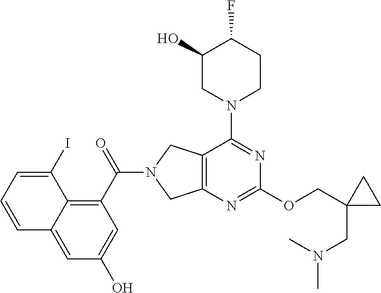 [SMILES: CN(C)CC1(COc2nc3c(c(N4CC[C@@H](F)[C@H](O)C4)n2)CN(C(=O)c2cc(O)cc4cccc(I)c24)C3)CC1] | (2-((1- ((dimethylamino)methyl)cyclopropyl) methoxy)-4-((3R,4R)-4-fluoro-3- hydroxypiperidin-1-yl)-5,7-dihydro- 6H-pyrrolo[3,4-d]pyrimidin-6-yl)(3- hydroxy-8-iodonaphthalen-1- yl)methanone | | 662 |
| 43 | 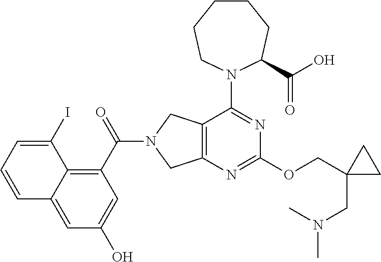 [SMILES: CN(C)CC1(COc2nc3c(c(N4CCCCC[C@H]4C(=O)O)n2)CN(C(=O)c2cc(O)cc4cccc(I)c24)C3)CC1] | (S)-1-(2-((1- ((dimethylamino)methyl)cyclopropyl) methoxy)-6-(3-hydroxy-8-iodo-1- naphthoyl)-6,7-dihydro-5H-pyrrolo [3,4-d]pyrimidin-4-yl)azepane- 2-carboxylic acid | | 686 |
| 44 | 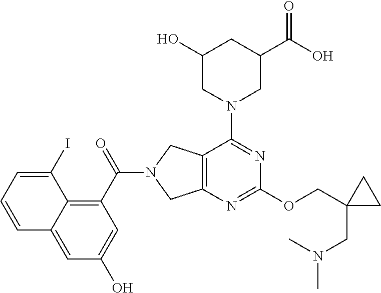 [SMILES: CN(C)CC1(COc2nc3c(c(N4CC(O)CC(C(=O)O)C4)n2)CN(C(=O)c2cc(O)cc4cccc(I)c24)C3)CC1] | 1-(2-((1- ((dimethylamino)methyl)cyclopropyl) methoxy)-6-(3-hydroxy-8-iodo-1- naphthoyl)-6,7-dihydro-5H-pyrrolo [3,4-d]pyrimidin-4-yl)-5- hydroxypiperidine-3-carboxylic acid | | 688 |
| 45 | 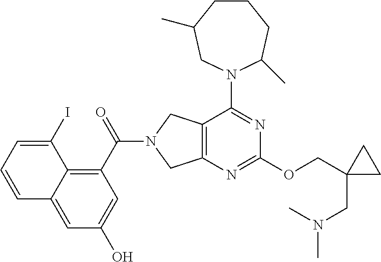 [SMILES: CC1CCCC(C)N(c2nc(OCC3(CN(C)C)CC3)nc3c2CN(C(=O)c2cc(O)cc4cccc(I)c24)C3)C1] | (2-((1- ((dimethylamino)methyl)cyclopropyl) methoxy)-4-(2,6-dimethylazepan- 1-yl)-5,7-dihydro-6H-pyrrolo[3,4-d] pyrimidin-6-yl)(3-hydroxy-8- iodonaphthalen-1- yl)methanone | | 670 |
| 46 | 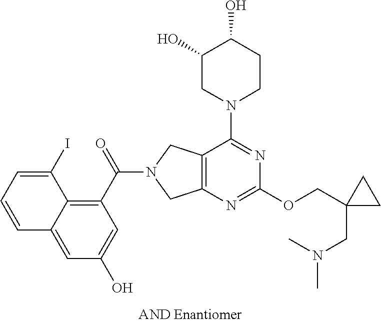 [SMILES: CN(C)CC1(COc2nc3c(c(N4CC[C@@H](O)[C@@H](O)C4)n2)CN(C(=O)c2cc(O)cc4cccc(I)c24)C3)CC1] | rac-(4-((3R,4S)-3,4- dihydroxypiperidin-1-yl)-2-((1- ((dimethylamino)methyl)cyclopropyl) methoxy)-5,7-dihydro-6H-pyrrolo[3,4- d]pyrimidin-6-yl)(3-hydroxy-8- iodonaphthalen-1-yl)methanone | | 660 |
| 47 | 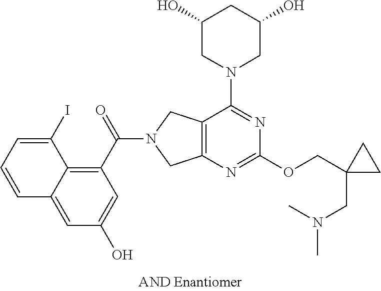 [SMILES: CN(C)CC1(COc2nc3c(c(N4C[C@H](O)C[C@H](O)C4)n2)CN(C(=O)c2cc(O)cc4cccc(I)c24)C3)CC1] | (4-((3S,5R)-3,5-dihydroxypiperidin-1- yl)-2-((1- ((dimethylamino)methyl)cyclopropyl) methoxy)-5,7-dihydro-6H-pyrrolo[3,4- d]pyrimidin-6-yl)(3-hydroxy-8- iodonaphthalen-1-yl)methanone | | 660 |
| 48 | 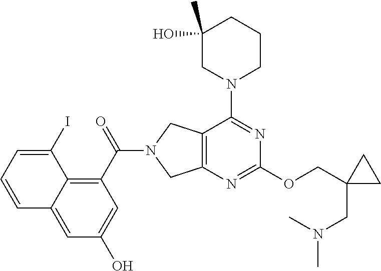 [SMILES: CN(C)CC1(COc2nc3c(c(N4CCC[C@@](C)(O)C4)n2)CN(C(=O)c2cc(O)cc4cccc(I)c24)C3)CC1] | (R)-(2-((1- ((dimethylamino)methyl)cyclopropyl) methoxy)-4-(3-hydroxy- 3-methylpiperidin- 1-yl)-5,7-dihydro-6H-pyrrolo[3,4- d]pyrimidin-6-yl)(3-hydroxy-8- iodonaphthalen-1-yl)methanone | | 658 |
| 49 | 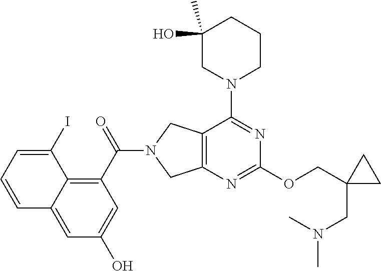 [SMILES: CN(C)CC1(COc2nc3c(c(N4CCC[C@](C)(O)C4)n2)CN(C(=O)c2cc(O)cc4cccc(I)c24)C3)CC1] | (S)-(2-((1- ((dimethylamino)methyl)cyclopropyl) methoxy)-4-(3-hydroxy- 3-methylpiperidin- 1-yl)-5,7-dihydro-6H-pyrrolo[3,4- d]pyrimidin-6-yl)(3-hydroxy-8- iodonaphthalen-1-yl)methanone | | 658 |
| 50 | 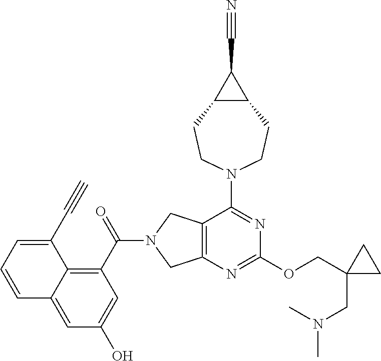 [SMILES: C#Cc1cccc2cc(O)cc(C(=O)N3Cc4nc(OCC5(CN(C)C)CC5)nc(N5CC[C@@H]6[C@@H](C#N)[C@@H]6CC5)c4C3)c12] | (1R,7S,8r)-4-(2-((1- ((dimethylamino)methyl)cyclopropyl) methoxy)-6-(8-ethynyl-3-hydroxy-1- naphthoyl)-6,7-dihydro-5H-pyrrolo [3,4-d]pyrimidin-4-yl)-4- azabicyclo[5.1.0]octane-8-carbonitrile | | 577 |
| 51 | 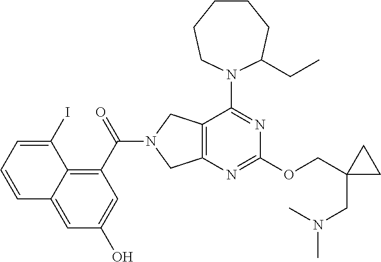 [SMILES: CCC1CCCCCN1c1nc(OCC2(CN(C)C)CC2)nc2c1CN(C(=O)c1cc(O)cc3cccc(I)c13)C2] | (2-((1- ((dimethylamino)methyl)cyclopropyl) methoxy)-4-(2-ethylazepan-1-yl)-5,7- dihydro-6H-pyrrolo[3,4-d]pyrimidin- 6-yl)(3-hydroxy-8-iodonaphthalen-1- yl)methanone | | 670 |
| 52 | 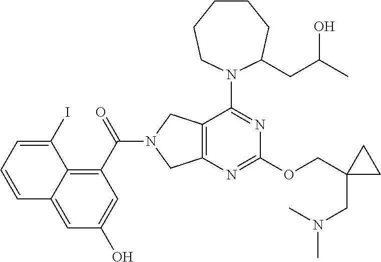 [SMILES: CC(O)CC1CCCCCN1c1nc(OCC2(CN(C)C)CC2)nc2c1CN(C(=O)c1cc(O)cc3cccc(I)c13)C2] | (2-((1- ((dimethylamino)methyl)cyclopropyl) methoxy)-4-(2-(2- hydroxypropyl)azepan-1- yl)-5,7-dihydro-6H-pyrrolo[3,4- d]pyrimidin-6-yl)(3-hydroxy-8- iodonaphthalen-1-yl)methanone | | 700 |
| 53 | 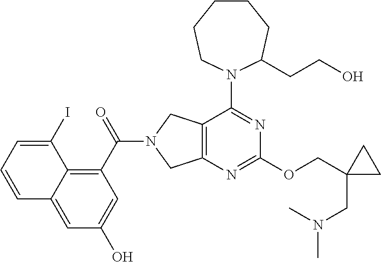 [SMILES: CN(C)CC1(COc2nc3c(c(N4CCCCCC4CCO)n2)CN(C(=O)c2cc(O)cc4cccc(I)c24)C3)CC1] | (2-((1- ((dimethylamino)methyl)cyclopropyl) methoxy)-4-(2-(2- hydroxyethyl)azepan-1- yl)-5,7-dihydro-6H-pyrrolo[3,4- d]pyrimidin-6-yl)(3-hydroxy-8- iodonaphthalen-1-yl)methanone | | 686 |
| 54 | 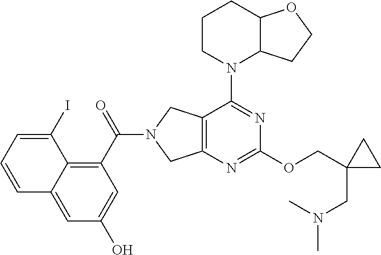 [SMILES: CN(C)CC1(COc2nc3c(c(N4CCCC5OCCC54)n2)CN(C(=O)c2cc(O)cc4cccc(I)c24)C3)CC1] | (2-((1- ((dimethylamino)methyl)cyclopropyl) methoxy)-4-(hexahydrofuro [3,2-b]pyridin-4(2H)-yl)- 5,7-dihydro-6H-pyrrolo[3,4- d]pyrimidin-6-yl)(3-hydroxy-8- iodonaphthalen-1-yl)methanone | | 670 |
| 55 | 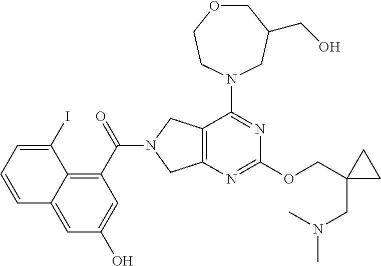 [SMILES: CN(C)CC1(COc2nc3c(c(N4CCOCC(CO)C4)n2)CN(C(=O)c2cc(O)cc4cccc(I)c24)C3)CC1] | (2-((1- ((dimethylamino)methyl)cyclopropyl) methoxy)-4-(6-(hydroxymethyl)-1,4- oxazepan-4-yl)-5,7-dihydro-6H- pyrrolo[3,4-d]pyrimidin-6-yl)(3- hydroxy-8-iodonaphthalen-1- yl)methanone | | 674 |
| 56 | 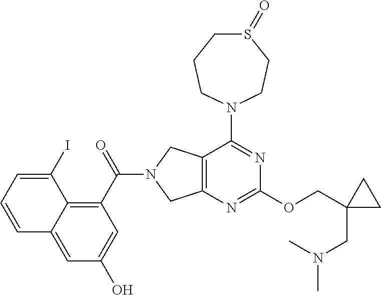 [SMILES: CN(C)CC1(COc2nc3c(c(N4CCCS(=O)CC4)n2)CN(C(=O)c2cc(O)cc4cccc(I)c24)C3)CC1] | (2-((1- ((dimethylamino)methyl)cyclopropyl) methoxy)-4-(1-oxido-1,4-thiazepan- 4-yl)-5,7-dihydro-6H-pyrrolo[3,4- d]pyrimidin-6-yl)(3-hydroxy-8- iodonaphthalen-1-yl)methanone | | 676 |
| 57 | 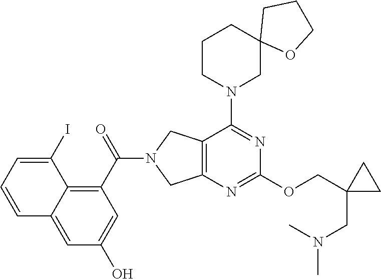 [SMILES: CN(C)CC1(COc2nc3c(c(N4CCCC5(CCCO5)C4)n2)CN(C(=O)c2cc(O)cc4cccc(I)c24)C3)CC1] | (2-((1- ((dimethylamino)methyl)cyclopropyl) methoxy)-4-(1-oxa-7-azaspiro[4.5] decan-7-yl)-5,7-dihydro- 6H-pyrrolo[3,4- d]pyrimidin-6-yl)(3-hydroxy-8- iodonaphthalen-1-yl)methanone | | 684 |
| 58 | 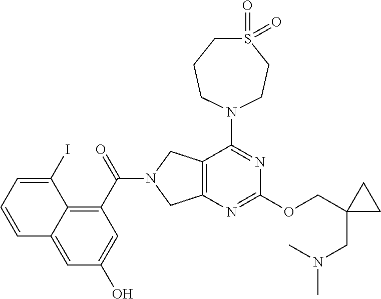 [SMILES: CN(C)CC1(COc2nc3c(c(N4CCCS(=O)(=O)CC4)n2)CN(C(=O)c2cc(O)cc4cccc(I)c24)C3)CC1] | (2-((1- ((dimethylamino)methyl)cyclopropyl) methoxy)-4-(1,1-dioxido-1,4- thiazepan-4-yl)-5,7-dihydro-6H- pyrrolo[3,4-d]pyrimidin-6-yl)(3- hydroxy-8-iodonaphthalen- 1-yl)methanone | | 692 |
| 59 | 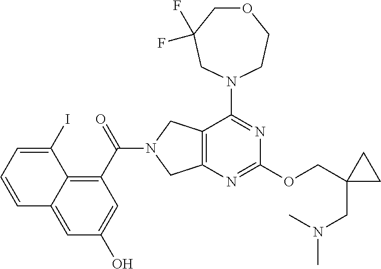 [SMILES: CN(C)CC1(COc2nc3c(c(N4CCOCC(F)(F)C4)n2)CN(C(=O)c2cc(O)cc4cccc(I)c24)C3)CC1] | (4-(6,6-difluoro-1,4-oxazepan-4-yl)-2- ((1-((dimethylamino)methyl) cyclopropyl)methoxy)-5,7-dihydro- 6H-pyrrolo[3,4- d]pyrimidin-6-yl)(3-hydroxy-8- iodonaphthalen-1-yl)methanone | | 680 |
| 60 | 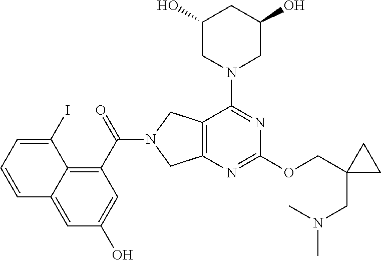 [SMILES: CN(C)CC1(COc2nc3c(c(N4C[C@H](O)C[C@@H](O)C4)n2)CN(C(=O)c2cc(O)cc4cccc(I)c24)C3)CC1] | (4-((3R,5R)-3,5-dihydroxypiperidin-1- yl)-2-((1- ((dimethylamino)methyl)cyclopropyl) methoxy)-5,7-dihydro-6H-pyrrolo[3,4- d]pyrimidin-6-yl)(3-hydroxy-8- iodonaphthalen-1-yl)methanone | | 660 |
| 61 | 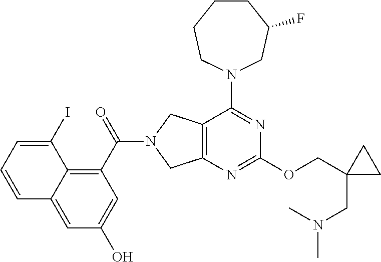 [SMILES: CN(C)CC1(COc2nc3c(c(N4CCCC[C@H](F)C4)n2)CN(C(=O)c2cc(O)cc4cccc(I)c24)C3)CC1] | (S)-(2-((1- ((dimethylamino)methyl)cyclopropyl) methoxy)-4-(3-fluoroazepan-1-yl)-5,7- dihydro-6H-pyrrolo[3,4-d]pyrimidin-6- yl)(3-hydroxy-8-iodonaphthalen-1- yl)methanone | | 660 |
| 62 | 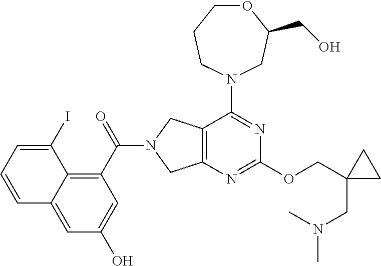 [SMILES: CN(C)CC1(COc2nc3c(c(N4CCCO[C@@H](CO)C4)n2)CN(C(=O)c2cc(O)cc4cccc(I)c24)C3)CC1] | (R)-(2-((1- ((dimethylamino)methyl)cyclopropyl) methoxy)-4-(2-(hydroxymethyl)-1,4- oxazepan-4-yl)-5,7-dihydro-6H- pyrrolo[3,4-d]pyrimidin-6-yl)(3- hydroxy-8-iodonaphthalen-1- yl)methanone | | 674 |
| 63 | 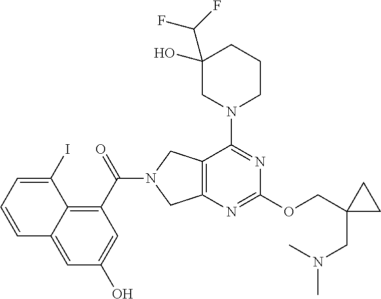 [SMILES: CN(C)CC1(COc2nc3c(c(N4CCCC(O)(C(F)F)C4)n2)CN(C(=O)c2cc(O)cc4cccc(I)c24)C3)CC1] | (4-(3-(difluoromethyl)-3- hydroxypiperidin-1-yl)-2-((1- ((dimethylamino)methyl)cyclopropyl) methoxy)-5,7-dihydro-6H-pyrrolo [3,4-d]pyrimidin-6-yl)(3-hydroxy-8- iodonaphthalen-1-yl)methanone | | 694 |
| 64 | 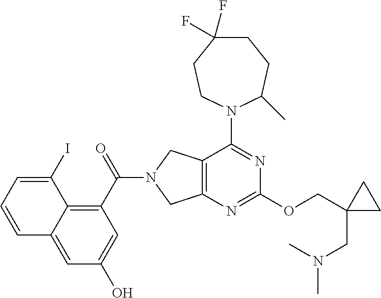 [SMILES: CC1CCC(F)(F)CCN1c1nc(OCC2(CN(C)C)CC2)nc2c1CN(C(=O)c1cc(O)cc3cccc(I)c13)C2] | (R)-(4-(5,5-difluoro-2-methylazepan- 1-yl)-2-((1-((dimethylamino) methyl)cyclopropyl) methoxy)-5,7-dihydro-6H-pyrrolo [3,4-d]pyrimidin-6-yl)(3-hydroxy-8- iodonaphthalen-1-yl)methanone | | 692 |
| 65 | 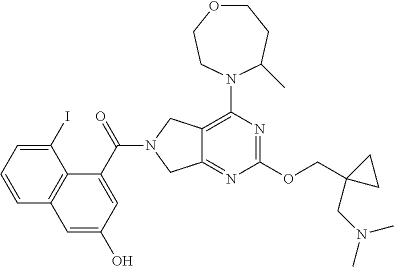 [SMILES: CC1CCOCCN1c1nc(OCC2(CN(C)C)CC2)nc2c1CN(C(=O)c1cc(O)cc3cccc(I)c13)C2] | (2-((1- ((dimethylamino)methyl)cyclopropyl) methoxy)-4-(5-methyl-1,4- oxazepan-4-yl)-5,7-dihydro-6H- pyrrolo[3,4-d]pyrimidin- 6-yl)(3-hydroxy-8-iodonaphthalen-1- yl)methanone | | 658 |
| 66 | 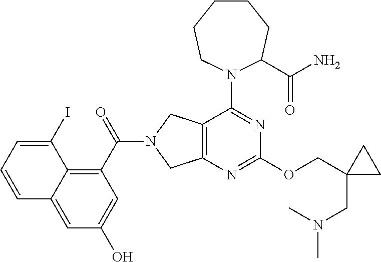 [SMILES: CN(C)CC1(COc2nc3c(c(N4CCCCCC4C(N)=O)n2)CN(C(=O)c2cc(O)cc4cccc(I)c24)C3)CC1] | 1-(2-((1- ((dimethylamino)methyl)cyclopropyl) methoxy)-6-(3-hydroxy-8-iodo-1- naphthoyl)-6,7-dihydro- 5H-pyrrolo[3,4- d]pyrimidin-4-yl)azepane- 2-carboxamide | | 685 |
| 67 | 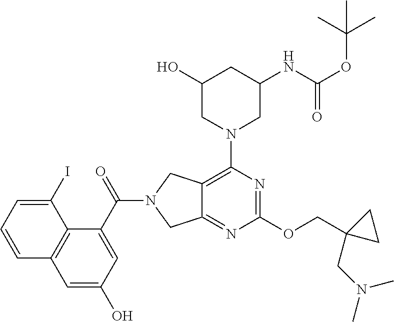 [SMILES: CN(C)CC1(COc2nc3c(c(N4CC(O)CC(NC(=O)OC(C)(C)C)C4)n2)CN(C(=O)c2cc(O)cc4cccc(I)c24)C3)CC1] | tert-butyl (1-(2-((1- ((dimethylamino)methyl)cyclopropyl) methoxy)-6-(3-hydroxy-8-iodo-1- naphthoyl)-6,7-dihydro-5H-pyrrolo [3,4-d]pyrimidin-4-yl)-5- hydroxypiperidin-3- yl)carbamate | | 759 |
| 68 | 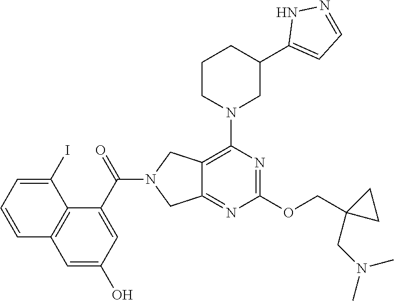 [SMILES: CN(C)CC1(COc2nc3c(c(N4CCCC(c5ccn[nH]5)C4)n2)CN(C(=O)c2cc(O)cc4cccc(I)c24)C3)CC1] | (4-(3-(1H-pyrazol-5-yl)piperidin- 1-yl)-2-((1-((dimethylamino) methyl)cyclopropyl)methoxy)-5,7- dihydro-6H-pyrrolo[3,4- d]pyrimidin-6-yl)(3-hydroxy-8- iodonaphthalen-1-yl)methanone | | 694 |
| 69 | 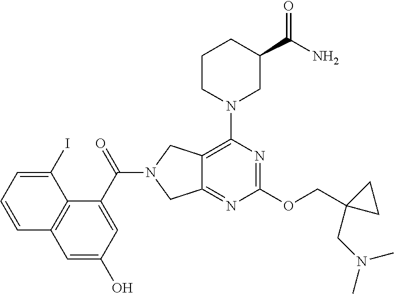 [SMILES: CN(C)CC1(COc2nc3c(c(N4CCC[C@@H](C(N)=O)C4)n2)CN(C(=O)c2cc(O)cc4cccc(I)c24)C3)CC1] | (R)-1-(2-((1- ((dimethylamino)methyl)cyclopropyl) methoxy)-6-(3-hydroxy-8-iodo-1- naphthoyl)-6,7-dihydro-5H-pyrrolo [3,4-d]pyrimidin-4-yl)piperidine-3- carboxamide | | 671 |
| 70 | 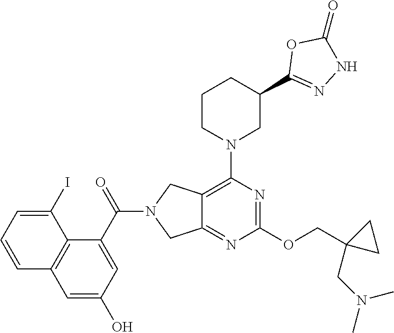 [SMILES: CN(C)CC1(COc2nc3c(c(N4CCC[C@@H](c5n[nH]c(=O)o5)C4)n2)CN(C(=O)c2cc(O)cc4cccc(I)c24)C3)CC1] | (R)-5-(1-(2-((1- ((dimethylamino)methyl)cyclopropyl) methoxy)-6-(3-hydroxy-8-iodo-1- naphthoyl)-6,7-dihydro-5H-pyrrolo [3,4-d]pyrimidin-4-yl)piperidin- 3-yl)-1,3,4- oxadiazol-2(3H)-one | | 712 |
| 71 | 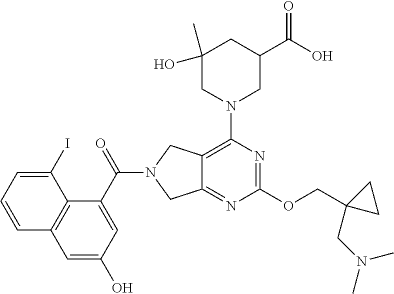 [SMILES: CN(C)CC1(COc2nc3c(c(N4CC(C(=O)O)CC(C)(O)C4)n2)CN(C(=O)c2cc(O)cc4cccc(I)c24)C3)CC1] | 1-(2-((1- ((dimethylamino)methyl)cyclopropyl) methoxy)-6-(3-hydroxy-8-iodo-1- naphthoyl)-6,7-dihydro-5H-pyrrolo [3,4-d]pyrimidin-4-yl)-5-hydroxy-5- methylpiperidine-3-carboxylic acid | | 702 |
| 72 | 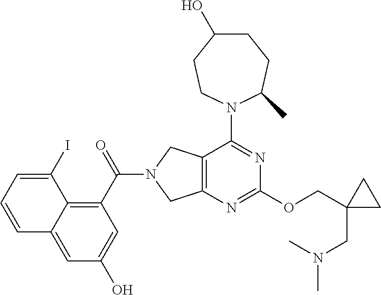 [SMILES: C[C@@H]1CCC(O)CCN1c1nc(OCC2(CN(C)C)CC2)nc2c1CN(C(=O)c1cc(O)cc3cccc(I)c13)C2] | (2-((1- ((dimethylamino)methyl)cyclopropyl) methoxy)-4-((2R)-5-hydroxy-2- methylazepan-1-yl)-5,7-dihydro-6H- pyrrolo[3,4-d]pyrimidin-6-yl)(3- hydroxy-8-iodonaphthalen-1- yl)methanone | 1H NMR (400 MHz, DMSO- d₆) δ 10.3-10.2 (m, 1H), 8.02- 7.96 (m, 1H), 7.88-7.81 (m, 1H), 7.29-7.23 (m, 1H), 7.18- 7.06 (m, 2H), 5.20-4.83 (m, 1H), 4.76-4.30 (m, 3H), 4.21- 3.83 (m, 5H), 3.00-2.80 (m, 1H), 2.30-2.18 (m, 8H), 2.15- 1.43 (m, 4H), 1.41-0.77 (m, | 672 |
| | | | 6H), 0.72-0.32 | |
| | | | (m, 4H). | |
| 73 | 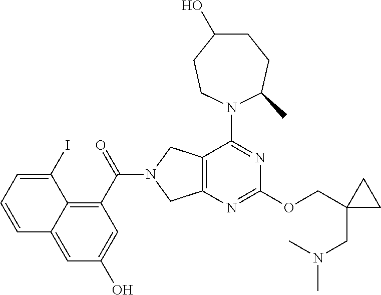 [SMILES: C[C@@H]1CCC(O)CCN1c1nc(OCC2(CN(C)C)CC2)nc2c1CN(C(=O)c1cc(O)cc3cccc(I)c13)C2] | (2-((1- ((dimethylamino)methyl)cyclopropyl) methoxy)-4-((2R)-5-hydroxy-2- methylazepan-1-yl)-5,7-dihydro-6H- pyrrolo[3,4-d]pyrimidin-6-yl)(3- hydroxy-8-iodonaphthalen-1- yl)methanone | | 672 |
| 74 | 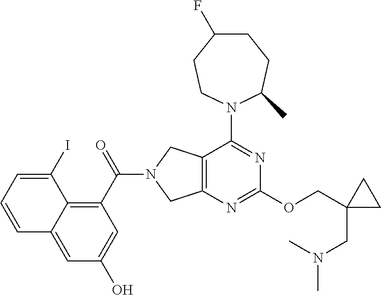 [SMILES: C[C@@H]1CCC(F)CCN1c1nc(OCC2(CN(C)C)CC2)nc2c1CN(C(=O)c1cc(O)cc3cccc(I)c13)C2] | (2-((1- ((dimethylamino)methyl)cyclopropyl) methoxy)-4-((2R)-5-fluoro-2- methylazepan-1-yl)-5,7-dihydro- 6H-pyrrolo[3,4-d]pyrimidin-6- yl)(3-hydroxy-8-iodonaphthalen- 1-yl)methanone | 1H NMR (400 MHz, DMSO- d₆) δ 10.2 (s, 1H), 8.02-7.94 (m, 1H), 7.87- 7.80 (m, 1H), 7.28-7.23 (m, 1H), 7.16-7.05 (m, 2H), 5.23- 4.84 (m, 1H), 4.80-4.30 (m, 3H), 4.22-3.95 (m, 4H), 2.96- 2.80 (m, 1H), 2.29-2.17 (m, 1H), 2.17-2.08 (m, 7H), 2.06- 1.60 (m, 4H), | 674 |
| | | | 1.59-0.85 (m, | |
| | | | 6H), 0.66-0.48 | |
| | | | (m, 2H), 0.43- | |
| | | | 0.28 (m, 2H). | |
| 75 | 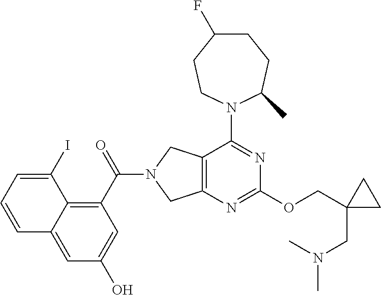 [SMILES: C[C@@H]1CCC(F)CCN1c1nc(OCC2(CN(C)C)CC2)nc2c1CN(C(=O)c1cc(O)cc3cccc(I)c13)C2] | (2-((1- ((dimethylamino)methyl)cyclopropyl) methoxy)-4-((2R)-5-fluoro-2- methylazepan-1-yl)-5,7-dihydro- 6H-pyrrolo[3,4- d]pyrimidin-6-yl)(3-hydroxy-8- iodonaphthalen-1-yl)methanone | | 674 |
| 76 | 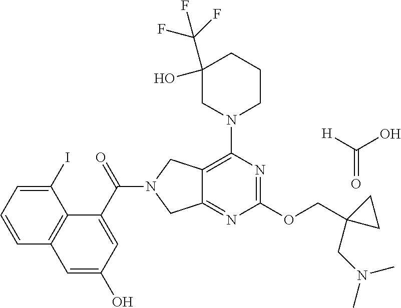 | (2-((1- ((dimethylamino)methyl)cyclopropyl) methoxy)-4-(3-hydroxy-3- (trifluoromethyl)piperidin-1-yl)-5,7- dihydro-6H-pyrrolo[3,4-d]pyrimidin-6- yl)(3-hydroxy-8-iodonaphthalen-1- yl)methanone formate | | 712 |
| 77 | 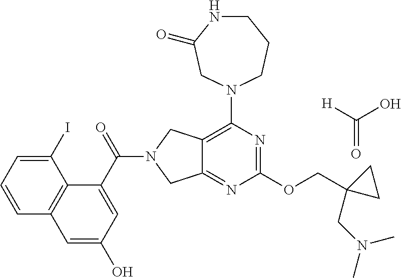 | 4-(2-((1- ((dimethylamino)methyl)cyclopropyl) methoxy)-6-(3-hydroxy-8-iodo-1- naphthoyl)-6,7-dihydro-5H-pyrrolo [3,4-d]pyrimidin-4-yl)-1,4-diazepan- 2-one formate | | 657 |
| 78 | 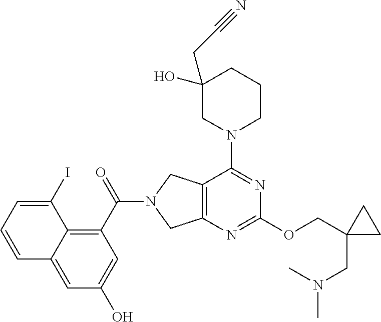 [SMILES: CN(C)CC1(COc2nc3c(c(N4CCCC(O)(CC#N)C4)n2)CN(C(=O)c2cc(O)cc4cccc(I)c24)C3)CC1] | 2-(1-(2-((1- ((dimethylamino)methyl)cyclopropyl) methoxy)-6-(3-hydroxy-8-iodo-1- naphthoyl)-6,7-dihydro-5H-pyrrolo [3,4-d]pyrimidin-4-yl)-3- hydroxypiperidin-3- yl)acetonitrile | 1H NMR (400 MHz, DMSO- d₆) δ 10.2 (s, 1H), 8.02-7.95 (m, 1H), 7.87- 7.79 (m, 1H), 7.27 (d, J = 2.6 Hz, 0.7H), 7.24 (d, J = 2.6 Hz, 0.3H), 7.15- 7.07 (m, 2H), 5.36 (d, J = 3.8 Hz, 0.7H), 5.21 (d, J = 5.2 Hz, 0.3H), 5.05- 4.85 (m, 1H) 4.75-4.39 (m, 1H), 4.19-3.92 (m, 4H), 3.81- 3.38 (m, | 683 |
| | | | 2H),2.71 (s,1H), | |
| | | | 2.21-2.06 (m, | |
| | | | 8H), 1.85-1.69 | |
| | | | (m, 2H), 1.67- | |
| | | | 1.51 (m, 2H), | |
| | | | 1.30-1.12 (m, | |
| | | | 3H), 0.60-0.46 | |
| | | | (m, 2H), 0.42- | |
| | | | 0.25 (m, 2H). | |
| 79 | 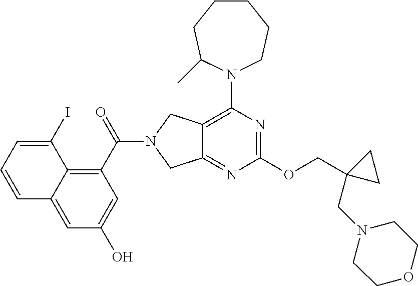 [SMILES: CC1CCCCCN1c1nc(OCC2(CN3CCOCC3)CC2)nc2c1CN(C(=O)c1cc(O)cc3cccc(I)c13)C2] | (3-hydroxy-8-iodonaphthalen-1- yl)(4-(2-methylazepan-1-yl)-2-((1- (morpholinomethyl)cyclopropyl) methoxy)-5,7-dihydro-6H-pyrrolo [3,4-d]pyrimidin-6-yl)methanone | | 698 |
| 80 | 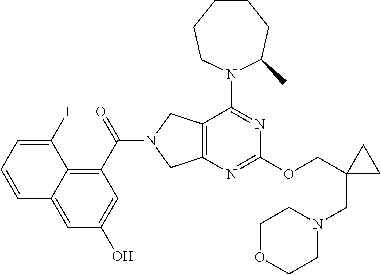 [SMILES: C[C@@H]1CCCCCN1c1nc(OCC2(CN3CCOCC3)CC2)nc2c1CN(C(=O)c1cc(O)cc3cccc(I)c13)C2] | (R)-(3-hydroxy-8-iodonaphthalen-1- yl)(4-(2-methylazepan-1-yl)-2-((1- (morpholinomethyl)cyclopropyl) methoxy)-5,7-dihydro-6H-pyrrolo [3,4-d]pyrimidin-6-yl)methanone | 1H NMR (400 MHz, DMSO- d₆) δ 10.3 (s, 1H), 8.02-7.96 (m, 1H), 7.86- 7.80 (m, 1H), 7.28-7.23 (m, 1H), 7.18-7.06 (m, 2H), 5.16- 4.86 (m, 1H), 4.82-4.33 (m, 3H), 4.22-3.96 (m, 4H), 3.56- 3.46 (m, 4H), 3.05-2.94 (m, 1H), 2.41-2.18 (m, 6H), 1.81- | 698 |
| | | | 1.41 (m, 4H), | |
| | | | 1.40-0.76 (m, | |
| | | | 7H), 0.60-0.47 | |
| | | | (m, 2H), 0.40- | |
| | | | 0.29 (m, 2H). | |
| 81 | 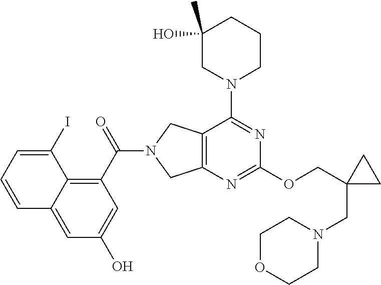 [SMILES: C[C@@]1(O)CCCN(c2nc(OCC3(CN4CCOCC4)CC3)nc3c2CN(C(=O)c2cc(O)cc4cccc(I)c24)C3)C1] | (R)-(4-(3-hydroxy-3-methylpiperidin- 1-yl)-2-((1-(morpholinomethyl) cyclopropyl)methoxy )-5,7-dihydro-6H-pyrrolo[3,4- d]pyrimidin-6-yl)(3-hydroxy-8- iodonaphthalen-1-yl)methanone | | 700 |

### Example 82: (4-(3-amino-5-hydroxypiperidin-1-yl)-2-((1-((dimethylamino)methyl)cyclopropyl)methoxy)-5,7-dihydro-6H-pyrrolo[3,4-d]pyrimidin-6-yl)(3-hydroxy-8-iodonaphthalen-1-yl)methanone (Ex. 82)

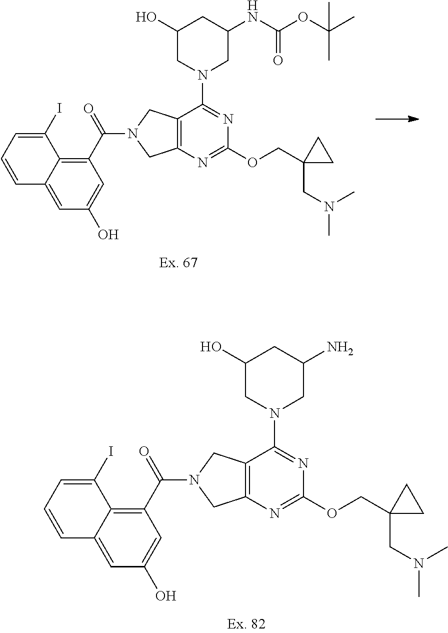 [SMILES: CN(C)CC1(COc2nc3c(c(N4CC(O)CC(NC(=O)OC(C)(C)C)C4)n2)CN(C(=O)c2cc(O)cc4cccc(I)c24)C3)CC1 ; CN(C)CC1(COc2nc3c(c(N4CC(N)CC(O)C4)n2)CN(C(=O)c2cc(O)cc4cccc(I)c24)C3)CC1]

To a solution of tert-butyl (1-(2-((1-((dimethylamino)methyl)cyclopropyl)methoxy)-6-(3-hydroxy-8-iodo-1-naphthoyl)-6,7-dihydro-5H-pyrrolo[3,4-d]pyrimidin-4-yl)-5-hydroxypiperidin-3-yl)carbamate (Ex. 67) (30 mg, 0.0395 mmol) in dichloromethane (5 mL) was added trifluoroacetic acid (5 mL) and the mixture was stirred for 1 h. Trifluoroacetic acid was removed under reduced pressure and the residue was purified by flash NH-silica gel chromatography (0-50%, methanol gradient in ethyl acetate) to give (4-(3-amino-5-hydroxypiperidin-1-yl)-2-((1-((dimethylamino)methyl)cyclopropyl)methoxy)-5,7-dihydro-6H-pyrrolo[3,4-d]pyrimidin-6-yl)(3-hydroxy-8-iodonaphthalen-1-yl)methanone (Ex. 82). ESI-MS m/z [M+H]⁺ 659.

### Example 83: N-(1-(2-((1-((dimethylamino)methyl)cyclopropyl)methoxy)-6-(3-hydroxy-8-iodo-1-naphthoyl)-6,7-dihydro-5H-pyrrolo[3,4-d]pyrimidin-4-yl)-5-hydroxypiperidin-3-yl)-1-methyl-1H-pyrazole-5-carboxamide (Ex. 83)

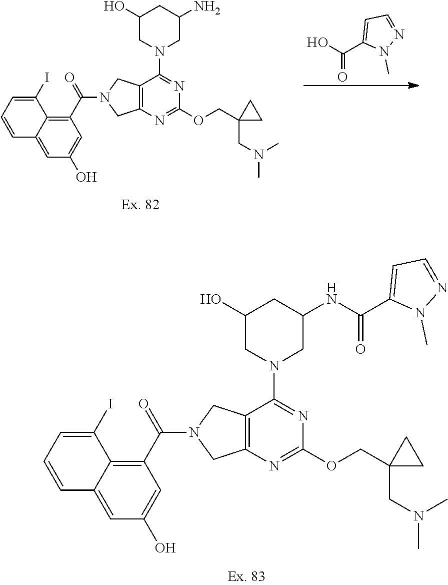

To a mixture of 1-methyl-1H-pyrazole-5-carboxylic acid (3.0 mg, 0.0237 mmol), 1-hydroxybenzotriazole monohydrate (2.9 mg, 0.0189 mmol), 1-(3-dimethylaminopropyl)-3-ethylcarbodiimide hydrochloride (3.6 mg, 0.0189 mmol) and N,N-diisopropylethylamine (0.006 mL, 0.0316 mmol) in N,N-dimethylformamide (0.3 mL) was added (4-(3-amino-5-hydroxypiperidin-1-yl)-2-((1-((dimethylamino)methyl)cyclopropyl)methoxy)-5,7-dihydro-6H-pyrrolo[3,4-d]pyrimidin-6-yl)(3-hydroxy-8-iodonaphthalen-1-yl)methanone (Ex. 82) (10 mg, 0.015 mmol), and the mixture was stirred at room temperature for 5 h. The mixture was diluted with DMSO (0.6 mL), and the product mixture was purified by reverse phase HPLC (MeCN/water with 0.1% formic acid) to give N-(1-(2-((1-((dimethylamino)methyl)cyclopropyl)methoxy)-6-(3-hydroxy-8-iodo-1-naphthoyl)-6,7-dihydro-5H-pyrrolo[3,4-d]pyrimidin-4-yl)-5-hydroxypiperidin-3-yl)-1-methyl-1H-pyrazole-5-carboxamide (Ex. 83). ESI-MS m/z [M+H]⁺ 767. ¹H NMR (400 MHZ, DMSO-d₆) δ 10.3 (s, 1H), 8.49-8.13 (m, 1H), 8.04-7.96 (m, 1H), 7.89-7.78 (m, 1H), 7.47-7.39 (m, 1H), 7.30-7.20 (m, 1H), 7.18-7.05 (m, 2H), 6.84-6.66 (m, 1H), 5.15-4.52 (m, 2H), 4.28-3.65 (m, 7H), 3.64-3.12 (m, 10H) 2.25-2.18 (m, 1H), 2.16 (s, 3H), 2.11 (s, 3H), 1.93-1.43 (m, 1H), 0.61-0.48 (m, 2H), 0.40-0.30 (m, 2H)

### Example 84-1: (2-((1-((dimethylamino)methyl)cyclopropyl)methoxy)-4-(2-methylazepan-1-yl)-5,7-dihydro-6H-pyrrolo[3,4-d]pyrimidin-6-yl)(3-hydroxy-8-iodonaphthalen-1-yl)methanone (Ex. 84-1)

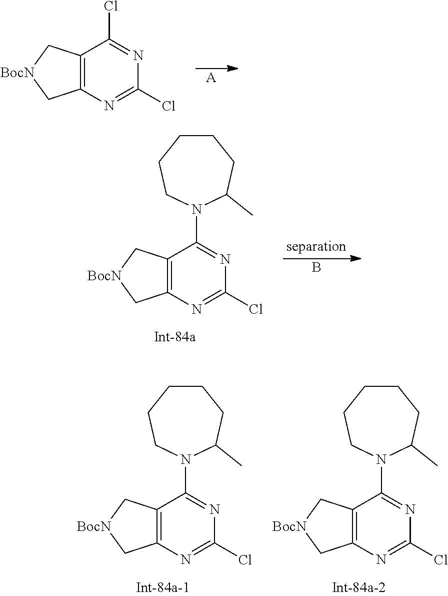

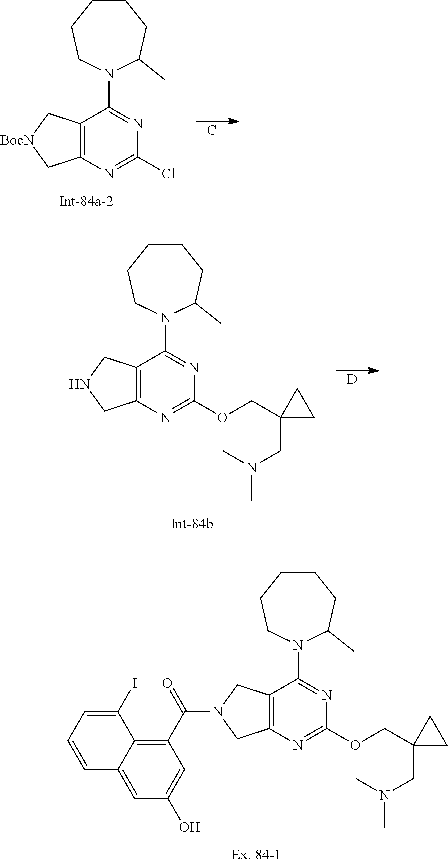

### Step A: tert-butyl 2-chloro-4-(2-methylazepan-1-yl)-5,7-dihydro-6H-pyrrolo[3,4-d]pyrimidine-6-carboxylate (Int-84a)

To a stirred solution of tert-butyl 2,4-dichloro-5,7-dihydro-6H-pyrrolo[3,4-d]pyrimidine-6-carboxylate (3.88 g, 13.4 mol) and 2-methylazepane hydrochloride (2.00 g, 13.4 mol) in DME (30 mL) was added N,N-diisopropylethylamine (6.82 mL, 40.1 mmol), and the mixture was warmed to 50° C. and stirred for 5 h. The mixture was cooled to room temperature diluted with ethyl acetate. The diluted mixture was washed with water and the mixture was concentrated under reduced pressure. The residue was purified by flash silica gel chromatography (0-40%, ethyl acetate gradient in hexane) to give tert-butyl 2-chloro-4-(2-methylazepan-1-yl)-5,7-dihydro-6H-pyrrolo[3,4-d]pyrimidine-6-carboxylate (Int-84a) (3.52 g). ESI-MS m/z [M+H]⁺ 367, 369.

### Step B: tert-butyl 2-chloro-4-(2-methylazepan-1-yl)-5,7-dihydro-6H-pyrrolo[3,4-d]pyrimidine-6-carboxylate (Int-84a-1 peak 1 and Int-84a-2 peak2) by Chiral HPLC

Racemic tert-butyl 2-chloro-4-(2-methylazepan-1-yl)-5,7-dihydro-6H-pyrrolo[3,4-d]pyrimidine-6-carboxylate (Int-84a) was separated by chiral HPLC (Column: Chiral Art SB 4.6 mmφ(YMC), hexane/EtOH 0.1% diethylamine) to give tert-butyl 2-chloro-4-(2-methylazepan-1-yl)-5,7-dihydro-6H-pyrrolo[3,4-d]pyrimidine-6-carboxylate (Int-84a-1, first elution). ESI-MS m/z [M+H]⁺ 367, 369). And tert-butyl 2-chloro-4-(2-methylazepan-1-yl)-5,7-dihydro-6H-pyrrolo[3,4-d]pyrimidine-6-carboxylate (Int-84a-2, second elution). ESI-MS m/z [M+H]⁺ 367, 369.

### Step C: N,N-dimethyl-1-(1-(((4-(2-methylazepan-1-yl)-6,7-dihydro-5H-pyrrolo[3,4-d]pyrimidin-2-yl)oxy)methyl)cyclopropyl)methanamine (Int-84b)

To a mixture of tert-butyl 2-chloro-4-(2-methylazepan-1-yl)-5,7-dihydro-6H-pyrrolo[3,4-d]pyrimidine-6-carboxylate (Int-84a-2 peak 2, second elution) (360 mg, 0.981 mmol), (1-((dimethylamino)methyl)cyclopropyl)methanol (254 mg, 1.96 mmol), dichloro[9,9-dimethyl-4,5-bis(diphenylphosphino)xanthene]palladium(II) (74 mg, 0.098 mmol) in toluene (10 mL) was added cesium carbonate (959 mg, 2.94 mmol), and the mixture was warmed to 130° C. and stirred for 3 h. The mixture was cooled to room temperature and filtered, and the filtrate was concentrated under reduced pressure. The residue was purified by flash silica gel chromatography (0-25%, methanol gradient in chloroform) to give the coupling product. To a solution of the coupling product in dichloromethane (2 mL) was added trifluoroacetic acid (2 mL) and the mixture was stirred for 1 h. Trifluoroacetic acid was removed under reduced pressure and the residue was purified by flash NH-silica gel chromatography (0-50%, methanol gradient in ethyl acetate) to give N,N-dimethyl-1-(1-(((4-(2-methylazepan-1-yl)-6,7-dihydro-5H-pyrrolo[3,4-d]pyrimidin-2-yl)oxy)methyl)cyclopropyl)methanamine (Int-84b) (180 mg). ESI-MS m/z [M+H]⁺ 360.

### Step D: Example 84-1: (2-((1-((dimethylamino)methyl)cyclopropyl)methoxy)-4-(2-methylazepan-1-yl)-5,7-dihydro-6H-pyrrolo[3,4-d]pyrimidin-6-yl)(3-hydroxy-8-iodonaphthalen-1-yl)methanone (Ex. 84)

To a mixture of 3-hydroxy-8-iodo-1-naphthoic acid (Int-T002) (105 mg, 0.334 mmol), 1-hydroxybenzotriazole monohydrate (51 mg, 0.334 mmol), 1-(3-dimethylaminopropyl)-3-ethylcarbodiimide hydrochloride (64 g, 0.334 mmol) and N,N-diisopropylethylamine (0.14 mL, 0.834 mmol) in N,N-dimethylformamide (1 mL) was added N,N-dimethyl-1-(1-(((4-(2-methylazepan-1-yl)-6, 7-dihydro-5H-pyrrolo[3,4-d]pyrimidin-2-yl)oxy)methyl)cyclopropyl)methanamine (Int-84b) (100 mg, 0.278 mmol), and the mixture was stirred at 40° C. for 2 h. The mixture was diluted with ethyl acetate and the diluted mixture was washed with water and the mixture was concentrated under reduced pressure. The residue was purified by flash NH-silica gel chromatography (0-60%, methanol gradient in ethyl acetate) to give (2-((1-((dimethylamino)methyl)cyclopropyl)methoxy)-4-(2-methylazepan-1-yl)-5,7-dihydro-6H-pyrrolo[3,4-d]pyrimidin-6-yl)(3-hydroxy-8-iodonaphthalen-1-yl)methanone (Ex. 84-1). ESI-MS m/z [M+H]⁺ 656. ¹H NMR (400 MHZ, DMSO-d₆) δ 10.3 (s, 1H), 8.02-7.98 (m, 1H), 7.88-7.80 (m, 1H), 7.29-7.24 (m, 1H), 7.19-7.09 (m, 2H), 5.15-5.04 (m, 1H), 4.96-4.88 (m, 1H), 4.70-4.38 (m, 1H), 4.22-4.00 (m, 5H), 3.21-2.80 (m, 3H), 2.26-2.10 (m, 1H), 2.15 (s, 3H), 2.11 (s, 3H), 1.85-0.90 (m, 8H), 0.59-0.50 (m, 2H), 0.39-0.31 (m, 2H).

### Example 84-2: (3-hydroxy-8-iodonaphthalen-1-yl)(2-((1-(hydroxymethyl)cyclopropyl)methoxy)-4-(2-methylazepan-1-yl)-5,7-dihydro-6H-pyrrolo[3,4-d]pyrimidin-6-yl)methanone (Ex. 84-2)

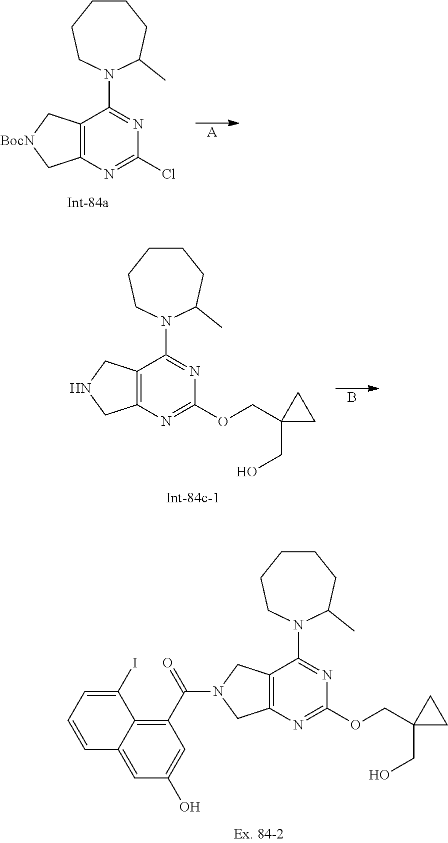

### Step A: (1-(((4-(2-methylazepan-1-yl)-6,7-dihydro-5H-pyrrolo[3,4-d]pyrimidin-2-yl)oxy)methyl)cyclopropyl)methanol (Int-84c-1)

To a mixture of tert-butyl 2-chloro-4-(2-methylazepan-1-yl)-5,7-dihydro-6H-pyrrolo[3,4-d]pyrimidine-6-carboxylate (Int-84a) (1.50 g, 4.09 mmol), 1,1-bis(hydroxymethyl)cyclopropane (1.25 g, 12.3 mmol), RuPhos Pd G3 (171 mg, 0.204 mmol) in 1,4-dioxane (15 mL) was added cesium carbonate (4.00 g, 12.3 mmol), and the mixture was warmed to 110° C. and stirred for 15 min. The mixture was cooled to room temperature and filtered, and the filtrate was concentrated under reduced pressure. The residue was purified by flash silica gel chromatography (40-80%, ethyl acetate gradient in hexane) to give the coupling product. To a solution of the coupling product in dichloromethane (5 mL) was added trifluoroacetic acid (5 mL) and the mixture was stirred for 1 h. Trifluoroacetic acid was removed under reduced pressure and the residue was purified by flash NH-silica gel chromatography (0-50%, methanol gradient in ethyl acetate) to give (1-(((4-(2-methylazepan-1-yl)-6,7-dihydro-5H-pyrrolo[3,4-d]pyrimidin-2-yl)oxy)methyl)cyclopropyl)methanol (Int-84c-1) (1.36 g). ESI-MS m/z [M+H]⁺ 333.

### Step B: (3-hydroxy-8-iodonaphthalen-1-yl)(2-((1-(hydroxymethyl)cyclopropyl)methoxy)-4-(2-methylazepan-1-yl)-5,7-dihydro-6H-pyrrolo[3,4-d]pyrimidin-6-yl)methanone (Ex. 84-2)

To a mixture of 3-hydroxy-8-iodo-1-naphthoic acid (Int-T002) (1.54 g, 4.91 mmol), 1-hydroxybenzotriazole monohydrate (751 mg, 4.91 mmol), 1-(3-dimethylaminopropyl)-3-ethylcarbodiimide hydrochloride (941 g, 4.91 mmol) and N,N-diisopropylethylamine (2.09 mL, 12.3 mmol) in N,N-dimethylformamide (15 mL) was added (1-(((4-(2-methylazepan-1-yl)-6,7-dihydro-5H-pyrrolo[3,4-d]pyrimidin-2-yl)oxy)methyl)cyclopropyl)methanol (Int-84c-1) (1.36 g, 4.09 mmol), and the mixture was stirred at room temperature for 2 h. The mixture was diluted with ethyl acetate and the diluted mixture was washed with water and the mixture was concentrated under reduced pressure. The residue was suspended in ethyl acetate (10 mL) and the solid was filtered and dried under reduced pressure to give (3-hydroxy-8-iodonaphthalen-1-yl)(2-((1-(hydroxymethyl)cyclopropyl)methoxy)-4-(2-methylazepan-1-yl)-5, 7-dihydro-6H-pyrrolo[3,4-d]pyrimidin-6-yl)methanone (Ex. 84-2) (1.45 g). ESI-MS m/z [M+H]⁺ 629.

### Example 85: (3-hydroxy-8-iodonaphthalen-1-yl)(2-((1-(((R)-3-methoxypiperidin-1-yl)methyl)cyclopropyl)methoxy)-4-(2-methylazepan-1-yl)-5,7-dihydro-6H-pyrrolo[3,4-d]pyrimidin-6-yl)methanone (Ex. 85)

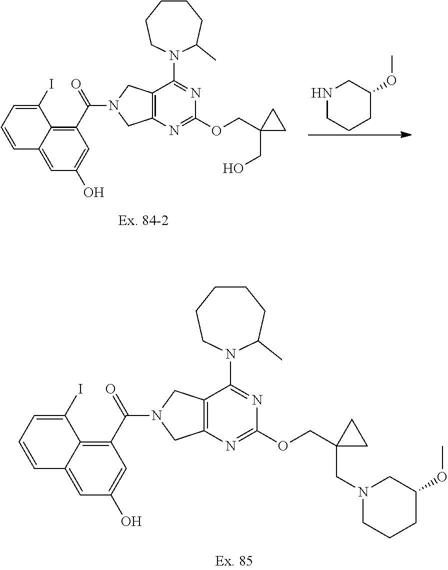

To a mixture of (3-hydroxy-8-iodonaphthalen-1-yl)(2-((1-(hydroxymethyl)cyclopropyl)methoxy)-4-(2-methylazepan-1-yl)-5,7-dihydro-6H-pyrrolo[3,4-d]pyrimidin-6-yl)methanone (Ex. 84-2) (20.0 mg, 0.0318 mmol) and N,N-diisopropylethylamine (27 mL, 0.159 mmol) in DMF (0.3 mL) was added ethanesulfonyl chloride (12.3 mg, 0.0955 mmol) at 0° C., and the mixture was stirred at 0° C. for 0.5 h. To the mixture was added (R)-3-methoxypiperidine (18.3 mg, 0.159 mmol) and the mixture was stirred at 70° C. for 1 h. Then methanol (0.1 mL) and aqueous NaOH (2M, 0.2 mL) was added to the mixture and the mixture was stirred at 70° C. for another 0.5 h. The mixture was cooled to room temperature and diluted with DMSO (0.6 mL), and the product mixture was purified by reverse phase HPLC (MeCN/water with 0.1% formic acid) to give (3-hydroxy-8-iodonaphthalen-1-yl)(2-((1-(((R)-3-methoxypiperidin-1-yl)methyl)cyclopropyl)methoxy)-4-(2-methylazepan-1-yl)-5,7-dihydro-6H-pyrrolo[3,4-d]pyrimidin-6-yl)methanone (Ex. 85). ESI-MS m/z [M+H]⁺ 726. ¹H NMR (400 MHz, DMSO-d₆) δ 10.2 (8, 1H), 8.04-7.98 (m, 1H) 7.88-7.82 (m, 1H), 7.30-7.26 (m, 1H), 7.19-7.09 (m, 2H), 5.15-5.03 (m, 1H), 4.98-4.87 (m, 1H), 4.70-4.35 (m, 2H) 4.21-3.99 (m, 5H), 3.23 (s, 1.5H), 3.28 (8, 1.5H), 3.15-2.78 (m, 2H), 2.31-2.11 (m, 2H), 2.00-0.95 (m, 24H), 0.61-0.51 (m, 2H), 0.40-0.31 (m, 2H).

Compounds in the table below were synthesized via a similar route as Ex. 85 using Ex. 84-2 and corresponding amines.

TABLE 2

| | | | | ESI-MS |
|---|---|---|---|---|
| Example | Structure | Name | NMR | [M + H]⁺ |
| 86 | 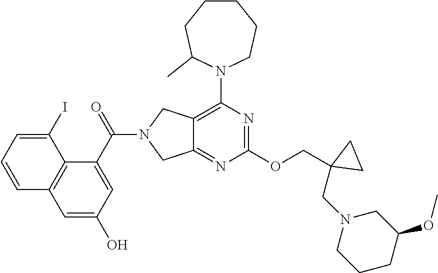 [SMILES: CO[C@H]1CCCN(CC2(COc3nc4c(c(N5CCCCCC5C)n3)CN(C(=O)c3cc(O)cc5cccc(I)c35)C4)CC2)C1] | (3-hydroxy-8-iodonaphthalen-1-yl)(2-((1- (((S)-3-methoxypiperidin-1- yl)methyl)cyclopropyl)methoxy)-4-(2- methylazepan-1-yl)-5,7-dihydro-6H- pyrrolo[3,4-d]pyrimidin-6-yl)methanone | | 726 |
| 87 | 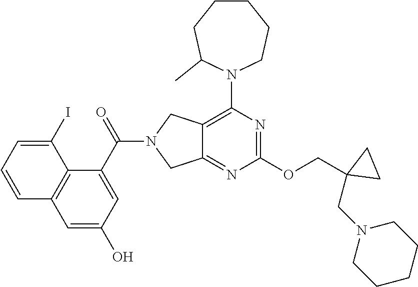 [SMILES: CC1CCCCCN1c1nc(OCC2(CN3CCCCC3)CC2)nc2c1CN(C(=O)c1cc(O)cc3cccc(I)c13)C2] | (3-hydroxy-8-iodonaphthalen-1-yl)(4-(2- methylazepan-1-yl)-2-((1-(piperidin-1- ylmethyl)cyclopropyl)methoxy)-5,7-dihydro- 6H-pyrrolo[3,4-d]pyrimidin-6-yl)methanone | | 696 |
| 88 | 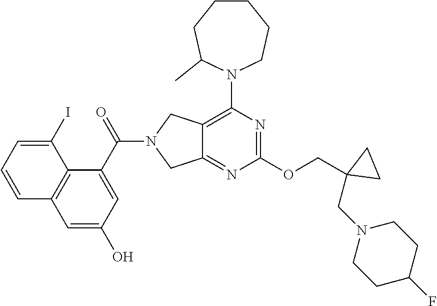 [SMILES: CC1CCCCCN1c1nc(OCC2(CN3CCC(F)CC3)CC2)nc2c1CN(C(=O)c1cc(O)cc3cccc(I)c13)C2] | (2-((1-((4-fluoropiperidin-1- yl)methyl)cyclopropyl)methoxy)-4-(2- methylazepan-1-yl)-5,7-dihydro-6H- pyrrolo[3,4-d]pyrimidin-6-yl)(3-hydroxy-8- iodonaphthalen-1-yl)methanone | | 714 |
| 89 | 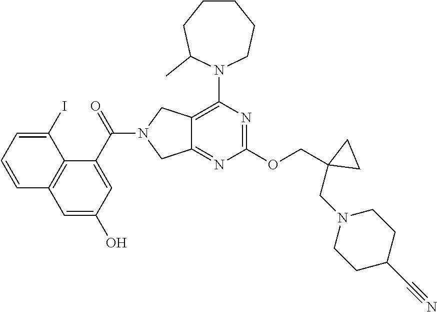 [SMILES: CC1CCCCCN1c1nc(OCC2(CN3CCC(C#N)CC3)CC2)nc2c1CN(C(=O)c1cc(O)cc3cccc(I)c13)C2] | 1-((1-(((6-(3-hydroxy-8-iodo-1-naphthoyl)-4- (2-methylazepan-1-yl)-6,7-dihydro-5H- pyrrolo[3,4-d]pyrimidin-2- yl)oxy)methyl)cyclopropyl)methyl)piperidine- 4-carbonitrile | | 721 |
| 90 | 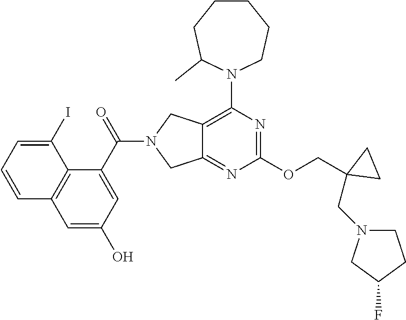 [SMILES: CC1CCCCCN1c1nc(OCC2(CN3CC[C@H](F)C3)CC2)nc2c1CN(C(=O)c1cc(O)cc3cccc(I)c13)C2] | (2-((1-(((S)-3-fluoropyrrolidin-1- yl)methyl)cyclopropyl)methoxy)-4-(2- methylazepan-1-yl)-5,7-dihydro-6H- pyrrolo[3,4-d]pyrimidin-6-yl)(3-hydroxy-8- iodonaphthalen-1-yl)methanone | | 700 |
| 91 | 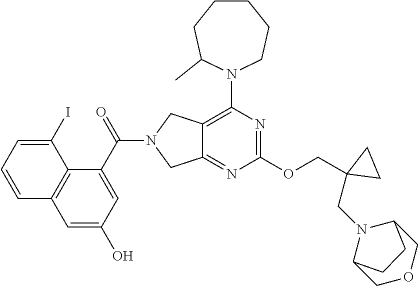 [SMILES: CC1CCCCCN1c1nc(OCC2(CN3C4CCC3COC4)CC2)nc2c1CN(C(=O)c1cc(O)cc3cccc(I)c13)C2] | (2-((1-((3-oxa-8-azabicyclo[3.2.1]octan-8- yl)methyl)cyclopropyl)methoxy)-4-(2- methylazepan-1-yl)-5,7-dihydro-6H- pyrrolo[3,4-d]pyrimidin-6-yl)(3-hydroxy-8- iodonaphthalen-1-yl)methanone | | 724 |
| 92 | 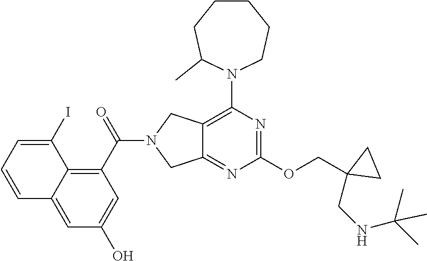 [SMILES: CC1CCCCCN1c1nc(OCC2(CNC(C)(C)C)CC2)nc2c1CN(C(=O)c1cc(O)cc3cccc(I)c13)C2] | (2-((1-((tert- butylamino)methyl)cyclopropyl)methoxy)-4- (2-methylazepan-1-yl)-5,7-dihydro-6H- pyrrolo[3,4-d]pyrimidin-6-yl)(3-hydroxy-8- iodonaphthalen-1-yl)methanone | | 684 |

### Example 84-2a: (R)-(3-hydroxy-8-iodonaphthalen-1-yl)(2-((1-(hydroxymethyl)cyclopropyl)methoxy)-4-(2-methylazepan-1-yl)-5,7-dihydro-6H-pyrrolo[3,4-d]pyrimidin-6-yl)methanone (Ex. 84-2a)

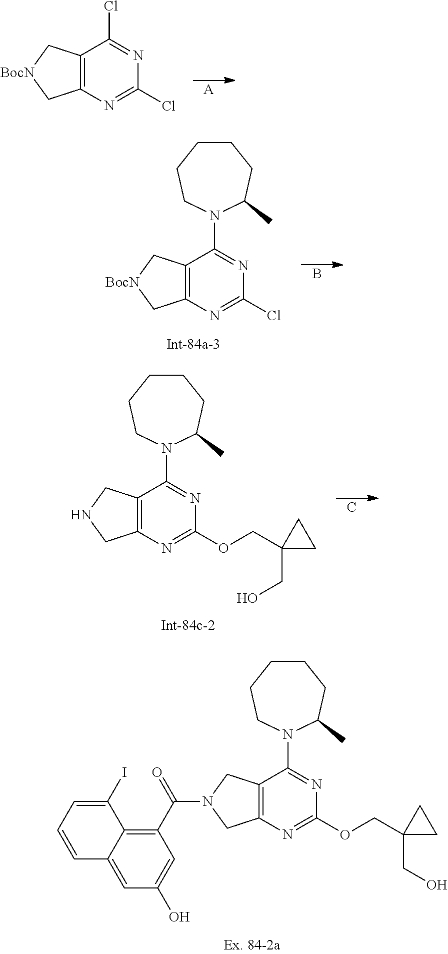

### Step A: tert-butyl (R)-2-chloro-4-(2-methylazepan-1-yl)-5,7-dihydro-6H-pyrrolo[3,4-d]pyrimidine-6-carboxylate (Int-84a-3)

To a stirred solution of tert-butyl 2,4-dichloro-5,7-dihydro-6H-pyrrolo[3,4-d]pyrimidine-6-carboxylate (2.13 g, 7.35 mol) and (R)-2-methylazepane hydrochloride (1.00 g, 6.68 mol) in DME (20 mL) was added N,N-diisopropylethylamine (3.41 mL, 20.0 mmol), and the mixture was warmed to 60° C. and stirred for 4 h. The mixture was cooled to room temperature diluted with ethyl acetate. The diluted mixture was washed with water and the mixture was concentrated under reduced pressure. The residue was purified by flash silica gel chromatography (0-40%, ethyl acetate gradient in hexane) to give tert-butyl (R)-2-chloro-4-(2-methylazepan-1-yl)-5,7-dihydro-6H-pyrrolo[3,4-d]pyrimidine-6-carboxylate (Int-84a-3) (1.92 g). ESI-MS m/z [M+H]⁺ 367, 369.

### Step B: (R)-(1-(((4-(2-methylazepan-1-yl)-6,7-dihydro-5H-pyrrolo[3,4-d]pyrimidin-2-yl)oxy)methyl)cyclopropyl)methanol (Int-84c-2)

To a mixture of tert-butyl (R)-2-chloro-4-(2-methylazepan-1-yl)-5,7-dihydro-6H-pyrrolo[3,4-d]pyrimidine-6-carboxylate (Int-84a-3) (920 mg, 2.51 mmol), 1,1-bis(hydroxymethyl)cyclopropane (768 mg, 7.52 mmol), RuPhos Pd G3 (52.4 mg, 0.0627 mmol) in 1,4-dioxane (15 mL) was added cesium carbonate (2.45 g, 7.52 mmol), and the mixture was warmed to 110° C. and stirred for 30 min. The mixture was cooled to room temperature and filtered, and the filtrate was concentrated under reduced pressure. The residue was purified by flash silica gel chromatography (40-80%, ethyl acetate gradient in hexane) to give the coupling product. To a solution of the coupling product in dichloromethane (5 mL) was added trifluoroacetic acid (5 mL) and the mixture was stirred for 1 h. Trifluoroacetic acid was removed under reduced pressure and the residue was purified by flash NH-silica gel chromatography (0-50%, methanol gradient in ethyl acetate) to give (R)-(1-(((4-(2-methylazepan-1-yl)-6,7-dihydro-5H-pyrrolo[3,4-d]pyrimidin-2-yl)oxy)methyl)cyclopropyl)methanol (Int-84c-2) (500 mg). ESI-MS m/z [M+H]⁺ 333.

### Step C: (R)-(3-hydroxy-8-iodonaphthalen-1-yl)(2-((1-(hydroxymethyl)cyclopropyl)methoxy)-4-(2-methylazepan-1-yl)-5,7-dihydro-6H-pyrrolo[3,4-d]pyrimidin-6-yl)methanone (Ex. 84-2a)

To a mixture of 3-hydroxy-8-iodo-1-naphthoic acid (Int-T002) (471 mg, 1.50 mmol), 1-hydroxybenzotriazole monohydrate (230 mg, 1.50 mmol), 1-(3-dimethylaminopropyl)-3-ethylcarbodiimide hydrochloride (288 mg, 1.50 mmol) and N,N-diisopropylethylamine (0.64 mL, 3.75 mmol) in N,N-dimethylformamide (5 mL) was added (R)-(1-(((4-(2-methylazepan-1-yl)-6,7-dihydro-5H-pyrrolo[3,4-d]pyrimidin-2-yl)oxy)methyl)cyclopropyl)methanol (Int-84c-2) (416 mg, 1.25 mmol), and the mixture was stirred at room temperature for 5 h. The mixture was diluted with ethyl acetate and the diluted mixture was washed with water and the mixture was concentrated under reduced pressure. The residue was purified by flash NH-silica gel chromatography (0-100%, ethyl acetate gradient in hexane) to give (R)-(3-hydroxy-8-iodonaphthalen-1-yl)(2-((1-(hydroxymethyl)cyclopropyl)methoxy)-4-(2-methylazepan-1-yl)-5,7-dihydro-6H-pyrrolo[3,4-d]pyrimidin-6-yl)methanone (Ex. 84-2a) (580 mg). ESI-MS m/z [M+H]⁺ 629.

### Example 93: (R)-(3-hydroxy-8-iodonaphthalen-1-yl)(2-((1-((4-methoxypiperidin-1-yl)methyl)cyclopropyl)methoxy)-4-(2-methylazepan-1-yl)-5,7-dihydro-6H-pyrrolo[3,4-d]pyrimidin-6-yl)methanone (Ex. 93)

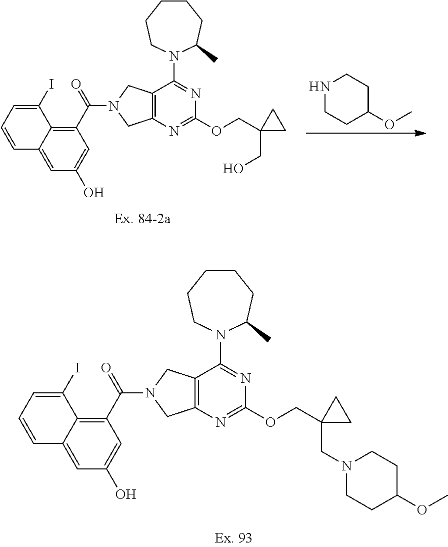

Example 93 was synthesized by a similar procedure as Ex. 85 using (R)-(3-hydroxy-8-iodonaphthalen-1-yl)(2-((1-(hydroxymethyl)cyclopropyl)methoxy)-4-(2-methylazepan-1-yl)-5,7-dihydro-6H-pyrrolo[3,4-d]pyrimidin-6-yl)methanone (Ex. 84-2a) and 4-methoxypiperidine. ESI-MS m/z [M+H]+ 726. ¹H NMR (400 MHZ, DMSO-d₆) δ 10.3 (s, 1H), 8.03-7.98 (m, 1H), 7.87-7.82 (m, 1H), 7.29-7.25 (m, 1H), 7.19-7.09 (m, 2H), 5.18-5.04 (m, 1H), 4.96-4.88 (m, 1H), 4.69-4.35 (m, 2H), 4.22-4.00 (m, 4H), 3.21 (s, 1.5H), 3.19 (s, 1.5H), 2.80-2.65 (m, 2H), 2.38-2.15 (m, 2H), 2.10-0.95 (m, 19H), 0.59-0.50 (m, 2H), 0.39-0.30 (m, 2H).

### Example 94: (R)-(2-((1-((1,4-oxazepan-4-yl)methyl)cyclopropyl)methoxy)-4-(2-methylazepan-1-yl)-5,7-dihydro-6H-pyrrolo[3,4-d]pyrimidin-6-yl)(3-hydroxy-8-iodonaphthalen-1-yl)methanone (Ex. 94)

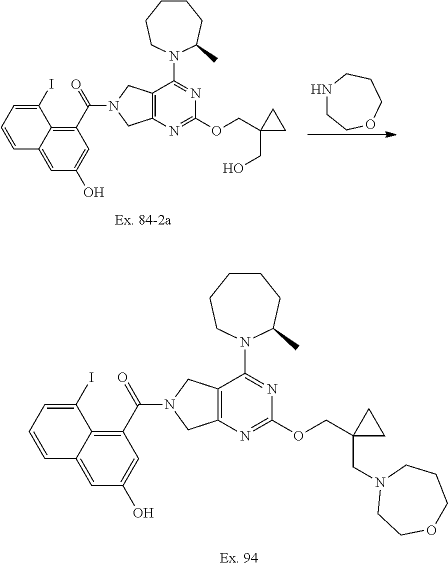 [SMILES: C[C@@H]1CCCCCN1c1nc(OCC2(CO)CC2)nc2c1CN(C(=O)c1cc(O)cc3cccc(I)c13)C2 ; C1CNCCOC1 ; C[C@@H]1CCCCCN1c1nc(OCC2(CN3CCCOCC3)CC2)nc2c1CN(C(=O)c1cc(O)cc3cccc(I)c13)C2]

Example 94 was synthesized by a similar procedure as Ex. 85 using (R)-(3-hydroxy-8-iodonaphthalen-1-yl)(2-((1-(hydroxymethyl)cyclopropyl)methoxy)-4-(2-methylazepan-1-yl)-5,7-dihydro-6H-pyrrolo[3,4-d]pyrimidin-6-yl)methanone (Ex. 84-2a) and 1,4-oxazepane. ESI-MS m/z [M+H]+ 712.

### Example 95: (R)-(4-(3-hydroxy-3-methylpiperidin-1-yl)-2-((1-(hydroxymethyl)cyclopropyl)methoxy)-5,7-dihydro-6H-pyrrolo[3,4-d]pyrimidin-6-yl)(3-hydroxy-8-iodonaphthalen-1-yl)methanone (Ex. 95)

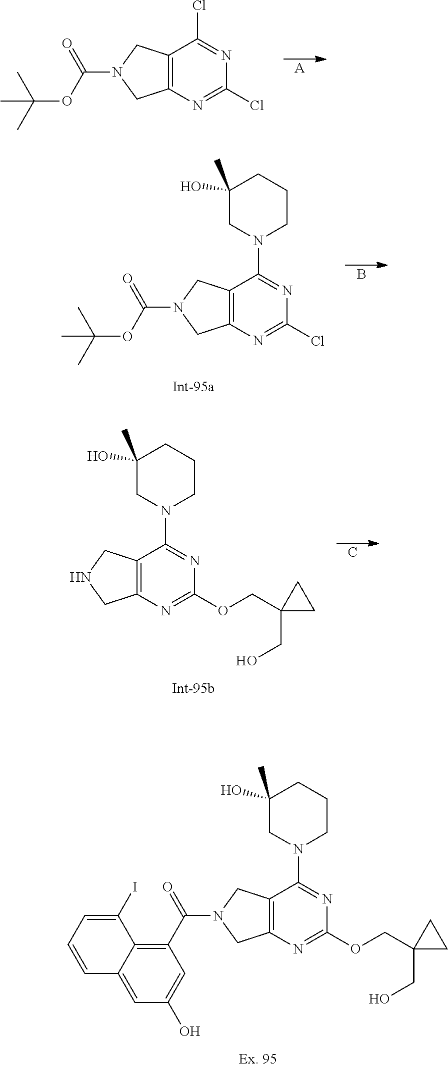

### Step A: tert-butyl (R)-2-chloro-4-(3-hydroxy-3-methylpiperidin-1-yl)-5,7-dihydro-6H-pyrrolo[3,4-d]pyrimidine-6-carboxylate (Int-95a)

To a solution of tert-butyl 2,4-dichloro-5,7-dihydro-6H-pyrrolo[3,4-d]pyrimidine-6-carboxylate (900 mg, 3.10 mmol) and (R)-3-methylpiperidin-3-ol hydrochloride (470 mg, 3.10 mmol) in N,N-dimethylacetamide (5.0 mL) was added N,N-diisopropylethylamine (1.62 mL, 9.31 mmol) at room temperature. After stirring the mixture at room temperature for 40 min, the reaction was quenched by the addition of H₂O. The reaction mixture was extracted with EtOAc, washed with brine, dried over Na₂SO₄, filtered and concentrated in vacuo. The residue was purified by flash silica gel chromatography (0-100%, EtOAc gradient in hexane) to afford tert-butyl (R)-2-chloro-4-(3-hydroxy-3-methylpiperidin-1-yl)-5,7-dihydro-6H-pyrrolo[3,4-d]pyrimidine-6-carboxylate (Int-95a) (1.12 g). ESI-MS m/z [M+H]⁺ 369, 371.

### Step B: (R)-1-(2-((1-(hydroxymethyl)cyclopropyl)methoxy)-6,7-dihydro-5H-pyrrolo[3,4-d]pyrimidin-4-yl)-3-methylpiperidin-3-ol (Int-95b)

To a solution of tert-butyl (R)-2-chloro-4-(3-hydroxy-3-methylpiperidin-1-yl)-5,7-dihydro-6H-pyrrolo[3,4-d]pyrimidine-6-carboxylate (Int-95a) (50.0 mg, 0.136 mmol), cyclopropane-1,1-diyldimethanol (41.5 mg, 0.407 mmol) and RuPhos Pd G3 (3.40 mg, 0.00407 mmol) in 1,4-dioxane (0.7 mL) was added cesium carbonate (132 mg, 0.407 mmol) at room temperature. After stirring the mixture at 100° C. for 1 h, the reaction mixture was cooled to room temperature, filtered and concentrated in vacuo. The residue was dissolved in EtOAc, washed with H₂O and brine, dried over Na₂SO₄, filtered and concentrated in vacuo to afford coupling product. To a solution of the coupling product in dichloromethane (2.6 mL) was added trifluoroacetic acid (1.0 mL). After stirring the mixture at room temperature for 3 h, the reaction mixture was concentrated in vacuo. The residue was purified by flash NH-silica gel chromatography (0-50%, MeOH gradient in EtOAc) to afford (R)-1-(2-((1-(hydroxymethyl)cyclopropyl)methoxy)-6, 7-dihydro-5H-pyrrolo[3,4-d]pyrimidin-4-yl)-3-methylpiperidin-3-ol (Int-95b) (37.5 mg). ESI-MS m/z [M+H]⁺ 335.

### Step C: Example 95 (R)-(4-(3-hydroxy-3-methylpiperidin-1-yl)-2-((1-(hydroxymethyl)cyclopropyl)methoxy)-5,7-dihydro-6H-pyrrolo[3,4-d]pyrimidin-6-yl)(3-hydroxy-8-iodonaphthalen-1-yl)methanone (Ex. 95)

To a solution of (R)-1-(2-((1-(hydroxymethyl)cyclopropyl)methoxy)-6,7-dihydro-5H-pyrrolo[3,4-d]pyrimidin-4-yl)-3-methylpiperidin-3-ol (Int-95b) (37.5 mg. 0.112 mmol), 3-hydroxy-8-iodo-1-naphthoic acid (Int-T002) (38.7 mg, 0.123 mmol), 1-(3-dimethylaminopropyl)-3-ethylcarbodiimide hydrochloride (23.6 mg, 0.123 mmol) and 1-hydroxybenzotriazole hydrate (18.9 mg, 0.123 mmol) in DMF (1.2 mL) was added N,N-diisopropylethylamine (0.0977 mL, 0.561 mmol) at room temperature. After stirring the mixture at room temperature for 15 h, the reaction was quenched by the addition of H₂O. The reaction mixture was extracted with EtOAc, washed with brine, dried over Na₂SO₄, filtered and concentrated in vacuo. The residue was purified by flash NH-silica gel chromatography (0-50%, MeOH gradient in EtOAc) and reverse phase HPLC (MeCN/water with 0.1% formic acid) to afford(R)-(4-(3-hydroxy-3-methylpiperidin-1-yl)-2-((1-(hydroxymethyl)cyclopropyl)methoxy)-5,7-dihydro-6H-pyrrolo[3,4-d]pyrimidin-6-yl)(3-hydroxy-8-iodonaphthalen-1-yl)methanone (Ex. 95). ESI-MS m/z [M+H]⁺ 631. ¹H NMR (400 MHz, DMSO-d₆) δ 10.3-10.2 (m, 1H), 8.02-7.96 (m, 1H), 7.87-7.80 (m, 1H), 7.29-7.24 (m, 1H), 7.16-7.08 (m, 2H), 5.06-4.83 (m, 1H), 4.68-4.34 (m, 4H), 4.26-3.96 (m, 4H), 3.90-3.64 (m, 1H), 3.57-3.44 (m, 1H), 3.25-2.96 (m, 2H), 1.85-1.38 (m, 4H), 1.13 (s, 3H), 0.80 (d, J=10.6 Hz, 1H), 0.52-0.39 (m, 4H).

### Example 96: (R)-(4-(3-hydroxy-3-methylpiperidin-1-yl)-2-((1-(piperidin-1-ylmethyl)cyclopropyl)methoxy)-5,7-dihydro-6H-pyrrolo[3,4-d]pyrimidin-6-yl)(3-hydroxy-8-iodonaphthalen-1-yl)methanone (Ex. 96)

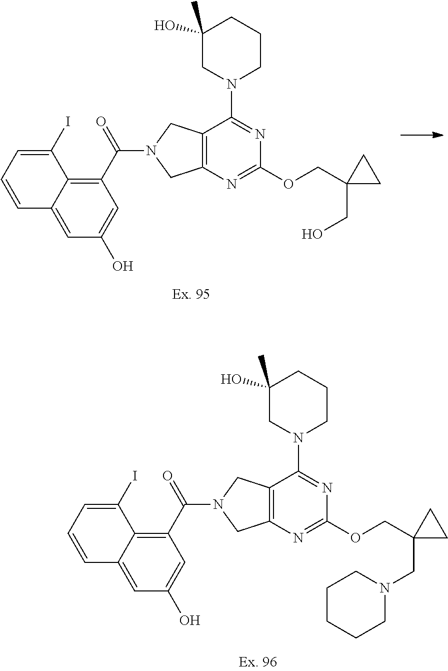 [SMILES: C[C@@]1(O)CCCN(c2nc(OCC3(CO)CC3)nc3c2CN(C(=O)c2cc(O)cc4cccc(I)c24)C3)C1 ; C[C@@]1(O)CCCN(c2nc(OCC3(CN4CCCCC4)CC3)nc3c2CN(C(=O)c2cc(O)cc4cccc(I)c24)C3)C1]

To a solution of (R)-(4-(3-hydroxy-3-methylpiperidin-1-yl)-2-((1-(hydroxymethyl)cyclopropyl)methoxy)-5,7-dihydro-6H-pyrrolo[3,4-d]pyrimidin-6-yl)(3-hydroxy-8-iodonaphthalen-1-yl)methanone (Ex. 95) (10.0 mg, 0.0159 mmol) in N,N-dimethylacetamide (0.3 mL) were added ethanesulfonyl chloride (0.00333 mL, 0.0352 mmol) and triethylamine (0.0111 mL, 0.0793 mmol) at 5° C. After stirring the mixture at 5° C. for 1 h, to the reaction mixture was added piperidine (0.00783 mL, 0.0793 mmol). After stirring the mixture at 75° C. for 2 h, 2.0 M aqueous solution of NaOH (0.2 mL) was added to the mixture and the mixture was stirred at 75° C. for 30 min. The reaction mixture was cooled to room temperature, diluted with DMSO (0.5 mL) and purified by reverse phase HPLC (MeCN/water with 0.1% formic acid) to afford (R)-(4-(3-hydroxy-3-methylpiperidin-1-yl)-2-((1-(piperidin-1-ylmethyl)cyclopropyl)methoxy)-5,7-dihydro-6H-pyrrolo[3,4-d]pyrimidin-6-yl)(3-hydroxy-8-iodonaphthalen-1-yl)methanone (Ex. 96). ESI-MS m/z [M+H]⁺ 698. ¹H NMR (400 MHZ, DMSO-d₆) δ 10.3 (s, 1H), 8.02-7.96 (m, 1H), 7.87-7.80 (m, 1H), 7.29-7.24 (m, 1H), 7.16-7.08 (m, 2H), 5.06-4.82 (m, 1H), 4.70-4.33 (m, 3H), 4.19-3.96 (m, 4H), 3.87-3.65 (m, 1H), 2.39-2.13 (m, 6H), 1.84-1.25 (m, 11H), 1.13 (s, 3H), 0.80 (d, J=10.4 Hz, 1H), 0.60-0.46 (m, 2H), 0.38-0.27 (m, 2H).

Compounds in the table below were synthesized via a similar route as Ex. 96 using Ex. 95 and corresponding amines.

TABLE 3

| | | | | ESI-MS |
|---|---|---|---|---|
| Example | Structure | Name | NMR | [M + H]⁺ |
| 97 | 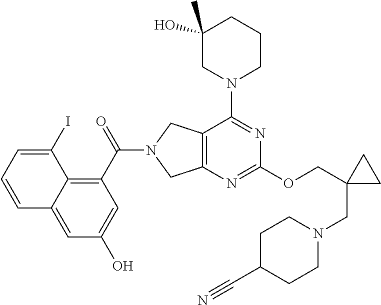 [SMILES: C[C@@]1(O)CCCN(c2nc(OCC3(CN4CCC(C#N)CC4)CC3)nc3c2CN(C(=O)c2cc(O)cc4cccc(I)c24)C3)C1] | (R)-1-((1-(((4-(3-hydroxy-3-methylpiperidin-1- yl)-6-(3-hydroxy-8-iodo-1-naphthoyl)-6,7- dihydro-5H-pyrrolo[3,4-d]pyrimidin-2- yl)oxy)methyl)cyclopropyl)methyl)piperidine-4- carbonitrile | | 723 |
| 98 | 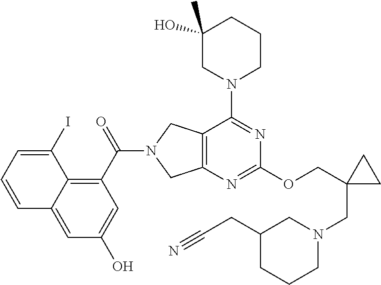 [SMILES: C[C@@]1(O)CCCN(c2nc(OCC3(CN4CCCC(CC#N)C4)CC3)nc3c2CN(C(=O)c2cc(O)cc4cccc(I)c24)C3)C1] | 2-(1-((1-(((4-((R)-3-hydroxy-3-methylpiperidin- 1-yl)-6-(3-hydroxy-8-iodo-1-naphthoyl)-6,7- dihydro-5H-pyrrolo[3,4-d]pyrimidin-2- yl)oxy)methyl)cyclopropyl)methyl)piperidin-3- yl)acetonitrile | | 737 |
| 99 | 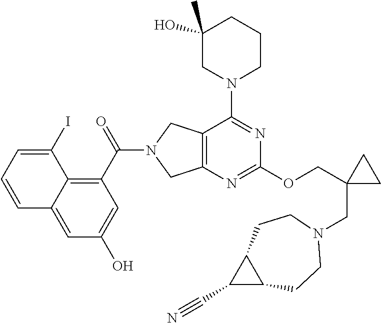 [SMILES: C[C@@]1(O)CCCN(c2nc(OCC3(CN4CC[C@@H]5[C@H](C#N)[C@@H]5CC4)CC3)nc3c2CN(C(=O)c2cc(O)cc4cccc(I)c24)C3)C1] | (1R,7S,8S)-4-((1-(((4-((R)-3-hydroxy-3- methylpiperidin-1-yl)-6-(3-hydroxy-8-iodo-1- naphthoyl)-6,7-dihydro-5H-pyrrolo[3,4- d]pyrimidin-2- yl)oxy)methyl)cyclopropyl)methyl)-4- azabicyclo[5.1.0]octane-8-carbonitrile | | 749 |
| 100 | 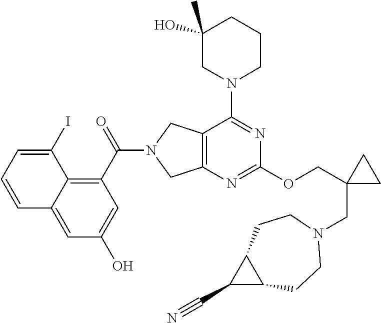 [SMILES: C[C@@]1(O)CCCN(c2nc(OCC3(CN4CC[C@@H]5[C@@H](C#N)[C@@H]5CC4)CC3)nc3c2CN(C(=O)c2cc(O)cc4cccc(I)c24)C3)C1] | (1R,7S,8R)-4-((1-(((4-((R)-3-hydroxy-3- methylpiperidin-1-yl)-6-(3-hydroxy-8-iodo-1- naphthoyl)-6,7-dihydro-5H-pyrrolo[3,4- d]pyrimidin-2- yl)oxy)methyl)cyclopropyl)methyl)-4- azabicyclo[5.1.0]octane-8-carbonitrile | | 749 |
| 101 | 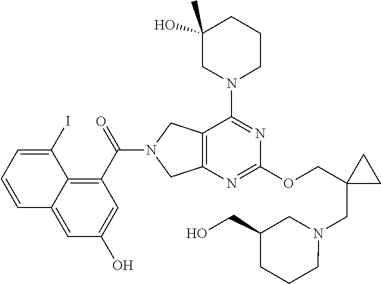 [SMILES: C[C@@]1(O)CCCN(c2nc(OCC3(CN4CCC[C@@H](CO)C4)CC3)nc3c2CN(C(=O)c2cc(O)cc4cccc(I)c24)C3)C1] | (4-((R)-3-hydroxy-3-methylpiperidin-1-yl)-2-((1- (((R)-3-(hydroxymethyl)piperidin-1- yl)methyl)cyclopropyl)methoxy)-5,7-dihydro- 6H-pyrrolo[3,4-d]pyrimidin-6-yl)(3-hydroxy-8- iodonaphthalen-1-yl)methanone | | 728 |
| 102 | 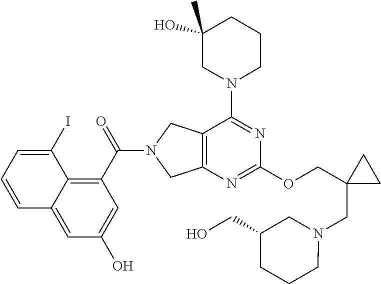 [SMILES: C[C@@]1(O)CCCN(c2nc(OCC3(CN4CCC[C@H](CO)C4)CC3)nc3c2CN(C(=O)c2cc(O)cc4cccc(I)c24)C3)C1] | (4-((R)-3-hydroxy-3-methylpiperidin-1-yl)-2-((1- (((S)-3-(hydroxymethyl)piperidin-1- yl)methyl)cyclopropyl)methoxy)-5,7-dihydro- 6H-pyrrolo[3,4-d]pyrimidin-6-yl)(3-hydroxy-8- iodonaphthalen-1-yl)methanone | | 728 |
| 103 | 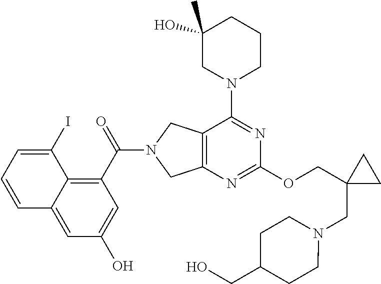 [SMILES: C[C@@]1(O)CCCN(c2nc(OCC3(CN4CCC(CO)CC4)CC3)nc3c2CN(C(=O)c2cc(O)cc4cccc(I)c24)C3)C1] | (R)-(4-(3-hydroxy-3-methylpiperidin-1-yl)-2-((1- ((4-(hydroxymethyl)piperidin-1- yl)methyl)cyclopropyl)methoxy)-5,7-dihydro- 6H-pyrrolo[3,4-d]pyrimidin-6-yl)(3-hydroxy-8- iodonaphthalen-1-yl)methanone | | 728 |
| 104 | 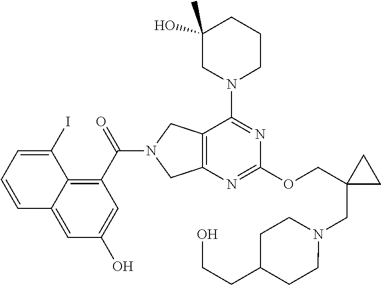 [SMILES: C[C@@]1(O)CCCN(c2nc(OCC3(CN4CCC(CCO)CC4)CC3)nc3c2CN(C(=O)c2cc(O)cc4cccc(I)c24)C3)C1] | (R)-(4-(3-hydroxy-3-methylpiperidin-1-yl)-2-((1- ((4-(2-hydroxyethyl)piperidin-1- yl)methyl)cyclopropyl)methoxy)-5,7-dihydro- 6H-pyrrolo[3,4-d]pyrimidin-6-yl)(3-hydroxy-8- iodonaphthalen-1-yl)methanone | | 742 |
| 105 | 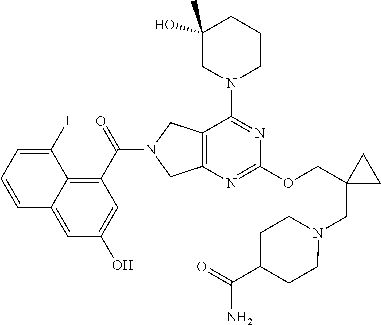 [SMILES: C[C@@]1(O)CCCN(c2nc(OCC3(CN4CCC(C(N)=O)CC4)CC3)nc3c2CN(C(=O)c2cc(O)cc4cccc(I)c24)C3)C1] | (R)-1-((1-(((4-(3-hydroxy-3-methylpiperidin-1- yl)-6-(3-hydroxy-8-iodo-1-naphthoyl)-6,7- dihydro-5H-pyrrolo[3,4-d]pyrimidin-2- yl)oxy)methyl)cyclopropyl)methyl)piperidine-4- carboxamide | | 741 |
| 106 | 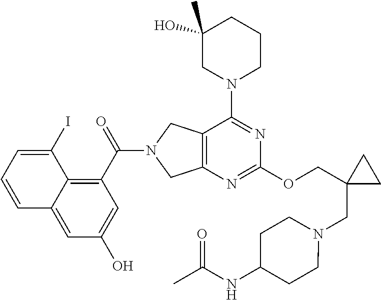 [SMILES: CC(=O)NC1CCN(CC2(COc3nc4c(c(N5CCC[C@@](C)(O)C5)n3)CN(C(=O)c3cc(O)cc5cccc(I)c35)C4)CC2)CC1] | (R)-N-(1-((1-(((4-(3-hydroxy-3-methylpiperidin- 1-yl)-6-(3-hydroxy-8-iodo-1-naphthoyl)-6,7- dihydro-5H-pyrrolo[3,4-d]pyrimidin-2- yl)oxy)methyl)cyclopropyl)methyl)piperidin-4- yl)acetamide | | 755 |
| 107 | 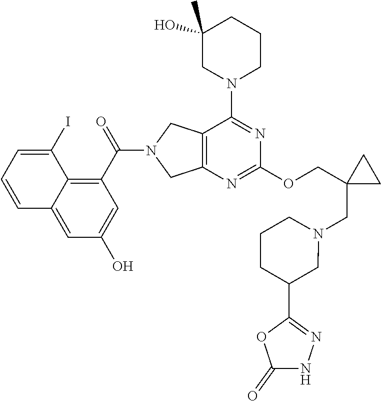 [SMILES: C[C@@]1(O)CCCN(c2nc(OCC3(CN4CCCC(c5n[nH]c(=O)o5)C4)CC3)nc3c2CN(C(=O)c2cc(O)cc4cccc(I)c24)C3)C1] | 5-(1-((1-(((4-((R)-3-hydroxy-3-methylpiperidin- 1-yl)-6-(3-hydroxy-8-iodo-1-naphthoyl)-6,7- dihydro-5H-pyrrolo[3,4-d]pyrimidin-2- yl)oxy)methyl)cyclopropyl)methyl)piperidin-3- yl)-1,3,4-oxadiazol-2(3H)-one | | 782 |

### (R)-(3-hydroxy-8-iodonaphthalen-1-yl)(2-((1-(hydroxymethyl)cyclopropyl)methoxy)-4-(3-methyl-3-((trimethylsilyl)oxy)piperidin-1-yl)-5,7-dihydro-6H-pyrrolo[3,4-d]pyrimidin-6-yl)methanone (Int-108e)

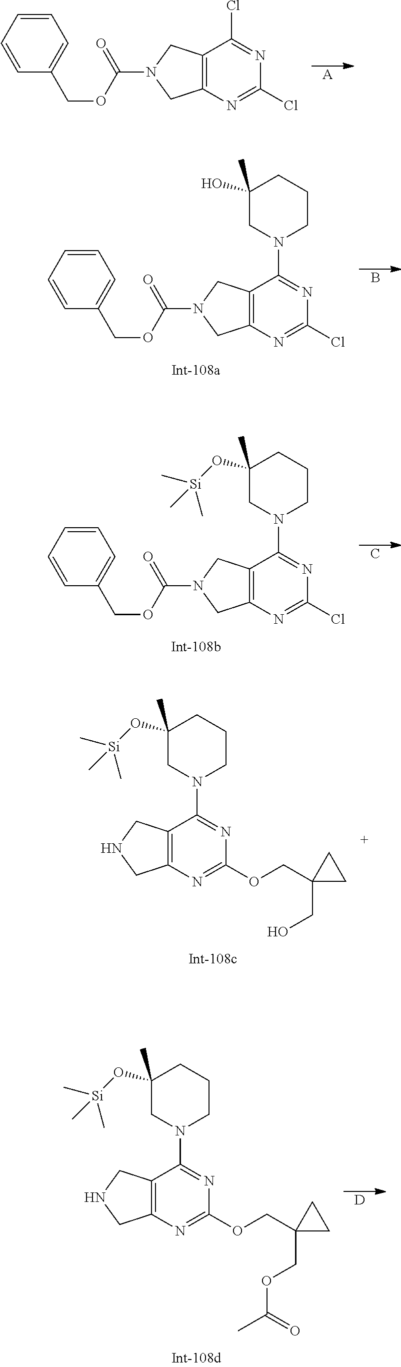

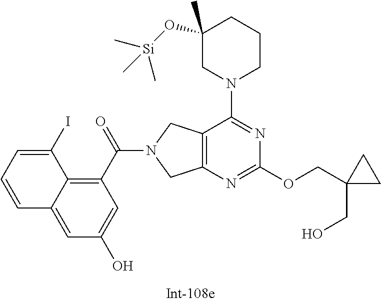 [SMILES: C[C@@]1(O[Si](C)(C)C)CCCN(c2nc(OCC3(CO)CC3)nc3c2CN(C(=O)c2cc(O)cc4cccc(I)c24)C3)C1]

### Step A: benzyl (R)-2-chloro-4-(3-hydroxy-3-methylpiperidin-1-yl)-5,7-dihydro-6H-pyrrolo[3,4-d]pyrimidine-6-carboxylate (Int-108a)

To a solution of benzyl 2,4-dichloro-5,7-dihydro-6H-pyrrolo[3,4-d]pyrimidine-6-carboxylate (300 mg, 0.925 mmol) and (R)-3-methylpiperidin-3-ol hydrochloride (140 mg, 0.925 mmol) in N,N-dimethylacetamide (5.0 mL) was added N,N-diisopropylethylamine (0.450 mL, 2.58 mmol) at room temperature. After stirring the mixture at room temperature for 30 min, the reaction was quenched by the addition of H₂O. The reaction mixture was extracted with EtOAc, washed with brine, dried over Na₂SO₄, filtered and concentrated in vacuo. The residue was purified by flash silica gel chromatography (0-50%, EtOAc gradient in hexane) to afford benzyl (R)-2-chloro-4-(3-hydroxy-3-methylpiperidin-1-yl)-5,7-dihydro-6H-pyrrolo[3,4-d]pyrimidine-6-carboxylate (Int-108a) (357 mg). ESI-MS m/z [M+H]⁺ 403, 405.

### Step B: benzyl (R)-2-chloro-4-(3-methyl-3-((trimethylsilyl)oxy)piperidin-1-yl)-5,7-dihydro-6H-pyrrolo[3,4-d]pyrimidine-6-carboxylate (Int-108b)

To a solution of benzyl (R)-2-chloro-4-(3-hydroxy-3-methylpiperidin-1-yl)-5,7-dihydro-6H-pyrrolo[3,4-d]pyrimidine-6-carboxylate (Int-108a) (357 mg, 0.885 mmol) in DMF (3.0 mL) were added imidazole (121 mg, 1.77 mmol) and chlorotrimethylsilane (0.340 mL, 2.68 mmol) at room temperature. After stirring the mixture at room temperature for 15 h, the reaction was quenched by the addition of H₂O. The reaction mixture was extracted with EtOAc, washed with brine, dried over Na₂SO₄, filtered and concentrated in vacuo. The residue was purified by flash silica gel chromatography (0-40%, EtOAc gradient in hexane) to afford benzyl (R)-2-chloro-4-(3-methyl-3-((trimethylsilyl)oxy)piperidin-1-yl)-5,7-dihydro-6H-pyrrolo[3,4-d]pyrimidine-6-carboxylate (Int-108b) (375 mg). ESI-MS m/z [M+H]⁺ 475, 477.

### Step C: (R)-(1-(((4-(3-methyl-3-((trimethylsilyl)oxy)piperidin-1-yl)-6, 7-dihydro-5H-pyrrolo[3,4-d]pyrimidin-2-yl)oxy)methyl)cyclopropyl)methanol (Int-108c) and (R)-(1-(((4-(3-methyl-3-((trimethylsilyl)oxy)piperidin-1-yl)-6,7-dihydro-5H-pyrrolo[3,4-d]pyrimidin-2-yl)oxy)methyl)cyclopropyl)methyl acetate (Int-108d)

To a solution of benzyl (R)-2-chloro-4-(3-methyl-3-((trimethylsilyl)oxy)piperidin-1-yl)-5,7-dihydro-6H-pyrrolo[3,4-d]pyrimidine-6-carboxylate (Int-108b) (375 mg, 0.788 mmol), cyclopropane-1, 1-diyldimethanol (242 mg, 2.36 mmol) and RuPhos Pd G3 (19.8 mg, 0.0236 mmol) in 1,4-dioxane (5.0 mL) was added cesium carbonate (771 mg, 2.36 mmol) at room temperature. After stirring the mixture at 100° C. for 1 h, the reaction mixture was cooled to room temperature, diluted with EtOAc, filtered and concentrated in vacuo. The residue was purified by flash NH-silica gel chromatography (50-100%, EtOAc gradient in hexane) to afford coupling product.

To a solution of coupling product in ethanol (20 mL) was added Pd(OH)₂/C (277 mg, 0.394 mmol). The reaction mixture was purged with H₂ gas and vacuum three times and stirred at room temperature for 1 h. The reaction mixture was filtered and concentrated in vacuo. The residue was purified by flash NH-silica gel chromatography (0-30%, MeOH gradient in EtOAc) to afford (R)-(1-(((4-(3-methyl-3-((trimethylsilyl)oxy)piperidin-1-yl)-6,7-dihydro-5H-pyrrolo[3,4-d]pyrimidin-2-yl)oxy)methyl)cyclopropyl)methanol (Int-108c) (40.5 mg). ESI-MS m/z [M+H]⁺ 407 and (R)-(1-(((4-(3-methyl-3-((trimethylsilyl)oxy)piperidin-1-yl)-6,7-dihydro-5H-pyrrolo[3,4-d]pyrimidin-2-yl)oxy)methyl)cyclopropyl)methyl acetate (Int-108d) (105 mg). ESI-MS m/z [M+H]⁺ 449.

### Step D: (R)-(3-hydroxy-8-iodonaphthalen-1-yl)(2-((1-(hydroxymethyl)cyclopropyl)methoxy)-4-(3-methyl-3-((trimethylsilyl)oxy)piperidin-1-yl)-5,7-dihydro-6H-pyrrolo[3,4-d]pyrimidin-6-yl)methanone (Int-108e)

To a solution of (R)-(1-(((4-(3-methyl-3-((trimethylsilyl)oxy)piperidin-1-yl)-6,7-dihydro-5H-pyrrolo[3,4-d]pyrimidin-2-yl)oxy)methyl)cyclopropyl)methanol (Int-108c) (40.5 mg, 0.0996 mmol), (R)-(1-(((4-(3-methyl-3-((trimethylsilyl)oxy)piperidin-1-yl)-6,7-dihydro-5H-pyrrolo[3,4-d]pyrimidin-2-yl)oxy)methyl)cyclopropyl)methyl acetate (Int-108d) (105 mg, 0.234 mmol), 3-hydroxy-8-iodo-1-naphthoic acid (Int-T002) (115 mg, 0.366 mmol), 1-(3-dimethylaminopropyl)-3-ethylcarbodiimide hydrochloride (69.3 mg, 0.361 mmol) and 1-hydroxybenzotriazole hydrate (57.0 mg, 0.372 mmol) in DMF (3.4 mL) was added N,N-diisopropylethylamine (0.174 mL, 0.999 mmol) at room temperature. After stirring the mixture at room temperature for 3 h, to the reaction mixture were added MeOH (1.0 mL) and 2.0 M aqueous solution of NaOH (1.0 mL). After stirring the mixture at room temperature for 20 min, the reaction was quenched by the addition of 2.0 M aqueous solution of HCl (1.0 mL). The reaction mixture was extracted with EtOAc, washed with brine, dried over Na₂SO₄, filtered and concentrated in vacuo. The residue was purified by flash NH-silica gel chromatography (0-40%, MeOH gradient in EtOAc) to afford (R)-(3-hydroxy-8-iodonaphthalen-1-yl)(2-((1-(hydroxymethyl)cyclopropyl)methoxy)-4-(3-methyl-3-((trimethylsilyl)oxy)piperidin-1-yl)-5,7-dihydro-6H-pyrrolo[3,4-d]pyrimidin-6-yl)methanone (Int-108e). ESI-MS m/z [M+H]⁺ 703.

### Example 108: (R)-(4-(3-hydroxy-3-methylpiperidin-1-yl)-2-((1-((4-methoxypiperidin-1-yl)methyl)cyclopropyl)methoxy)-5,7-dihydro-6H-pyrrolo[3,4-d]pyrimidin-6-yl)(3-hydroxy-8-iodonaphthalen-1-yl)methanone (Ex. 108)

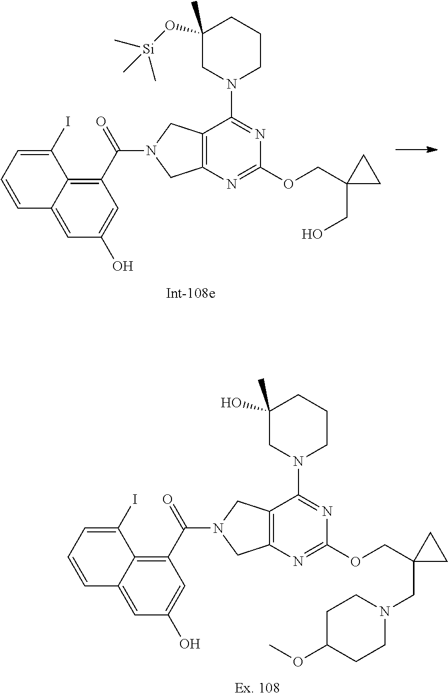 [SMILES: C[C@@]1(O[Si](C)(C)C)CCCN(c2nc(OCC3(CO)CC3)nc3c2CN(C(=O)c2cc(O)cc4cccc(I)c24)C3)C1 ; COC1CCN(CC2(COc3nc4c(c(N5CCC[C@@](C)(O)C5)n3)CN(C(=O)c3cc(O)cc5cccc(I)c35)C4)CC2)CC1]

To a solution of (R)-(3-hydroxy-8-iodonaphthalen-1-yl)(2-((1-(hydroxymethyl)cyclopropyl)methoxy)-4-(3-methyl-3-((trimethylsilyl)oxy)piperidin-1-yl)-5,7-dihydro-6H-pyrrolo[3,4-d]pyrimidin-6-yl)methanone (Int-108e) (22.0 mg, 0.0313 mmol) in N,N-dimethylacetamide (0.4 mL) were added ethanesulfonyl chloride (0.00890 mL, 0.0939 mmol) and triethylamine (0.0220 mL, 0.157 mmol) at 5° C. After stirring the mixture at 5° C. for 15 min, to the reaction mixture was added 4-methoxypiperidine (0.0194 mL, 0.157 mmol). After stirring the mixture at 60° C. for 3 h, 2.0 M aqueous solution of NaOH (0.1 mL) was added to the mixture and the mixture was stirred at 60° C. for 30 min. To the reaction mixture was added 6.0 M aqueous solution of HCl (0.1 mL) and the reaction stirred 60° C. for 10 min. The reaction mixture was cooled to room temperature, diluted with DMSO (0.5 mL) and purified by reverse phase HPLC (MeCN/water with 0.1% formic acid) to afford (R)-(4-(3-hydroxy-3-methylpiperidin-1-yl)-2-((1-((4-methoxypiperidin-1-yl)methyl)cyclopropyl)methoxy)-5,7-dihydro-6H-pyrrolo[3,4-d]pyrimidin-6-yl)(3-hydroxy-8-iodonaphthalen-1-yl)methanone (Ex. 108). ESI-MS m/z [M+H]⁺ 728.

Compounds in the table below were synthesized via a similar route as Ex. 108 using Int-108e and corresponding amines.

TABLE 4

| | | | | ESI-MS |
|---|---|---|---|---|
| Example | Structure | Name | NMR | [M + H]⁺ |
| 109 | 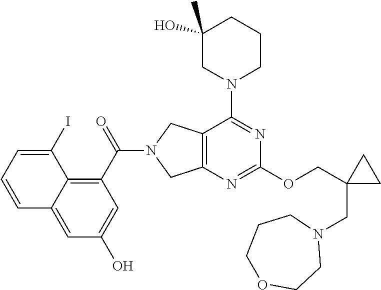 [SMILES: C[C@@]1(O)CCCN(c2nc(OCC3(CN4CCCOCC4)CC3)nc3c2CN(C(=O)c2cc(O)cc4cccc(I)c24)C3)C1] | (R)-(2-((1-((1,4-oxazepan-4 yl)methyl)cyclopropyl)methoxy)-4-(3-hydroxy-3- methylpiperidin-1-yl)-5,7-dihydro-6H-pyrrolo[3,4- d]pyrimidin-6-yl)(3-hydroxy-8-iodonaphthalen-1- yl)methanone | | 714 |
| 110 | 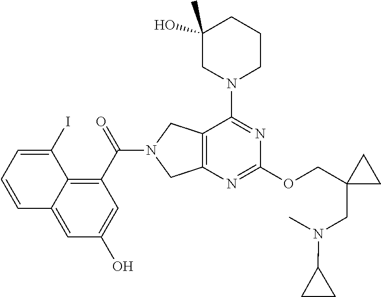 [SMILES: CN(CC1(COc2nc3c(c(N4CCC[C@@](C)(O)C4)n2)CN(C(=O)c2cc(O)cc4cccc(I)c24)C3)CC1)C1CC1] | (R)-(2-((1- ((cyclopropyl(methyl)amino)methyl)cyclo- propyl)methoxy)-4-(3-hydroxy-3-methyl- piperidin-1-yl)-5,7-dihydro-6H- pyrrolo[3,4-d]pyrimidin-6-yl)(3-hydroxy-8- iodonaphthalen-1-yl)methanone | | 684 |
| 111 | 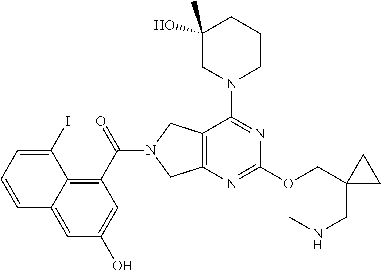 [SMILES: CNCC1(COc2nc3c(c(N4CCC[C@@](C)(O)C4)n2)CN(C(=O)c2cc(O)cc4cccc(I)c24)C3)CC1] | (R)-(4-(3-hydroxy-3-methylpiperidin-1-yl)-2-((1- ((methylamino)methyl)cyclopropyl)methoxy)-5,7- dihydro-6H-pyrrolo[3,4-d]pyrimidin-6-yl)(3- hydroxy-8-iodonaphthalen-1-yl)methanone | | 644 |

### Example 112: (2-((1-(((R)-3-fluoropyrrolidin-1-yl)methyl)cyclopropyl)methoxy)-4-((R)-3-hydroxy-3-methylpiperidin-1-yl)-5,7-dihydro-6H-pyrrolo[3,4-d]pyrimidin-6-yl)(3-hydroxy-8-iodonaphthalen-1-yl)methanone (Ex. 112)

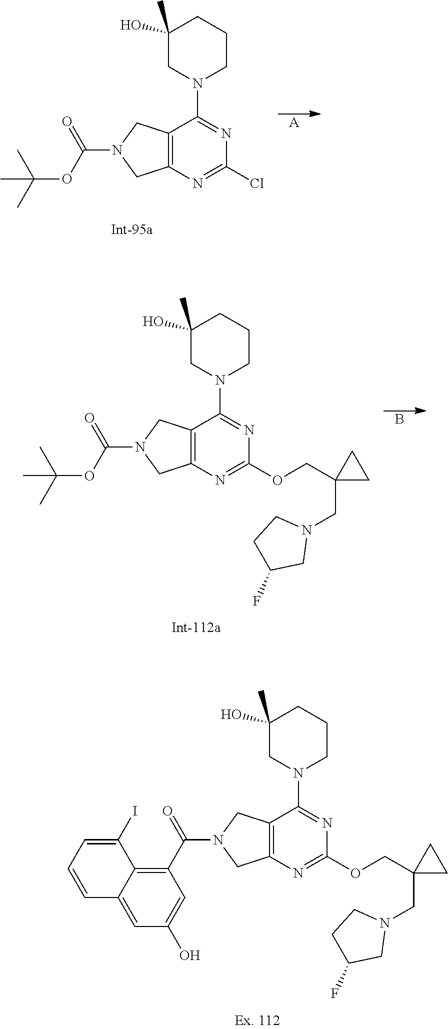

### Step A: tert-butyl 2-((1-(((R)-3-fluoropyrrolidin-1-yl)methyl)cyclopropyl)methoxy)-4-((R)-3-hydroxy-3-methylpiperidin-1-yl)-5,7-dihydro-6H-pyrrolo[3,4-d]pyrimidine-6-carboxylate (Int-112a)

To a solution of tert-butyl (R)-2-chloro-4-(3-hydroxy-3-methylpiperidin-1-yl)-5,7-dihydro-6H-pyrrolo[3,4-d]pyrimidine-6-carboxylate (Int-95a) (100 mg, 0.271 mmol), (R)-(1-((3-fluoropyrrolidin-1-yl)methyl)cyclopropyl)methanol (93.9 mg, 0.542 mmol) and RuPhos Pd G3 (6.80 mg, 0.00813 mmol) in 1,4-dioxane (1.5 mL) was added cesium carbonate (265 mg, 0.813 mmol) at room temperature. After stirring the mixture at 100° C. for 3 h, the reaction mixture was cooled to room temperature, filtered and concentrated in vacuo. The residue was purified by flash NH-silica gel chromatography (50-100%, EtOAc gradient in hexane) to afford tert-butyl 2-((1-(((R)-3-fluoropyrrolidin-1-yl)methyl)cyclopropyl)methoxy)-4-((R)-3-hydroxy-3-methylpiperidin-1-yl)-5,7-dihydro-6H-pyrrolo[3,4-d]pyrimidine-6-carboxylate (Int-112a) (156 mg). ESI-MS m/z [M+H]⁺ 506.

### Step B: Example 112 (2-((1-(((R)-3-fluoropyrrolidin-1-yl)methyl)cyclopropyl)methoxy)-4-((R)-3-hydroxy-3-methylpiperidin-1-yl)-5,7-dihydro-6H-pyrrolo[3,4-d]pyrimidin-6-yl)(3-hydroxy-8-iodonaphthalen-1-yl)methanone (Ex. 112)

To a solution of tert-butyl 2-((1-(((R)-3-fluoropyrrolidin-1-yl)methyl)cyclopropyl)methoxy)-4-((R)-3-hydroxy-3-methylpiperidin-1-yl)-5,7-dihydro-6H-pyrrolo[3,4-d]pyrimidine-6-carboxylate (Int-112a) (137 mg, 0.271 mmol) in chloroform (3.0 mL) was added 4.0 M dioxane solution of HCl (0.68 mL). After stirring the mixture at room temperature for 4 days, the reaction mixture was concentrated in vacuo to afford amine product.

To a solution of amine product, 3-hydroxy-8-iodo-1-naphthoic acid (Int-T002) (93.7 mg, 0.298 mmol), 1-(3-dimethylaminopropyl)-3-ethylcarbodiimide hydrochloride (57.2 mg, 0.298 mmol) and 1-hydroxybenzotriazole hydrate (45.7 mg, 0.298 mmol) in DMF (3.0 mL) was added N,N-diisopropylethylamine (0.472 mL, 2.71 mmol) at room temperature. After stirring the mixture at room temperature for 24 h, the reaction was quenched by the addition of H₂O. The reaction mixture was extracted with EtOAc, washed with brine, dried over Na₂SO₄, filtered and concentrated in vacuo. The residue was purified by reverse phase HPLC (MeCN/water with 0.1% formic acid) to afford (2-((1-(((R)-3-fluoropyrrolidin-1-yl)methyl)cyclopropyl)methoxy)-4-((R)-3-hydroxy-3-methylpiperidin-1-yl)-5,7-dihydro-6H-pyrrolo[3,4-d]pyrimidin-6-yl)(3-hydroxy-8-iodonaphthalen-1-yl)methanone (Ex. 112). ESI-MS m/z [M+H]⁺ 702. ¹H NMR (400 MHz, DMSO-d₆) δ 10.3 (s, 1H), 8.02-7.96 (m, 1H), 7.87-7.80 (m, 1H), 7.29-7.23 (m, 1H), 7.16-7.07 (m, 2H), 5.26-4.83 (m, 2H), 4.68-4.33 (m, 2H), 4.20-3.93 (m, 4H), 3.87-3.65 (m, 1H), 2.87-2.68 (m, 2H), 2.34-2.22 (m, 2H), 2.16-1.95 (m, 1H), 1.91-1.70 (m, 2H), 1.68-1.40 (m, 3H), 1.34-1.20 (m, 2H), 1.13 (s, 3H), 0.89-0.75 (m, 3H), 0.60-0.47 (m, 2H), 0.45-0.31 (m, 2H).

Compounds in the table below were synthesized via a similar route as Ex. 112 using Int-95a and corresponding amino alcohols.

TABLE 5

| | | | | ESI-MS |
|---|---|---|---|---|
| Example | Structure | Name | NMR | [M + H]⁺ |
| 113 | 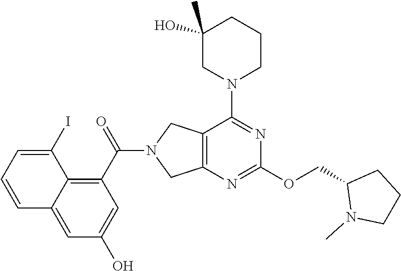 [SMILES: CN1CCC[C@H]1COc1nc2c(c(N3CCC[C@@](C)(O)C3)n1)CN(C(=O)c1cc(O)cc3cccc(I)c13)C2] | (4-((R)-3-hydroxy-3- methylpiperidin-1-yl)-2-(((S)-1- methylpyrrolidin-2-yl)methoxy)- 5,7-dihydro-6H-pyrrolo[3,4- d]pyrimidin-6-yl)(3-hydroxy-8- iodonaphthalen-1-yl)methanone | | 644 |
| 114 | 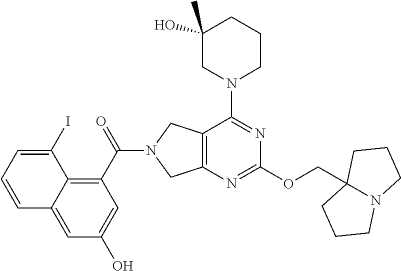 [SMILES: C[C@@]1(O)CCCN(c2nc(OCC34CCCN3CCC4)nc3c2CN(C(=O)c2cc(O)cc4cccc(I)c24)C3)C1] | (R)-(4-(3-hydroxy-3- methylpiperidin-1-yl)-2- ((tetrahydro-1H-pyrrolizin-7a(5H)- yl)methoxy)-5,7-dihydro-6H- pyrrolo[3,4-d]pyrimidin-6-yl)(3- hydroxy-8-iodonaphthalen-1- yl)methanone | | 670 |
| 115 | 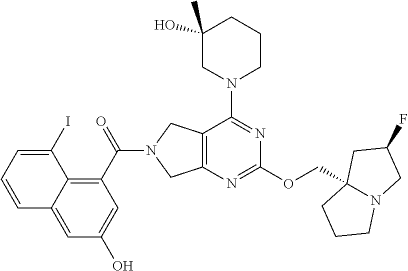 [SMILES: C[C@@]1(O)CCCN(c2nc(OC[C@@]34CCCN3C[C@H](F)C4)nc3c2CN(C(=O)c2cc(O)cc4cccc(I)c24)C3)C1] | (2-(((2S,7aR)-2-fluorotetrahydro- 1H-pyrrolizin-7a(5H)-yl)methoxy)- 4-((R)-3-hydroxy-3- methylpiperidin-1-yl)-5,7-dihydro- 6H-pyrrolo[3,4-d]pyrimidin-6-yl)(3- hydroxy-8-iodonaphthalen-1- yl)methanone | | 688 |
| 116 | 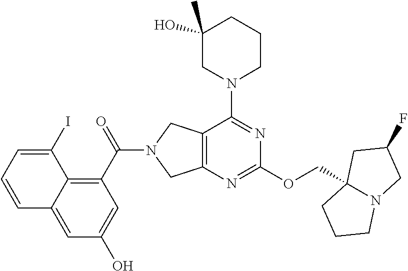 [SMILES: C[C@@]1(O)CCCN(c2nc(OC[C@@]34CCCN3C[C@H](F)C4)nc3c2CN(C(=O)c2cc(O)cc4cccc(I)c24)C3)C1] | (2-(((2R,7aS)-2-fluorotetrahydro- 1H-pyrrolizin-7a(5H)-yl)methoxy)- 4-((R)-3-hydroxy-3- methylpiperidin-1-yl)-5,7-dihydro- 6H-pyrrolo[3,4-d]pyrimidin-6-yl)(3- hydroxy-8-iodonaphthalen-1- yl)methanone | ¹H NMR (400 MHz, DMSO-d₆) δ 10.3 (s, 1H), 8.02-7.97 (m, 1H), 7.86-7.80 (m, 1H), 7.28-7.24 (m, 1H), 7.16-7.07 (m, 2H), 5.30 (d, J = 19.3 Hz, 0.5H), 5.16 (d, J = 16.7 Hz, 0.5H), 5.05-4.84 (m, 1H) 4.68-4.34 (m, 2H), 4.20-3.74 (m, 4H), 3.49 (d, J = 13.1 Hz, 1H), 3.11-2.89 (m, 4H), 2.85-2.73 (m, 1H), 2.10-1.87 (m, 3H), 1.86-1.37 (m, 7H), 1.13 (s, 3H), 0.86-0.76 (m, 2H). | 688 |

### Example 117: (2-(((R)-2,2-difluoro-1-(hydroxymethyl)cyclopropyl)methoxy)-4-((R)-3-hydroxy-3-methylpiperidin-1-yl)-5,7-dihydro-6H-pyrrolo[3,4-d]pyrimidin-6-yl)(3-hydroxy-8-iodonaphthalen-1-yl)methanone (Ex 117)

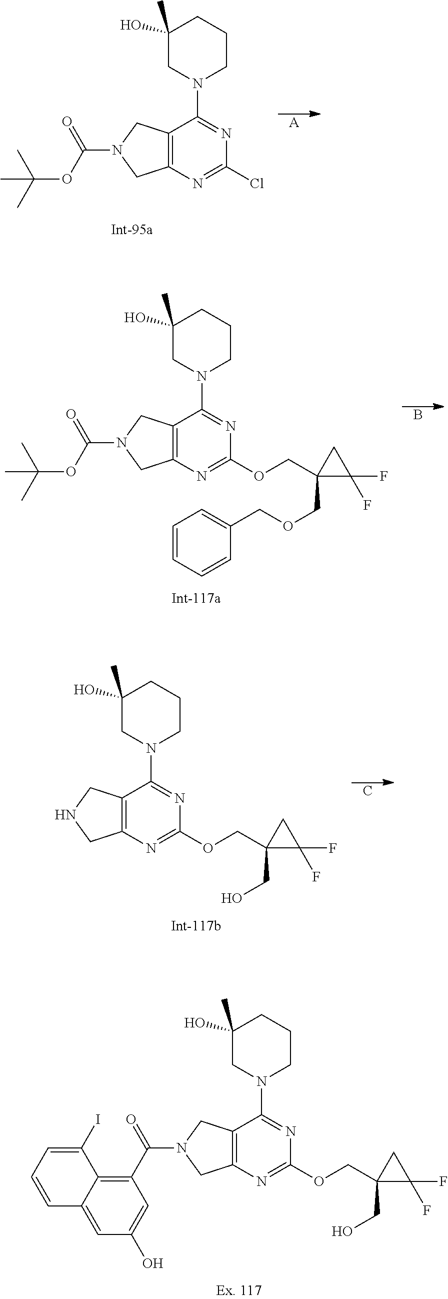

### Step A: tert-butyl 2-(((R)-1-((benzyloxy)methyl)-2,2-difluorocyclopropyl)methoxy)-4-((R)-3-hydroxy-3-methylpiperidin-1-yl)-5,7-dihydro-6H-pyrrolo[3,4-d]pyrimidine-6-carboxylate (Int-117a)

To a solution of tert-butyl (R)-2-chloro-4-(3-hydroxy-3-methylpiperidin-1-yl)-5,7-dihydro-6H-pyrrolo[3,4-d]pyrimidine-6-carboxylate (Int-95a) (400 mg, 1.08 mmol), (S)-(1-((benzyloxy)methyl)-2,2-difluorocyclopropyl)methanol (Int-UU5-2) (371 mg, 1.63 mmol) and RuPhos Pd G3 (27.2 mg, 0.0325 mmol) in 1,4-dioxane (5.0 mL) was added cesium carbonate (1.06 g, 3.25 mmol) at room temperature. After stirring the mixture at 100° C. for 6 h, the reaction mixture was cooled to room temperature, filtered and concentrated in vacuo. The residue was purified by flash silica gel chromatography (10-100%, EtOAc gradient in hexane) to afford tert-butyl 2-(((R)-1-((benzyloxy)methyl)-2,2-difluorocyclopropyl)methoxy)-4-((R)-3-hydroxy-3-methylpiperidin-1-yl)-5,7-dihydro-6H-pyrrolo[3,4-d]pyrimidine-6-carboxylate (Int-117a) (466 mg). ESI-MS m/z [M+H]⁺ 561.

### Step B: (R)-1-(2-(((R)-2,2-difluoro-1-(hydroxymethyl)cyclopropyl)methoxy)-6,7-dihydro-5H-pyrrolo[3,4-d]pyrimidin-4-yl)-3-methylpiperidin-3-ol (Int-117b)

To a solution of tert-butyl 2-(((R)-1-((benzyloxy)methyl)-2,2-difluorocyclopropyl)methoxy)-4-((R)-3-hydroxy-3-methylpiperidin-1-yl)-5,7-dihydro-6H-pyrrolo[3,4-d]pyrimidine-6-carboxylate (Int-117a) (466 mg, 0.830 mmol) in ethanol (5.0 mL) was added Pd(OH)₂/C (400 mg, 0.570 mmol). The reaction mixture was purged with H₂ gas and vacuum three times and stirred at room temperature for 2 h. The reaction mixture was filtered and concentrated in vacuo. To a solution of the resulted product in dichloromethane (8.0 mL) was added trifluoroacetic acid (1.6 mL). After stirring the mixture at room temperature for 2 h, the reaction mixture was concentrated in vacuo. The residue was purified by flash NH-silica gel chromatography (0-50%, MeOH gradient in EtOAc) to afford (R)-1-(2-(((R)-2,2-difluoro-1-(hydroxymethyl)cyclopropyl)methoxy)-6,7-dihydro-5H-pyrrolo[3,4-d]pyrimidin-4-yl)-3-methylpiperidin-3-ol (Int-117b) (259 mg). ESI-MS m/z [M+H]⁺ 371.

### Step C: Example 117 (2-(((R)-2,2-difluoro-1-(hydroxymethyl)cyclopropyl)methoxy)-4-((R)-3-hydroxy-3-methylpiperidin-1-yl)-5,7-dihydro-6H-pyrrolo[3,4-d]pyrimidin-6-yl)(3-hydroxy-8-iodonaphthalen-1-yl)methanone (Ex. 117)

To a solution of (R)-1-(2-(((R)-2,2-difluoro-1-(hydroxymethyl)cyclopropyl)methoxy)-6, 7-dihydro-5H-pyrrolo[3,4-d]pyrimidin-4-yl)-3-methylpiperidin-3-ol (Int-117b) (259 mg, 0.699 mmol), 3-hydroxy-8-iodo-1-naphthoic acid (Int-T002) (241 mg, 0.769 mmol), 1-(3-dimethylaminopropyl)-3-ethylcarbodiimide hydrochloride (147 mg, 0.769 mmol) and 1-hydroxybenzotriazole hydrate (118 mg, 0.769 mmol) in DMF (7.0 mL) was added N,N-diisopropylethylamine (0.609 mL, 3.49 mmol) at room temperature. After stirring the mixture at room temperature for 9 h, the reaction was quenched by the addition of H₂O. The reaction mixture was extracted with EtOAc, washed with brine, dried over Na₂SO₄, filtered and concentrated in vacuo. The residue was purified by flash NH-silica gel chromatography (0-50%, MeOH gradient in EtOAc) and reverse phase HPLC (MeCN/water with 0.1% formic acid) to afford (2-(((R)-2,2-difluoro-1-(hydroxymethyl)cyclopropyl)methoxy)-4-((R)-3-hydroxy-3-methylpiperidin-1-yl)-5,7-dihydro-6H-pyrrolo[3,4-d]pyrimidin-6-yl)(3-hydroxy-8-iodonaphthalen-1-yl)methanone (Ex. 117). ESI-MS m/z [M+H]⁺ 667. ¹H NMR (400 MHZ, DMSO-d₆) δ 10.3 (s, 1H), 8.02-7.96 (m, 1H), 7.87-7.80 (m, 1H), 7.29-7.24 (m, 1H), 7.17-7.07 (m, 2H), 5.10-4.84 (m, 2H), 4.71-4.48 (m, 2H), 4.48-4.30 (m, 2H), 4.27-4.11 (m, 2H), 4.08-3.67 (m, 1H), 3.62-3.49 (m, 2H), 3.10-2.98 (m, 1H), 1.86-1.24 (m, 7H), 1.14 (s, 3H), 0.86-0.76 (m, 1H).

### Example 118: (2-(((R)-1-((dimethylamino)methyl)-2,2-difluorocyclopropyl)methoxy)-4-((R)-3-hydroxy-3-methylpiperidin-1-yl)-5,7-dihydro-6H-pyrrolo[3,4-d]pyrimidin-6-yl)(3-hydroxy-8-iodonaphthalen-1-yl)methanone (Ex. 118)

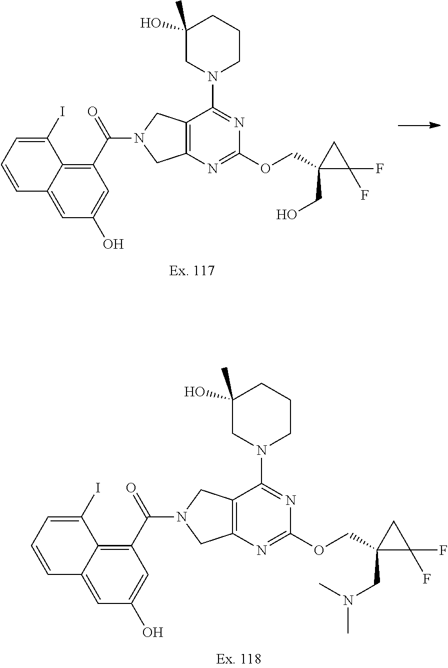 [SMILES: C[C@@]1(O)CCCN(c2nc(OC[C@]3(CO)CC3(F)F)nc3c2CN(C(=O)c2cc(O)cc4cccc(I)c24)C3)C1 ; CN(C)C[C@@]1(COc2nc3c(c(N4CCC[C@@](C)(O)C4)n2)CN(C(=O)c2cc(O)cc4cccc(I)c24)C3)CC1(F)F]

To a solution of (2-(((R)-2,2-difluoro-1-(hydroxymethyl)cyclopropyl)methoxy)-4-((R)-3-hydroxy-3-methylpiperidin-1-yl)-5,7-dihydro-6H-pyrrolo[3,4-d]pyrimidin-6-yl)(3-hydroxy-8-iodonaphthalen-1-yl)methanone (Ex. 117) (29.1 mg, 0.0437 mmol) in N,N-dimethylacetamide (0.45 mL) were added ethanesulfonyl chloride (0.00869 mL, 0.0917 mmol) and triethylamine (0.0614 mL, 0.437 mmol) at 5° C. After stirring the mixture at 5° C. for 15 min, to the reaction mixture was added 2.0 M THF solution of dimethylamine (0.220 mL, 0.440 mmol). After stirring the mixture at 75° C. for 3 h, 2.0 M aqueous solution of NaOH (0.4 mL) was added to the mixture and the mixture was stirred at 75° C. for 30 min. The reaction mixture was cooled to room temperature, diluted with DMSO (0.4 mL) and purified by reverse phase HPLC (MeCN/water with 0.1% formic acid) to afford (2-(((R)-1-((dimethylamino)methyl)-2,2-difluorocyclopropyl)methoxy)-4-((R)-3-hydroxy-3-methylpiperidin-1-yl)-5, 7-dihydro-6H-pyrrolo[3,4-d]pyrimidin-6-yl)(3-hydroxy-8-iodonaphthalen-1-yl)methanone (Ex. 118). ESI-MS m/z [M+H]⁺ 694. ¹H NMR (400 MHZ, DMSO-d₆) δ 10.3 (s, 1H), 8.02-7.95 (m, 1H), 7.87-7.80 (m, 1H), 7.29-7.23 (m, 1H), 7.16-7.07 (m, 2H), 5.07-4.86 (m, 1H), 4.72-3.99 (m, 6H), 3.91-3.67 (m, 1H), 3.60-3.47 (m, 1H), 3.13-2.98 (m, 1H), 2.25 (d, J=12.7 Hz, 1H), 2.19-2.07 (m, 6H), 1.85-1.35 (m, 6H), 1.14 (s, 3H), 0.94-0.75 (m, 2H).

Compounds in the table below were synthesized via a similar route as Ex. 118 using Ex. 117 and corresponding amines.

TABLE 6

| | | | | ESI-MS |
|---|---|---|---|---|
| Example | Structure | Name | NMR | [M + H]⁺ |
| 119 | 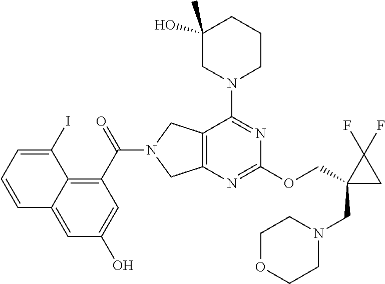 [SMILES: C[C@@]1(O)CCCN(c2nc(OC[C@@]3(CN4CCOCC4)CC3(F)F)nc3c2CN(C(=O)c2cc(O)cc4cccc(I)c24)C3)C1] | (2-(((R)-2,2-difluoro-1- (morpholinomethyl)cyclopropyl)methoxy)-4-((R)- 3-hydroxy-3-methylpiperidin-1-yl)-5,7-dihydro- 6H-pyrrolo[3,4-d]pyrimidin-6-yl)(3-hydroxy-8- iodonaphthalen-1-yl)methanone | | 736 |
| 120 | 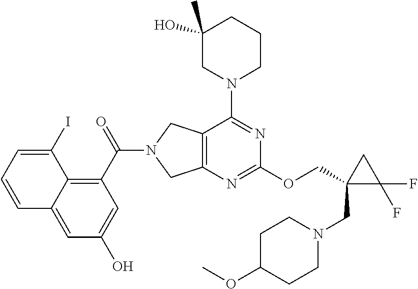 [SMILES: COC1CCN(C[C@@]2(COc3nc4c(c(N5CCC[C@@](C)(O)C5)n3)CN(C(=O)c3cc(O)cc5cccc(I)c35)C4)CC2(F)F)CC1] | (2-(((R)-2,2-difluoro-1-((4-methoxypiperidin-1- yl)methyl)cyclopropyl)methoxy)-4-((R)-3- hydroxy-3-methylpiperidin-1-yl)-5,7-dihydro-6H- pyrrolo[3,4-d]pyrimidin-6-yl)(3-hydroxy-8- iodonaphthalen-1-yl)methanone | | 764 |
| 121 | 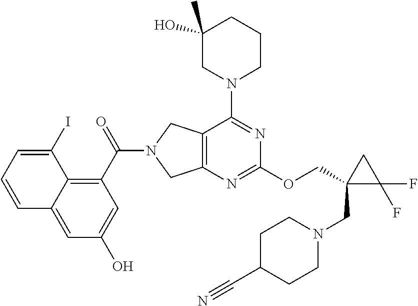 [SMILES: C[C@@]1(O)CCCN(c2nc(OC[C@]3(CN4CCC(C#N)CC4)CC3(F)F)nc3c2CN(C(=O)c2cc(O)cc4cccc(I)c24)C3)C1] | 1-(((R)-2,2-difluoro-1-(((4-((R)-3-hydroxy-3- methylpiperidin-1-yl)-6-(3-hydroxy-8-iodo-1- naphthoyl)-6, 7-dihydro-5H-pyrrolo[3,4- d]pyrimidin-2- yl)oxy)methyl)cyclopropyl)methyl)piperidine-4- carbonitrile | | 759 |
| 122 | 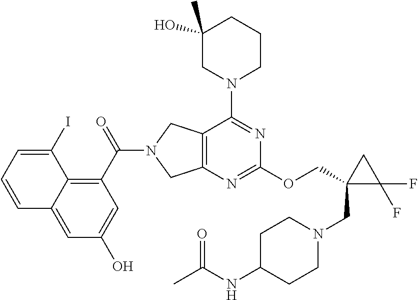 [SMILES: CC(=O)NC1CCN(C[C@@]2(COc3nc4c(c(N5CCC[C@@](C)(O)C5)n3)CN(C(=O)c3cc(O)cc5cccc(I)c35)C4)CC2(F)F)CC1] | N-(1-(((R)-2,2-difluoro-1-(((4-((R)-3-hydroxy-3- methylpiperidin-1-yl)-6-(3-hydroxy-8-iodo-1- naphthoyl)-6,7-dihydro-5H-pyrrolo[3,4- d]pyrimidin-2- yl)oxy)methyl)cyclopropyl)methyl)piperidin-4- yl)acetamide | | 791 |
| 123 | 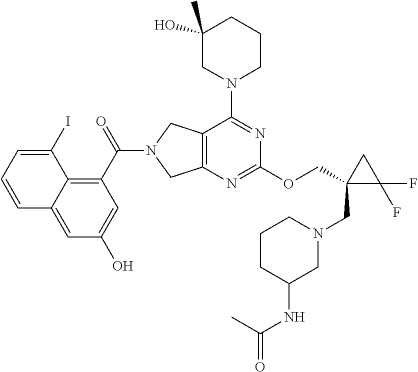 [SMILES: CC(=O)NC1CCCN(C[C@@]2(COc3nc4c(c(N5CCC[C@@](C)(O)C5)n3)CN(C(=O)c3cc(O)cc5cccc(I)c35)C4)CC2(F)F)C1] | N-(1-(((R)-2,2-difluoro-1-(((4-((R)-3-hydroxy-3- methylpiperidin-1-yl)-6-(3-hydroxy-8-iodo-1- naphthoyl)-6,7-dihydro-5H-pyrrolo[3,4- d]pyrimidin-2- yl)oxy)methyl)cyclopropyl)methyl)piperidin-3- yl)acetamide | | 791 |

### Example 124: (2-(((R)-1-((dimethylamino)methyl)-2,2-difluorocyclopropyl)methoxy)-4-((R)-2-methylazepan-1-yl)-5,7-dihydro-6H-pyrrolo[3,4-d]pyrimidin-6-yl)(3-hydroxy-8-iodonaphthalen-1-yl)methanone (Ex. 124)

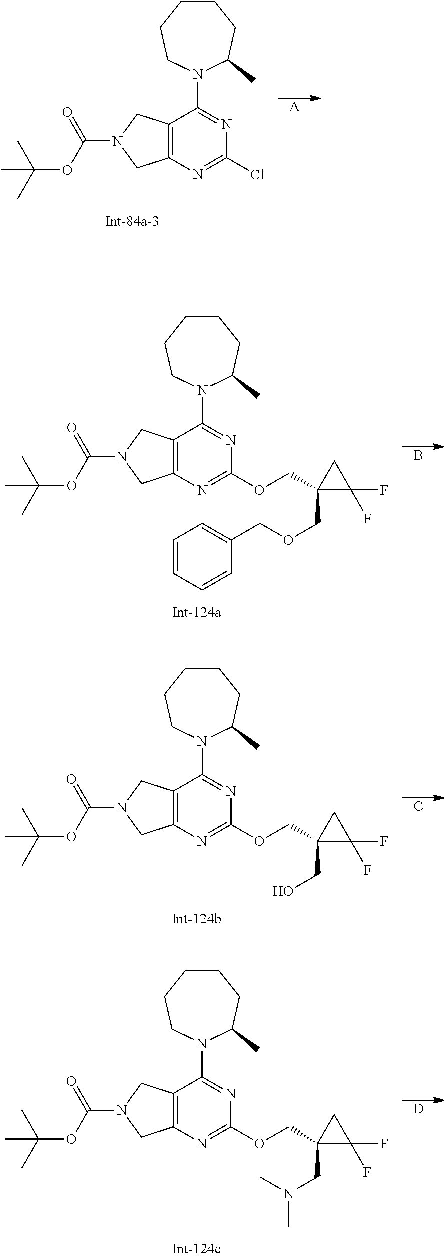

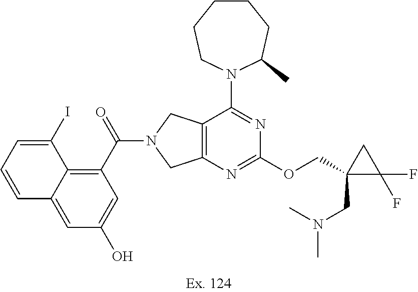 [SMILES: C[C@@H]1CCCCCN1c1nc(OC[C@]2(CN(C)C)CC2(F)F)nc2c1CN(C(=O)c1cc(O)cc3cccc(I)c13)C2]

### Step A: tert-butyl 2-(((R)-1-((benzyloxy)methyl)-2,2-difluorocyclopropyl)methoxy)-4-((R)-2-methylazepan-1-yl)-5,7-dihydro-6H-pyrrolo[3,4-d]pyrimidine-6-carboxylate (Int-124a)

To a solution of tert-butyl (R)-2-chloro-4-(2-methylazepan-1-yl)-5,7-dihydro-6H-pyrrolo[3,4-d]pyrimidine-6-carboxylate (Int-84a-3) (200 mg, 0.545 mmol), (S)-(1-((benzyloxy)methyl)-2,2-difluorocyclopropyl)methanol (Int-UU5-2) (187 mg, 0.818 mmol) and RuPhos Pd G3 (13.7 mg, 0.0164 mmol) in 1,4-dioxane (1.5 mL) was added cesium carbonate (533 mg, 1.64 mmol) at room temperature. After stirring the mixture at 100° C. for 5 h, the reaction mixture was cooled to room temperature, filtered and concentrated in vacuo. The residue was purified by flash silica gel chromatography (0-50%, EtOAc gradient in hexane) to afford tert-butyl 2-(((R)-1-((benzyloxy)methyl)-2,2-difluorocyclopropyl)methoxy)-4-((R)-2-methylazepan-1-yl)-5,7-dihydro-6H-pyrrolo[3,4-d]pyrimidine-6-carboxylate (Int-124a) (305 mg). ESI-MS m/z [M+H]⁺ 559.

### Step B: tert-butyl 2-(((R)-2,2-difluoro-1-(hydroxymethyl)cyclopropyl)methoxy)-4-((R)-2-methylazepan-1-yl)-5,7-dihydro-6H-pyrrolo[3,4-d]pyrimidine-6-carboxylate (Int-124b)

To a solution of tert-butyl 2-(((R)-1-((benzyloxy)methyl)-2,2-difluorocyclopropyl)methoxy)-4-((R)-2-methylazepan-1-yl)-5,7-dihydro-6H-pyrrolo[3,4-d]pyrimidine-6-carboxylate (Int-124a) (305 mg, 0.545 mmol) in ethanol (5.5 mL) was added Pd(OH)₂/C (191 mg, 0.273 mmol). The reaction mixture was purged with H₂ gas and vacuum three times and stirred at room temperature for 2 h. The reaction mixture was filtered and concentrated in vacuo. The residue was purified by flash NH-silica gel chromatography (30-100%, EtOAc gradient in hexane) to afford tert-butyl 2-(((R)-2,2-difluoro-1-(hydroxymethyl)cyclopropyl)methoxy)-4-((R)-2-methylazepan-1-yl)-5,7-dihydro-6H-pyrrolo[3,4-d]pyrimidine-6-carboxylate (Int-124b) (253 mg). ESI-MS m/z [M+H]⁺ 469.

### Step C: tert-butyl 2-(((R)-1-((dimethylamino)methyl)-2,2-difluorocyclopropyl)methoxy)-4-((R)-2-methylazepan-1-yl)-5,7-dihydro-6H-pyrrolo[3,4-d]pyrimidine-6-carboxylate (Int-124c)

To a solution of tert-butyl 2-(((R)-2,2-difluoro-1-(hydroxymethyl)cyclopropyl)methoxy)-4-((R)-2-methylazepan-1-yl)-5,7-dihydro-6H-pyrrolo[3,4-d]pyrimidine-6-carboxylate (Int-124b) (253 mg, 0.539 mmol) in N,N-dimethylacetamide (5.0 mL) were added ethanesulfonyl chloride (0.0562 mL, 0.593 mmol) and triethylamine (0.379 mL, 2.70 mmol) at 5° C. After stirring the mixture at 5° C. for 10 min, to the reaction mixture was added 2.0 M THF solution of dimethylamine (1.35 mL, 2.70 mmol). After stirring the mixture at 60° C. for 14 h, the reaction was quenched by the addition of H₂O. The reaction mixture was extracted with EtOAc, washed with brine, dried over Na₂SO₄, filtered and concentrated in vacuo. The residue was purified by flash NH-silica gel chromatography (30-100%, EtOAc gradient in hexane) to afford tert-butyl 2-(((R)-1-((dimethylamino)methyl)-2,2-difluorocyclopropyl)methoxy)-4-((R)-2-methylazepan-1-yl)-5,7-dihydro-6H-pyrrolo[3,4-d]pyrimidine-6-carboxylate (Int-124c) (216 mg). ESI-MS m/z [M+H]⁺ 496.

### Step D: Example 124 (2-(((R)-1-((dimethylamino)methyl)-2,2-difluorocyclopropyl)methoxy)-4-((R)-2-methylazepan-1-yl)-5,7-dihydro-6H-pyrrolo[3,4-d]pyrimidin-6-yl)(3-hydroxy-8-iodonaphthalen-1-yl)methanone (Ex. 124)

To a solution of tert-butyl 2-(((R)-1-((dimethylamino)methyl)-2,2-difluorocyclopropyl)methoxy)-4-((R)-2-methylazepan-1-yl)-5,7-dihydro-6H-pyrrolo[3,4-d]pyrimidine-6-carboxylate (Int-124c) (216 mg, 0.435 mmol) in chloroform (4.0 mL) was added 4.0 M dioxane solution of HCl (2.2 mL). After stirring the mixture at room temperature for 30 min, the reaction mixture was concentrated in vacuo to afford amine product.

To a solution of amine product, 3-hydroxy-8-iodo-1-naphthoic acid (Int-T002) (97.0 mg, 0.309 mmol), 1-(3-dimethylaminopropyl)-3-ethylcarbodiimide hydrochloride (59.2 mg, 0.309 mmol) and 1-hydroxybenzotriazole hydrate (47.3 mg, 0.309 mmol) in DMF (3.0 mL) was added N,N-diisopropylethylamine (0.244 mL, 1.40 mmol) at room temperature. After stirring the mixture at room temperature for 13 h, the reaction was quenched by the addition of H₂O. The reaction mixture was extracted with EtOAc, washed with brine, dried over Na₂SO₄, filtered and concentrated in vacuo. The residue was purified by reverse phase HPLC (MeCN/water with 0.1% formic acid) to afford (2-(((R)-1-((dimethylamino)methyl)-2,2-difluorocyclopropyl)methoxy)-4-((R)-2-methylazepan-1-yl)-5,7-dihydro-6H-pyrrolo[3,4-d]pyrimidin-6-yl)(3-hydroxy-8-iodonaphthalen-1-yl)methanone (Ex. 124). ESI-MS m/z [M+H]⁺ 692. ¹H NMR (400 MHz, DMSO-d₆) δ 10.2 (s, 1H), 8.03-7.96 (m, 1H), 7.87-7.80 (m, 1H), 7.29-7.23 (m, 1H), 7.19-7.07 (m, 2H), 5.16-4.85 (m, 1H), 4.79-4.00 (m, 5H), 3.80-3.55 (m, 1H), 3.05-2.85 (m, 1H), 2.37-2.20 (m, 2H), 2.17-2.06 (m, 6H), 1.96-1.56 (m, 4H), 1.55-1.25 (m, 4H), 1.25-0.52 (m, 6H).

### Example 125-1: (3-amino-8-iodonaphthalen-1-yl)(2-(((R)-1-((dimethylamino)methyl)-2,2-difluorocyclopropyl)methoxy)-4-((R)-3-hydroxy-3-methylpiperidin-1-yl)-5,7-dihydro-6H-pyrrolo[3,4-d]pyrimidin-6-yl)methanone (Ex. 125)

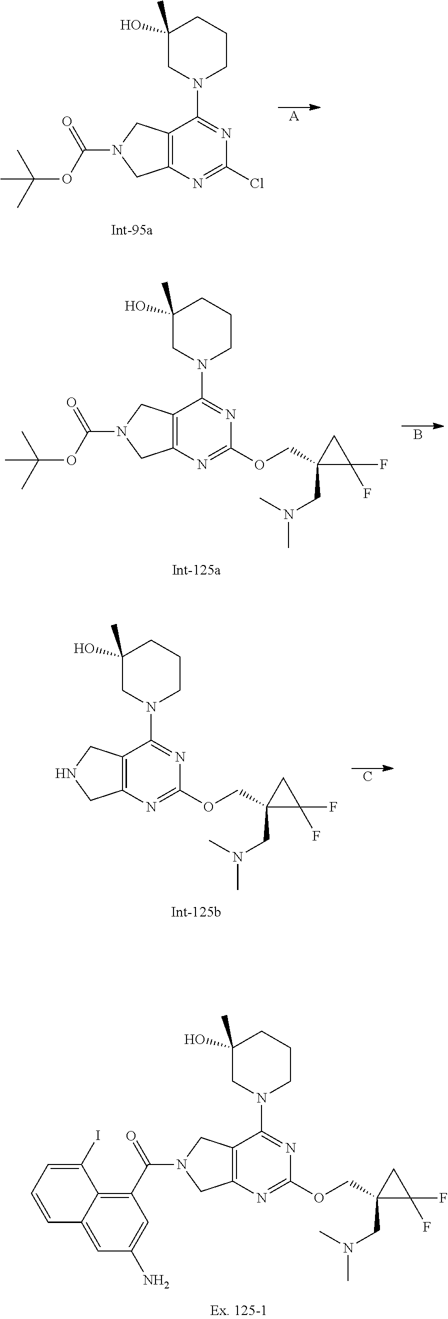

### Step A: tert-butyl 2-(((R)-1-((dimethylamino)methyl)-2,2-difluorocyclopropyl)methoxy)-4-((R)-3-hydroxy-3-methylpiperidin-1-yl)-5,7-dihydro-6H-pyrrolo[3,4-d]pyrimidine-6-carboxylate (Int-125a)

To a solution of tert-butyl (R)-2-chloro-4-(3-hydroxy-3-methylpiperidin-1-yl)-5,7-dihydro-6H-pyrrolo[3,4-d]pyrimidine-6-carboxylate (Int-95a) (100 mg, 0.271 mmol), (R)-(1-((dimethylamino)methyl)-2,2-difluorocyclopropyl)methanol (Int-UU8-1) hydrochloride (82.0 mg, 0.407 mmol) and RuPhos Pd G3 (6.80 mg, 0.00813 mmol) in 1,4-dioxane (1.5 mL) were added cesium carbonate (265 mg, 0.813 mmol) and N,N-diisopropylethylamine (0.0944 mL, 0.542 mmol) at room temperature. After stirring the mixture at 100° C. for 14 h, the reaction mixture was cooled to room temperature, filtered and concentrated in vacuo. The residue was purified by flash NH-silica gel chromatography (20-100%, EtOAc gradient in hexane) to afford tert-butyl 2-(((R)-1-((dimethylamino)methyl)-2,2-difluorocyclopropyl)methoxy)-4-((R)-3-hydroxy-3-methylpiperidin-1-yl)-5,7-dihydro-6H-pyrrolo[3,4-d]pyrimidine-6-carboxylate (Int-125a) (104 mg). ESI-MS m/z [M+H]⁺ 498.

### Step B: (R)-1-(2-(((R)-1-((dimethylamino)methyl)-2,2-difluorocyclopropyl)methoxy)-6,7-dihydro-5H-pyrrolo[3,4-d]pyrimidin-4-yl)-3-methylpiperidin-3-ol (Int-125b)

To a solution of tert-butyl 2-(((R)-1-((dimethylamino)methyl)-2,2-difluorocyclopropyl)methoxy)-4-((R)-3-hydroxy-3-methylpiperidin-1-yl)-5,7-dihydro-6H-pyrrolo[3,4-d]pyrimidine-6-carboxylate (Int-125a) (104 mg, 0.210 mmol) in dichloromethane (2.0 mL) was added trifluoroacetic acid (0.4 mL). After stirring the mixture at room temperature for 2 h, the reaction mixture was concentrated in vacuo. The residue was purified by flash NH-silica gel chromatography (0-50%, MeOH gradient in EtOAc) to afford (R)-1-(2-(((R)-1-((dimethylamino)methyl)-2,2-difluorocyclopropyl)methoxy)-6,7-dihydro-5H-pyrrolo[3,4-d]pyrimidin-4-yl)-3-methylpiperidin-3-ol (Int-125b) (72.8 mg). ESI-MS m/z [M+H]⁺ 398.

### Step C: Example 125-1 (3-amino-8-iodonaphthalen-1-yl)(2-(((R)-1-((dimethylamino)methyl)-2,2-difluorocyclopropyl)methoxy)-4-((R)-3-hydroxy-3-methylpiperidin-1-yl)-5,7-dihydro-6H-pyrrolo[3,4-d]pyrimidin-6-yl)methanone (Ex. 125-1)

To a solution of (R)-1-(2-(((R)-1-((dimethylamino)methyl)-2,2-difluorocyclopropyl)methoxy)-6,7-dihydro-5H-pyrrolo[3,4-d]pyrimidin-4-yl)-3-methylpiperidin-3-ol (Int-125b) (6.00 mg, 0.0151 mmol), 3-amino-8-iodo-naphthalene-1-carboxylic acid (Int-T001) (5.20 mg, 0.0166 mmol), 1-(3-dimethylaminopropyl)-3-ethylcarbodiimide hydrochloride (3.18 mg, 0.0166 mmol) and 1-hydroxybenzotriazole hydrate (2.54 mg, 0.0166 mmol) in DMF (0.15 mL) was added N,N-diisopropylethylamine (0.0131 mL, 0.0755 mmol) at room temperature. After stirring the mixture at room temperature for 14 h, the reaction was quenched by the addition of H₂O. The reaction mixture was extracted with EtOAc, washed with brine, dried over Na₂SO₄, filtered and concentrated in vacuo. The residue was purified by reverse phase HPLC (MeCN/water with 0.1% formic acid) to afford (3-amino-8-iodonaphthalen-1-yl)(2-(((R)-1-((dimethylamino)methyl)-2,2-difluorocyclopropyl)methoxy)-4-((R)-3-hydroxy-3-methylpiperidin-1-yl)-5,7-dihydro-6H-pyrrolo[3,4-d]pyrimidin-6-yl)methanone (Ex. 125-1). ESI-MS m/z [M+H]⁺ 693. ¹H NMR (400 MHz, DMSO-d₆) δ 7.82-7.77 (m, 1H), 7.67-7.60 (m, 1H), 7.03-6.96 (m, 2H), 6.94-6.89 (m, 1H), 5.69 (s, 2H), 5.06-4.80 (m, 1H), 4.65-4.00 (m, 6H), 3.57-3.47 (m, 2H), 2.18-2.06 (m, 6H), 1.84-1.35 (m, 6H), 1.25-1.19 (m, 2H), 1.14 (s, 3H), 0.89-0.76 (m, 2H).

### Example 125-2: (3-amino-8-iodonaphthalen-1-yl)(2-((1-((dimethylamino)methyl)cyclopropyl)methoxy)-4-(2-methylazepan-1-yl)-5,7-dihydro-6H-pyrrolo[3,4-d]pyrimidin-6-yl)methanone (Ex. 125-2)

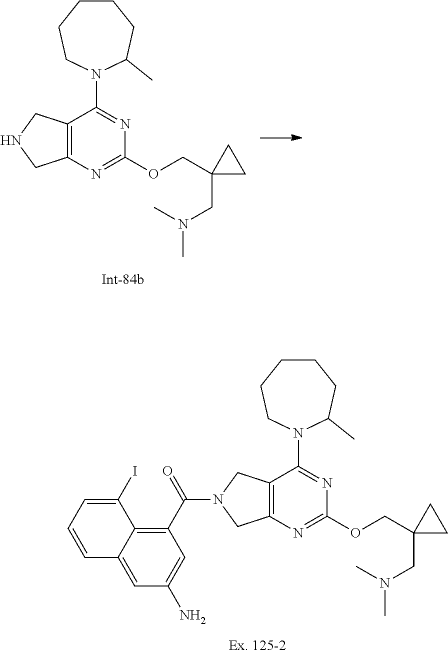 [SMILES: CC1CCCCCN1c1nc(OCC2(CN(C)C)CC2)nc2c1CNC2 ; CC1CCCCCN1c1nc(OCC2(CN(C)C)CC2)nc2c1CN(C(=O)c1cc(N)cc3cccc(I)c13)C2]

To a mixture of 3-amino-8-iodo-1-naphthoic acid (Int-T001) (52 mg, 0.167 mmol), 1-hydroxybenzotriazole monohydrate (26 mg, 0.167 mmol), 1-(3-dimethylaminopropyl)-3-ethylcarbodiimide hydrochloride (32 mg, 0.167 mmol) and N,N-diisopropylethylamine (0.071 mL, 0.417 mmol) in N,N-dimethylformamide (2 mL) was added N,N-dimethyl-1-(1-(((4-(2-methylazepan-1-yl)-6,7-dihydro-5H-pyrrolo[3,4-d]pyrimidin-2-yl)oxy)methyl)cyclopropyl)methanamine (Int-84b) (50 mg, 0.139 mmol), and the mixture was stirred at room temperature for 3 h. The mixture was diluted with ethyl acetate and the diluted mixture was washed with water and the mixture was concentrated under reduced pressure. The residue was purified by reverse phase HPLC (MeCN/water with 0.1% formic acid) to give (3-amino-8-iodonaphthalen-1-yl)(2-((1-((dimethylamino)methyl)cyclopropyl)methoxy)-4-(2-methylazepan-1-yl)-5,7-dihydro-6H-pyrrolo[3,4-d]pyrimidin-6-yl)methanone (Ex. 125-2) (30 mg). ESI-MS m/z [M+H]⁺ 655.

### Example 126: (2-((1-((dimethylamino)methyl)cyclopropyl)methoxy)-4-(2-methylazepan-1-yl)-5,7-dihydro-6H-pyrrolo[3,4-d]pyrimidin-6-yl)(8-ethynyl-3-hydroxynaphthalen-1-yl)methanone (Ex. 126)

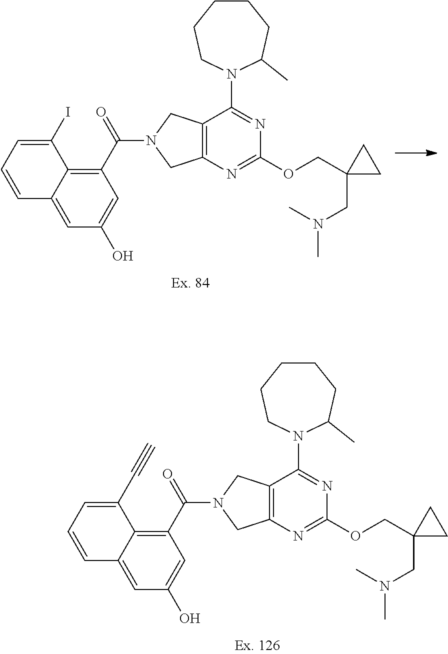 [SMILES: CC1CCCCCN1c1nc(OCC2(CN(C)C)CC2)nc2c1CN(C(=O)c1cc(O)cc3cccc(I)c13)C2 ; C#Cc1cccc2cc(O)cc(C(=O)N3Cc4nc(OCC5(CN(C)C)CC5)nc(N5CCCCCC5C)c4C3)c12]

To a solution of Copper(I) iodide (2.9 mg, 0.0153 mmol), bis(triphenylphosphine)palladium(II) dichloride (11 mg, 0.0153 mmol), (2-((1-((dimethylamino)methyl)cyclopropyl)methoxy)-4-(2-methylazepan-1-yl)-5,7-dihydro-6H-pyrrolo[3,4-d]pyrimidin-6-yl)(3-hydroxy-8-iodonaphthalen-1-yl)methanone (Ex. 84) (100 mg, 0.0153 mmol), triethylamine (0.107 mL, 0.763 mmol) in N,N-dimethylformamide (3 mL) was added Triisopropylsilylacetylene (0.102 mL, 0.458 mmol). The vessel was evacuated and backfilled with nitrogen, and the mixture was stirred at 95° C. for 0.5 h. The mixture was diluted with ethyl acetate and the diluted mixture was washed with water and the mixture was concentrated under reduced pressure. The residue was purified by flash NH-silica gel chromatography (0-40%, methanol gradient in ethyl acetate) to give the coupling product.

To a solution of the coupling product in tetrahydrofuran (2 mL) was added tetrabutylammonium fluoride (0.229 mL, 0.229 mmol, 1M in tetrahydrofuran) and the mixture was stirred at room temperature for 0.5 h. The mixture was diluted with ethyl acetate and the diluted mixture was washed with water and the mixture was concentrated under reduced pressure. The residue was purified by reverse phase HPLC (MeCN/water with 0.1% formic acid) to give (2-((1-((dimethylamino)methyl)cyclopropyl)methoxy)-4-(2-methylazepan-1-yl)-5,7-dihydro-6H-pyrrolo[3,4-d]pyrimidin-6-yl)(8-ethynyl-3-hydroxynaphthalen-1-yl)methanone (Ex. 126). ESI-MS m/z [M+H]⁺ 554. ¹H NMR (400 MHZ, DMSO-d₆) δ 10.2 (s, 1H), 7.89-7.84 (m, 1H), 7.58-7.52 (m, 1H), 7.48-7.39 (m, 1H), 7.31-7.26 (m, 1H), 7.12-7.06 (m, 1H), 5.20-4.80 (m, 2H), 4.61-4.30 (m, 2H), 4.17-3.95 (m, 4H), 3.13-2.75 (m, 1H), 2.25-2.08 (m, 2H), 2.16 (s, 3H), 2.11 (s, 3H), 2.00-0.95 (m, 12H), 0.59-0.48 (m, 2H), 0.39-0.31 (m, 2H).

Compounds in the table below were synthesized via a similar route as Ex. 126 using the corresponding iodides samples.

TABLE 7

| | | | | ESI-MS |
|---|---|---|---|---|
| Example | Structure | Name | NMR | [M + H]⁺ |
| 127 | 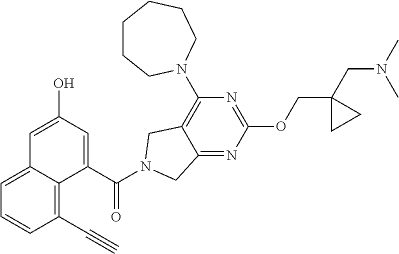 [SMILES: C#Cc1cccc2cc(O)cc(C(=O)N3Cc4nc(OCC5(CN(C)C)CC5)nc(N5CCCCCC5)c4C3)c12] | (4-(azepan-1-yl)-2- ((1-((dimethyl- amino)methyl)cyclo- propyl)methoxy)-5,7- dihydro-6H-pyrrolo[3,4- d]pyrimidin-6- yl)(8-ethynyl-3- hydroxynaphthalen-1- yl)methanone | | 540 |
| 128 | 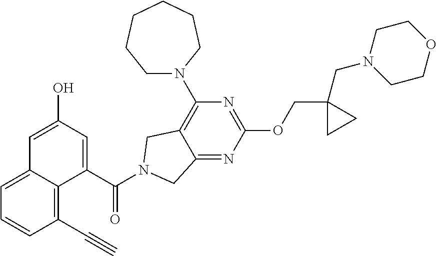 [SMILES: C#Cc1cccc2cc(O)cc(C(=O)N3Cc4nc(OCC5(CN6CCOCC6)CC5)nc(N5CCCCCC5)c4C3)c12] | (4-(azepan-1-yl)-2-((1- (morpholinomethyl)cyclo- propyl)methoxy)-5,7- dihydro-6H-pyrrolo[3,4- d]pyrimidin-6-yl)(8- ethynyl-3-hydroxy- naphthalen- 1-yl)methanone | | 582 |
| 129 | 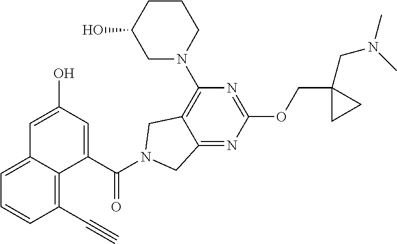 [SMILES: C#Cc1cccc2cc(O)cc(C(=O)N3Cc4nc(OCC5(CN(C)C)CC5)nc(N5CCC[C@@H](O)C5)c4C3)c12] | (R)-(2-((1-((dimethyl- amino)methyl)cyclo- propyl)methoxy)- 4-(3-hydroxypiperidin-1-yl)- 5,7-dihydro-6H- pyrrolo[3,4-d]pyrimidin-6- yl)(3-hydroxy-8- iodonaphthalen-1- yl)methanone | | 644 |
| 130 | 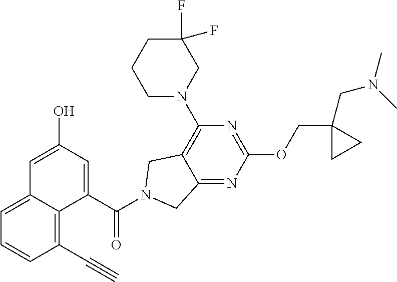 [SMILES: C#Cc1cccc2cc(O)cc(C(=O)N3Cc4nc(OCC5(CN(C)C)CC5)nc(N5CCCC(F)(F)C5)c4C3)c12] | (4-(3,3-difluoropiperidin-1- yl)-2-((1-((dimethyl- amino)methyl)cyclo- propyl)methoxy)- 5,7-dihydro-6H-pyrrolo[3,4- d]pyrimidin-6- yl)(3-hydroxy-8- iodonaphthalen-1- yl)methanone | | 664 |
| 131 | 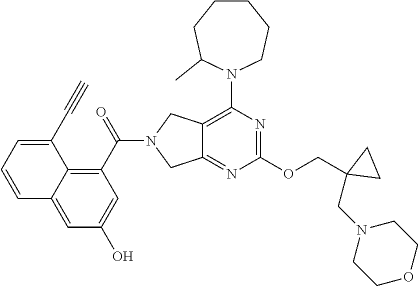 [SMILES: C#Cc1cccc2cc(O)cc(C(=O)N3Cc4nc(OCC5(CN6CCOCC6)CC5)nc(N5CCCCCC5C)c4C3)c12] | (8-ethynyl-3-hydroxy- naphthalen-1-yl)(4-(2- methylazepan-1-yl)-2-((1- (morpholinomethyl)cyclo- propyl)methoxy)-5,7- dihydro-6H-pyrrolo[3,4- d]pyrimidin-6- yl)methanone | | 596 |
| 132 | 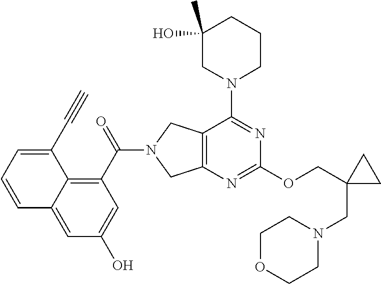 [SMILES: C#Cc1cccc2cc(O)cc(C(=O)N3Cc4nc(OCC5(CN6CCOCC6)CC5)nc(N5CCC[C@@](C)(O)C5)c4C3)c12] | (R)-(8-ethynyl-3- hydroxynaphthalen-1-yl)(4- (3-hydroxy- 3-methylpiperidin- 1-yl)-2-((1- (morpholinomethyl)cyclo- propyl)methoxy)-5,7- dihydro-6H-pyrrolo[3,4- d]pyrimidin-6- yl)methanone | | 598 |
| 133 | 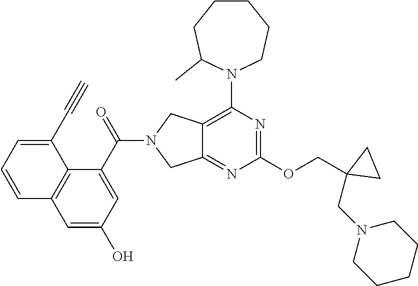 [SMILES: C#Cc1cccc2cc(O)cc(C(=O)N3Cc4nc(OCC5(CN6CCCCC6)CC5)nc(N5CCCCCC5C)c4C3)c12] | (8-ethynyl-3- hydroxynaphthalen-1- yl)(4-(2- methylazepan-1-yl)-2- ((1-(piperidin-1- ylmethyl)cyclo- propyl)methoxy)-5,7- dihydro-6H-pyrrolo[3,4- d]pyrimidin-6-yl)methanone | | 594 |
| 134 | 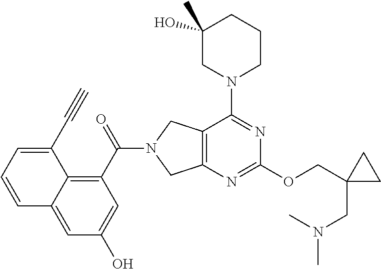 [SMILES: C#Cc1cccc2cc(O)cc(C(=O)N3Cc4nc(OCC5(CN(C)C)CC5)nc(N5CCC[C@@](C)(O)C5)c4C3)c12] | (R)-(2-((1-((dimethyl- amino)methyl)cyclo- propyl)methoxy)- 4-(3-hydroxy-3- methylpiperidin-1-yl)-5,7- dihydro-6H-pyrrolo[3,4- d]pyrimidin-6-yl)(8- ethynyl-3- hydroxynaphthalen-1- yl)methanone | | 556 |
| 135 | 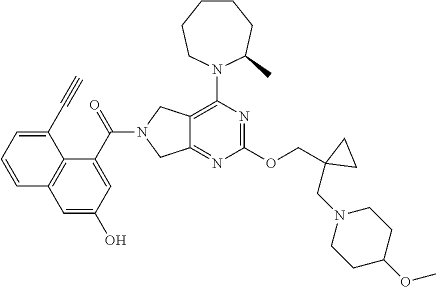 [SMILES: C#Cc1cccc2cc(O)cc(C(=O)N3Cc4nc(OCC5(CN6CCC(OC)CC6)CC5)nc(N5CCCCC[C@H]5C)c4C3)c12] | (R)-(8-ethynyl-3- hydroxynaphthalen-1-yl)(2- ((1-((4-methoxypiperidin-1- yl)methyl)cyclo- propyl)methoxy)-4-(2- methylazepan-1-yl)-5,7- dihydro-6H- pyrrolo[3,4-d]pyrimidin-6- yl)methanone | ¹H NMR (400 MHz, DMSO-d₆) δ 10.2 (s, 1H), 7.99- 7.84 (m, 1H), 7.58- 7.51 (m, 1H), 7.48- 7.40 (m, 1H), 7.30- 7.26 (m, 1H), 7.12- 7.06 (m, 1H), 5.19- 4.75 (m, 2H), 4.61- 4.30 (m, 2H), 4.20- 3.95 (m, 4H), 3.21 (s, 1.5H), 3.19 (s, 1.5H), 3.15-3.08 (m, 2H), 2.35-2.11 (m, 2H) 2.10-0.95 (m, 20H), 0.60- 0.49 (m, 2H), 0.40- 0.29 (m, 2H) | 624 |
| 136 | 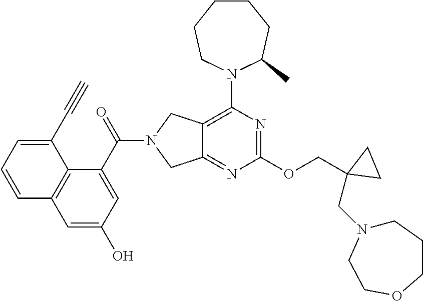 [SMILES: C#Cc1cccc2cc(O)cc(C(=O)N3Cc4nc(OCC5(CN6CCCOCC6)CC5)nc(N5CCCCC[C@H]5C)c4C3)c12] | (R)-(2-((1-((1,4- oxazepan-4- yl)methyl)cyclo- propyl)methoxy)-4-(2- methylazepan-1-yl)-5,7- dihydro-6H- pyrrolo[3,4-d]pyrimidin-6- yl)(8-ethynyl-3- hydroxynaphthalen-1- yl)methanone | | 610 |
| 137 | 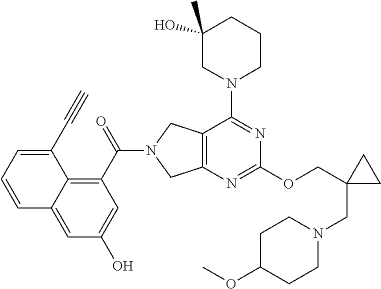 [SMILES: C#Cc1cccc2cc(O)cc(C(=O)N3Cc4nc(OCC5(CN6CCC(OC)CC6)CC5)nc(N5CCC[C@@](C)(O)C5)c4C3)c12] | (R)-(8-ethynyl-3- hydroxynaphthalen-1- yl)(4-(3-hydroxy-3- methylpiperidin-1-yl)- 2-((1-((4-methoxypiperidin- 1-yl)methyl)cyclo- propyl)methoxy)-5,7- dihydro-6H-pyrrolo[3,4- d]pyrimidin-6-yl)methanone | | 626 |
| 138 | 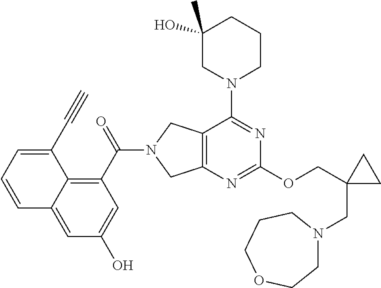 [SMILES: C#Cc1cccc2cc(O)cc(C(=O)N3Cc4nc(OCC5(CN6CCCOCC6)CC5)nc(N5CCC[C@@](C)(O)C5)c4C3)c12] | (R)-(2-((1-((1,4-oxazepan-4- yl)methyl)cyclo- propyl)methoxy)-4-(3- hydroxy-3-methylpiperidin- 1-yl)-5,7-dihydro-6H- pyrrolo[3,4-d]pyrimidin-6- yl)(8-ethynyl-3- hydroxynaphthalen-1- yl)methanone | | 612 |
| 139 | 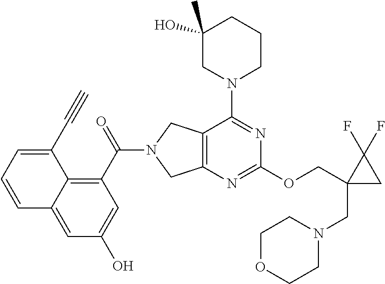 [SMILES: C#Cc1cccc2cc(O)cc(C(=O)N3Cc4nc(OCC5(CN6CCOCC6)CC5(F)F)nc(N5CCC[C@@](C)(O)C5)c4C3)c12] | (2-(((R)-2,2-difluoro-1- (morpholinomethyl)cyclo- propyl)methoxy)-4- ((R)-3-hydroxy-3- methylpiperidin-1-yl)-5,7- dihydro-6H-pyrrolo[3,4- d]pyrimidin-6-yl)(8- ethynyl-3-hydroxynaphthalen- 1-yl)methanone | | 634 |
| 140 | 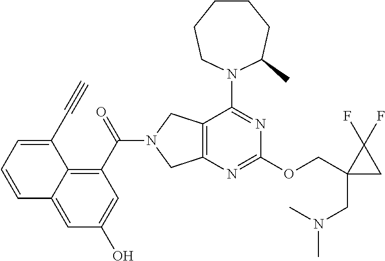 [SMILES: C#Cc1cccc2cc(O)cc(C(=O)N3Cc4nc(OCC5(CN(C)C)CC5(F)F)nc(N5CCCCC[C@H]5C)c4C3)c12] | (2-(((R)-1- ((dimethylamino)methyl)- 2,2-difluorocyclo- propyl)methoxy)-4-((R)-2- methylazepan-1-yl)-5,7- dihydro-6H- pyrrolo[3,4-d]pyrimidin- 6-yl)(8-ethynyl-3- hydroxynaphthalen-1- yl)methanone | ¹H NMR (400 MHz, DMSO-d₆) δ 10.2 (s, 1H), 7.89- 7.82 (m, 1H), 7.57- 7.49 (m, 1H), 7.47- 7.37 (m, 1H), 7.30- 7.24 (m, 1H), 7.08- 7.03 (m, 1H), 5.11- 4.75 (m, 1H), 4.62- 3.95 (m, 6H), 3.75- 2.60 (m, 1H), 2.92- 2.80 (m, 1H), 2.23 (d, J = 12.6 Hz, 1H) 2.17-2.05 (m, 6H), 1.82-0.90 (m, 15H). | 590 |
| 141 | 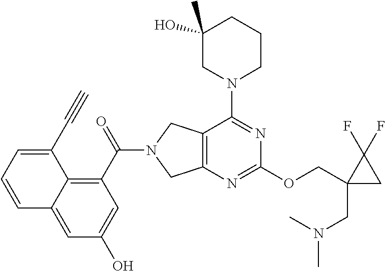 [SMILES: C#Cc1cccc2cc(O)cc(C(=O)N3Cc4nc(OCC5(CN(C)C)CC5(F)F)nc(N5CCC[C@@](C)(O)C5)c4C3)c12] | (2-(((R)-1-((dimethyl- amino)methyl)-2,2- difluorocyclo- propyl)methoxy)-4-((R)-3- hydroxy-3-methylpiperidin- 1-yl)-5,7-dihydro- 6H-pyrrolo[3,4-d]pyrimidin- 6-yl)(8-ethynyl-3- hydroxynaphthalen-1- yl)methanone | ¹H NMR (400 MHz, DMSO-d₆) δ 10.2-10.1 (m, 1H), 7.89-7.82 (m, 1H), 7.57-7.50 (m, 1H), 7.47-7.41 (m, 1H), 7.30-7.25 (m, 1H), 7.09-7.03 (m, 1H), 5.01-4.73 (m, 1H), 4.67-3.93 (m, 8H), 3.63-3.48 (m, 1H), 3.20-2.93 (m, 1H), 2.24 (d, J = 12.7 Hz, 1H), 2.17-2.05 (m, 6H), 1.83-1.35 (m, 7H), 1.14 (s, 3H), 0.76 (d, J = 6.3 Hz, 1H). | 592 |
| 142 | 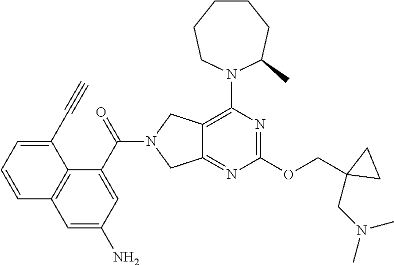 [SMILES: C#Cc1cccc2cc(N)cc(C(=O)N3Cc4nc(OCC5(CN(C)C)CC5)nc(N5CCCCC[C@H]5C)c4C3)c12] | (R)-(3-amino-8-ethynyl- naphthalen-1-yl)(2- ((1-((dimethyl- amino)methyl)cyclo- propyl)methoxy)- 4-(2-methylazepan-1-yl)- 5,7-dihydro-6H- pyrrolo[3,4-d]pyrimidin- 6-yl)methanone | | 553 |
| 143 | 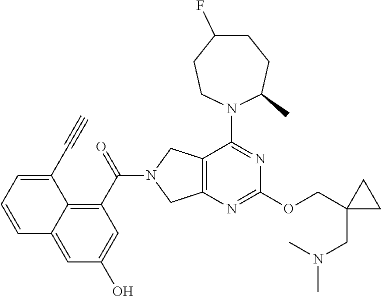 [SMILES: C#Cc1cccc2cc(O)cc(C(=O)N3Cc4nc(OCC5(CN(C)C)CC5)nc(N5CCC(F)CC[C@H]5C)c4C3)c12] | (2-((1-((dimethyl- amino)methyl)cyclo- propyl)methoxy)- 4-((2R)-5-fluoro-2- methylazepan-1-yl)-5,7- dihydro-6H-pyrrolo[3,4- d]pyrimidin-6-yl)(8- ethynyl-3-hydroxy- naphthalen- 1-yl)methanone | | 572 |

### 7-(8-bromo-3-(methoxymethoxy)naphthalen-1-yl)-2-((1-((dimethylamino)methyl)cyclopropyl)methoxy)-5,6,7,8-tetrahydropyrido[3,4-d]pyrimidin-4-ol (Int-144e-1) and Int-144e-2 isomer (mixture)

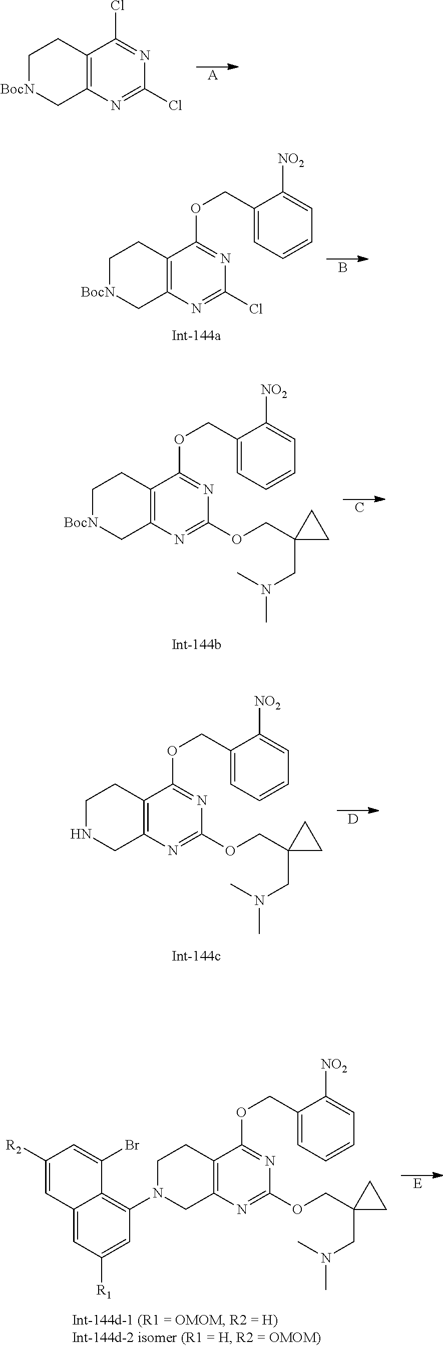

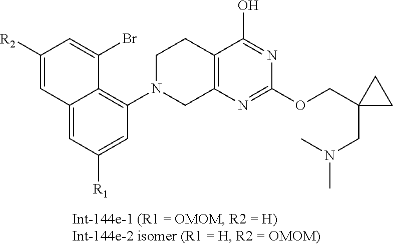

### Step A: tert-butyl 2-chloro-4-((2-nitrobenzyl)oxy)-5,8-dihydropyrido[3,4-d]pyrimidine-7(6H)-carboxylate (Int-144a)

To a stirred solution of tert-butyl 2,4-dichloro-5,8-dihydropyrido[3,4-d]pyrimidine-7(6H)-carboxylate (20.0 g, 65.7 mmol) and (2-nitrophenyl)methanol (13.1 g, 85.5 mmol) in toluene (200 mL) was added cesium carbonate (42.9 g, 0.131 mmol), and the mixture was warmed to 80° C. and stirred for 3 h. The mixture was cooled to room temperature and diluted with ethyl acetate (200 mL). The mixture was washed twice with water (200 mL), and the organic layer was concentrated under reduced pressure. MeCN (300 mL) and water (60 mL) were added to the residue and the mixture was suspended at 80° C. for 1 h. The suspension was cooled to room temperature and the solid was filtered and dried under reduced pressure to give tert-butyl 2-chloro-4-((2-nitrobenzyl)oxy)-5,8-dihydropyrido[3,4-d]pyrimidine-7(6H)-carboxylate (Int-144a) (15.7 g). ESI-MS m/z [M+H]⁺ 421, 423.

### Step B: tert-butyl 2-((1-((dimethylamino)methyl)cyclopropyl)methoxy)-4-((2-nitrobenzyl)oxy)-5,8-dihydropyrido[3,4-d]pyrimidine-7(6H)-carboxylate (Int-144b)

To a mixture of tert-butyl 2-chloro-4-((2-nitrobenzyl)oxy)-5,8-dihydropyrido[3,4-d]pyrimidine-7(6H)-carboxylate (Int-144a) (1.96 g, 4.66 mmol), (1-((dimethylamino)methyl)cyclopropyl)methanol (1.20 g, 9.29 mmol) and dichloro[9,9-dimethyl-4,5-bis(diphenylphosphino)xanthene]palladium(II) (352 mg, 0.466 mmol) in toluene (20 mL) was added cesium carbonate (4.55 g, 14.0 mmol), and the mixture was warmed to 110° C. and stirred for 3 h. The mixture was cooled to room temperature and filtered, and the filtrate was concentrated under reduced pressure. The residue was purified by flash silica gel chromatography (0-25%, MeOH gradient in chloroform). The residue obtained was purified by flash NH-silica gel chromatography (0-80%, ethyl acetate gradient in hexane) to give tert-butyl 2-((1-((dimethylamino)methyl)cyclopropyl)methoxy)-4-((2-nitrobenzyl)oxy)-5,8-dihydropyrido[3,4-d]pyrimidine-7(6H)-carboxylate (Int-144b) (1.90 g). ESI-MS m/z [M+H]⁺ 514.

### Step C: N,N-dimethyl-1-(1-(((4-((2-nitrobenzyl)oxy)-5,6,7,8-tetrahydropyrido[3,4-d]pyrimidin-2-yl)oxy)methyl)cyclopropyl)methanamine (Int-144c)

To a solution of tert-butyl 2-((1-((dimethylamino)methyl)cyclopropyl)methoxy)-4-((2-nitrobenzyl)oxy)-5,8-dihydropyrido[3,4-d]pyrimidine-7(6H)-carboxylate (Int-144b) (2.20 g, 4.28 mmol) in dichloromethane (20 mL) was added trifluoroacetic acid (10 mL) and the mixture was stirred at room temperature for 1 h. Trifluoroacetic acid was removed under reduced pressure, and the residue was purified by flash NH-silica gel chromatography (0-20%, MeOH gradient in ethyl acetate) to give N,N-dimethyl-1-(1-(((4-((2-nitrobenzyl)oxy)-5,6,7,8-tetrahydropyrido[3,4-d]pyrimidin-2-yl)oxy)methyl)cyclopropyl)methanamine (Int-144c) (1.64 g). ESI-MS m/z [M+H]⁺ 414.

### Step D: 1-(1-(((7-(8-bromo-3-(methoxymethoxy)naphthalen-1-yl)-4-((2-nitrobenzyl)oxy)-5,6,7,8-tetrahydropyrido[3,4-d]pyrimidin-2-yl)oxy)methyl)cyclopropyl)-N,N-dimethylmethanamine (Int-144d-1) and Int-144d-2 isomer (mixture)

N,N-dimethyl-1-(1-(((4-((2-nitrobenzyl)oxy)-5,6,7,8-tetrahydropyrido[3,4-d]pyrimidin-2-yl)oxy)methyl)cyclopropyl)methanamine (Int-144c) (1.65 g, 3.99 mmol), 1,8-dibromo-3-(methoxymethoxy)naphthalene (Int-W002) (2.76 g, 7.98 mmol), dichloro[9,9-dimethyl-4,5-bis(diphenylphosphino)xanthene]palladium(II) (905 mg, 1.20 mmol) and cesium carbonate (3.90 g, 12.0 mmol) in toluene (16 mL) were added in sealed tube. The reaction vessel was evacuated and backfilled with nitrogen three times. The mixture was stirred at 125° C. for 14 h, and the mixture was cooled to room temperature. The mixture was filtered, and the filtrate was concentrated under reduced pressure. The residue was purified by flash silica gel chromatography (0-25%, MeOH gradient in chloroform). The residue obtained was purified by flash NH-silica gel chromatography (0-8%, ethyl acetate gradient in hexane) to give 1-(1-(((7-(8-bromo-3-(methoxymethoxy)naphthalen-1-yl)-4-((2-nitrobenzyl)oxy)-5,6,7,8-tetrahydropyrido[3,4-d]pyrimidin-2-yl)oxy)methyl)cyclopropyl)-N,N-dimethylmethanamine (Int-144d-1) as a mixture of Int-144d-2 isomer (400 mg). ESI-MS m/z [M+H]⁺ 678, 680.

### Step E: 7-(8-bromo-3-(methoxymethoxy)naphthalen-1-yl)-2-((1-((dimethylamino)methyl)cyclopropyl)methoxy)-5,6,7,8-tetrahydropyrido[3,4-d]pyrimidin-4-ol (Int-144e-1) and isomer(mixture) (Int-144e-2)

To a mixture of 1-(1-(((7-(8-bromo-3-(methoxymethoxy)naphthalen-1-yl)-4-((2-nitrobenzyl)oxy)-5,6,7,8-tetrahydropyrido[3,4-d]pyrimidin-2-yl)oxy)methyl)cyclopropyl)-N,N-dimethylmethanamine (Int-144d-1) and Int-144d-2 isomer (255 mg, 0.376 mmol) and iron (powder, 210 mg, 3.76 mmol) in tetrahydrofuran (6.0 mL) was added hydrochloric acid (0.5 M, 6.0 mL), and the mixture was stirred at room temperature for 2 h. The mixture was filtered, and the filtrate was concentrated under reduce pressure to dryness. The residue was purified by flash NH-silica gel chromatography (0-100%, MeOH gradient in ethyl acetate) to give 7-(8-bromo-3-(methoxymethoxy)naphthalen-1-yl)-2-((1-((dimethylamino)methyl)cyclopropyl)methoxy)-5,6,7,8-tetrahydropyrido[3,4-d]pyrimidin-4-ol (Int-144e-1) as a mixture of Int-144e-2 isomer (180 mg). ESI-MS m/z [M+H]⁺ 543, 545.

### 7-(8-bromo-3-(methoxymethoxy)naphthalen-1-yl)-2-((1-(morpholinomethyl)cyclopropyl)methoxy)-5,6,7,8-tetrahydropyrido[3,4-d]pyrimidin-4-ol (Int-144f-1) and Int-144f-2 isomer (mixture)

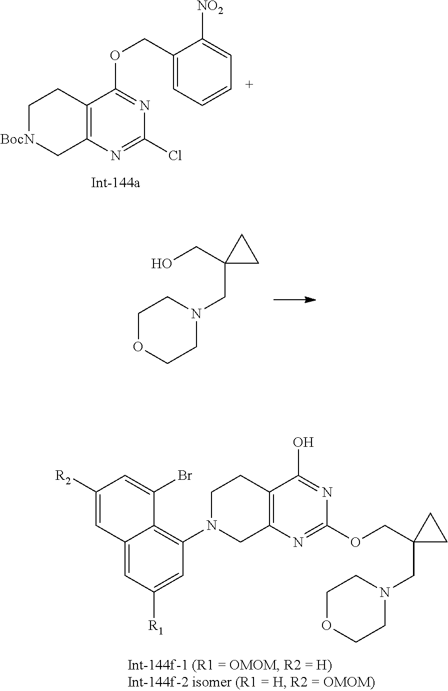

A mixture of Int-144f-1 and Int-144f-2 isomer was synthesized via a similar route as 7-(8-bromo-3-(methoxymethoxy)naphthalen-1-yl)-2-((1-((dimethylamino)methyl)cyclopropyl)methoxy)-5,6,7,8-tetrahydropyrido[3,4-d]pyrimidin-4-ol (Int-144e-1) using (1-(morpholinomethyl)cyclopropyl)methanol. ESI-MS m/z [M+H]⁺ 585, 587.

### Example 144: (R)-1-(7-(8-bromo-3-hydroxynaphthalen-1-yl)-2-((1-((dimethylamino)methyl)cyclopropyl)methoxy)-5,6,7,8-tetrahydropyrido[3,4-d]pyrimidin-4-yl)piperidin-3-ol (Ex. 144)

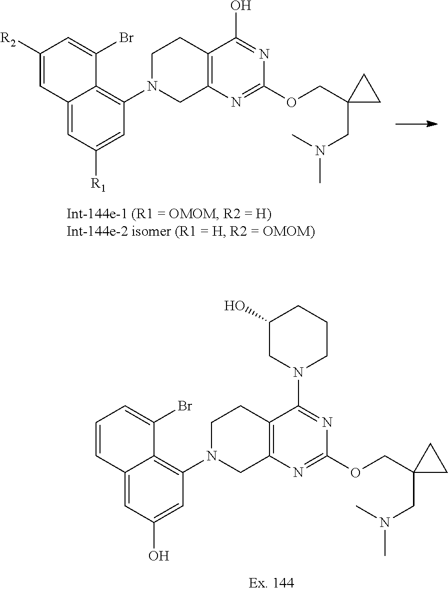

To a solution of 7-(8-bromo-3-(methoxymethoxy)naphthalen-1-yl)-2-((1-((dimethylamino)methyl)cyclopropyl)methoxy)-5,6,7,8-tetrahydropyrido[3,4-d]pyrimidin-4-ol (Int-144e-1) (33 mg, 0.0607 mmol) and N,N-diisopropylethylamine (0.031 mL, 0.182 mmol) in dichloromethane (1 mL) was added p-toluenesulfonyl chloride (18 mg, 0.0911 mmol), and the mixture was stirred at room temperature for 3 h. (R)-piperidin-3-ol (20 mg, 0.198 mmol) was added to the mixture, and the mixture was stirred at 60° C. for 3 h. Solvent was removed under reduced pressure, and hydrochloride (4 M in 1,4-dioxane, 2 mL) was added to the residue, and the mixture was evaporated to dryness. The residue was purified by reverse phase HPLC (MeCN/water with 0.1% formic acid) to give (R)-1-(7-(8-bromo-3-hydroxynaphthalen-1-yl)-2-((1-((dimethylamino)methyl)cyclopropyl)methoxy)-5,6,7,8-tetrahydropyrido[3,4-d]pyrimidin-4-yl)piperidin-3-ol (Ex. 144). ESI-MS m/z [M+H]⁺ 582, 584. ¹H NMR (400 MHZ, DMSO-d₆) δ 9.94 (s, 1H), 7.73-7.68 (m, 1H), 7.56-7.25 (m, 1H), 7.23-7.18 (m, 1H), 6.98-6.95 (m, 1H), 6.91-6.87 (m, 1H), 4.65 (s, 0.5H), 4.42 (s, 0.5H), 4.15-4.00 (m, 4H), 3.62-3.05 (m, 4H), 2.63-2.52 (m, 2H), 2.25-2.19 (m, 2H), 2.16 (s, 6H), 1.95-1.40 (m, 5H), 1.28-1.22 (m, 1H), 1.15 (s, 1.5H), 1.07 (s, 1.5H), 0.58-0.55 (m, 2H), 0.38-0.35 (m, 2H).

Compounds in the table below were synthesized via a similar route as Ex. 144 using 7-(8-bromo-3-(methoxymethoxy)naphthalen-1-yl)-2-((1-((dimethylamino)methyl)cyclopropyl)methoxy)-5,6,7,8-tetrahydropyrido[3,4-d]pyrimidin-4-ol (Int-144e-1) or 7-(8-bromo-3-(methoxymethoxy)naphthalen-1-yl)-2-((1-(morpholinomethyl)cyclopropyl)methoxy)-5,6,7,8-tetrahydropyrido[3,4-d]pyrimidin-4-ol (Int-144f-1) and corresponding amines.

TABLE 8

| | | | | ESI-MS |
|---|---|---|---|---|
| Example | Structure | Name | NMR | [M + H]⁺ |
| 145 | 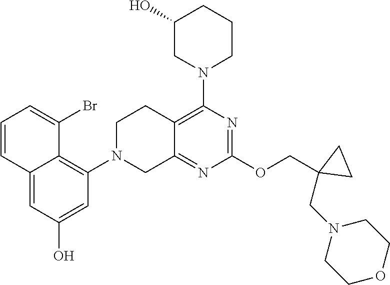 [SMILES: Oc1cc(N2CCc3c(nc(OCC4(CN5CCOCC5)CC4)nc3N3CCC[C@@H](O)C3)C2)c2c(Br)cccc2c1] | (R)-1-(7-(8-bromo-3- hydroxynaphthalen-1-yl)-2- ((1-(morpholinomethyl)cyclo- propyl)methoxy)- 5,6,7,8-tetrahydropyrido[3,4- d]pyrimidin-4- yl)piperidin-3-ol | | 624, 626 |
| 146 | 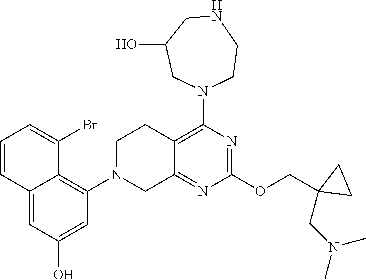 [SMILES: CN(C)CC1(COc2nc3c(c(N4CCNCC(O)C4)n2)CCN(c2cc(O)cc4cccc(Br)c24)C3)CC1] | 1-(7-(8-bromo-3- hydroxynaphthalen-1-yl)-2-((1- ((dimethylamino)methyl)cyclo- propyl)methoxy)- 5,6,7,8-tetrahydropyrido[3,4- d]pyrimidin-4-yl)-1,4- diazepan-6-ol | | 597, 599 |
| 147 | 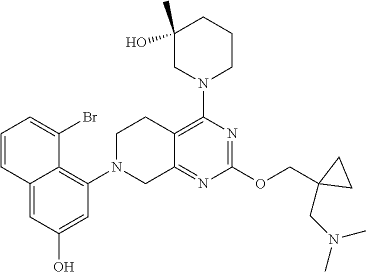 [SMILES: CN(C)CC1(COc2nc3c(c(N4CCC[C@@](C)(O)C4)n2)CCN(c2cc(O)cc4cccc(Br)c24)C3)CC1] | (R)-1-(7-(8-bromo-3- hydroxynaphthalen-1-yl)-2-((1- ((dimethylamino)methyl)cyclo- propyl)methoxy)- 5,6,7,8-tetrahydropyrido[3,4- d]pyrimidin-4-yl)-3- methylpiperidin-3-ol | ¹H NMR (400 MHz, DMSO-d₆) δ 9.94 (s, 1H), 7.73-7.68 (m, 1H), 7.56-7.25 (m, 1H), 7.23- 7.18 (m, 1H), 6.98-6.95 (m, 1H), 6.91-6.87 (m, 1H), 4.65 (s, 0.5H), 4.42 (s, 0.5H), 4.15-4.00 (m, 4H), 3.62-3.05 (m, 4H), 2.63-2.52 (m, 2H), 2.25- 2.19 (m, 2H), 2.16 (s, 6H), 1.95-1.40 (m, 5H), 1.28-1.22 (m, 1H), 1.15 (s, 1.5H), 1.07 (s, 1.5H), 0.58-0.55 (m, 2H), 0.38- 0.35 (m, 2H) | 596, 598 |
| 148 | 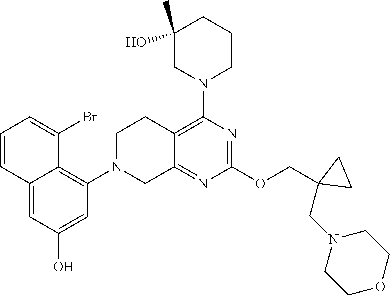 [SMILES: C[C@@]1(O)CCCN(c2nc(OCC3(CN4CCOCC4)CC3)nc3c2CCN(c2cc(O)cc4cccc(Br)c24)C3)C1] | (R)-1-(7-(8-bromo-3- hydroxynaphthalen-1-yl)-2- ((1-(morpholinomethyl)cyclo- propyl)methoxy)- 5,6,7,8-tetrahydropyrido[3,4- d]pyrimidin-4-yl)-3- methylpiperidin-3-ol | | 638, 640 |

### (3R)-3-methyl-1-(7-(6-methyl-1-(tetrahydro-2H-pyran-2-yl)-5-(trifluoromethyl)-1H-indazol-4-yl)-2-(methylthio)-5,6,7,8-tetrahydropyrido[3,4-d]pyrimidin-4-yl)piperidin-3-ol (Int-149e)

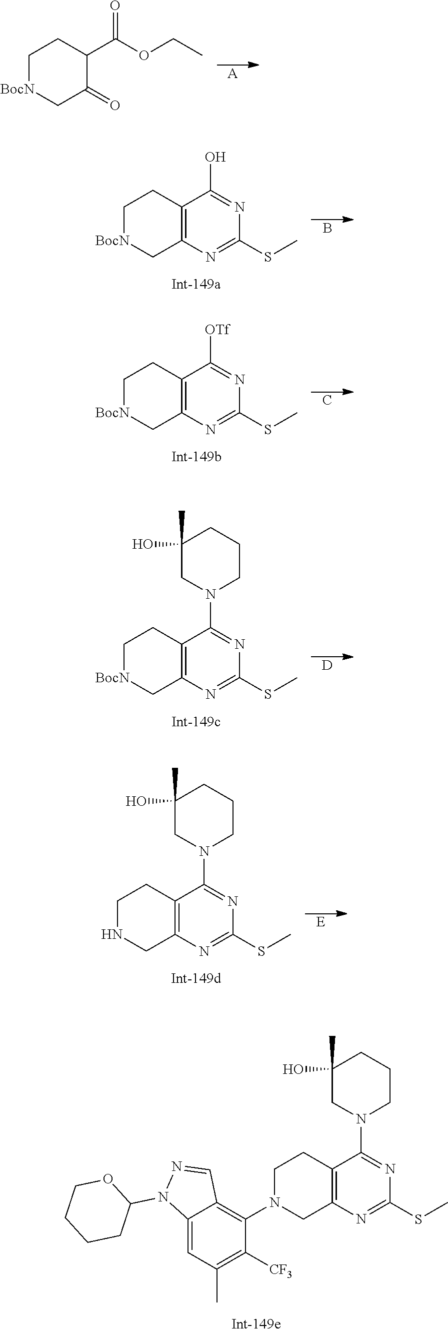

### Step A: tert-butyl 4-hydroxy-2-(methylthio)-5,8-dihydropyrido[3,4-d]pyrimidine-7(6H)-carboxylate (Int-149a)

To a solution of 1-(tert-butyl) 4-ethyl 3-oxopiperidine-1,4-dicarboxylate (10.0 g, 36.9 mmol) and S-methylisothiourea sulfate (20.5 g, 73.7 mmol) in methanol (200 mL) was added sodium methoxide (9.96 g, 184 mmol) and the mixture was stirred at 60° C. for 4 h. The reaction mixture was cooled to room temperature, then hydrochloric acid (6 M, 20.0 mL, 120 mmol) was added to the mixture. Methanol was removed under reduced pressure, and water (150 mL) and ethyl acetate (150 mL) were added to the residue. The precipitate was filtered and rinsed with ethyl acetate (30 mL) and dried to give tert-butyl 4-hydroxy-2-(methylthio)-5,8-dihydropyrido[3,4-d]pyrimidine-7(6H)-carboxylate (Int-149a) (4.70 g). ESI-MS m/z [M+H]⁺ 298.

### Step B: tert-butyl 2-(methylthio)-4-(((trifluoromethyl)sulfonyl)oxy)-5,8-dihydropyrido[3,4-d]pyrimidine-7(6H)-carboxylate (Int-149b)

To a solution of tert-butyl 4-hydroxy-2-(methylthio)-5,8-dihydropyrido[3,4-d]pyrimidine-7(6H)-carboxylate (Int-149a) (2.00 g, 6.73 mmol) and N,N-diisopropylethylamine (3.43 mL, 20.2 mmol) in dichloromethane (20 mL) was added trifluoromethanesulfonic anhydride (1.10 mL, 6.73 mmol) at 0° C., and the mixture was stirred at room temperature for 1 h. The mixture was diluted with ethyl acetate and the diluted mixture was washed with water and the mixture was concentrated under reduced pressure. The residue was purified by flash silica gel chromatography (0-30%, ethyl acetate gradient in hexane) to give tert-butyl 2-(methylthio)-4-(((trifluoromethyl)sulfonyl)oxy)-5,8-dihydropyrido[3,4-d]pyrimidine-7(6H)-carboxylate (Int-149b) (1.50 g). ESI-MS m/z [M+H]⁺ 430.

### Step C: tert-butyl (R)-4-(3-hydroxy-3-methylpiperidin-1-yl)-2-(methylthio)-5,8-dihydropyrido[3,4-d]pyrimidine-7(6H)-carboxylate (Int-149c)

To a solution of tert-butyl 2-(methylthio)-4-(((trifluoromethyl)sulfonyl)oxy)-5,8-dihydropyrido[3,4-d]pyrimidine-7(6H)-carboxylate (Int-149b) (600 mg, 1.40 mmol) and (R)-3-methylpiperidin-3-ol hydrochloride (233 mg, 1.54 mmol) in DMA (10 mL) was added N,N-diisopropylethylamine (3.43 mL, 20.2 mmol), and the mixture was stirred at room temperature for 3 h. The mixture was diluted with ethyl acetate and the diluted mixture was washed with water and the mixture was concentrated under reduced pressure. The residue was purified by flash silica gel chromatography (0-80%, ethyl acetate gradient in hexane) to tert-butyl (R)-4-(3-hydroxy-3-methylpiperidin-1-yl)-2-(methylthio)-5,8-dihydropyrido[3,4-d]pyrimidine-7(6H)-carboxylate (Int-149c) (570 mg). ESI-MS m/z [M+H]⁺ 395.

### Step D: (R)-3-methyl-1-(2-(methylthio)-5,6,7,8-tetrahydropyrido[3,4-d]pyrimidin-4-yl)piperidin-3-ol (Int-149d)

To a solution of tert-butyl (R)-4-(3-hydroxy-3-methylpiperidin-1-yl)-2-(methylthio)-5,8-dihydropyrido[3,4-d]pyrimidine-7(6H)-carboxylate (Int-149c) (570 mg, 1.44 mmol) in dichloromethane (5 mL) was added trifluoroacetic acid (5 mL) and the mixture was stirred at room temperature for 1 h. Trifluoroacetic acid was removed under reduced pressure, and the residue was purified by flash NH-silica gel chromatography (0-20%, MeOH gradient in ethyl acetate) to give (R)-3-methyl-1-(2-(methylthio)-5,6,7,8-tetrahydropyrido[3,4-d]pyrimidin-4-yl)piperidin-3-ol (Int-149d) (430 mg). ESI-MS m/z [M+H]⁺ 295.

### Step E: (3R)-3-methyl-1-(7-(6-methyl-1-(tetrahydro-2H-pyran-2-yl)-5-(trifluoromethyl)-1H-indazol-4-yl)-2-(methylthio)-5,6,7,8-tetrahydropyrido[3,4-d]pyrimidin-4-yl)piperidin-3-ol (Int-149e)

To a mixture of (R)-3-methyl-1-(2-(methylthio)-5,6,7,8-tetrahydropyrido[3,4-d]pyrimidin-4-yl)piperidin-3-ol (Int-149d) (150 mg, 0.509 mmol), 4-bromo-6-methyl-1-(tetrahydro-2H-pyran-2-yl)-5-(trifluoromethyl)-1H-indazole (Int-HHH5) (278 mg, 0.764 mmol), RuPhos Pd G3 (42.6 mg, 0.0509 mmol) in 1,4-dioxane (2.0 mL) was added cesium carbonate (498 mg, 1.53 mmol), and the mixture was warmed to 100° C. and stirred for 2 h. The mixture was cooled to room temperature and filtered, and the filtrate was concentrated under reduced pressure. The residue was purified by flash silica gel chromatography (10-70%, ethyl acetate gradient in hexane) to give (3R)-3-methyl-1-(7-(6-methyl-1-(tetrahydro-2H-pyran-2-yl)-5-(trifluoromethyl)-1H-indazol-4-yl)-2-(methylthio)-5,6,7,8-tetrahydropyrido[3,4-d]pyrimidin-4-yl)piperidin-3-ol (Int-149e) (175 mg). ESI-MS m/z [M+H]⁺ 577.

### Example 149: (R)-1-(2-((1-((dimethylamino)methyl)cyclopropyl)methoxy)-7-(6-methyl-5-(trifluoromethyl)-1H-indazol-4-yl)-5,6,7,8-tetrahydropyrido[3,4-d]pyrimidin-4-yl)-3-methylpiperidin-3-ol formate (Ex. 149)

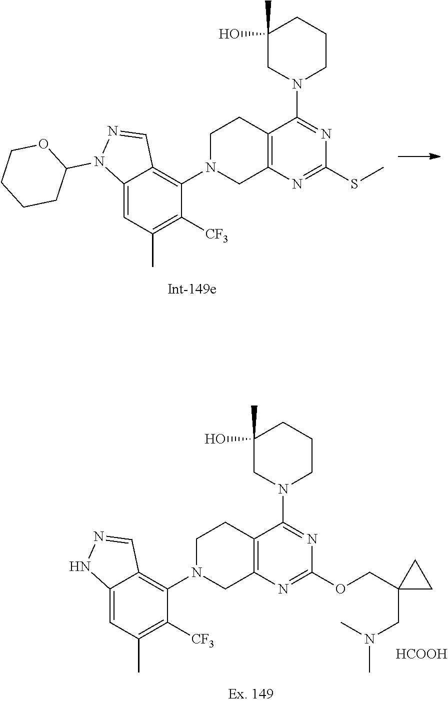 [SMILES: CSc1nc2c(c(N3CCC[C@@](C)(O)C3)n1)CCN(c1c(C(F)(F)F)c(C)cc3c1cnn3C1CCCCO1)C2 ; Cc1cc2[nH]ncc2c(N2CCc3c(nc(OCC4(CN(C)C)CC4)nc3N3CCC[C@@](C)(O)C3)C2)c1C(F)(F)F ; "HCOOH"]

To a solution of (3R)-3-methyl-1-(7-(6-methyl-1-(tetrahydro-2H-pyran-2-yl)-5-(trifluoromethyl)-1H-indazol-4-yl)-2-(methylthio)-5,6,7,8-tetrahydropyrido[3,4-d]pyrimidin-4-yl)piperidin-3-ol (Int-149e) (15 mg, 0.0260 mmol) in ethyl acetate (3 mL) was added 3-chloroperoxybenzoic acid (6.4 mg, 0.0260 mmol) at 0° C., the mixture was stirred at 0° C. for 0.5 h. Water (3 mL) was added to the mixture, and the mixture was washed with saturated sodium bicarbonate solution (3 mL). The solvent was removed under reduced pressure. To a solution of the crude product and (1-((dimethylamino)methyl)cyclopropyl)methanol (10 mg, 0.078 mmol) in tetrahydrofuran (1 mL) was added potassium tert-butoxide (1.0 M in THF, 0.039 mL) at 0° C., the mixture was stirred at 0° C. for 0.5 h. The mixture was diluted with ethyl acetate and the diluted mixture was washed with water and the mixture was concentrated under reduced pressure. Trifluoroacetic acid (0.5 mL) was added to the residue and the mixture was stirred at room temperature for 1 h. Trifluoroacetic acid was removed under reduced pressure and the residue was purified by reverse phase HPLC (MeCN/water with 0.1% formic acid) to give (R)-1-(2-((1-((dimethylamino)methyl)cyclopropyl)methoxy)-7-(6-methyl-5-(trifluoromethyl)-1H-indazol-4-yl)-5,6,7,8-tetrahydropyrido[3,4-d]pyrimidin-4-yl)-3-methylpiperidin-3-ol formate (Ex. 149). ESI-MS m/z [M+H]⁺ 574. ¹H NMR (400 MHZ, DMSO-d₆) δ 13.4 (s, 1H), 8.32 (s, 1H), 7.36 (s, 1H), 4.60-4.44 (m, 1H), 4.24-4.04 (m, 4H), 3.62-3.20 (m, 9H), 2.90-2.60 (m, 2H), 2.58-2.54 (m, 3H), 2.38-2.10 (m, 4H), 1.90-1.75 (m, 1H), 1.62-1.53 (m, 4H), 1.12 (s, 3H), 0.79-0.37 (m, 4H).

Compounds in the table below were synthesized via a similar route as Ex. 149 using the corresponding amino alcohols.

TABLE 9

| | | | | ESI-MS |
|---|---|---|---|---|
| Example | Structure | Name | NMR | [M + H]⁺ |
| 150 | 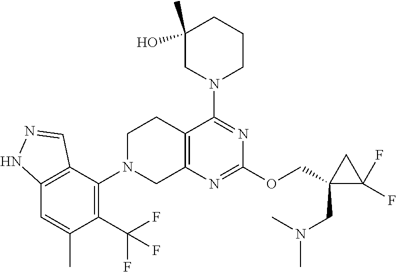 [SMILES: Cc1cc2[nH]ncc2c(N2CCc3c(nc(OC[C@]4(CN(C)C)CC4(F)F)nc3N3CCC[C@@](C)(O)C3)C2)c1C(F)(F)F] | (R)-1-(2-(((R)-1-((dimethylamino)methyl)- 2,2-difluorocyclopropyl)methoxy)-7-(6- methyl-5-(trifluoromethyl)-1H-indazol-4-yl)- 5,6,7,8-tetrahydropyrido[3,4-d]pyrimidin-4- yl)-3-methylpiperidin-3-ol | | 610 |
| 151 | 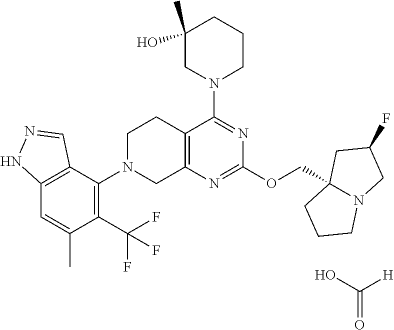 | (R)-1-(2-(((2R,7aS)-2-fluorotetrahydro-1H- pyrrolizin-7a(5H)-yl)methoxy)-7-(6-methyl-5- (trifluoromethyl)-1H-indazol-4-yl)-5,6,7,8- tetrahydropyrido[3,4-d]pyrimidin-4-yl)-3- methylpiperidin-3-ol | | 604 |

### Assays

### Procedure for SOS-Catalyzed Nucleotide Exchange Assay

Recombinant KRAS G12C (amino acids 1-169, SEQ ID NO:1), KRAS G12D (amino acids 1-169, SEQ ID NO:2), KRAS G12V (amino acids 1-169, SEQ ID NO:3) and SOS1 (amino acids 564-1049, SEQ ID NO:4) proteins were expressed in E. coli and purified by affinity chromatography. To prepare each BODIPY FL GDP-bound KRAS mutant protein, 50 μM KRAS mutant proteins were incubated with 0.5 mM BODIPY FL GDP (Invitrogen, G22360) in a loading buffer (20 mM Tris-HCl (pH 7.5), 50 mM NaCl, 1 mM DTT and 2.5 mM EDTA) for 1 hour on ice. After the incubation, MgCl₂ was added to a final concentration of 10 mM, followed by incubation at room temperature for 30 minutes. The mixtures were allowed to pass through a NAP-5 column to remove free nucleotides and purified BODIPY FL GDP-bound KRAS G12C, G12D and G12V proteins were used for compound evaluation.

For the measurement of the inhibitory activity of compounds on GDP-GTP exchange rate of recombinant KRAS mutants, each BODIPY FL GDP-bound KRAS mutant protein (Version 1: 25 nM, Version 2: 2.5 nM) was incubated with various concentrations of compound in a reaction buffer (20 mM Tris-HCl (pH 7.5), 100 mM NaCl, 1 mM MgCl₂, 2 mM DTT, 0.1% Tween 20) at 25° C. for 1 hour. After the incubation, recombinant SOS1 and GMPPNP (Jena Bioscience GmbH, NU-401) were added and incubated at room temperature for 30 minutes to proceed SOS1-dependent GDP-GTP exchange reaction on KRAS mutants. Replacement of BODIPY FL GDP by Guanosine-5′-[(β,γ)-imido]triphosphate, Tetralithium salt (GMPPNP) was measured by calculating the ratio of fluorescence intensities of BODIPY FL before and after the exchange reaction. Inhibition % was calculated by setting the fluorescence ratio from the reaction without test compound (DMSO control) and the fluorescence ratio from the reaction without SOS1 and GMPPNP as 0% and 100% inhibition, respectively. Dose response curves were analyzed using a 4-parameter logistic model to calculate IC₅₀ values. Table A shows the results.

TABLE A

| | Version | Version | Version | Version | Version | Version |
|---|---|---|---|---|---|---|
| | 1 G12C | 1 G12D | 1 G12V | 2 G12C | 2 G12D | 2 G12V |
| Ex. | IC₅₀ | IC₅₀ | IC₅₀ | IC₅₀ | IC₅₀ | IC₅₀ |
| 1 | 48.8 | 85.7 | 53.0 | 39.3 | 98.8 | 58.5 |
| 2 | 73.9 | 653.3 | 84.0 | 216.8 | 656.2 | 129.8 |
| 3 | 86.6 | 186.7 | 106.0 | 238.7 | 174.5 | 171.8 |
| 4 | 92.9 | 162.2 | 94.0 | 210.6 | 157.2 | 161.0 |
| 5 | 69.3 | 430.9 | 110.0 | 330.9 | 679.0 | 322.2 |
| 6 | 43.0 | 438.7 | 58.0 | 125.6 | 772.7 | 85.7 |
| 7 | 118.3 | 170.4 | 75.0 | 208.6 | 191.5 | 136.4 |
| 8 | 90.4 | 275.1 | 125.0 | | | |
| 9 | 97.2 | 114.2 | 156.0 | 217.4 | 168.3 | 491.7 |
| 10 | 38.2 | 62.8 | 43.0 | | | |
| 11 | 29.8 | 38.8 | 46.0 | 39.6 | 34.9 | 46.6 |
| 12 | 79.6 | 739.4 | 104.0 | 266.5 | 1038.2 | 146.3 |
| 13 | 26.5 | 315.8 | 38.0 | | | |
| 14 | 71.3 | 180.3 | 101.0 | | | |
| 15 | 61.3 | 101.0 | 73.0 | | | |
| 16 | 38.2 | 22.8 | 25.0 | 46.4 | 9.5 | 26.5 |
| 17 | 24.3 | 37.7 | 28.0 | 16.1 | 14.0 | 18.3 |
| 18 | 93.6 | 305.4 | 116.0 | 91.4 | 145.0 | 127.5 |
| 19 | 56.3 | 136.0 | 106.0 | 42.9 | 84.1 | 79.1 |
| 20 | 21.2 | 20.5 | 22.0 | 14.9 | 9.1 | 11.9 |
| 21 | 45.3 | 40.1 | 39.0 | | | |
| 22 | 44.8 | 136.6 | 33.0 | 34.4 | 68.3 | 31.3 |
| 23 | 111.6 | 92.2 | 76.0 | 112.7 | 81.2 | 73.7 |
| 24 | 89.0 | 582.7 | 95.0 | 172.7 | 544.6 | 201.6 |
| 25 | 77.0 | 49.8 | 88.0 | 144.8 | 20.1 | 99.3 |
| 26 | 49.8 | 247.9 | 42.0 | 67.3 | 147.9 | 43.7 |
| 27 | 166.3 | 433.0 | 81.0 | 504.3 | 533.2 | 208.4 |
| 28 | 47.5 | 32.8 | 37.0 | 20.8 | 11.2 | 15.3 |
| 29 | 59.1 | 108.7 | 54.0 | 46.2 | 47.4 | 42.8 |
| 30 | 85.9 | 264.6 | 89.0 | 169.2 | 217.0 | 116.3 |
| 31 | 164.0 | 333.8 | 102.0 | 183.3 | 275.7 | 66.3 |
| 32 | 45.0 | 43.9 | 47.0 | 43.9 | 26.2 | 53.4 |
| 33 | 58.3 | 43.3 | 48.0 | 36.3 | 29.1 | 30.8 |
| 34 | 39.3 | 22.2 | 34.0 | 14.2 | 14.9 | 17.7 |
| 35 | 142.7 | 172.7 | 99.0 | 219.6 | 208.8 | 151.4 |
| 36 | 64.2 | 70.2 | 55.0 | 81.9 | 49.9 | 59.2 |
| 37 | 34.3 | 87.5 | 35.0 | 14.3 | 56.8 | 31.9 |
| 38 | 96.3 | 92.0 | 113.0 | 90.0 | 49.4 | 85.1 |
| 39 | 69.6 | 66.9 | 58.0 | 15.9 | 17.1 | 18.3 |
| 40 | 39.3 | 257.3 | 31.0 | 14.8 | 134.2 | 12.9 |
| 41 | | | | 175.2 | 167.9 | 86.3 |
| 42 | | | | 55.3 | 238.6 | 96.6 |
| 43 | | | | 144.1 | 407.5 | 90.9 |
| 44 | | | | 8.3 | 150.4 | 8.7 |
| 45 | | | | 27.6 | 35.8 | 25.9 |
| 46 | | | | 85.0 | 19.6 | 86.8 |
| 47 | | | | 20.8 | 38.0 | 21.0 |
| 48 | | | | 2.8 | 1.1 | 2.3 |
| 49 | | | | 171.4 | 86.2 | 169.0 |
| 50 | | | | 11.2 | 61.5 | 6.1 |
| 51 | | | | 55.0 | 78.4 | 91.1 |
| 52 | | | | 27.3 | 34.0 | 23.9 |
| 53 | | | | 52.9 | 44.9 | 70.3 |
| 54 | | | | 69.2 | 481.8 | 113.1 |
| 55 | | | | 32.2 | 85.2 | 116.0 |
| 56 | | | | 59.3 | 9.8 | 107.3 |
| 57 | | | | 168.8 | 77.7 | 163.4 |
| 58 | | | | 85.3 | 389.8 | 189.6 |
| 59 | | | | 7.6 | 20.1 | 14.4 |
| 60 | | | | 5.5 | 56.2 | 9.8 |
| 61 | | | | 21.2 | 53.1 | 23.4 |
| 62 | | | | 7.3 | 48.2 | 13.4 |
| 63 | | | | 4.8 | 3.5 | 5.4 |
| 64 | | | | 4.6 | 20.2 | 7.9 |
| 65 | | | | 9.2 | 50.9 | 16.7 |
| 66 | | | | 90.2 | 245.9 | 139.4 |
| 67 | | | | 59.2 | 467.2 | 222.3 |
| 68 | | | | 59.3 | 715.4 | 345.3 |
| 69 | | | | 23.2 | 55.2 | 35.7 |
| 70 | | | | 20.9 | 461.3 | 79.6 |
| 71 | | | | 13.1 | 15.9 | 14.5 |
| 72 | | | | 21.6 | 142.9 | 33.1 |
| 73 | | | | 14.9 | 6.6 | 16.1 |
| 74 | | | | 1.8 | 2.4 | 1.7 |
| 75 | | | | 14.7 | 118.7 | 22.6 |
| 76 | | | | 6.4 | 14.4 | 11.6 |
| 77 | | | | 28.3 | 44.0 | 89.2 |
| 78 | | | | 3.2 | 1.2 | 2.2 |
| 79 | 89.7 | 95.0 | 129.0 | 51.7 | 46.5 | 66.2 |
| 80 | 50.1 | 76.1 | 88.0 | 36.4 | 34.1 | 41.1 |
| 81 | | | | 2.4 | 1.4 | 2.7 |
| 82 | | | | 61.3 | 161.6 | 138.0 |
| 83 | | | | 29.4 | 269.9 | 132.6 |
| 84-1 | 23.8 | 16.5 | 19.0 | 5.2 | 3.9 | 4.8 |
| 84-2 | | | | 4400 | 5000 | 3600 |
| 85 | | | | 61.8 | 61.2 | 83.4 |
| 86 | | | | 102.2 | 96.2 | 100.8 |
| 87 | | | | 17.2 | 13.1 | 19.0 |
| 88 | | | | 41.0 | 33.5 | 40.9 |
| 89 | | | | 48.1 | 46.1 | 66.4 |
| 90 | | | | 68.7 | 55.3 | 78.6 |
| 91 | | | | 33.4 | 61.2 | 102.9 |
| 92 | | | | 37.7 | 97.8 | 74.9 |
| 93 | | | | 5.0 | 5.3 | 5.8 |
| 94 | | | | 7.8 | 9.9 | 9.9 |
| 95 | | | | 61.4 | 120.6 | 129.8 |
| 96 | | | | 2.3 | 1.6 | 2.3 |
| 97 | | | | 2.0 | 1.4 | 1.8 |
| 98 | | | | 2.5 | 1.8 | 2.8 |
| 99 | | | | 3.7 | 2.8 | 3.7 |
| 100 | | | | 3.1 | 2.2 | 2.7 |
| 101 | | | | 1.9 | 1.1 | 1.7 |
| 102 | | | | 1.9 | 1.2 | 2.1 |
| 103 | | | | 2.2 | 1.6 | 2.3 |
| 104 | | | | 2.1 | 1.6 | 2.2 |
| 105 | | | | 1.7 | 1.1 | 1.6 |
| 106 | | | | 1.8 | 1.1 | 1.6 |
| 107 | | | | 3.3 | 2.8 | 3.9 |
| 108 | | | | 1.3 | 0.9 | 0.9 |
| 109 | | | | 1.4 | 1.0 | 1.1 |
| 110 | | | | 5.3 | 3.3 | 8.4 |
| 111 | | | | 6.5 | 5.5 | 7.6 |
| 112 | | | | 2.1 | 1.7 | 2.2 |
| 113 | | | | 13.7 | 20.0 | 26.0 |
| 114 | | | | 2.8 | 2.6 | 2.8 |
| 115 | | | | 5.6 | 6.4 | 8.4 |
| 116 | | | | 2.6 | 2.2 | 2.8 |
| 117 | | | | 13.3 | 17.9 | 19.1 |
| 118 | | | | 1.5 | 1.0 | 1.2 |
| 119 | | | | 7.4 | 11.4 | 13.4 |
| 120 | | | | 1.6 | 0.6 | 1.4 |
| 121 | | | | 6.0 | 3.3 | 7.7 |
| 122 | | | | 1.7 | 0.8 | 1.3 |
| 123 | | | | 4.0 | 2.0 | 5.0 |
| 124 | | | | 5.5 | 10.8 | 12.3 |
| 125-1 | | | | 41.3 | 50.8 | 51.9 |
| 126 | 18.7 | 10.8 | 14.0 | 1.6 | 1.2 | 1.7 |
| 127 | 35.5 | 38.1 | 35.0 | 28.0 | 12.3 | 26.1 |
| 128 | 74.0 | 105.0 | 59.0 | 150.0 | 71.5 | 92.5 |
| 129 | 30.9 | 27.5 | 25.0 | 18.8 | 23.4 | 17.2 |
| 130 | 36.8 | 53.8 | 35.0 | 36.1 | 36.3 | 34.7 |
| 131 | 43.8 | 38.3 | 36.0 | 6.8 | 6.8 | 6.8 |
| 132 | | | | 2.1 | 1.3 | 2.1 |
| 133 | | | | 3.9 | 3.0 | 4.7 |
| 134 | | | | 1.8 | 1.2 | 2.0 |
| 135 | | | | 1.4 | 1.3 | 1.9 |
| 136 | | | | 1.7 | 1.6 | 2.4 |
| 137 | | | | 1.8 | 1.3 | 0.9 |
| 138 | | | | 1.6 | 0.9 | 1.4 |
| 139 | | | | 5.6 | 3.6 | 10.5 |
| 140 | | | | 1.5 | 1.1 | 1.9 |
| 141 | | | | 1.3 | 0.5 | 0.9 |
| 142 | | | | 96.5 | 265.4 | 129.9 |
| 143 | | | | 1.3 | 1.1 | 0.8 |
| 144 | 29.6 | 38.7 | 28.0 | 27.2 | 20.7 | 31.1 |
| 145 | 97.4 | 71.2 | 104.0 | 99.3 | 52.8 | 101.8 |
| 146 | 69.4 | 101.2 | 117.0 | 154.6 | 71.0 | 133.7 |
| 147 | | | | 3.5 | 1.8 | 4.1 |
| 148 | | | | 9.8 | 5.0 | 13.0 |
| 149 | | | | 98.1 | 112.0 | 178.8 |
| 150 | | | | 73.9 | 77.6 | 86.2 |
| 151 | | | | 79.4 | 98.1 | 117.7 |

### Procedure for Cellular Anti-Proliferation Assay

SW620 cells (ATCC®, CCL-227™), containing homozygous KRAS-G12V activating mutation, and MIA PaCa-2 cells (provided by Sumitomo Dainippon Pharma Co., Ltd.) containing homozygous KRAS-G12C activating mutation, were cultured in T175 flasks in growth medium (RPMI medium 1640 (Fujifilm Wako Pure Chemical Corporation, 187-02705) containing 10% fetal bovine serum (HyClone, SH30910.03)). The cells were harvested in growth medium after Trypsin/EDTA (Nacalai Tesque, 32777-44) digestion and were seeded in PrimeSurface® 384-well U bottom microplates (Sumitomo Bakelite Co., Ltd., MS-9384W) at each density of 250 cells/well, and incubated at 37° C., 5% CO₂ overnight.

The test compound was diluted with DMSO to give a concentration 500 times the final concentration. The resultant solution of the test compound in DMSO was diluted with the growth medium used for suspending cells and added to each well of the cell-culture plate to give a DMSO final concentration of 0.2% followed by incubation at 37° C., 5% CO₂ for 3 days.

To all cells were added with CellTiter-Glo® 3D Reagent (Promega, G9683) and mixed for 10 minutes. Thirty minutes after mixing, luminescence was measured on an EnVision plate reader (PerkinElmer). Dose response curves were analyzed using a 4-parameter logistic model to calculate IC₅₀ values. Table B shows the results.

TABLE B

| Ex. | MIA PaCa-2 IC₅₀ | SW620 IC₅₀ |
|---|---|---|
| 1 | 2712.1 | |
| 2 | 9287.5 | |
| 3 | 6950.7 | |
| 4 | 3942.0 | |
| 5 | 7818.5 | |
| 7 | 10000.0 | |
| 8 | 10000.0 | |
| 10 | 1530.2 | |
| 11 | 1141.7 | |
| 12 | 4745.8 | |
| 13 | 10000.0 | |
| 14 | 10000.0 | |
| 15 | 10000.0 | |
| 16 | 10000.0 | |
| 17 | 8722.3 | 10000.0 |
| 18 | 2385.3 | |
| 19 | 3363.3 | |
| 20 | 10000.0 | |
| 21 | 8055.8 | |
| 22 | 2988.3 | |
| 23 | 2017.2 | |
| 26 | 3302.1 | |
| 28 | 6036.0 | |
| 32 | 2909.1 | |
| 34 | 3472.2 | |
| 36 | 2793.5 | |
| 37 | 5189.3 | |
| 38 | 4646.5 | |
| 39 | 5851.6 | |
| 40 | 2800.2 | |
| 41 | 10000.0 | |
| 42 | 10000.0 | |
| 43 | 10000.0 | |
| 44 | 10000.0 | |
| 45 | 2081.7 | |
| 46 | 10000.0 | |
| 47 | 10000.0 | |
| 48 | 3641.1 | 6248.6 |
| 49 | 10000.0 | |
| 50 | 3150.3 | |
| 55 | 10000.0 | |
| 56 | 10000.0 | |
| 59 | 1011.2 | |
| 61 | 1637.7 | |
| 62 | 9391.0 | |
| 63 | 2471.7 | 8599.2 |
| 64 | 1280.5 | |
| 65 | 1099.3 | |
| 66 | 10000.0 | |
| 76 | 3303.2 | 3469.4 |
| 77 | 10000.0 | 10000.0 |
| 78 | 9518.8 | 10000.0 |
| 79 | 1217.0 | |
| 80 | 1115.5 | |
| 81 | 613.3 | |
| 84-1 | 753.8 | 1162.6 |
| 85 | 2807.4 | |
| 86 | 2809.0 | |
| 87 | 1087.5 | |
| 88 | 1576.4 | |
| 89 | 1545.7 | |
| 90 | 1968.2 | |
| 91 | 2313.4 | |
| 93 | 1124.1 | 1726.3 |
| 94 | 686.5 | |
| 95 | 10000.0 | 10000.0 |
| 96 | 1272.5 | 1996.4 |
| 97 | 687.1 | 1005.9 |
| 98 | 735.1 | 476.0 |
| 99 | 2000.0 | 1551.3 |
| 100 | 1305.9 | 1321.7 |
| 101 | 2000.0 | 2000.0 |
| 102 | 2000.0 | 2000.0 |
| 103 | 9344.5 | 10000.0 |
| 104 | 10000.0 | 9880.8 |
| 105 | 10000.0 | 10000.0 |
| 106 | 8761.2 | 10000.0 |
| 107 | 2000.0 | 2000.0 |
| 108 | 362.0 | |
| 109 | 520.1 | 1318.4 |
| 110 | 489.7 | |
| 111 | 10000.0 | 10000.0 |
| 112 | 567.8 | 1365.8 |
| 113 | 9469.6 | |
| 114 | 8446.1 | |
| 115 | 1617.3 | |
| 116 | 657.5 | |
| 117 | 5406.2 | 9923.0 |
| 118 | 112.2 | 304.1 |
| 119 | 354.7 | |
| 120 | 123.8 | 260.7 |
| 121 | 254.3 | 1417.1 |
| 122 | 721.9 | 2833.6 |
| 123 | 393.7 | 2262.1 |
| 124 | 463.7 | |
| 126 | 280.7 | 488.3 |
| 127 | 2514.0 | |
| 128 | 1527.6 | |
| 129 | 10000.0 | |
| 130 | 2093.2 | |
| 131 | 555.6 | |
| 132 | 813.1 | 954.0 |
| 133 | 416.4 | |
| 134 | 5757.1 | 8470.8 |
| 135 | 300.5 | 549.8 |
| 136 | 413.6 | 539.0 |
| 137 | 563.5 | |
| 138 | 425.1 | |
| 139 | 575.6 | |
| 140 | 207.5 | 475.4 |
| 141 | 87.2 | 215.4 |
| 142 | 10000.0 | 8868.3 |
| 143 | 82.6 | 300.8 |
| 144 | 1392.0 | |
| 145 | 2479.1 | 3753.3 |
| 146 | 10000.0 | |
| 147 | 1286.9 | 1703.9 |
| 148 | 2724.9 | 2284.9 |
| 149 | 10000.0 | 10000.0 |
| 150 | 10000.0 | 10000.0 |
| 151 | 10000.0 | 10000.0 |

SEQUENCES

SEQ ID NO: 1 - Recombinant KRAS G12C
MASSHHHHHHSSENLYFQGMTEYKLVVVGACGVGKSALTIQLIQN
HFVDEYDPTIEDSYRKQVVIDGETCLLDILDTAGQEEYSAMRDQYM
RTGEGFLCVFAINNTKSFEDIHHYREQIKRVKDSEDVPMVLVGNKC
DLPSRTVDTKQAQDLARSYGIPFIETSAKTRQGVDDAFYTLVREIRK
HKEK
SEQ ID NO: 2 - Recombinant KRAS G12D
MASSHHHHHHSSENLYFQGMTEYKLVVVGADGVGKSALTIQLIQN
HFVDEYDPTIEDSYRKQVVIDGETCLLDILDTAGQEEYSAMRDQYM
RTGEGFLCVFAINNTKSFEDIHHYREQIKRVKDSEDVPMVLVGNKC
DLPSRTVDTKQAQDLARSYGIPFIETSAKTRQGVDDAFYTLVREIRK
HKEK
SEQ ID NO: 3 - Recombinant KRAS G12V
MGSSHHHHHHSSGLVPRGSHMASMTGGQQMGRGSENLYFQGMTE
YKLVVVGAVGVGKSALTIQLIQNHFVDEYDPTIEDSYRKQVVIDGE
TCLLDILDTAGQEEYSAMRDQYMRTGEGFLCVFAINNTKSFEDIHH
YREQIKRVKDSEDVPMVLVGNKCDLPSRTVDTKQAQDLARSYGIPF
IETSAKTRQGVDDAFYTLVREIRKHKEK
SEQ ID NO: 4 - SOS1
GEEQMRLPSADVYRFAEPDSEENIIFEENMQPKAGIPIIKAGTVIKLIE
RLTYHMYADPNFVRTFLTTYRSFCKPQELLSLIIERFEIPEPEPTEAD
RIAIENGDQPLSAELKRFRKEYIQPVQLRVLNVCRHWVEHHFYDFE
RDAYLLQRMEEFIGTVRGKAMKKWVESITKIIQRKKIARDNGPGHN
ITFQSSPPTVEWHISRPGHIETFDLLTLHPIEIARQLTLLESDLYRAVQ
PSELVGSVWTKEDKEINSPNLLKMIRHTTNLTLWFEKCIVETENLEE
RVAVVSRIIEILQVFQELNNFNGVLEVVSAMNSSPVYRLDHTFEQIP
SRQKKILEEAHELSEDHYKKYLAKLRSINPPCVPFFGIYLTNILKTEE
GNPEVLKRHGKELINFSKRRKVAEITGEIQQYQNQPYCLRVESDIKR
FFENLNPMGNSMEKEFTDYLFNKSLEIEPRNPKPLPRFPKKYSYPLK
SPGVRPSNPRPGT

1. A compound of Formula (I) 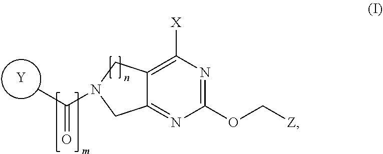
wherein:
X is selected from the group consisting of
(i) a 6- to 9-membered monocyclic- or fused bicyclic- or bridged bicyclic-heterocycloalkyl, wherein said heterocycloalkyl is saturated and contains 1 to 2 heteroatoms selected from the group consisting of N, S, and O;
(ii) an 8- to 10-membered spiroheterocycloalkyl, wherein said spiroheterocycloalkyl is saturated and contains 1 to 2 heteroatoms selected from the group consisting of N and O; 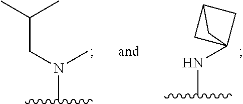
wherein, when X is (i) or (ii), X is unsubstituted or independently substituted by 1 to 4 R^{X} substituents selected from the group consisting of halo, hydroxy, C₁-C₆ alkyl, C₁-C₃ hydroxyalkyl, C₁-C₆ fluoroalkyl, carboxy, carbamoyl, C₁-C₃ carboxyalkyl, oxo, cyano, cyanomethyl, amino, pyrazolyl, oxadiazolonyl, —NHC(O)C₁-C₃alkoxyC₁-C₃alkyl, —NHC(O) C₁-C₃alkoxyC₆-C₁₀aryl, C₁-C₃alkoxy, methoxy(C₁-C₃)alkyl, amino(C₁-C₃)alkyl, C₁-C₃ alkylamino(C₁-C₃)alkyl, C₁-C₃ dialkylamino, C₁-C₃ dialkylamino(C₁-C₃)alkyl, and NHC(O)C₅-C₁₀heteroaryl, where heteroaryl may be substituted by C₁-C₃alkyl;
Ring Y is a 9- to 10-membered bicyclic ring system, wherein the ring system is partially unsaturated or aromatic, and wherein Ring Y contains 0 to 2 nitrogen heteroatoms;
wherein Ring Y is unsubstituted or independently substituted by 1 to 4 R^{y} substituents selected from the group consisting of halo, hydroxy, amino, C₁-C₃ alkyl, C₂-C₃ alkynyl, and C₁-C₃ fluoroalkyl;
Z is selected from the group consisting of
(i) a 5- to 8-membered monocyclic- or bicyclic-heterocycloalkyl, wherein said heterocycloalkyl is saturated and contains 1 nitrogen heteroatom and wherein said heterocycloalkyl is unsubstituted or substituted with 1 substituent R^{ZHC} selected from the group consisting of halo, C₁-C₃ alkyl, and methylene(C₁-C₃ alkyl)(C₁-C₃ alkyl)carbamate; 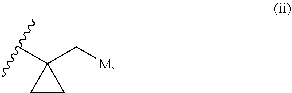
wherein M is selected from the group consisting of hydroxy, C₁-C₃ dialkylamino, and C₁-C₄ alkylamino, and wherein the cyclopropyl group is unsubstituted or substituted with up to 2 halo groups; 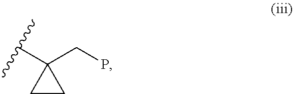
wherein P is a 5- to 8-membered monocyclic- or fused bicyclic- or bridged bicyclic-heterocycloalkyl, wherein said heterocycloalkyl is saturated and contains 1 to 2 heteroatoms selected from the group consisting of N and O, wherein said heterocycloalkyl is unsubstituted or substituted with 1 R^{P} substituent selected from the group consisting of halo, hydroxy, C₁-C₃ hydroxyalkyl, C₁-C₃ cyanoalkyl, carbamoyl, C₁-C₃ alkoxy, cyano, —NHC(O) C₁-C₃alkyl, and oxadiazolonyl, and wherein the cyclopropyl group is unsubstituted or substituted with up to 2 halo groups;
subscript m is 0 or 1; and
subscript n is 1 or 2,
or a pharmaceutically acceptable salt thereof.

2. The compound of claim 1 or the pharmaceutically acceptable salt thereof, wherein Ring Y is

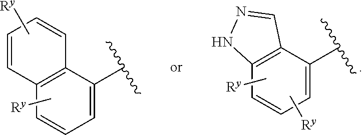

3. The compound of claim 2 or the pharmaceutically acceptable salt thereof, wherein Ring Y is

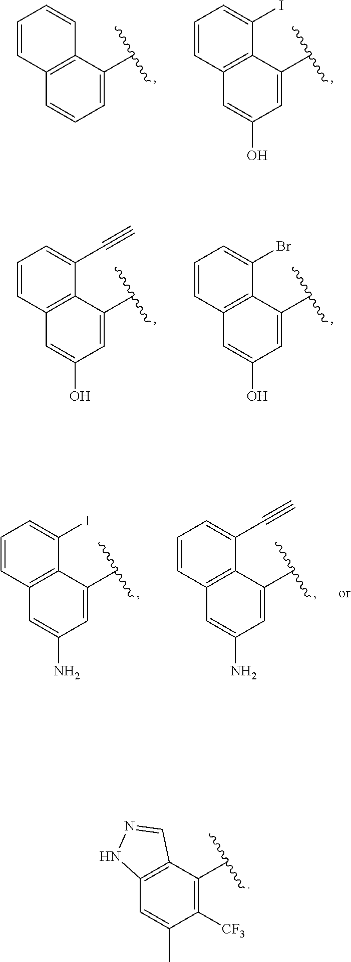

4. The compound of claim 1 or the pharmaceutically acceptable salt thereof, wherein X is 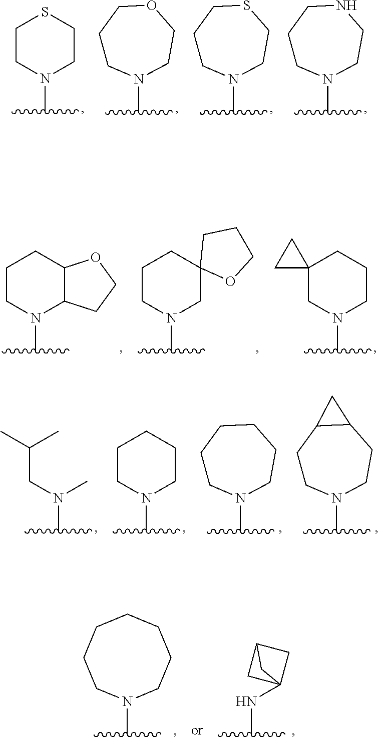
and wherein X is unsubstituted or independently substituted by 1 to 4 R^{X} substituents.

5. The compound of claim 4 or the pharmaceutically acceptable salt thereof, wherein X is substituted by 1 to 4 R^{X} substituents selected from the group consisting of halo, hydroxy, C₁-C₆ alkyl, C₁-C₃ hydroxyalkyl, C₁-C₆ fluoroalkyl, carboxy, carbamoyl, C₁-C₃ carboxyalkyl, oxo, cyano, cyanomethyl, amino, pyrazolyl, oxadiazolonyl, —NHC(O)C₁-C₃alkoxyC₁-C₃alkyl, —NHC(O)C₁-C₃alkoxyC₆-C₁₀aryl, and NHC(O)C₅-C₁₀heteroaryl, where heteroaryl may be substituted by C₁-C₃alkyl.

6. The compound of claim 5 or the pharmaceutically acceptable salt thereof, wherein X is

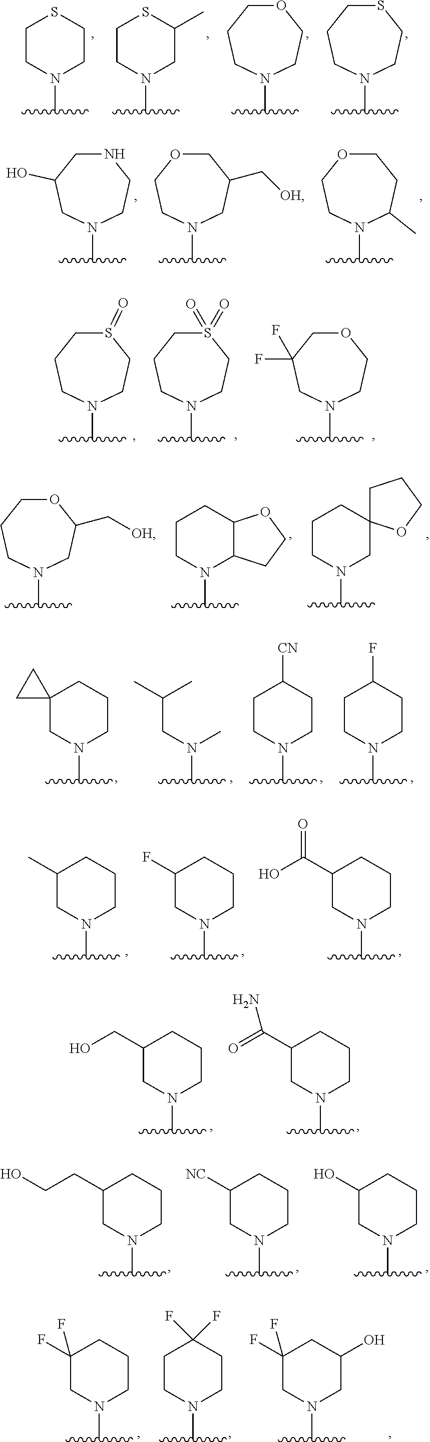

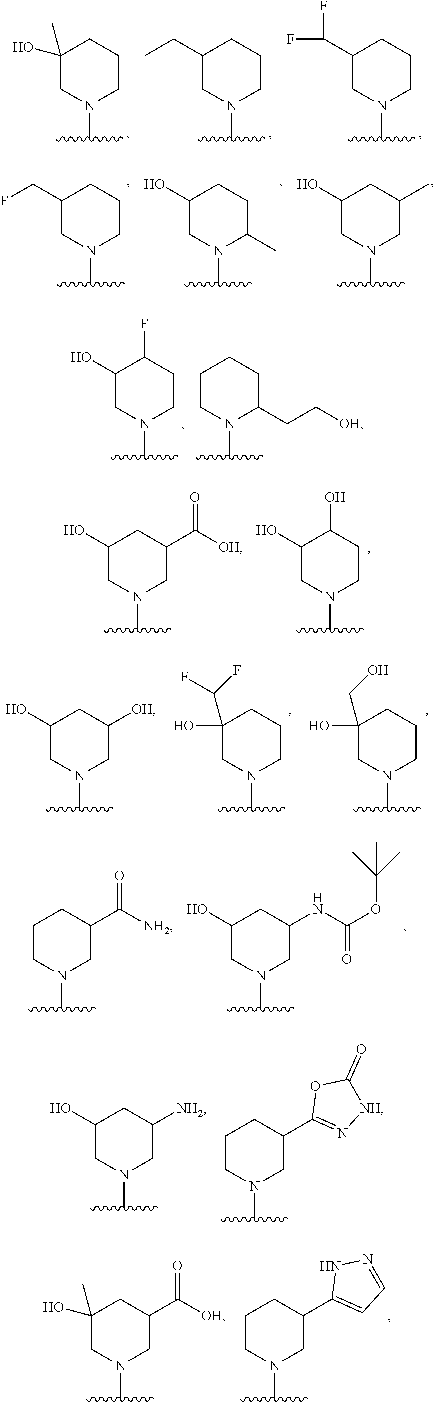

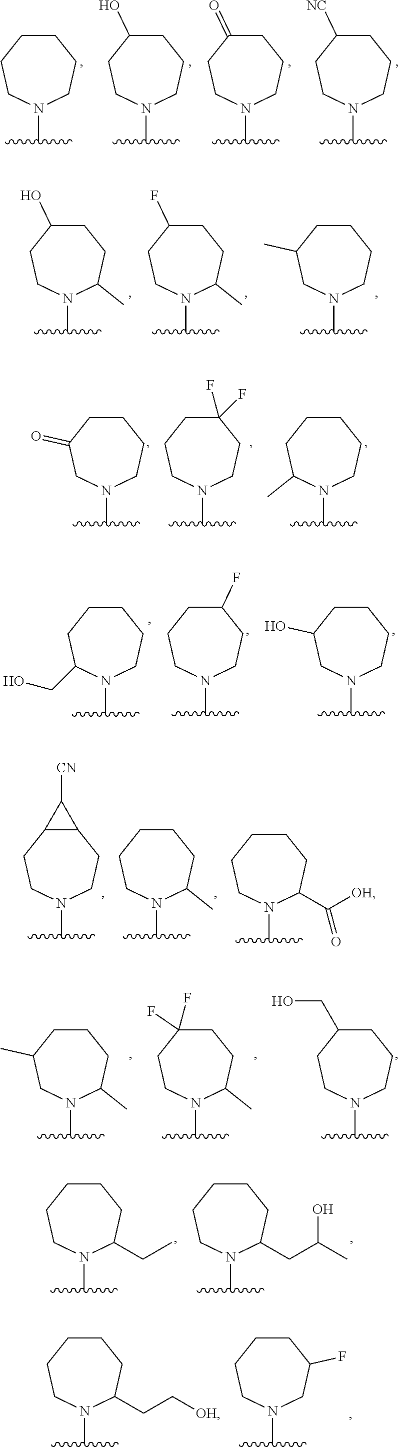

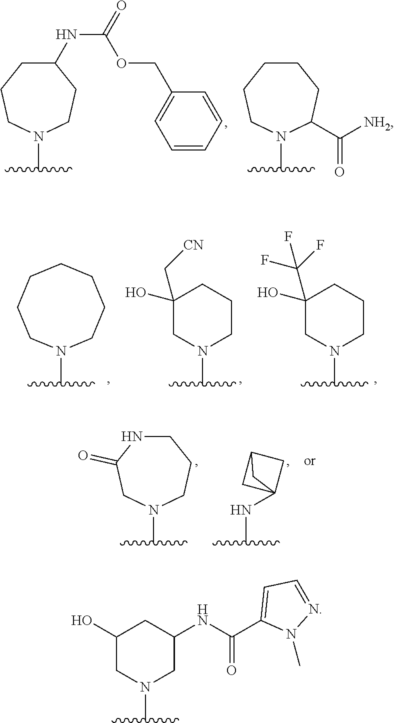

7. The compound of claim 1 or the pharmaceutically acceptable salt thereof, wherein X is 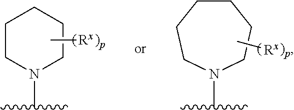
wherein subscript p is 0, 1, or 2.

8. The compound of claim 1 or the pharmaceutically acceptable salt thereof, wherein X is

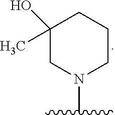 [SMILES: *N1CCCC(C)(O)C1]

9. The compound of claim 1 or the pharmaceutically acceptable salt thereof, wherein Z is

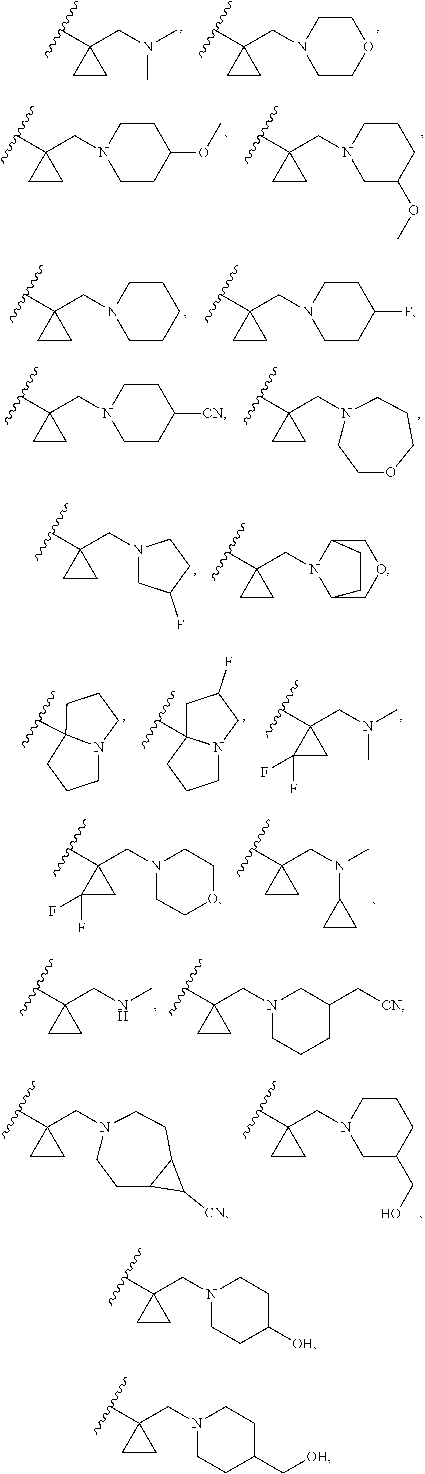

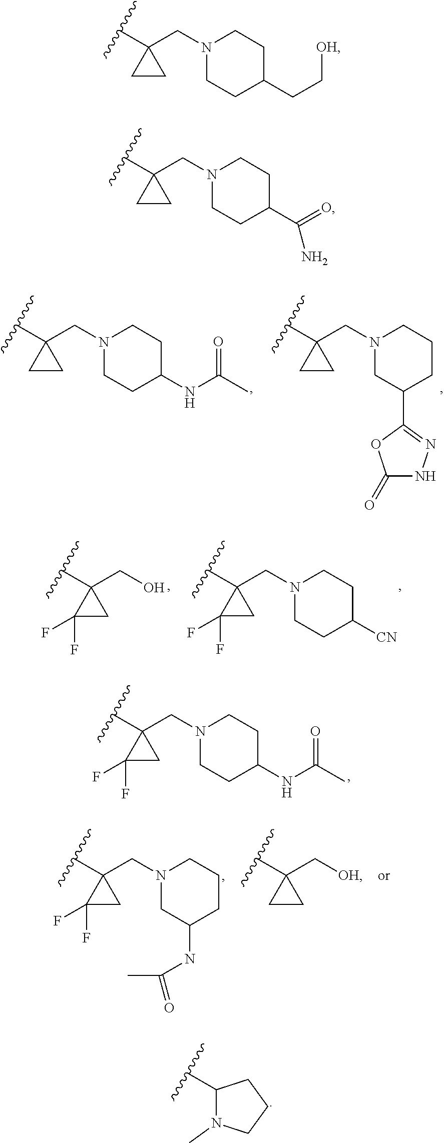

10. The compound of claim 1 or the pharmaceutically acceptable salt thereof, wherein subscript m is 1.

11. The compound of claim 1 or the pharmaceutically acceptable salt thereof, wherein subscript n is 1.

12. The compound of claim 1 or the pharmaceutically acceptable salt thereof, wherein subscript n is 2.

13. The compound of claim 1 selected from Examples 1-151 or the pharmaceutically acceptable salt thereof.

14. A pharmaceutical composition comprising the compound of claim 1 or the pharmaceutically acceptable salt thereof, and a pharmaceutically acceptable carrier.

15. A pharmaceutical composition comprising the compound of claim 1 or the pharmaceutically acceptable salt thereof, an additional anti-cancer agent, and a pharmaceutically acceptable carrier.

16. A method of inhibiting KRAS-G12D protein comprising contacting KRAS-G12D protein with the compound of claim 1, or the pharmaceutically acceptable salt thereof, to inhibit the activity of the KRAS-G12D protein.

17. A method of inhibiting KRAS-G12C protein comprising contacting KRAS-G12C protein with the compound of claim 1, or the pharmaceutically acceptable salt thereof, to inhibit the activity of the KRAS-G12C protein.

18. A method of inhibiting KRAS-G12V protein comprising contacting KRAS-G12V protein with the compound of claim 1, or the pharmaceutically acceptable salt thereof, to inhibit the activity of the KRAS-G12V protein.

19. A method of treating cancer comprising administering a therapeutically effective amount of the compound of claim 1, or the pharmaceutically acceptable salt thereof, to a subject in need of such treatment.

20. The method of claim 19, further comprising administering an additional active agent to the subject.

21.-27. (canceled)
